(19) United States
(12) Patent Application Publication
(10) Pub. No.: US 2026/0055415 A1
(43) Pub. Date: Feb. 26, 2026

(54) TARGETING NEURONAL SIRPA FOR TREATMENT AND PREVENTION OF NEUROLOGICAL DISORDERS

(71) Applicant: Baylor College of Medicine, Houston, TX (US)

(72) Inventors: Melanie Samuel, Houston, TX (US); Danye Jiang, Houston, TX (US)

(73) Assignee: Baylor College of Medicine, Houston, TX (US)

(21) Appl. No.: 18/703,956

(22) PCT Filed: Oct. 27, 2022
(86) PCT No.: PCT/US2022/078804

§ 371 Date: Apr. 23, 2024

Related U.S. Application Data
(60) Provisional application No. 63/272,974, filed on Oct. 28, 2021.

(51) Int. Cl. C12N 15/113; A61K 31/37; A61K 31/397; A61K 38/17; A61K 38/46; A61K 40/11; A61K 40/15; A61K 40/31; A61P 25/28; C07K 16/28; C12N 15/86

(52) U.S. Cl. CPC C12N 15/1138; A61K 31/37; A61K 31/397; A61K 38/177; A61K 38/1774; A61K 38/465; A61K 40/11; A61K 40/15; A61K 40/31; A61P 25/28; C07K 16/2803; C07K 16/2896; C12N 15/86; C12N 2750/14143

(57) ABSTRACT

Provided herein are methods and compositions for prevention of, reduction of risk of, amelioration of, and/or treatment of neurological disorders related to synaptopathies through manipulation of SIRPα and/or CD47.

### CROSS-REFERENCE TO RELATED APPLICATIONS

This application claims priority to U.S. Provisional Patent Application Ser. No. 63/272,974, filed Oct. 28, 2021, which is incorporated by reference herein in its entirety.

### BACKGROUND

### I. Technical Field

This disclosure relates to the field of neurological disease, aging biology, neurology, genetics, medicine, neural development, and therapeutic treatment methods.

### II. Background

Microglia are the resident immune cells in the CNS and display remarkably defined windows of engulfment which align precisely with periods of neuron growth and remodeling. Microglia are highly ameboid and phagocytic during neuron refinement, become more quiescent as neurons mature, and can be pathologically reactivated in disease. Because phagocytic microglia engulf neural material, understanding the neuronal cues that temporally control microglia engulfment offers new opportunities to alter pathological outcomes. Over the past 20 years, the field has identified unexpected immune-associated cues that determine local microglia-mediated removal of particular synapses. Herein an unexpected new paradigm is added to the field which has therapeutic implications.

### BRIEF SUMMARY

Provided herein are methods of treating, protecting against, or reducing the risk of one or more neurological disorders. In some embodiments, the methods comprise administration of one or more compositions that modulate neuronal SIRPα.

In some embodiments, the methods comprise administration of one or more compositions that increase one or more of neuronal SIRPα gene activity, neuronal SIRPα protein levels, or neuronal SIRPα protein activity.

In some embodiments, the methods comprise administration of one or more compositions that decrease one or more of neuronal SIRPα gene activity, neuronal SIRPα protein levels, or neuronal SIRPα protein activity.

In some embodiments, a neurological disorder is a central nervous system disorder (CNS) and/or a peripheral nervous system disorder (PNS).

In some embodiments, treatment comprises administration of a composition that comprises at least one inhibitory oligonucleotide. In some embodiments, an inhibitory oligonucleotide comprises small interfering RNA (siRNA), microRNA (miRNA), inhibitory antisense oligonucleotides (ASOs), or a combination thereof. In some embodiments, an inhibitory oligonucleotide has at least 80% sequence identity to a portion of, or a sequence complementary to, any of SEQ ID NOs: 1, 2, 7, or 8.

In some embodiments, the methods comprise administration of one or more compositions that comprise a transgene. In some embodiments, a transgene encodes part or all of a SIRPα gene product. In some embodiments, a transgene encodes a protein at least 80% identical to a portion of any of SEQ ID NOs: 9-10. In some embodiments, a transgene encodes part or all of a CD47 gene product. In some embodiments, a transgene encodes a protein at least 80% identical to a portion of any of SEQ ID NOs: 3-6.

In some embodiments, a composition comprises at least one antibody or Fc fusion protein. In some embodiments an antibody or Fc fusion protein is an anti-CD47 antibody, anti-SIRPα, or SIRPα-Fc fusion protein. In some embodiments, an anti-CD47 antibody is Magrolimab, Hu5F9-G4, CC-90002. TTI-621, ALX148, SRF231, SHR-1603. IBI188, or a combination thereof. In some embodiments, an anti-SIRPα antibody is ADU-1805, humanized AB21 (hAB21), humanized 1H9. BI 765063 (OSE-172), or a combination thereof.

In some embodiments, at least one inhibitory oligonucleotide, transgene, antibody, and/or Fc fusion protein is administered in the form of a nucleic acid vector.

In some embodiments, a composition comprises at least one CD47 and/or SIRPα inhibitor. In some embodiments, a CD47 and/or SIRPα inhibitor is one or more of Velcro-CD47 (N3612) antagonist, small molecule RRx-001, and/or small molecule 4Mu.

In some embodiments, a composition comprises at least one cell, including at least one immune cell, including an immune cell that expresses an engineered antigen receptor, such as a chimeric antigen receptor (CAR). In some embodiments, a cell is a CAR NK-cell or CAR T-cell.

In some embodiments, a composition comprises one or more nucleases. In some embodiments, a nuclease is a clustered regularly interspaced short palindromic repeats (CRISPR) associated (Cas) protein. In some embodiments, a Cas protein is a type I, type II, type III, type IV, type V, or type VI nuclease.

In some embodiments, a composition comprises one or more retroviruses. In some embodiments, a retrovirus is an Adeno Associated Virus (AAV). In some embodiments, an AAV is AAV2/1, AAV2/2, AAV2/5, or AAV2/9.

In some embodiments, a neurological disorder is characterized by loss of or gain of synapses. In some embodiments, a neurological disorder is selected from major depressive disorder, schizophrenia, Alzheimer's disease, Huntington disease, Parkinson's disease, amyotrophic lateral sclerosis (ALS), demyelinating diseases (e.g., multiple sclerosis (MS)), aging, and a combination thereof.

In some embodiments, a neurological disorder is characterized by aberrant synapse pruning. In some embodiments, a neurological disorder is selected from autism spectrum disorders (ASDs), down syndrome, hyperekplexia, epilepsy, or other developmental neurological disorders (e.g., applicable rare but impactful neurological diseases, e.g., applicable diseases described by the National Institute of Neurological Disorders and Stroke).

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

Unless otherwise specified, statistical analysis was performed using t-tests or ANOVA depending on the number of groups and time points, with p<0.05 considered statistically significant. Bonferroni post-hoc analyses were performed after detecting significant ANOVA effects. Where appropriate, nonparametric analyses were performed when data failed normality or equal variance tests. Non-significant results are depicted with “ns”, while significantly different results are left unlabeled or represented by one or more asterisk.

Unless otherwise specified, assays and analyses were performed on murine models.

Unless otherwise specified, SIRPα^{R-NEURON} refers to animals in which SIRPα has been genetically removed from retina neurons specifically using either Six3^{Cre} or Chx10^{Cre} crossed to a SIRPα^{F/F} line, followed by intercrosses of the progeny to generate Six3^{Cre}; SIRPα^{F/F} or Chx10^{Cre}; SIRPα^{F/F} animals; this line is also referred to as SIRPαNEURON. Unless otherwise specified, SIRPαMICROGLIA refers to animals in which SIRPα has been genetically removed from microglia specifically using a TNFRSF11A^{Cre} line (also known as Rank^{Cre}) followed by intercrosses of the progeny to generate TNFRSF11A^{Cre}; SIRPα^{F/F} animals. Unless otherwise specified, SIRPαPAN NEURON refers to animals in which SIRPα has been genetically removed from all CNS neurons using a Nestin^{Cre} line (followed by intercrosses of the progeny to generate Nestin^{Cre}; SIRPα^{F/F} animals).

FIG. 1A-E, Retinas provide an approachable and readily manipulatable model for studying neuron development and microglial mediated refinement. (A) Cartoon representation of retina organization; abbreviations listed are utilized throughout the Figures presented herein, Cone Photoreceptors (Cone), Rod Photoreceptors (Rod), Microglial cells (Microglia), Horizontal cells (HC). Bipolar cells (BC), Retinal Ganglion cells (RGC), Outer Nuclear Layer (ONL). Outer Plexiform Layer (OPL). Inner Nuclear Layer (INL). Inner Plexiform Layer (IPL), and Ganglion Cell Layer (GCL). (B) Representation of ordered adult retinal synapses. (C) Antibodies and viral vectors are commercially available for selectively transducing and/or staining retinal cells such as cone cells, rod cells, bipolar cells, etc. (D) Genes associated with retinal development and/or structure can be directly manipulated using techniques such as CRISPR/Cas, AAV mediated transduction, transformation, cre knockout, etc. (E) Retina function can be directly measured using electroretinography (ERG).

FIG. 2A-C. Microglia activity parallels retina synapse development. (A) Representation of murine retina development with images taken at P2, P6, P9, and P14 of age; top row displays DAPI staining for nuclei (shown in blue) and immunofluorescent staining for vesicular glutamate transporter (Vglut1) (shown in white) facilitating observation of synapse localization and development; bottom row displays microglia that were identified using a Cx3crl^{GFP/+} reporter line showing GFP positive microglia (shown in white). (B) Depicts microglia activity at P2, P6, P9, and P14 of age; top row depicts microglia morphological development over time; middle row illustrates activated microglia cells, with CD68 immunofluorescent staining (shown in red) and DAPI staining for nuclei (shown in white); bottom row illustrates phagocytosis by microglia with MER proto-oncogene, tyrosine kinase (MER) immunofluorescent staining (shown in turquoise) and DAPI staining for nuclei (shown in white); microglia CD68 expression and phagocytic events in the retina peak between P2 and P14 of age. (C) Depicts immunofluorescent and DAPI staining of mouse retina at day P1, P2, P5, P6, P8, P9, P12, or P17 of age, correlating microglia structure and location (Iba1) with IPL and/or OPL synaptogenesis and eye-opening; microglia location, phagocytic state and numbers correspond with retina synapse refinement.

FIG. 3A-H, SIRPα expression is in spatiotemporal alignment with synapse refinement. (A) Depicts retina development with images taken at P2, P6, P9, and P14 of age; DAPI staining for nuclei (shown in white) and immunofluorescent staining for SIRPα (shown in magenta) highlights SIRPα protein localization to the OPL and the IPL. (A1) Is graphical representation of quantitative PCR (qPCR) data displaying relative SIRPα mRNA expression levels (Y axis) in developing retina at age P2, P6, P9, and P14 (X axis). (A2) Is a heatplot representation of single cell sequencing cell specific SIRPα levels during retinal development (see Clark et al., 2019). (B) Depicts immunoblot measurements of whole retina SIRPα protein expression at P4, P6, P9, and P14 of age, with Glyceraldehyde 3-phosphate dehydrogenase (GAPDH) acting as a biological loading control. (C) Depicts immunoblot measurements of whole retina SIRPα protein levels in SIRPα^{F/F} animals with (+) or without (−) neuron specific Six3^{Cre} expression, with GAPDH acting as a biological loading control; loss of neuronal SIRPα significantly reduced whole retina SIRPα protein levels. (D) Depicts immunoblot measurements of whole retina SIRPα protein levels in SIRPα^{F/F} animals with (+) or without (−) microglia specific TnfrsfllaCre expression, with GAPDH acting as a biological loading control; loss of microglia SIRPα did not significantly reduce whole retina SIRPα protein levels. (E) Depicts immunofluorescent staining showing SIRPα protein localization (SIRPα shown in magenta) at day P14 of retinal development, co-staining (shown in green) of cones (stained with anti-cone specific mouse cone arrestin (mCAR)), rod terminals (stained with anti-post-synaptic density protein 95 (PSD95), aka Discs Large MAGUK Scaffold Protein 4), cone bipolar cells (stained with anti-Secretagogin (SCGN)), or rod bipolar cells (stained with anti-Protein kinase C alpha (PKCa)) is shown; presynaptic (Pre), postsynaptic (Post); SIRPα co-localizes with cone and rod presynaptic (arrows) but not postsynaptic markers in outer retina. (F) Stochastic Optical Reconstruction Microscopy (STORM) results with staining for RIBEYE (shown in turquoise) and SIRPα (shown in magenta) show precise SIRPα protein localization within the synaptic cleft. (G) STORM results with staining for Calbindin (shown in turquoise) and SIRPα (shown in magenta) confirmed precise SIRPα protein localization within the synaptic cleft. (H) Fluorescent In situ hybridization (FISH; e.g., RNAscope) of SIRPα transcripts (RNAscope shown in white, DAPI staining for nuclei shown in blue) coupled with Iba1 immunofluorescent staining (shown in green) for microglia at days P2, P6, P9, and P14 of age confirmed that neuronal produced SIRPα is in spatiotemporal alignment with synapse refinement.

FIG. 4A-E, Neurons produce the majority of SIRPα. (A) Depicts immunofluorescent staining (TdTomato left panel shown in red, Iba1 middle panel shown in green, and merged right panel) for SIRPα conditional knockout animals with reduction of SIRPα in microglia (top row) or neurons (bottom row) (for genotype information, see Table 1. SIRPαNEURON animals have retinal neuronal specific loss of SIRPα, while SIRPαMICROGLIA animals have microglia specific loss of SIRPα). (B) Depicts immunofluorescent staining of SIRPα (shown in magenta) or microglia Iba1 (shown in green) at day P9 of age, left panels depict control animals, middle panels depict SIRPαNEURON animals, and right panels depict SIRPα^{MICROGLIA} animals. (C) Depicts quantification of SIRPα protein levels, the Y axis represents fluorescence intensity fold change relative to control, the X axis represents genotype; SIRPαNEURON animals showed significantly lower SIRPα levels when compared to controls. (D) Depicts immunofluorescent staining of SIRPα (shown in magenta) or microglia Iba1 (shown in green) at day P8 of age, comparing microglia cell depletion animals (Cx3crl^{CreER}; Rosa26^{iDIR}, tamoxifen dosed on day P1, P5, and P7, and diphtheria toxin dosed on days P4, P6, and P8) with control animals; significant reduction in microglia levels were observed (top-row) while no significant reduction in total SIRPα levels were observed (bottom-row). (E) Depicts quantification of microglia cell abundance from assays as shown in FIG. 4 D, significant reductions in microglial density were observed in microglia cell knockout animals (Cx3crl^{CreER}; Rosa26^{iDTR}).

FIG. 5A-M, Loss of neuronal SIRPα results in marked changes in microglial morphology, activity, and activation, leading to a reduction of microglia mediated phagocytosis and a subsequent retention of synapses. (A) Depicts a cartoon representation of activated compared to resting microglia, and immunofluorescent staining of microglia cells Iba1 (shown in green) in control animals (left panel). SIRPα^{NEURON} animals (middle panel), or SIRPα^{MICROGLIA} animals (right panel) at day P9 of age, zoomed subsets of each image are shown for each genotype; hallmarks of microglia quiescence relative to control animals are readily apparent in SIRPα^{NEURON} animals. (B) Depicts quantification of process endpoints per cell relative to control (Y axis) in control animals, SIRPα^{NEURON} animals, or SIRPα^{MICROGLIA} animals (X axis) at day P9 of age; SIRPα^{NEURON} animals displayed significantly increased process endpoints per cell when compared to controls, while SIRPα^{MICROGLIA} animals did not. (C) Depicts quantification of total process length per cell relative to control (Y axis) in control animals. SIRPα^{NEURON} animals, or SIRPα^{MICROGLIA} animals (X axis) at day P9 of age; SIRPα ^{NEURON} animals displayed significantly increased process length per cell when compared to controls, while SIRPα^{MICROGLIA} animals did not. (D) Depicts quantification of microglia cell soma size (μm²) (Y axis) in control animals, SIRPαR-NEURON animals, or SIRPα^{MICROGLIA} animals (X axis) at day P9 of age; SIRPα^{NEURON} animals displayed significantly decreased soma size when compared to controls, while SIRPαMICROGLIA animals did not. (E) Depicts immunofluorescent staining of microglia cells using Iba1 (shown in green) and/or CD68 (shown in red) in control animals (left panels), SIRPα^{NEURON} animals (middle panel), or SIRPα^{MICROGLIA} animals (right panel) at day P9 of age; reduction of microglia phagocytic cups (circled with white dashed lines) in SIRPα^{NEURON} animals are readily apparent when compared to control or SIRPα^{MICROGLIA} animals. (F) Depicts quantification of microglia phagocytic cups per cell relative to control (Y axis) in control animals, SIRPα^{NEURON} animals, or SIRPα^{MICROGLIA} animals at day P9 of age; SIRPα^{NEURON} animals displayed significantly decreased total phagocytic cup per cell levels when compared to controls, while SIRPα^{MICROGLIA} animals did not. (G) Depicts quantification of the percentage of microglia in a population (Y axis) that displayed or did not display phagocytic cups in control animals, SIRPα^{NEURON} animals, or SIRPα^{MICROGLIA} animals (X axis) at day P9 of age; SIRPα^{NEURON} animals displayed significantly decreased percentages of microglia cells with phagocytic cups when compared to controls, while SIRPα^{MICROGLIA} animals did not. (H) Depicts immunofluorescent staining of activated microglia cells CD68 (shown in red) in control animals (left panel). SIRPα^{NEURON} animals (middle panel), or SIRPα MICROGLIA animals (right panel) at day P9 of age; reduction of microglia CD68 expression in SIRPα^{NEURON} animals is readily apparent when compared to control or SIRPα^{MICROGLIA} animals. (I) Depicts quantification of microglial expressed CD68 fluorescence intensity relative to control (Y axis) in control animals, SIRPα^{NEURON} animals, or SIRPα^{MICROGLIA} animals (X axis) at day P9 of age; SIRPα^{NEURON} animals displayed significantly decreased CD68 fluorescence intensity when compared to controls, while SIRPα^{MICROGLIA} animals did not. (J) Depicts 3D reconstructions of immunofluorescent staining of activated microglia cells CD68 (shown in red) and Iba1 (shown in green) in control animals (left panels). SIRPα^{NEURON} animals (middle panels), or SIRPα^{MICROGLIA} animals (right panels) at day P9 of age; reduction of microglia CD68 localized expression volume when compared to total cell volume in SIRPα^{NEURON} animals is readily apparent when compared to control or SIRPα^{MICROGLIA} animals. (K) Depicts quantification of microglial expressed CD68 localized expression volume when compared to total cell volume (Y axis) in control animals, SIRPα^{NEURON} animals, or SIRPα^{MICROGLIA} animals (X axis) at day P9 of age; SIRPα^{NEURON} animals displayed significantly decreased CD68 cell volume percentage when compared to controls, while SIRPα^{MICROGLIA} animals did not. (L) Depicts immunofluorescent staining of day P22 control or SIRPα^{NEURON} animals stained with PSD95 (top row) or RIBEYE (bottom row); an under pruning of synapses in SIRPα^{NEURON} animals compared to control animals is apparent, loss of neuronal SIRPα resulted in a preservation of synapses when compared to controls. (M) Depicts results from scotopic ERG of control animals, SIRPα^{NEURON} animals, or SIRPα^{MICROGLIA} animals; a decrease in photoreceptor function, and/or interneuron function is observed in SIRPα^{NEURON} animals when compared to controls, suggesting loss of neuronal SIRPα during development leads to impaired retinal function due to synapse retention.

FIG. 6A-H, CD47 is present in emerging synapses at high levels. (A) Depicts CD47 expression patterns using immunofluorescent staining of CD47 in developing mouse retina at P6, P9, and P14 of age. (B) Depicts CD47 expression patterns using immunofluorescent staining of CD47 (shown in turquoise) and SIRPα (shown in magenta) at day P14 of age, highlighted is the highly overlapping expression profiles for the two molecules in the OPL. (C) Depicts CD47 expression patterns using immunofluorescent staining of CD47 (shown in turquoise) in emerging synapses, with counterstaining (shown in magenta) of rod terminals (PSD95), cone bipolar cells (SCGN), or horizontal cells (Calbindin); CD47 was determined to be abundant in emerging synapses. (D) Depicts fluorescent in situ hybridization (FISH) of CD47 transcripts using FISH (e.g., RNAscope) (shown in white), with immunofluorescent counterstaining of Calbindin (shown in magenta) and/or microglia Iba1 (shown in green) at day P4, P6, and P9 of age; CD47 was determined to be enriched specifically within postsynaptic neurons. (E) Depicts CD47 gene product expression patterns using FISH (e.g., RNAscope) of CD47 (shown in white), with counterstaining (shown in blue) of AP-2 family transcription factors (AP2; left panel) or RNA binding protein mRNA processing factor (RBPMS; right panel); CD47 was determined to be enriched specifically within postsynaptic neurons. (F) Depicts STORM results of staining for SIRPα (shown in magenta) and co-staining (shown in green) with CD47 (left panels) or Ribeye (right panels), results confirmed precise SIRPα and CD47 protein localization within the synaptic cleft. (G) Depicts microglia Iba1 or CD68 expression patterns using immunofluorescent staining of CD47 or CD68 in wildtype control animals or CD47^{-/-} null animals; CD47^{-/-} null mice show relatively normal morphology and CD68 levels. (H) Depicts quantification of soma size, number of endpoints per cell, total process length per cell, and CD68 fluorescence intensity when comparing wildtype control animals to CD47^{-/-} null animals; soma size is unchanged, there is a modest but significant reduction in process length, and a small but not significant reduction in microglia endpoints and CD68 fluorescence intensity.

FIG. 7A-K, Increasing the levels of (e.g., “promotion of”) neuronal SIRPα in the retina temporally extends microglia activation. (A) Schematic representation of electroporation and transformation procedures followed by analysis of microglia and/or neuronal cell phenotypes; retina were transformed with pCAG-GFP and pCAG-SIRPα, with pCAG-GFP and pCAG-CD47, or with control pCAG-GFP only. (B) Depicts retinal pCAG-GFP and pCAG-SIRPα expression patterns as shown by GFP fluorescence and SIRPα immunofluorescent staining. (C) Depicts GFP expression and immunofluorescent staining of microglia markers Iba1 (shown in green) or CD68 (shown in red), for control animals (left panels), animals transformed with pCAG-GFP and pCAG-SIRPα (“SIRPα+GFP”, middle panels), or animals transformed with pCAG-GFP and pCAG CD47 (“CD47+GFP”, right panels) at day P21 of age; animals transformed with pCAG-SIRPα displayed striking levels of CD68 expression. (D) Depicts quantification of microglial process lengths fold change relative to control (Y axis) in control animals, pCAG-SIRPα (“SIRPα”) animals, or pCAG-CD47 (“CD47”) animals (X axis) at day P21 of age; pCAG-SIRPα animals displayed significantly shorter process lengths per cell when compared to control animals. (E) Depicts quantification of microglia soma size (Y axis; measured in μm²) in control animals, pCAG-SIRPα (“SIRPα”) animals, or pCAG-CD47 (“CD47”) animals (X axis) at day P21 of age; pCAG-SIRPα animals displayed significantly larger soma size when compared to control animals. (F) Depicts quantification of CD68 fluorescent intensity fold change relative to control (Y axis) in control animals, pCAG-SIRPα (“SIRPα”) animals, or pCAG-CD47 (“CD47”) animals (X axis) at day P21 of age; pCAG-SIRPα animals displayed significantly more CD68 fluorescence intensity when compared to control animals. (G) Depicts 3D reconstruction of fluorescence imaging and/or immunofluorescent staining of activated microglia cells in control animals (left panel) or pCAG-SIRPα animals (right panel), phagocytic events are displayed; engulfed neural material is assessed as the GFP positive, neuron derived material found within the microglia volume following 3D reconstruction of individual cells; Iba1 is shown in green, CD68 is shown in red, and GFP is shown in blue. (H) Depicts quantification of percentage of microglia engulfment events (Y axis) in control animals or pCAG-SIRPα (“SIRPα”) animals (X axis) at day P21 of age; percentage of microglia volume occupied by GFP-labeled neuronal materials in microglia was significantly greater in pCAG-SIRPα animals when compared to control animals. (I) Depicts GFP expression (shown in white, left panel), microglia Iba1 immunofluorescent staining (shown in green, second to left panel), microglia CD68 immunofluorescent staining (shown in red, second to right panel), and merged channels (right panel) in pCAG-GFP and pCAG-SIRPα transformed animals (“SIRPα OE”) at day P21 of age; animals transformed with pCAG-GFP and pCAG-SIRPα displayed localized/regional GFP and CD68 expression patterns specific to sites of neuronal transfection (e.g., to the right of the superimposed dashed line); results showed that the effects of neuronal SIRPα promotion on microglia activities were local and corresponded with the regions of increased SIRPα. (J) Depicts a reduction in ribeye levels in the outer retina when SIRPα is overexpressed in neurons, consistent with increased microglial (as shown in FIG. 7G and quantified in 7H). (K) Quantification of immunofluorescent images shown in 7J displayed a significant reduction in number of ribeye synapses when compared to controls.

FIG. 8A-D. Modulation of neuronal SIRPα and neuronal CD47 changes microglia activation. (A) Depicts GFP expression (top row) and immunofluorescent staining of microglia markers Iba1 (shown in green; middle row) or CD68 (shown in red; bottom row), for control animals transformed with pCAG-GFP (“control”; left panels), animals transformed with pCAG-GFP and pCAG-SIRPα (“SIRPα+GFP” also described in FIG. 8B-D as “SIRPα”; second to left panels), animals transformed with pCAG-GFP and pCAG-CD47 (“CD47+GFP” also described in FIG. 8B-D as “CD47”; second to right panels), or animals transformed with pCAG-GFP pCAG-SIRPα and pCAG-CD47 (“SIRPα+CD47+GFP” also described in FIG. 8B-D as “SIRPα and CD47”; right panels), at day P9 of age. (B) Depicts quantification of microglia cell total process lengths (Y axis; measured in μm), in control animals, SIRPα animals, CD47 animals, and SIRPα and CD47 animals (X axis) at day P9 of age; CD47 animals displayed significantly longer total process length when compared to control animals, while SIRPα animals or SIRPα and CD47 animals were not significantly different from controls. (C) Depicts quantification of microglial process endpoints (Y axis), in control animals, SIRPα animals, CD47 animals, and SIRPα and CD47 animals (X axis) at day P9 of age; CD47 animals display significantly more endpoints when compared to control animals; SIRPα animals or SIRPα and CD47 animals were not significantly different from controls. (D) Depicts quantification of microglial soma size (Y axis; measured in μm²), in control animals, SIRPα animals, CD47 animals, and SIRPα and CD47 animals (X axis) at day P9 of age; CD47 animals displayed significantly smaller soma size when compared to control animals, while SIRPα animals or SIRPα and CD47 animals were not significantly different from controls.

FIG. 9A-C. Displays SIRPα, and phagocytic microglia (Iba1 or CD68) levels in the dorsal lateral geniculate nucleus (dLGN), and provides results for assessment of microglia-mediated engulfment in SIRPαPAN-NEURON and SIRPα^{MICROGLIA} animals. (A) dLGN neurons produce high levels of SIRPα as shown by immunofluorescent staining for SIRPα and microglia (Iba1), which reveal high neuronal and dim microglia co-localization at day P5 and P9 of age. (B) Depicts immunofluorescent images with staining for SIRPα (middle panels), microglia (Iba1; left panels) and their overlap (right panels) in control animals (top panels), SIRPαPAN-NEURON animals (middle panels), and SIRPα^{MICROGLIA} animals (bottom panels); strikingly reduced levels of SIRPα are apparent in SIRPαPAN-NEURON animals when compared to controls or SIRPα^{MICROGLIA} animals; the data suggest the role of neurons for producing the majority of SIRPα as described in the retina is conserved in the brain. (C) Depicts immunofluorescent images with staining for microglia (Iba1; left panels) and phagocytic microglia (CD68 and Iba1; right panels) in control animals (top panels), SIRPαPAN-NEURON animals (middle panels), and SIRPα^{MICROGLIA} animals (bottom panels); strikingly reduced levels of CD68 are apparent in SIRPαPAN-NEURON animals when compared to controls or SIRP α^{MICROGLIA} animals; the data suggest the role of neuronal SIRPα in influencing microglia phagocytic state as described in the retina is conserved in the brain.

FIG. 10, Is a graphical abstract depicting certain findings disclosed herein.

FIG. 11A-J, Retinal neuron refinement coincided with heightened microglia phagocytosis. (A) Schematic of adult retina. Rods (R) and cones (C) in the outer nuclear layer (ONL) synapse onto bipolar cells (BC) and horizontal cells (HC) in the inner nuclear layer (INL), forming a thin synaptic band called outer plexiform layer (OPL). Bipolar cells and amacrine cells (AC) relay signals to retinal ganglion cells (RGC) in the inner plexiform layer (IPL). RGCs reside in the ganglion cell layer (GCL), and their axons form the optic nerve which projects to the brain. Microglia (M) occupy the synaptic layers. (B) Generation of retinal synaptic layers. Vglut1-labeled inner retina synapses (white) were present at P2. At P5-P6, Vglut1⁺photoreceptor terminals were visible in the OPL. At P9, both layers continued to be refined. Synaptogenesis largely completed by P14. Scale bars. 50 μm. (C) Microglia (white) migration to the synaptic layers. Scale bars, 50 μm. (D) Representative wholemount images of P6, P9, and P14 OPL microglia in Cx3crl^{GFP/+} mice. Scale bars. 25 μm. (E-G) Developmental time course of wildtype (WT) microglial morphology. Quantifications of process length (E), process endpoints (F), and number of phagocytic cups per microglia (G), n=7 for P6, n=7 for P9, n=6 for P14. Data were compared using one-way ANOVA with posthoc Bonferroni correction. (H) Schematic of OPL synaptogenesis. (I) Representative retinal cross-sections showing WT P6, P9, and P14 Iba1+OPL microglia (green), CD68+lysosomes (red), and merge (yellow). Scale bars. 25 μm. (J) Quantification depicting the percentages of P6, P9, and P14 WT CD68⁺microglia, n=7 for P6, n=7 for P9, and n=4 for P14. Data were compared using one-way ANOVA with posthoc Bonferroni correction. Data from (E) to (J) were pooled from two independent experiments. All data are shown as the mean±SEM. * p<0.05. ** p<0.01. ** p<0.0001. See also FIG. 1, FIG. 2, and FIG. 18.

FIG. 12A-K, Neuronal SIRPα was enriched during periods of peak microglia phagocytosis. (A) Representative images showing P6, P9, and P14 WT SIRPα staining (magenta) in the synaptic layers. Scale bars, 50 μm (top) and 25 μm (bottom). See also FIG. 19 A-B. (B) Representative images showing little SIRPα signal in Iba1+microglia (green). Scale bars. 25 μm and 10 μm (insects). See also FIG. 19 C. (C) Representative images showing colocalization of SIRPα (magenta) and Vglut1⁺photoreceptor terminals (cyan) in the OPL. Scale bars, 25 μm. See also FIG. 19 C. (D) Representative images showing colocalization of SIRPα (magenta) with cone (mCAR) and rod (PSD95) terminals (green). Scale bars. 10 μm. See also FIG. 19 D. (E) Representative images showing SIRPα (magenta) with horizontal cell (Calbindin) and cone bipolar cell (SCGN) terminals (green). Scale bars, 10 μm. See also FIG. 19 D. (F) Schematic of microglial SIRPα deficiency model (SIRPα^{MICROGLIA}). Example images showing staining of SIRPα (magenta), microglia (Iba1, green), and OPL synapses (RIBEYE, cyan) in this model at P9. Scale bars, 25 μm and 10 μm (insets). See also FIG. 19 F. (G) Levels of SIRPα fluorescence in OPL in SIRPα^{MICROGLIA} relative to controls, n=6 per group. Data were compared using an unpaired t-test. (H) Representative immunoblot image and quantification of SIRPα in whole retina from P9 WT and SIRPα^{MICROGLIA} mice, n=3 per group. Data were compared using an unpaired t-test. (I) Schematic of neuronal SIRPα deficiency model (SIRPα: NEURON). Example images showing staining of SIRPα (magenta), microglia (Iba1. green), and OPL synapses (RIBEYE, cyan) in this model at P9. Scale bars, 25 μm and 10 μm (insets). See also FIG. 19 F. (J) Levels of SIRPα fluorescence in OPL in SIRPα^{NEURON} mice relative to controls, n=4 control and 5 SIRPα^{NEURON}. Data were compared using an unpaired 1-test. (K) Representative immunoblot image and quantification of SIRPα in whole retina from P9 W′T and SIRPα^{NEURON}, n=3 per group. Data were compared using an unpaired t-test. Data from (H) and (K) were obtained from one experiment. (G) and (J) were pooled from two independent experiments. All data are presented as the mean±SEM. ** p<0.01. **** p<0.0001. ns, not significant. See also FIG. 19, and FIG. 20.

FIG. 13A-N. Microglia phagocytosis was impaired in neuronal SIRPα-deficient mice. (A) Representative images of control, SIRPα^{NEURON}, and SIRPα^{MICROGLIA} OPL microglia at P9. Scale bars, 100 μm (top) and 50 μm (below). (B-D) Quantifications of microglia process endpoints (B), process length (C), and soma size (D) in P9 control. SIRPα^{NEURON}, and SIRPα^{MICROGLIA} mice, n=8 control. 4 SIRPα^{NEURON}, and 3 SIRPα^{MICROGLIA} mice, one-way ANOVA with posthoc Bonferroni correction. (E-F) Representative images showing the lysosomal marker CD68 in microglia in P9 control. SIRPα^{NEURON}, and SIRPα^{MICROGLIA} mice. Scale bars, 100 μm and 20 μm (insets). (F) Bar graphs depicting the levels of CD68 staining in control, SIRPα^{NEURON}, and SIRPα^{MICROGLIA} animals, n=8 control, 4 SIRPα^{NEURON}, and 3 SIRPα^{MICROGLIA}, one-way ANOVA with posthoc Bonferroni correction. (G-H) Representative 3D reconstructions of control, SIRPα^{NEURON}, and SIRPα^{MICROGLIA} microglia (green) with internalized CD68+lysosomes (red). Scale 10 μm. (H) Graph showing percent volume of CD68+lysosome in microglia from P9 SIRPα^{NEURON} and SIRPα^{MICROGLIA} mice relative to control, n=8 control, 4 SIRPα^{NEURON}, 3 SIRPα^{MICROGLIA} mice, one-way ANOVA with posthoc Bonferroni correction. (I-J) Representative images of phagocytic cups (arrowheads) in control, SIRPα^{NEURON}, and SIRPα^{MICROGLIA} microglia (green). Scale bars. 20 μm. The graphs depict the number of phagocytic cups per microglia (I). Data were compared using two-way ANOVA with posthoc Bonferroni correction. See also FIG. 20 B. (K-L) Representative 3D reconstructions of control, SIRPα^{NEURON}, and SIRPαMICROGLIA microglia (gray) with internalized GFP⁺ neuronal material (green). Scale bars. 10 μm. (L) Graph showing percent volume of GFP-labeled neuronal material in microglia from P9 SIRPα^{NEURON}, and SIRP α^{MICROGLIA} mice relative to control, n=3 control, 4 SIRPα^{NEURON}, 3 SIRPα^{MICROGLIA} mice. Data were compared using one-way ANOVA with posthoc Bonferroni correction. (M-N) Flow cytometry gating and quantification of microglial phagocytosis of pHrodo-red594 conjugated yeast particles in (M) SIRPα^{NEURON}; Cx3crl^{GFP/+} (n=20) and SIRPαF/F; Cx3crl^{GFP/+} (n=16) retinas as well as (N) SIRPα^{MICROGLIA}; Cx3crl^{GFP/+} (n=16) and SIRPαl/F; Cx3crl^{GFP/+} (n-12) retinas at P9. * p<0.05, unpaired t-test. See also FIG. 20 D-E. Data from (B) to (J) were obtained from one experiment. Data in (L) to (N) were pooled from three independent experiments. All data are presented as the mean±SEM. * p<0.05. ** p<0.01. **** p<0.0001, ns, not significant. See also FIG. 20.

FIG. 14A-L, Neuronal SIRPα was required for synapse refinement and circuit function in the retina. (A) Representative images of RIBEYE+OPL ribbon synapses in control and SIRPα^{NEURON} retinas. Scale bars, 10 μm. (B-C) Graphs depicting the number of OPL ribbon synapses (B) and RIBEYE intensity (C) in P9 SIRPα^{NEURON} mice relative to controls, n=5 per group, unpaired t-test. (D) Representative images of RIBEYE-labeled OPL ribbon synapses in control and SIRPα^{MICROGLIA} retinas. Scale bars, 10 μm. (E-F) Graphs depicting the number of OPL ribbon synapses (E) and RIBEYE intensity (F) at P9 in SIRPα^{MICROGLIA} mice relative to controls, n>4 per group, unpaired t-test. (G) Representative traces of scotopic recording from control and SIRPα^{NEURON} mice. (H-I) Quantifications of the amplitudes of the scotopic a-wave and b-wave in control and SIRPα ^{NEURON} mice, n=7 per group, paired t-test. (J) Representative traces of scotopic recording from control and SIRPα^{MICROGLIA} mice. (K-L) Quantifications of the amplitudes of the scotopic a-wave and b-wave in control and SIRPα^{MICROGLIA} mice, n=7 per group, paired t-test. Data were obtained from two to three independent experiments. All data are presented as the mean±SEM. * p<0.05, ns, not significant.

FIG. 15A-P, Prolonging neuronal SIRPα expression extended microglia phagocytosis. (A) Schematic illustration of in vivo electroporation. See also FIG. 21A. (B) Representative confocal and 3D reconstructed images of GFP-expressing cells (white), Iba1+microglia (green), and CD68+lysosomes (red) in control (GFP only) and SIRPα+GFP retinas at P21, viewed in wholemount. Scale bars, 50 μm and 25 μm (insets). See also FIG. 21 B. (C-D) Quantifications of microglial morphology, including process length (C) and soma size (D). in control and SIRPα+GFP groups, n=10 control, 8 SIRPα+GFP mice, unpaired t-test. (E-F) Quantification of the levels of CD68 staining (E) and internalized CD68+lysosome volume (F) in SIRPα+GFP versus control groups, n=10 control, 8 SIRPα+GFP mice, unpaired 1-test. (G) Representative confocal images showing borders of the electroporated retinal patch (GFP. white, border indicated by the dotted line), microglia (Iba1, green) morphology, and the levels of CD68 staining (red) in control and SIRPα+GFP regions. Scale bars, 50 μm. (H-I) Quantifications of microglia process length (H) and CD68 staining levels (I) inside and outside GFP control transfected regions, n=3 per group, unpaired t-test. (J-K) Quantifications of microglia process length (J) and CD68 staining levels (K) inside and outside SIRPα+GFP transfected regions, n=4 per group, unpaired t-test. (L-M) Representative 3D-reconstructed images of P21 Iba1+microglia (gray), internalized GFP-labeled neuronal material (green), and CD68+lysosomes (red) in control and SIRPα+GFP regions (L), and graph showing percent volume of GFP+material in microglia from these groups (M). Scale bars. 20 μm, n=3 per group, unpaired t-test. (N) Representative images of RIBEYE-labeled OPL ribbon synapses in control and SIRPα+GFP groups. Scale bars, 10 μm. (O-P) Graphs depicting the number of OPL ribbon synapses (( ) and RIBEYE intensity (P) in P21 control and SIRPα+GFP groups. n=3 control and 5 SIRPα+GFP mice, unpaired t-test. Data were pooled from at least three independent experiments. All data are presented as the mean±SEM. * p<0.05. ** p<0.01. **** p<0.0001, ns, not significant. See also FIG. 21.

FIG. 16A-F. Neuronal SIRPα was juxtaposed with CD47 at synapses during development. (A) Representative images showing P6, P9, and P14 WT CD47 staining (cyan) in retinal synaptic layers. Scale bars. 50 μm (top) and 25 μm (bottom). See also FIG. 22A. (B) Representative images showing the juxtaposition of CD47 (cyan) with photoreceptor terminals (Vglut1 and PSD95, magenta) as well as colocalization with cone bipolar cell (SCGN) and horizontal cell (Calbindin) terminals (magenta). Scale bars. 10 μm. See also FIG. 22 B. (C) Representative images of smFISH for Cd47 mRNA (white) combined with IHC for horizontal cell marker Calbindin (magenta) and microglia marker Iba1 (green). Scale bars, 25 μm and 5 μm (insets). See also FIG. 22 C. (D) Representative images showing CD47 colocalization with SIRPα at P6, P9, and P14 in WT retinas. Scale bars, 25 μm. (E-F) Images showing examples of CD47 colocalization with SIRPα (right) and RIBEYE colocalization with SIRPα (left) in P14 retina using Stochastic Optical Reconstruction Microscopy (STORM). In (F), co-localization between SIRPα and CD47 is depicted in white. Scale bars, 2 μm (top) and 500 nm (bottom). See also FIG. 22.

FIG. 17A-N. Neuronal SIRPα and CD47 functioned together to limit microglial phagocytosis. (A) Representative images of Iba1+microglia (green) and CD68+lysosomes (red) in control and CD47 knockout mice. Scale bars. 50 μm and 25 μm (insets). (B-D) Quantifications of microglial morphology and levels of activation, including process endpoints (B), soma size (C), and levels of CD68 staining (D), n=5 per group, unpaired 1-test. See also FIG. 23A. (E) Representative images of Iba1+microglia (green) and CD68+lysosomes (red) in control and SIRPα/CD47 neuron-specific double knockout mice (SIRPα^{NEURON}., CD47NEURON). Scale bars. 50 μm and 25 μm (insets). (F-H) Quantifications of microglial morphology and levels of activation, including process endpoints (F), soma size (G), and levels of CD68 staining (H), n=3 per group, unpaired t-test. See also FIG. 23 B. (I) Representative confocal images of GFP-expressing cells (white). Iba1-labeled microglia (green), and CD68+lysosomes (red) in control (GFP only), CD47+GFP. SIRPα+CD47+GFP, and SIRPα+GFP retinas, viewed in wholemount. Scale bars, 50 μm. (J-N) Quantifications of microglial morphology and CD68 levels, including process length (J), process endpoints (K), soma size (L), levels of CD68 staining (M), and phagocytic cups per cell (N), n=8 control, 9 SIRPα+CD47+GFP, 7 CD47+GFP, and 6 SIRPα+GFP mice, one-way ANOVA with posthoc Bonferroni correction. Data from (F) to (H) were obtained from one experiment. All other data were pooled from two to three independent experiments. All data are presented as the mean+SEM. * p<0.05, ** p<0.01, ns, not significant. See also FIG. 23.

FIG. 18A-C. Microglia localization to synapse layers coincided with synapse emergence, (see also FIG. 11). (A) Quantification of microglia spatial distribution between the inner plexiform (IPL) and outer plexiform (OPL) layers at P6, P9, and P14, n=3 mice for P6, n=4 for P9, n=4 for P14. (B) Quantification of process endpoints per microglia at P6, P9, and P14 in WT retinas, n=8 mice for P6, n=7 for P9, n=6 for P14, one-way ANOVA with posthoc Bonferroni correction. (C) Quantification of percent microglia with phagocytic cups at P6, P9, and P14 in WT retinas, n=7 for P6, n=8 for P9, n=7 for P14, one-way ANOVA with posthoc Bonferroni correction. Data were pooled from two to three independent experiments. All data are presented as the mean±SEM. **** p<0.0001, ns, not significant.

FIG. 19A-H. Neurons produced the majority of SIRPα. (see also FIG. 12 and FIG. 13). (A) Quantification of the mean SIRPα fluorescence intensity in OPL at P6, P9, P14 and P21 in WT mice, n=5 for P6, n=4 for P9, n=3 for P14, n=3 for P21. Data were compared using two-way ANOVA with posthoc Bonferroni correction. (B) Representative confocal image showing SIRPα expression (magenta) in the OPL at 14 weeks. Scale bars, 25 μm. (C) Quantification of the degree of colocalization between SIRPα and synapses (Vglut1) or microglia (Iba1) in P6, P9, and P14 WT retinas using Manders' coefficients (MCC), n=3 per timepoint for Vglut1, n=4 per timepoint for Iba1 quantifications. Data were compared using two-way ANOVA with posthoc Bonferroni correction. (D) Quantification of the degree of colocalization between SIRPα and presynaptic markers (mCAR for cone terminals, PSD95 for rod terminals) or postsynaptic markers (Calbindin for horizontal cell dendrites, SCGN for cone bipolar cell dendrites) in P14 WT retinas using Manders' coefficients (MCC), n=4 for mCAR, n=3 for PSD95, n=5 for Calbindin, and n=3 for SCGN. Data were compared using two-way ANOVA with posthoc Bonferroni correction. (E) Representative fluorescence in situ hybridization (RNAscope) images of SIRPα and microglia marker Iba1 in P2, P6, P9, and P14 WT retinas. Scale bars, 50 μm. (F) Representative confocal images of tdTomato expression (red) upon Cre activity in P9 SIRPα^{F/F}; TNFRSFILA^{Cre}; Ail4 and SIRPα^{F/F}; Six3^{Cre}; Ail4 retinas stained with microglia marker Iba1 (green). Scale bars, 10 μm. (G) Microglia ablation paradigm. Tamoxifen (TMX) and diphtheria toxin (DT) were administrated at the indicated times (top). Representative confocal images and quantification of microglia number in P8 wholemount Cx3crI^{CreER}; Rosa26^{iDTR}and control retinas. This model depleted the majority (˜96%) of microglia in P8 retinas. (H) Representative confocal images of SIRPα staining (magenta) in P9 Cx3crl^{CreER}; Rosa26^{iDTR}and control retinas stained with microglia marker Iba1 (green). Scale bars, 25 μm. Data in (A) to (D) were pooled from 2-3 independent experiments. Data in (G) were obtained from one experiment. All data are presented as the mean±SEM. * p<0.05. *** p<0.001. **** p<0.0001, ns, not significant

FIG. 20A-E, Additional quantification, validation of electroporation, and gating strategies for microglia engulfment (see also FIG. 13). (A) qRT-PCR for selected phagocytic pathway related genes in whole retina when neuronal SIRPα is removed. Values represent fold change in mRNA expression levels relative to the levels detected in controls for each gene following normalization to β-actin, n=3 control, 4 SIRPα^{NEURON} mice, unpaired t-test. (B) Percent microglia with and without phagocytic cups in control. SIRPα^{NEURON}, and SIRPα^{MICROGLIA} microglia, n=8 control, 4 SIRPα^{NEURON}, 3 SIRPα^{MICROGLIA} mice. Data were compared using two-way ANOVA with posthoc Bonferroni correction. (C) Representative confocal images following electroporation at P0 with plasmids overexpressing pCAG-GFP showing that in vivo electroporation mainly targets photoreceptors, with minimal expression in bipolar cells and Muller glia. Microglia and astrocytes are not targeted by electroporation. Scale bars. 50 μm. (D) Validation of flow cytometry gating for Cx3cr1-GFP microglia shows that the majority of GFP+ cells are CD11b+CD45low microglia. (E) Flow cytometry gating for microglial phagocytosis in Cx3cr1-GFP-positive microglia from SIRPα^{NEURON}; Cx3crl^{GFP/+} and SIRPαf/F; Cx3crl^{GFP/+} retinas following incubation with yeast particles conjugated to pHrodo red. Data from (A) were pooled from two independent experiments. Data from (B) were obtained from one experiment. All data are presented as the mean±SEM. * p<0.05, **** p<0.0001, ns, not significant.

FIG. 21A-C. Validation of SIRPα overexpression following in vivo retinal electroporation (see also FIG. 15). (A) Representative images of staining for GFP and SIRPα at P21 in WT retinas following electroporation with plasmids overexpressing GFP or GFP+SIRPα. Scale bars. 25 μm. (B) Representative immunofluorescence images of 3D surface rendering in FIG. 15 B of Iba1-labeled microglia (green) containing CD68-labeled lysosomes (red) at P21 in WT retinas following electroporation with plasmids overexpressing GFP or GFP+SIRPα. Scale bars. 25 μm. (C) Representative immunofluorescence images of 3D surface rendering in FIG. 15 L of Iba1-labeled microglia (gray) containing CD68-labeled lysosomes (red) and engulfed GFP-labeled neural material (green) at P21 in WT retinas following electroporation with plasmids overexpressing GFP or GFP+SIRPα. Scale bars, 25 μm.

FIG. 22A-C, CD47 was expressed in postsynaptic cells (see also FIG. 16). (A) Representative confocal images of CD47 expression in P2 and adult WT retinas. Scale bars, 50 μm. (B) Quantification of the degree of colocalization between CD47 and presynaptic markers (Vglut1 and PSD95 for photoreceptor terminals) or postsynaptic markers (Calbindin for horizontal cell dendrites. SCGN for cone bipolar cell dendrites) at P14 in WT retinas using Manders' coefficients (MCC), n=3 for Vglut1, n=7 for PSD95, n=6 for SCGN, n=7 for Calbindin. Data were compared using two-way ANOVA with posthoc Bonferroni correction. (C) Representative smFISH images of Cd47 mRNA with IHC staining for inner retina neurons (AP2 for amacrine cells, red; RBPMS for ganglion cells, green) at P9 in WT retinas. Scale bars. 50 μm and 10 μm (insets). Data from (B) were pooled from two to three independent experiments. All data are presented as the mean±SEM. ** p<0.01. **** p<0.0001, ns, not significant.

FIG. 23A-D, Neuronal SIRPα limited inhibitory CD47 signaling to microglia (see also FIG. 17). (A) Additional quantifications of microglial morphology and levels of activation, including process length, number of phagocytic cups per cell, and percentage of microglia with cups in CD47 null mice and controls, n=5 mice per group, unpaired 1-test. (B) Additional quantifications of microglial morphology and levels of activation, including process length, number of phagocytic cups per cell, and percentage of microglia with cups in SIRPα/CD47 double knockout mice and controls, n=3 mice per group, unpaired t-test. (C) Representative confocal images of GFP-expressing cells (white). Iba1+microglia (green), and CD68+lysosomes (red) in GFP and CD47+GFP electroporated SIRPα^{MICROGLIA} retinas, viewed in wholemount. Scale bars, 50 μm, n=3 mice per group, unpaired t-test. (D) Representative confocal images of GFP-expressing cells (white), Iba1+microglia (green), and CD68+lysosomes (red) in GFP and SIRPα+GFP electroporated SIRPα^{NEURON} retinas, viewed in wholemount, n=6 control and 4 SIRPα^{NEURON} mice, unpaired t-test. Data from (B) were obtained from one experiment. All other data were pooled from two to three independent experiments. All data are presented as the mean±SEM. ** p<0.01, *** p<0.001, ns, not significant.

### DETAILED DESCRIPTION

Use of the one or more compositions may be employed based on methods described herein. Other embodiments are discussed throughout this application. Any embodiment discussed with respect to one aspect of the disclosure applies to other aspects of the disclosure as well and vice versa. The embodiments in the Example section are understood to be embodiments that are applicable to all aspects of the technology described herein.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this Detailed Description.

### I. Neurological Disorders

In some embodiments, technologies described herein are suitable for prevention of, treatment of, or reduction of risk of, one or more neurological disorders. In certain embodiments, a neurological disorder is a central nervous system disorder (CNS). In certain embodiments, a neurological disorder is a peripheral nervous system disorder (PNS).

In some embodiments, the retina (e.g., a mammalian retina, e.g., a murine retina) provides an approachable model to define molecular mechanisms delineating neuronal projection, expansion, synapse formation, and/or refinement. In some embodiments, the retina provides an approachable and representative model to characterize microglial activation, microglial phagocytosis of neuronal projections, and/or refinement of neuronal systems.

In some embodiments, comparisons between populations (e.g., cells, subjects, animals, treatment groups, etc.) are relative to appropriate control groups. One skilled in the art will recognize appropriate control groups to which comparisons can be and/or are drawn.

Numerous neurological disorders are characterized in part by aberrant microglial activation and/or phagocytosis coupled with or without synaptopathies (e.g., brain disorders that have arisen from synaptic dysfunction; in its broadest definition, a synaptopathy is any perturbation in which aberrant mechanisms correlate with synaptic dysfunction regardless of pathophysiological origin).

Synapses constitute the basic information transfer units in the nervous system and can be divided into two groups: electrical and chemical synapses. Electrical synapses allow for the direct transfer of charged ions and small molecules through pores known as gap junctions, mostly found in glial cells (reviewed in Rouach et al. 2002). Within the chemical synapse, electrical activity is unidirectionally transferred from one neuron (pre-synaptic terminal) to another (post-synaptic terminal) through chemical mediators. Action potentials travel along axons to induce release of neurotransmitters from vesicles in the pre-synaptic bouton into the synaptic cleft; the activation of specific ionotropic receptors by the neurotransmitters is then again transduced into an electrical signal that depolarizes the post-synaptic cell and is transmitted downstream.

Synapse function involves highly specialized molecular machineries at the pre- and post-synapses. However, its homeostasis and plasticity also require the contribution of surrounding glia end-feet, the extracellular matrix (ECM), and microglia. Chemical synapses transduce either excitatory or inhibitory signals that increase or decrease the likelihood of firing action potentials in target cells, respectively. The most abundant excitatory neurotransmitter in the CNS is glutamate. GABA is the main inhibitory neurotransmitter in the adult forebrain, whereas glycine mediates inhibitory neurotransmission mainly in the brainstem and spinal cord (see e.g., Fogarty et al. 2016).

In some embodiments, the present disclosure provides technologies suitable for treatment of a CNS disease, wherein said CNS disease is a CNS infectious disease, a CNS degenerative disease, a CNS auto-immune disease, a CNS tumor disease, a CNS genetic disease, a cerebrovascular disease, a CNS injury, and/or a CNS structural defect.

In certain embodiments, the present disclosure provides technologies suitable for modification of neuronal SIRPα gene activity.

In certain embodiments, the present disclosure provides technologies suitable for modification of neuronal SIRPα protein. In some embodiments, neuronal SIRPα localizes to synapses as they emerge.

In certain embodiments, the present disclosure provides technologies suitable for modification of neuronal SIRPα protein levels and/or activity.

In certain embodiments, the present disclosure provides technologies suitable for modification of neuronal SIRPα ligand CD47.

In certain embodiments, the present disclosure provides technologies suitable for modification of SIRPα that is also produced in microglia.

In certain embodiments, the present disclosure provides technologies suitable for modification of neuronal SIRPα that is primarily produced in neurons and present in neuronal synapses.

In certain embodiments, the present disclosure provides technologies suitable for neuronal SIRPα reduction (e.g., transcriptional downregulation, translational downregulation, protein removal, protein inhibition, etc.). In some embodiments, neuronal SIRPα reduction results in marked changes in microglia morphology.

In certain embodiments, the present disclosure provides technologies suitable for neuronal SIRPα promotion (e.g., transcriptional upregulation, translational upregulation, protein stabilization, protein activation, etc.). In some embodiments, neuronal SIRPα promotion results in marked changes in microglia morphology.

In certain embodiments, the present disclosure provides technologies suitable for neuronal SIRPα modification and subsequent downstream microglia modifications including alterations to activation state and/or phagocytic capabilities.

In certain embodiments, the present disclosure provides technologies suitable for neuronal SIRPα modification and subsequent downstream neuron synapse modifications including but not limited to alterations to synapse number, synapse distribution, synapse function, and any combination thereof.

In certain embodiments, the present disclosure provides technologies suitable for reduction of neuronal SIRPα, wherein reduction of neuronal SIRPα decreases the number of localized activated microglial cells.

In certain embodiments, the present disclosure provides technologies suitable for reduction of neuronal SIRPα, wherein reduction of neuronal SIRPα increases the number of microglial process endpoints per cell.

In certain embodiments, the present disclosure provides technologies suitable for reduction of neuronal SIRPα, wherein reduction of neuronal SIRPα increases microglial total process length.

In certain embodiments, the present disclosure provides technologies suitable for reduction of neuronal SIRPα, wherein reduction of neuronal SIRPα decreases microglial soma size.

In certain embodiments, the present disclosure provides technologies suitable for reduction of neuronal SIRPα, wherein reduction of neuronal SIRPα decreases the number of microglial phagocytic cups.

In certain embodiments, the present disclosure provides technologies suitable for reduction of neuronal SIRPα, wherein reduction of neuronal SIRPα increases the number of microglial cells that do not display phagocytic cups.

In certain embodiments, the present disclosure provides technologies suitable for reduction of neuronal SIRPα, wherein reduction of neuronal SIRPα decreases microglial CD68 levels.

In certain embodiments, the present disclosure provides technologies suitable for reduction of neuronal SIRPα, wherein reduction of neuronal SIRPα decreases microglial amoeboid morphology levels.

In certain embodiments, the present disclosure provides technologies suitable for reduction of neuronal SIRPα, wherein reduction of neuronal SIRPα increases microglial ramified morphology levels.

In certain embodiments, the present disclosure provides technologies suitable for reduction of neuronal SIRP, wherein reduction of neuronal SIRPα increases retention of synapses.

In certain embodiments, the present disclosure provides technologies suitable for reduction of neuronal SIRPα, wherein reduction of neuronal SIRPα increases retention of OPL synapses.

In certain embodiments, the present disclosure provides technologies suitable for reduction of neuronal SIRPα, wherein reduction of neuronal SIRPα reduces scotopic photoreceptor functions.

In certain embodiments, the present disclosure provides technologies suitable for reduction of neuronal SIRPα, wherein phenotypes produced by reduction of SIRPα are specific to reduction of neuronal SIRPα, and not reduction of microglial SIRPα.

In certain embodiments, the present disclosure provides technologies suitable for promotion of neuronal SIRPα, wherein promotion of neuronal SIRPα activates microglial cells.

In certain embodiments, the present disclosure provides technologies suitable for promotion of neuronal SIRPα, wherein promotion of neuronal SIRPα increases microglial CD68 levels.

In certain embodiments, the present disclosure provides technologies suitable for promotion of neuronal SIRPα, wherein promotion of neuronal SIRPα increases microglial soma size.

In certain embodiments, the present disclosure provides technologies suitable for promotion of neuronal SIRPα, wherein promotion of neuronal SIRPα increases synapse engulfment by microglial cells.

In some embodiments, the present disclosure provides technologies suitable for modification (e.g., reduction of or promotion of) of neuronal SIRPα and associated morphological and/or phenotypic changes in neurons and/or microglial cells are localized (e.g., temporally and/or spatially) to regions that were modified.

In certain embodiments, the present disclosure provides technologies suitable for promotion of neuronal CD47, wherein promotion of neuronal CD47 modulates microglial activation. In certain embodiments, promotion of neuronal CD47 reduces microglial phagocytosis. In certain embodiments, promotion of SIRPα can inhibit CD47 reduction of microglial phagocytosis.

In certain embodiments, the present disclosure provides technologies suitable for promotion of neuronal CD47, wherein promotion of neuronal CD47 increases microglial process length.

In certain embodiments, the present disclosure provides technologies suitable for promotion of neuronal CD47, wherein promotion of neuronal CD47 increases microglial process endpoints.

In certain embodiments, the present disclosure provides technologies suitable for promotion of neuronal CD47, wherein promotion of neuronal CD47 reduces microglial soma size.

In certain embodiments, the present disclosure provides technologies suitable for reduction of neuronal CD47, wherein reduction of neuronal CD47 increases retention of synapses.

In certain embodiments, the present disclosure provides technologies suitable for neuronal CD47 modification and subsequent downstream neuron synapse modifications including but not limited to alterations to synapse number, synapse distribution, synapse function, or any combination thereof.

### Neurodegenerative Disorders

In some embodiments, a neurological disorder is characterized in part by neurodegeneration. Increasing evidence demonstrates the importance of synapse dysfunction and/or synapse loss in several neurodegenerative disorders (e.g., major depressive disorder (MDD), schizophrenia, Alzheimer's disease, Huntington disease, Parkinson's disease, amyotrophic lateral sclerosis (ALS), demyelinating diseases (e.g., Multiple Sclerosis (MS)), aging, etc.).

In some embodiments, a disorder that is reduced in likelihood, is prevented, has its symptoms ameliorated, and/or is treated using teachings described herein is Major Depressive Disorder. In some embodiments, a disorder that is reduced in likelihood, is prevented, has its symptoms ameliorated, and/or is treated using teachings described herein is Schizophrenia. In some embodiments, a disorder that is reduced in likelihood, is prevented, has its symptoms ameliorated, and/or is treated using teachings described herein is Alzheimer's disease. In some embodiments, a disorder that is reduced in likelihood, is prevented, has its symptoms ameliorated, and/or is treated using teachings described herein is Huntington disease. In some embodiments, a disorder that is reduced in likelihood, is prevented, has its symptoms ameliorated, and/or is treated using teachings described herein is Parkinson's disease. In some embodiments, a disorder that is reduced in likelihood, is prevented, has its symptoms ameliorated, and/or is treated using teachings described herein is Amyotrophic lateral sclerosis. In some embodiments, a disorder that is reduced in likelihood, is prevented, has its symptoms ameliorated, and/or is treated using teachings described herein is a demyelination disorder (e.g., Multiple Sclerosis). In some embodiments, a disorder that is reduced in likelihood, is prevented, has its symptoms ameliorated, and/or is treated using teachings described herein is aging. In some embodiments, aging is considered broadly, and encompasses age related neurodegeneration that occurs any time after development has ceased, e.g., any time after the age of 25, 30, 35, 40, 45, 50, 55, 60, 65, 70, 75, 80, 85, or above. In certain embodiments, aging related neurodegeneration is that which occurs in individuals older than 60, 65, or 70.

### Aberrant Phagocytosis and Neurodevelopmental Disorders

In some embodiments, a neurological disorder is characterized in part by aberrant microglial mediated phagocytosis and related synaptic abnormalities. Increasing evidence demonstrates the importance of synapse dysfunction as a major determinant of several neurodevelopmental diseases (e.g., autism spectrum disorders (ASDs), Down syndrome, Hyperekplexia (startle disease), epilepsy, other developmental neurological disorders (e.g., applicable rare but impactful neurological diseases, e.g., applicable diseases described by the National Institute of Neurological Disorders and Stroke), etc.). In some embodiments, a disorder that is reduced in likelihood, is prevented, has it's symptoms ameliorated, and/or is treated using teachings described herein are ASDs. In some embodiments, a disorder that is reduced in likelihood, is prevented, has it's symptoms ameliorated, and/or is treated using teachings described herein is Down Syndrome. In some embodiments, a disorder that is reduced in likelihood, is prevented, has it's symptoms ameliorated, and/or is treated using teachings described herein is Hyperekplexia. In some embodiments, a disorder that is reduced in likelihood, is prevented, has it's symptoms ameliorated, and/or is treated using teachings described herein is epilepsy. In some embodiments, a disorder that is reduced in likelihood, is prevented, has it's symptoms ameliorated, and/or is treated using teachings described herein is a developmental neurological disorders (e.g., applicable rare but impactful neurological diseases, e.g., applicable diseases described by the National Institute of Neurological Disorders and Stroke).

### Cluster of Differentiation 47 (CD47)

In some embodiments, a protein, polypeptide, or oligonucleotide that is modified (including by activity and/or expression) according to the present disclosure is a Cluster of Differentiation 47 (CD47) gene product. As defined herein, a gene product is any molecule created using a noted gene as a template, e.g., a polypeptide fragment, a domain, a complete protein, a protein variant, a non-coding RNA, a regulatory RNA, an mRNA transcript, etc.

CD47, known as cluster of differentiation 47 or IAP (integrin associated protein), is a widely expressed transmembrane glycoprotein of 50 kDa belonging to the immunoglobulin (Ig) superfamily, which possesses 5 transmembrane domains of interaction. Through interactions with its ligands such as SIRPα. TSP-1, integrins, and SHPS-1, CD47 regulates numerous functions like cell adhesion, proliferation, apoptosis, migration, homeostasis, phagocytosis via macrophages—“don't eat me signal”, neutrophils migration, and T-cells, B-cells and dendritic cells activation. Moreover, several studies have shown that CD47 receptor expression is significantly increased in a variety of diseases, including non-Hodgkin's lymphoma (NHL), acute myeloid leukemia (AML), acute lymphoblastic leukemia (ALL), chronic myeloid leukemia, multiple myeloma, bladder cancer, Gaucher disease, Multiple Sclerosis and stroke among others.

-CD47 Transcript Variant 1 cDNA sequence-
SEQ ID NO: 1
GCAGCCTGGGCAGTGGGTCCTGCCTGTGACGCGCGGCGGCGGTCGGTCCTGCCTGTAACGGC
GGCGGCGGCTGCTGCTCCGGACACCTGCGGCGGCGGCGGCGACCCCGCGGCGGGCGCGGAGA
TGTGGCCCCTGGTAGCGGCGCTGTTGCTGGGCTCGGCGTGCTGCGGATCAGCTCAGCTACTA
TTTAATAAAACAAAATCTGTAGAATTCACGTTTTGTAATGACACTGTCGTCATTCCATGCTT
TGTTACTAATATGGAGGCACAAAACACTACTGAAGTATACGTAAAGTGGAAATTTAAAGGAA
GAGATATTTACACCTTTGATGGAGCTCTAAACAAGTCCACTGTCCCCACTGACTTTAGTAGT
GCAAAAATTGAAGTCTCACAATTACTAAAAGGAGATGCCTCTTTGAAGATGGATAAGAGTGA
TGCTGTCTCACACACAGGAAACTACACTTGTGAAGTAACAGAATTAACCAGAGAAGGTGAAA
CGATCATCGAGCTAAAATATCGTGTTGTTTCATGGTTTTCTCCAAATGAAAATATTCTTATT
GTTATTTTCCCAATTTTTGCTATACTCCTGTTCTGGGGACAGTTTGGTATTAAAACACTTAA
ATATAGATCCGGTGGTATGGATGAGAAAACAATTGCTTTACTTGTTGCTGGACTAGTGATCA
CTGTCATTGTCATTGTTGGAGCCATTCTTTTCGTCCCAGGTGAATATTCATTAAAGAATGCT
ACTGGCCTTGGTTTAATTGTGACTTCTACAGGGATATTAATATTACTTCACTACTATGTGTT
TAGTACAGCGATTGGATTAACCTCCTTCGTCATTGCCATATTGGTTATTCAGGTGATAGCCT
ATATCCTCGCTGTGGTTGGACTGAGTCTCTGTATTGCGGCGTGTATACCAATGCATGGCCCT
CTTCTGATTTCAGGTTTGAGTATCTTAGCTCTAGCACAATTACTTGGACTAGTTTATATGAA
ATTTGTGGCTTCCAATCAGAAGACTATACAACCTCCTAGGAAAGCTGTAGAGGAACCCCTTA
ATGCATTCAAAGAATCAAAAGGAATGATGAATGATGAATAACTGAAGTGAAGTGATGGACTC
CGATTTGGAGAGTAGTAAGACGTGAAAGGAATACACTTGTGTTTAAGCACCATGGCCTTGAT
GATTCACTGTTGGGGAGAAGAAACAAGAAAAGTAACTGGTTGTCACCTATGAGACCCTTACG
TGATTGTTAGTTAAGTTTTTATTCAAAGCAGCTGTAATTTAGTTAATAAAATAATTATGATC
TATGTTGTTTGCCCAATTGAGATCCAGTTTTTTGTTGTTATTTTTAATCAATTAGGGGCAAT
AGTAGAATGGACAATTTCCAAGAATGATGCCTTTCAGGTCCTAGGGCCTCTGGCCTCTAGGT
AACCAGTTTAAATTGGTTCAGGGTGATAACTACTTAGCACTGCCCTGGTGATTACCCAGAGA
TATCTATGAAAACCAGTGGCTTCCATCAAACCTTTGCCAACTCAGGTTCACAGCAGCTTTGG
GCAGTTATGGCAGTATGGCATTAGCTGAGAGGTGTCTGCCACTTCTGGGTCAATGGAATAAT
AAATTAAGTACAGGCAGGAATTTGGTTGGGAGCATCTTGTATGATCTCCGTATGATGTGATA
TTGATGGAGATAGTGGTCCTCATTCTTGGGGGTTGCCATTCCCACATTCCCCCTTCAACAAA
CAGTGTAACAGGTCCTTCCCAGATTTAGGGTACTTTTATTGATGGATATGTTTTCCTTTTAT
TCACATAACCCCTTGAAACCCTGTCTTGTCCTCCTGTTACTTGCTTCTGCTGTACAAGATGT
AGCACCTTTTCTCCTCTTTGAACATGGTCTAGTGACACGGTAGCACCAGTTGCAGGAAGGAG
CCAGACTTGTTCTCAGAGCACTGTGTTCACACTTTTCAGCAAAAATAGCTATGGTTGTAACA
TATGTATTCCCTTCCTCTGATTTGAAGGCAAAAATCTACAGTGTTTCTTCACTTCTTTTCTG
ATCTGGGGCATGAAAAAAGCAAGATTGAAATTTGAACTATGAGTCTCCTGCATGGCAACAAA
ATGTGTGTCACCATCAGGCCAACAGGCCAGCCCTTGAATGGGGATTTATTACTGTTGTATCT
ATGTTGCATGATAAACATTCATCACCTTCCTCCTGTAGTCCTGCCTCGTACTCCCCTTCCCC
TATGATTGAAAAGTAAACAAAACCCACATTTCCTATCCTGGTTAGAAGAAAATTAATGTTCT
GACAGTTGTGATCGCCTGGAGTACTTTTAGACTTTTAGCATTCGTTTTTTACCTGTTTGTGG
ATGTGTGTTTGTATGTGCATACGTATGAGATAGGCACATGCATCTTCTGTATGGACAAAGGT
GGGGTACCTACAGGAGAGCAAAGGTTAATTTTGTGCTTTTAGTAAAAACATTTAAATACAAA
GTTCTTTATTGGGTGGAATTATATTTGATGCAAATATTTGATCACTTAAAACTTTTAAAACT
TCTAGGTAATTTGCCACGCTTTTTGACTGCTCACCAATACCCTGTAAAAATACGTAATTCTT
CCTGTTTGTGTAATAAGATATTCATATTTGTAGTTGCATTAATAATAGTTATTTCTTAGTCC
ATCAGATGTTCCCGTGTGCCTCTTTTATGCCAAATTGATTGTCATATTTCATGTTGGGACCA
AGTAGTTTGCCCATGGCAAACCTAAATTTATGACCTGCTGAGGCCTCTCAGAAAACTGAGCA
TACTAGCAAGACAGCTCTTCTTGAAAAAAAAAATATGTATACACAAATATATACGTATATCT
ATATATACGTATGTATATACACACATGTATATTCTTCCTTGATTGTGTAGCTGTCCAAAATA
ATAACATATATAGAGGGAGCTGTATTCCTTTATACAAATCTGATGGCTCCTGCAGCACTTTT
TCCTTCTGAAAATATTTACATTTTGCTAACCTAGTTTGTTACTTTAAAAATCAGTTTTGATG
AAAGGAGGGAAAAGCAGATGGACTTGAAAAAGATCCAAGCTCCTATTAGAAAAGGTATGAAA
ATCTTTATAGTAAAATTTTTTATAAACTAAAGTTGTACCTTTTAATATGTAGTAAACTCTCA
TTTATTTGGGGTTCGCTCTTGGATCTCATCCATCCATTGTGTTCTCTTTAATGCTGCCTGCC
TTTTGAGGCATTCACTGCCCTAGACAATGCCACCAGAGATAGTGGGGGAAATGCCAGATGAA
ACCAACTCTTGCTCTCACTAGTTGTCAGCTTCTCTGGATAAGTGACCACAGAAGCAGGAGTC
CTCCTGCTTGGGCATCATTGGGCCAGTTCCTTCTCTTTAAATCAGATTTGTAATGGCTCCCA
AATTCCATCACATCACATTTAAATTGCAGACAGTGTTTTGCACATCATGTATCTGTTTTGTC
CCATAATATGCTTTTTACTCCCTGATCCCAGTTTCTGCTGTTGACTCTTCCATTCAGTTTTA
TTTATTGTGTGTTCTCACAGTGACACCATTTGTCCTTTTCTGCAACAACCTTTCCAGCTACT
TTTGCCAAATTCTATTTGTCTTCTCCTTCAAAACATTCTCCTTTGCAGTTCCTCTTCATCTG
TGTAGCTGCTCTTTTGTCTCTTAACTTACCATTCCTATAGTACTTTATGCATCTCTGCTTAG
TTCTATTAGTTTTTTGGCCTTGCTCTTCTCCTTGATTTTAAAATTCCTTCTATAGCTAGAGC
TTTTCTTTCTTTCATTCTCTCTTCCTGCAGTGTTTTGCATACATCAGAAGCTAGGTACATAA
GTTAAATGATTGAGAGTTGGCTGTATTTAGATTTATCACTTTTTAATAGGGTGAGCTTGAGA
GTTTTCTTTCTTTCTGTTTTTTTTTTTTGTTTTTTTTTTTTTTTTTTTTTTTTTTTTTTTTG
ACTAATTTCACATGCTCTAAAAACCTTCAAAGGTGATTATTTTTCTCCTGGAAACTCCAGGT
CCATTCTGTTTAAATCCCTAAGAATGTCAGAATTAAAATAACAGGGCTATCCCGTAATTGGA
AATATTTCTTTTTTCAGGATGCTATAGTCAATTTAGTAAGTGACCACCAAATTGTTATTTGC
ACTAACAAAGCTCAAAACACGATAAGTTTACTCCTCCATCTCAGTAATAAAAATTAAGCTGT
AATCAACCTTCTAGGTTTCTCTTGTCTTAAAATGGGTATTCAAAAATGGGGATCTGTGGTGT
ATGTATGGAAACACATACTCCTTAATTTACCTGTTGTTGGAAACTGGAGAAATGATTGTCGG
GCAACCGTTTATTTTTTATTGTATTTTATTTGGTTGAGGGATTTTTTTATAAACAGTTTTAC
TTGTGTCATATTTTAAAATTACTAACTGCCATCACCTGCTGGGGTCCTTTGTTAGGTCATTT
TCAGTGACTAATAGGGATAATCCAGGTAACTTTGAAGAGATGAGCAGTGAGTGACCAGGCAG
TTTTTCTGCCTTTAGCTTTGACAGTTCTTAATTAAGATCATTGAAGACCAGCTTTCTCATAA
ATTTCTCTTTTTGAAAAAAAGAAAGCATTTGTACTAAGCTCCTCTGTAAGACAACATCTTAA
ATCTTAAAAGTGTTGTTATCATGACTGGTGAGAGAAGAAAACATTTTGTTTTTATTAAATGG
AGCATTATTTACAAAAAGCCATTGTTGAGAATTAGATCCCACATCGTATAAATATCTATTAA
CCATTCTAAATAAAGAGAACTCCAGTGTTGCTATGTGCAAGATCCTCTCTTGGAGCTTTTTT
GCATAGCAATTAAAGGTGTGCTATTTGTCAGTAGCCATTTTTTTGCAGTGATTTGAAGACCA
AAGTTGTTTTACAGCTGTGTTACCGTTAAAGGTTTTTTTTTTTATATGTATTAAATCAATTT
ATCACTGTTTAAAGCTTTGAATATCTGCAATCTTTGCCAAGGTACTTTTTTATTTAAAAAAA
AACATAACTTTGTAAATATTACCCTGTAATATTATATATACTTAATAAAACATTTTAAGCTA
TTTTGTTGGGCTATTTCTATTGCTGCTACAGCAGACCACAAGCACATTTCTGAAAAATTTAA
TTTATTAATGTATTTTTAAGTTGCTTATATTCTAGGTAACAATGTAAAGAATGATTTAAAAT
ATTAATTATGAATTTTTTGAGTATAATACCCAATAAGCTTTTAATTAGAGCAGAGTTTTAAT
TAAAAGTTTTAAATCAGTCCAA
-CD47 Transcript Variant 1 nucleic acid coding sequence-
SEQ ID NO: 2
ATGTGGCCCCTGGTAGCGGCGCTGTTGCTGGGCTCGGCGTGCTGCGGATCAGCTCAGCTACT
ATTTAATAAAACAAAATCTGTAGAATTCACGTTTTGTAATGACACTGTCGTCATTCCATGCT
TTGTTACTAATATGGAGGCACAAAACACTACTGAAGTATACGTAAAGTGGAAATTTAAAGGA
AGAGATATTTACACCTTTGATGGAGCTCTAAACAAGTCCACTGTCCCCACTGACTTTAGTAG
TGCAAAAATTGAAGTCTCACAATTACTAAAAGGAGATGCCTCTTTGAAGATGGATAAGAGTG
ATGCTGTCTCACACACAGGAAACTACACTTGTGAAGTAACAGAATTAACCAGAGAAGGTGAA
ACGATCATCGAGCTAAAATATCGTGTTGTTTCATGGTTTTCTCCAAATGAAAATATTCTTAT
TGTTATTTTCCCAATTTTTGCTATACTCCTGTTCTGGGGACAGTTTGGTATTAAAACACTTA
AATATAGATCCGGTGGTATGGATGAGAAAACAATTGCTTTACTTGTTGCTGGACTAGTGATC
ACTGTCATTGTCATTGTTGGAGCCATTCTTTTCGTCCCAGGTGAATATTCATTAAAGAATGC
TACTGGCCTTGGTTTAATTGTGACTTCTACAGGGATATTAATATTACTTCACTACTATGTGT
TTAGTACAGCGATTGGATTAACCTCCTTCGTCATTGCCATATTGGTTATTCAGGTGATAGCC
TATATCCTCGCTGTGGTTGGACTGAGTCTCTGTATTGCGGCGTGTATACCAATGCATGGCCC
TCTTCTGATTTCAGGTTTGAGTATCTTAGCTCTAGCACAATTACTTGGACTAGTTTATATGA
AATTTGTGGCTTCCAATCAGAAGACTATACAACCTCCTAGGAAAGCTGTAGAGGAACCCCTT
AATGCATTCAAAGAATCAAAAGGAATGATGAATGATGAATAA
-CD47 Isoform 1 amino acid sequence-
SEQ ID NO: 3
MWPLVAALLLGSACCGSAQLLFNKTKSVEFTFCNDTVVIPCFVINMEAQNTTEVYVKWKFKG
RDIYTFDGALNKSTVPTDFSSAKIEVSQLLKGDASLKMDKSDAVSHTGNYTCEVTELTREGE
TIIELKYRVVSWFSPNENILIVIFPIFAILLFWGQFGIKTLKYRSGGMDEKTIALLVAGLVI
TVIVIVGAILFVPGEYSLKNATGLGLIVISTGILILLHYYVFSTAIGLTSFVIAILVIQVIA
YILAVVGLSLCIAACIPMHGPLLISGLSILALAQLLGLVYMKFVASNQKTIQPPRKAVEEPL
NAFKESKGMMNDE
-CD47 Isoform 2 amino acid sequence-
SEQ ID NO: 4
MWPLVAALLLGSACCGSAQLLFNKIKSVEFTFCNDTVVIPCFVTNMEAQNTTEVYVKWKFKG
RDIYTFDGALNKSTVPTDFSSAKIEVSQLLKGDASLKMDKSDAVSHTGNYTCEVTELTREGE
TIIELKYRVVSWFSPNENILIVIFPIFAILLFWGQFGIKTLKYRSGGMDEKTIALLVAGLVI
TVIVIVGAILFVPGEYSLKNATGLGLIVISIGILILLHYYVFSTAIGLISFVIAILVIQVIA
YILAVVGLSLCIAACIPMHGPLLISGLSILALAQLLGLVYMKFVASNQKTIQPPRNN
-CD47 Isoform 3 amino acid sequence-
SEQ ID NO: 5
MWPLVAALLLGSACCGSAQLLENKTKSVEFTFCNDTVVIPCFVINMEAQNTTEVYVKWKFKG
RDIYTFDGALNKSTVPTDFSSAKIEVSQLLKGDASLKMDKSDAVSHTGNYTCEVTELTREGE
TIIELKYRVVSWFSPNENILIVIFPIFAILLFWGQFGIKTLKYRSGGMDEKTIALLVAGLVI
TVIVIVGAILFVPGEYSLKNATGLGLIVISTGILILLHYYVFSTAIGLTSFVIAILVIQVIA
YILAVVGLSLCIAACIPMHGPLLISGLSILALAQLLGLVYMKFVASNQKTIQPPRKAVEEPL
NE
-Velcro-CD47 (N3612) amino acid sequence-
SEQ ID NO: 6
WQPPLLFNKTKSVEFTFGNDTVVIPCFVTNMEAQNTTEVYVKWKFKGRDIYTFDGQANKSTV
PTDFSSAKIEVSQLLKGDASLKMDKSDAVSHTGNYTCEVTELTREGETIIELKYRVVS

### Signal Regulatory Protein Alpha (SIRPα)

In some embodiments, a protein, polypeptide, or oligonucleotide that is modified (including by activity and/or expression) according to the present disclosure is Signal Regulatory Protein Alpha (SIRPα), also known as CD172.

SIRPα is an inhibitory receptor with high-affinity interaction for CD47 in brain cells and other tissues. This protein belongs to the SIRP family of receptors which comprise SIRPα, SIRPβ, SIRPγ, and soluble SIRPδ members.

The expression of SIRPα has been demonstrated to occur on the surface of microglial cells, hippocampal neurons, oligodendrocytes, and astrocytes. In certain aspects, it is likely that interactions between SIRPα and CD47 are crucial for cell-to-cell communication in the brain both in normal and pathological conditions. Extracellular portions of SIRPα consist of three IgSF domains, including two membrane-proximal IgC domains and one membrane-distal IgV domain (N-terminal). Previous research has shown that the N-terminal IgV domain of SIRPα binds to CD47. It is thought that receptors in the immunoglobulin superfamily react with the ‘turning off’ signal and thus modulate microglia activation, migration, and phagocytosis. This regulation occurs through a cytoplasmic-domain, immuno-receptor tyrosine-based inhibition motif (ITIM), whereas receptor activation during microglia regulation occurs by a cytoplasmic-domain ITAM. It is notable that the cytoplasmic region of SIRPα, as one of the receptors that recognize the off-signal, consists of two ITIM with four strongly conserved tyrosine residues. In addition, SIRPα ligation via CD47 would induce phosphorylation of these tyrosine residues.

-Signal Regulatory Protein Alpha (SIRPα) Transcript Variant 1 cDNA
sequence-
SEQ ID NO: 7
TCCGGCCCGCACCCACCCCCAAGAGGGGCCTTCAGCTTTGGGGCTCAGAGGCACGACCTCCT
GGGGAGGGTTAAAAGGCAGACGCCCCCCCGCCCCCCGCGCCCCCGCGCCCCGACTCCTTCGC
CGCCTCCAGCCTCTCGCCAGTGGGAAGCGGGGAGCAGCCGCGCGGCCGGAGTCCGGAGGCGA
GGGGAGGTCGGCCGCAACTTCCCCGGTCCACCTTAAGAGGACGATGTAGCCAGCTCGCAGCG
CTGACCTTAGAAAAACAAGTTTGCGCAAAGTGGAGCGGGGACCCGGCCTCTGGGCAGCCCCG
GCGGCGCTTCCAGTGCCTTCCAGCCCTCGCGGGCGGCGCAGCCGCGGCCCATGGAGCCCGCC
GGCCCGGCCCCCGGCCGCCTCGGGCCGCTGCTCTGCCTGCTGCTCGCCGCGTCCTGCGCCTG
GTCAGGAGTGGCGGGTGAGGAGGAGCTGCAGGTGATTCAGCCTGACAAGTCCGTGTTGGTTG
CAGCTGGAGAGACAGCCACTCTGCGCTGCACTGCGACCTCTCTGATCCCTGTGGGGCCCATC
CAGTGGTTCAGAGGAGCTGGACCAGGCCGGGAATTAATCTACAATCAAAAAGAAGGCCACTT
CCCCCGGGTAACAACTGTTTCAGACCTCACAAAGAGAAACAACATGGACTTTTCCATCCGCA
TCGGTAACATCACCCCAGCAGATGCCGGCACCTACTACTGTGTGAAGTTCCGGAAAGGGAGC
CCCGATGACGTGGAGTTTAAGTCTGGAGCAGGCACTGAGCTGTCTGTGCGCGCCAAACCCTC
TGCCCCCGTGGTATCGGGCCCTGCGGCGAGGGCCACACCTCAGCACACAGTGAGCTTCACCT
GCGAGTCCCACGGCTTCTCACCCAGAGACATCACCCTGAAATGGTTCAAAAATGGGAATGAG
CTCTCAGACTTCCAGACCAACGTGGACCCCGTAGGAGAGAGCGTGTCCTACAGCATCCACAG
CACAGCCAAGGTGGTGCTGACCCGCGAGGACGTTCACTCTCAAGTCATCTGCGAGGTGGCCC
ACGTCACCTTGCAGGGGGACCCTCTTCGTGGGACTGCCAACTTGTCTGAGACCATCCGAGTT
CCACCCACCTTGGAGGTTACTCAACAGCCCGTGAGGGCAGAGAACCAGGTGAATGTCACCTG
CCAGGTGAGGAAGTTCTACCCCCAGAGACTACAGCTGACCTGGTTGGAGAATGGAAACGTGT
CCCGGACAGAAACGGCCTCAACCGTTACAGAGAACAAGGATGGTACCTACAACTGGATGAGC
TGGCTCCTGGTGAATGTATCTGCCCACAGGGATGATGTGAAGCTCACCTGCCAGGTGGAGCA
TGACGGGCAGCCAGCGGTCAGCAAAAGCCATGACCTGAAGGTCTCAGCCCACCCGAAGGAGC
AGGGCTCAAATACCGCCGCTGAGAACACTGGATCTAATGAACGGAACATCTATATTGTGGTG
GGTGTGGTGTGCACCTTGCTGGTGGCCCTACTGATGGCGGCCCTCTACCTCGTCCGAATCAG
ACAGAAGAAAGCCCAGGGCTCCACTTCTTCTACAAGGTTGCATGAGCCCGAGAAGAATGCCA
GAGAAATAACACAGGACACAAATGATATCACATATGCAGACCTGAACCTGCCCAAGGGGAAG
AAGCCTGCTCCCCAGGCTGCGGAGCCCAACAACCACACGGAGTATGCCAGCATTCAGACCAG
CCCGCAGCCCGCGTCGGAGGACACCCTCACCTATGCTGACCTGGACATGGTCCACCTCAACC
GGACCCCCAAGCAGCCGGCCCCCAAGCCTGAGCCGTCCTTCTCAGAGTACGCCAGCGTCCAG
GTCCCGAGGAAGTGAATGGGACCGTGGTTTGCTCTAGCACCCATCTCTACGCGCTTTCTTGT
CCCACAGGGAGCCGCCGTGATGAGCACAGCCAACCCAGTTCCCGGAGGGCTGGGGCGGTGCA
GGCTCTGGGACCCAGGGGCCAGGGTGGCTCTTCTCTCCCCACCCCTCCTTGGCTCTCCAGCA
CTTCCTGGGCAGCCACGGCCCCCTCCCCCCACATTGCCACATACCTGGAGGCTGACGTTGCC
AAACCAGCCAGGGAACCAACCTGGGAAGTGGCCAGAACTGCCTGGGGTCCAAGAACTCTTGT
GCCTCCGTCCATCACCATGTGGGTTTTGAAGACCCTCGACTGCCTCCCCGATGCTCCGAAGC
CTGATCTTCCAGGGTGGGGAGGAGAAAATCCCACCTCCCCTGACCTCCACCACCTCCACCAC
CACCACCACCACCACCACCACCACTACCACCACCACCCAACTGGGGCTAGAGTGGGGAAGAT
TTCCCCTTTAGATCAAACTGCCCCTTCCATGGAAAAGCTGGAAAAAAACTCTGGAACCCATA
TCCAGGCTTGGTGAGGTTGCTGCCAACAGTCCTGGCCTCCCCCATCCCTAGGCTAAAGAGCC
ATGAGTCCTGGAGGAGGAGAGGACCCCTCCCAAAGGACTGGAGACAAAACCCTCTGCTTCCT
TGGGTCCCTCCAAGACTCCCTGGGGCCCAACTGTGTTGCTCCACCCGGACCCATCTCTCCCT
TCTAGACCTGAGCTTGCCCCTCCAGCTAGCACTAAGCAACATCTCGCTGTGGACGCCTGTAA
ATTACTGAGAAATGTGAAACGTGCAATCTTGAAACTGAGGTGTTAGAAAACTTGATCTGTGG
TGTTTTGTTTTGTTTTTTTTCTTAAAACAACAGCAACGTGATCTTGGCTGTCTGTCATGTGT
TGAAGTCCATGGTTGGGTCTTGTGAAGTCTGAGGTTTAACAGTTTGTTGTCCTGGAGGGATT
TTCTTACAGCGAAGACTTGAGTTCCTCCAAGTCCCAGAACCCCAAGAATGGGCAAGAAGGAT
CAGGTCAGCCACTCCCTGGAGACACAGCCTTCTGGCTGGGACTGACTTGGCCATGTTCTCAG
CTGAGCCACGCGGCTGGTAGTGCAGCCTTCTGTGACCCCGCTGTGGTAAGTCCAGCCTGCCC
AGGGCTGCTGAGGGCTGCCTCTTGACAGTGCAGTCTTATCGAGACCCAATGCCTCAGTCTGC
TCATCCGTAAAGTGGGGATAGTGAAGATGACACCCCTCCCCACCACCTCTCATAAGCACTTT
AGGAACACACAGAGGGTAGGGATAGTGGCCCTGGCCGTCTATCCTACCCCTTTAGTGACCGC
CCCCATCCCGGCTTTCTGAGCTGATCCTTGAAGAAGAAATCTTCCATTTCTGCTCTCAAACC
CTACTGGGATCAAACTGGAATAAATTGAAGACAGCCAGGGGGATGGTGCAGCTGTGAAGCTC
GGGCTGATTCCCCCTCTGTCCCAGAAGGTTGGCCAGAGGGTGTGACCCAGTTACCCTTTAAC
CCCCACCCTTCCAGTCGGGTGTGAGGGCCTGACCGGGCCCAGGGCAAGCAGATGTCGCAAGC
CCTATTTATTCAGTCTTCACTATAACTCTTAGAGTTGAGACGCTAATGTTCATGACTCCTGG
CCTTGGGATGCCCAAGGGATTTCTGGCTCAGGCTGTAAAAGTAGCTGAGCCATCCTGCCCAT
TCCTGGAGGTCCTACAGGTGAAACTGCAGGAGCTCAGCATAGACCCAGCTCTCTGGGGGATG
GTCACCTGGTGATTTCAATGATGGCATCCAGGAATTAGCTGAGCCAACAGACCATGTGGACA
GCTTTGGCCAGAGCTCCCGTGTGGCATCTGGGAGCCACAGTGACCCAGCCACCTGGCTCAGG
CTAGTTCCAAATTCCAAAAGATTGGCTTGTAAACCTTCGTCTCCCTCTCTTTTACCCAGAGA
CAGCACATACGTGTGCACACGCATGCACACACACATTCAGTATTTTAAAAGAATGTTTTCTT
GGTGCCATTTTCATTTTATTTTATTTTTTAATTCTTGGAGGGGGAAATAAGGGAATAAGGCC
AAGGAAGATGTATAGCTTTAGCTTTAGCCTGGCAACCTGGAGAATCCACATACCTTGTGTAT
TGAACCCCAGGAAAAGGAAGAGGTCGAACCAACCCTGCGGAAGGAGCATGGTTTCAGGAGTT
TATTTTAAGACTGCTGGGAAGGAAACAGGCCCCATTTTGTATATAGTTGCAACTTAAACTTT
TTGGCTTGCAAAATATTTTTGTAATAAAGATTTCTGGGTAATAATGA
-SIRPα Transcript Variant 1 nucleic acid coding sequence-
SEQ ID NO: 8
ATGGAGCCCGCCGGCCCGGCCCCCGGCCGCCTCGGGCCGCTGCTCTGCCTGCTGCTCGCCGC
GTCCTGCGCCTGGTCAGGAGTGGCGGGTGAGGAGGAGCTGCAGGTGATTCAGCCTGACAAGT
CCGTGTTGGTTGCAGCTGGAGAGACAGCCACTCTGCGCTGCACTGCGACCTCTCTGATCCCT
GTGGGGCCCATCCAGTGGTTCAGAGGAGCTGGACCAGGCCGGGAATTAATCTACAATCAAAA
AGAAGGCCACTTCCCCCGGGTAACAACTGTTTCAGACCTCACAAAGAGAAACAACATGGACT
TTTCCATCCGCATCGGTAACATCACCCCAGCAGATGCCGGCACCTACTACTGTGTGAAGTTC
CGGAAAGGGAGCCCCGATGACGTGGAGTTTAAGTCTGGAGCAGGCACTGAGCTGTCTGTGCG
CGCCAAACCCTCTGCCCCCGTGGTATCGGGCCCTGCGGCGAGGGCCACACCTCAGCACACAG
TGAGCTTCACCTGCGAGTCCCACGGCTTCTCACCCAGAGACATCACCCTGAAATGGTTCAAA
AATGGGAATGAGCTCTCAGACTTCCAGACCAACGTGGACCCCGTAGGAGAGAGCGTGTCCTA
CAGCATCCACAGCACAGCCAAGGTGGTGCTGACCCGCGAGGACGTTCACTCTCAAGTCATCT
GCGAGGTGGCCCACGTCACCTTGCAGGGGGACCCTCTTCGTGGGACTGCCAACTTGTCTGAG
ACCATCCGAGTTCCACCCACCTTGGAGGTTACTCAACAGCCCGTGAGGGCAGAGAACCAGGT
GAATGTCACCTGCCAGGTGAGGAAGTTCTACCCCCAGAGACTACAGCTGACCTGGTTGGAGA
ATGGAAACGTGTCCCGGACAGAAACGGCCTCAACCGTTACAGAGAACAAGGATGGTACCTAC
AACTGGATGAGCTGGCTCCTGGTGAATGTATCTGCCCACAGGGATGATGTGAAGCTCACCTG
CCAGGTGGAGCATGACGGGCAGCCAGCGGTCAGCAAAAGCCATGACCTGAAGGTCTCAGCCC
ACCCGAAGGAGCAGGGCTCAAATACCGCCGCTGAGAACACTGGATCTAATGAACGGAACATC
TATATTGTGGTGGGTGTGGTGTGCACCTTGCTGGTGGCCCTACTGATGGCGGCCCTCTACCT
CGTCCGAATCAGACAGAAGAAAGCCCAGGGCTCCACTTCTTCTACAAGGTTGCATGAGCCCG
AGAAGAATGCCAGAGAAATAACACAGGACACAAATGATATCACATATGCAGACCTGAACCTG
CCCAAGGGGAAGAAGCCTGCTCCCCAGGCTGCGGAGCCCAACAACCACACGGAGTATGCCAG
CATTCAGACCAGCCCGCAGCCCGCGTCGGAGGACACCCTCACCTATGCTGACCTGGACATGG
TCCACCTCAACCGGACCCCCAAGCAGCCGGCCCCCAAGCCTGAGCCGTCCTTCTCAGAGTAC
GCCAGCGTCCAGGTCCCGAGGAAGTGA
-SIRPα Isoform 1 amino acid sequence-
SEQ ID NO: 9
MEPAGPAPGRLGPLLCLLLAASCAWSGVAGEEELQVIQPDKSVLVAAGETATLRCTATSLIP
VGPIQWFRGAGPGRELIYNQKEGHFPRVITVSDLIKRNNMDFSIRIGNITPADAGTYYCVKF
RKGSPDDVEFKSGAGTELSVRAKPSAPVVSGPAARATPQHTVSFTCESHGFSPRDITLKWFK
NGNELSDFQTNVDPVGESVSYSIHSTAKVVLTREDVHSQVICEVAHVTLQGDPLRGTANLSE
TIRVPPTLEVTQQPVRAENQVNVTCQVRKFYPQRLQLTWLENGNVSRTETASTVTENKDGTY
NWMSWLLVNVSAHRDDVKLTCQVEHDGQPAVSKSHDLKVSAHPKEQGSNTAAENTGSNERNI
YIVVGVVCTLLVALLMAALYLVRIRQKKAQGSTSSTRLHEPEKNAREITQDINDITYADLNL
PKGKKPAPQAAEPNNHTEYASIQTSPQPASEDTLTYADLDMVHLNRTPKQPAPKPEPSFSEY
ASVQVPRK
-SIRPα Isoform 2 amino acid sequence-
SEQ ID NO: 10
MEPAGPAPGRLGPLLCLLLAASCAWSGVAGEEELQVIQPDKSVLVAAGETATLRCTATSLIP
VGPIQWFRGAGPGRELIYNQKEGHFPRVTTVSDLTKRNNMDFSIRIGNITPADAGTYYCVKF
RKGSPDDVEFKSGAGTELSVRAKPSAPVVSGPAARATPQHTVSFTCESHGFSPRDITLKWFK
NGNELSDFQTNVDPVGESVSYSIHSTAKVVLTREDVHSQVICEVAHVTLQGDPLRGTANLSE
TIRVPPTLEVTQQPVRAENQVNVTCQVRKFYPQRLQLTWLENGNVSRTETASTVTENKDGTY
NWMSWLLVNVSAHRDDVKLTCQVEHDGQPAVSKSHDLKVSAHPKEQGSNTAAENTGSNERNI
YIVVGVVCTLLVALLMAALYLVRIRQKKAQGSTSSTRLHEPEKNAREITQVQSLDINDITYA
DLNLPKGKKPAPQAAEPNNHTEYASIQTSPQPASEDILTYADLDMVHLNRTPKQPAPKPEPS
FSEYASVQVPRK

II. Therapeutic compositions-Proteins, Polypeptides, Oligonucleotides, and Vectors

In some embodiments, contemplated herein is the use of compositions and/or methods suitable for modifying SIRPα and/or CD47 gene activity in the nervous system. As used herein, “gene activity” refers to creation of transcriptional and/or translational products produced using the noted gene as a template. For example, SIRPα gene activity includes but is not limited to transcription of coding and/or non-coding transcript variants, transcription of intron and/or 5′ or 3′ untranslated region embedded regulatory elements, translation of an encoded transcript, etc. Additionally, for example, a gene activity modifier may include a transcriptional or translational inhibitor and/or promoter.

In some embodiments, contemplated herein is the use of compositions and/or methods suitable for modifying SIRPα and/or CD47 protein levels. In some embodiments, compositions and/or methods are suitable for modifying SIRPα and/or CD47 protein levels specifically in the nervous system. In some embodiments, compositions and/or methods are suitable for modifying SIRPα and/or CD47 protein levels specifically in neurons.

In some embodiments, contemplated herein is the use of compositions and/or methods suitable for modifying SIRPα and/or CD47 protein activity. In some embodiments, compositions and/or methods are suitable for modifying SIRPα and/or CD47 protein activity specifically in the nervous system. In some embodiments, compositions and/or methods are suitable for modifying SIRPα and/or CD47 protein activity specifically in neurons.

As described herein, the term “modifying” can include promotion or inhibition of the modified character. For example, a modifier may be described to either increase or decrease the level and/or activity of the modified character.

### A. Inhibitory Oligonucleotides

In some embodiments, disclosed are compositions and methods that relate to the use of inhibitory oligonucleotides that inhibit the gene expression of SIRPα and/or CD47. In some embodiments, such an inhibitory oligonucleotide specifically inhibits neuronal SIRPα and/or CD47 gene expression.

In some embodiments, disclosed are compositions and methods that relate to the use of inhibitory oligonucleotides that inhibit an inhibitor of gene expression of SIRPα and/or CD47 (e.g., said inhibitory oligonucleotide acts in a promotive manner for SIRPα and/or CD47 gene activity). In some embodiments, such an inhibitory oligonucleotide specifically inhibits a negative regulator of SIRPα and/or CD47 gene activity.

In some embodiments, inhibitory oligonucleotides can include but is not limited to siRNA (small interfering RNA), short hairpin RNA (shRNA), double-stranded RNA, an antisense oligonucleotide (ASO), a ribozyme, and an oligonucleotide encoding any thereof. An inhibitory oligonucleotide may inhibit the transcription of a gene or prevent the translation of a gene transcript in a cell. An inhibitory oligonucleotide acid may be from 16 to 1000 nucleotides long, and in certain embodiments from 18 to 100 nucleotides long. An inhibitory oligonucleotide may have at least or may have at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 50, 60, 70, 80, or 90 (or any range derivable therein) nucleotides. An inhibitory oligonucleotide may be DNA, RNA, or a cDNA that encodes an inhibitory RNA.

As used herein, “isolated” means altered or removed from the natural state through human intervention. For example, an siRNA naturally present in a living animal is not “isolated,” but a synthetic siRNA, or an siRNA partially or completely separated from the coexisting materials of its natural state is “isolated.” An isolated siRNA can exist in substantially purified form, or can exist in a non-native environment such as, for example, a cell into which the siRNA has been delivered.

Inhibitory oligonucleotides are well known in the art. For example, siRNA and double-stranded RNA have been described in U.S. Pat. Nos. 6,506,559 and 6,573,099, as well as in U.S. Patent Publications 2003/0051263, 2003/0055020, 2004/0265839, 2002/0168707, 2003/0159161, and 2004/0064842, all of which are herein incorporated by reference in their entirety.

Particularly, in some embodiments, an inhibitory oligonucleotide may be capable of decreasing the expression of SIRPα and/or CD47 (e.g., neuronal SIRPα and/or neuronal CD47) by at least 10%, 20%, 30%, or 40%, more particularly by at least 50%, 60%, or 70%. and most particularly by at least 75%, 80%, 90%, 95%, 99%, or more, or any range or value in between the foregoing. Alternatively in other embodiments, an inhibitory oligonucleotide may be capable of decreasing the expression of a SIRPα and/or CD47 (e.g., neuronal SIRPα and/or neuronal CD47) negative regulator by at least 10%, 20%, 30%, or 40%, more particularly by at least 50%, 60%, or 70%, and most particularly by at least 75%, 80%, 90%, 95%, 99%, or more, or any range or value in between the foregoing.

In some embodiments, the oligonucleotide is at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 99.1%, 99.2%, 99.3%, 99.4%, 99.5%, 99.6%, 99.7%. 99.8%, 99.9%, or 100% identical or complementary to a region of any of SEQ ID NOs: 1, 2, 7, or 8. In some embodiments, the region is a region having, having at least, or having at most 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides, or any range derivable therein, starting at any position of any of SEQ ID NOs: 1, 2, 7, or 8.

In further embodiments, there are synthetic inhibitory oligonucleotides that are SIRPα and/or CD47 inhibitors, or SIRPα and/or CD47 negative regulator inhibitors. An inhibitory oligonucleotide may be between 17 to 25 nucleotides in length and comprises a 5′ to 3′ sequence that is at least 90% complementary to the 5′ to 3′ sequence of a mature target mRNA. In certain embodiments, an inhibitory oligonucleotide molecule is 17, 18, 19, 20, 21, 22, 23, 24, or 25 nucleotides in length, or any range derivable therein. Moreover, an inhibitory oligonucleotide molecule has a sequence (from 5′ to 3′) that is or is at least 90, 91, 92, 93, 94, 95, 96, 97, 98, 99, 99.1, 99.2, 99.3, 99.4, 99.5, 99.6, 99.7, 99.8, 99.9 or 100% complementary. or any range derivable therein, to the 5′ to 3′ sequence of a mature target mRNA, particularly a mature, naturally occurring mRNA. One of skill in the art can use a portion of a probe sequence that is complementary to the sequence of a mature mRNA as the sequence for an mRNA inhibitor. Moreover, that portion of the probe sequence can be altered so that it is still 90% complementary to the sequence of a mature mRNA.

In some embodiments, the inhibitory oligonucleotide is an analog and may include modifications, particularly modifications that increase nuclease resistance, improve binding affinity, and/or improve binding specificity. For example, when the sugar portion of a nucleoside or nucleotide is replaced by a carbocyclic moiety, it is no longer a sugar. Moreover, when other substitutions, such a substitution for the inter-sugar phosphodiester linkage are made, the resulting material is no longer a true species. All such compounds are considered to be analogs. Throughout this specification, reference to the sugar portion of a nucleic acid species shall be understood to refer to either a true sugar or to a species taking the structural place of the sugar of wild type nucleic acids. Moreover, reference to inter-sugar linkages shall be taken to include moieties serving to join the sugar or sugar analog portions in the fashion of wild type nucleic acids.

In some embodiments, the present disclosure concerns modified oligonucleotides. e.g., oligonucleotide analogs or oligonucleosides, and methods for effecting the modifications. These modified oligonucleotides and oligonucleotide analogs may exhibit increased chemical and/or enzymatic stability relative to their naturally occurring counterparts. Extracellular and intracellular nucleases generally do not recognize and therefore do not bind to the backbone-modified compounds. When present as the protonated acid form, the lack of a negatively charged backbone may facilitate cellular penetration.

In some embodiments, modified internucleoside linkages are intended to replace naturally-occurring phosphodiester-5′-methylene linkages with four atom linking groups to confer nuclease resistance and enhanced cellular uptake to the resulting compound.

In some embodiments, modifications may be achieved using solid supports which may be manually manipulated or used in conjunction with a DNA synthesizer using methodology commonly known to those skilled in DNA synthesizer art. Generally, as a non-limiting example, the procedure involves functionalizing the sugar moieties of two nucleosides which will be adjacent to one another in the selected sequence. In a 5′ to 3′ sense, an “upstream” synthon such as structure H is modified at its terminal 3′ site, while a “downstream” synthon such as structure H1 is modified at its terminal 5′ site.

In some embodiments, oligonucleosides linked by hydrazines, hydroxylarnines, and other linking groups can be protected by a dimethoxytrityl group at the 5′-hydroxyl and activated for coupling at the 3′-hydroxyl with cyanoethyldiisopropyl-phosphite moieties. These compounds can be inserted into any desired sequence by standard, solid phase, automated DNA synthesis techniques. For example, one of the more popular processes is the phosphoramidite technique. In some embodiments, oligonucleotides containing a uniform backbone linkage can be synthesized by use of CPG-solid support and standard nucleic acid synthesizing machines such as Applied Biosystems Inc. 380B and 394 and Milligen/Biosearch 7500 and 8800s. For example, the initial nucleotide (number 1 at the 3′-terminus) is attached to a solid support such as controlled pore glass. In sequence specific order, each new nucleotide is attached either by manual manipulation or by the automated synthesizer system.

In some embodiments, free amino groups can be alkylated with, for example acetone and sodium cyanoboro hydride in acetic acid. The alkylation step can be used to introduce other, useful, functional molecules on the macromolecule. Such useful functional molecules include but are not limited to reporter molecules. RNA cleaving groups, groups for improving the pharmacokinetic properties of an oligonucleotide, and groups for improving the pharmacodynamic properties of an oligonucleotide. Such molecules can be attached to or conjugated to the macromolecule via attachment to the nitrogen atom in the backbone linkage. Alternatively, such molecules can be attached to pendent groups extending from a hydroxyl group of the sugar moiety of one or more of the nucleotides. Examples of such other useful functional groups are provided by WO1993007883, which is herein incorporated by reference. and in other of the above-referenced patent applications.

Solid supports may include any of those known in the art for polynucleotide synthesis, including controlled pore glass (CPG), oxalyl controlled pore glass. TentaGel Support—an aminopolyethyleneglycol derivatized support or Poros—a copolymer of polystyrene/divinylbenzene. Attachment and cleavage of nucleotides and oligonucleotides can be effected via standard procedures. As used herein, the term solid support further includes any linkers (e.g., long chain alkyl amines and succinyl residues) used to bind a growing oligonucleoside to a stationary phase such as CPG. In some embodiments, the oligonucleotide may be further defined as having one or more locked nucleotides, ethylene bridged nucleotides. peptide nucleic acids, or a 5′ (E)-vinyl-phosphonate (VP) modification. In some embodiments, an inhibitory oligonucleotide has one or more phosphorothioated DNA or RNA bases.

### B. Proteins and Polypeptides

In some embodiments, disclosed herein are compositions and methods that relate to the use of proteins or polypeptides that inhibit activity and/or protein levels of SIRPα and/or CD47. In some embodiments, such a protein or polypeptide specifically inhibits activity and/or protein levels or neuronal SIRPα and/or neuronal CD47.

In some embodiments, disclosed herein are compositions and methods that relate to the use of proteins or polypeptides that inhibit activity and/or protein levels of an inhibitor of SIRPα and/or CD47 protein activity and/or levels (e.g., said protein or polypeptide acts in a promotive manner for SIRPα and/or CD47 protein activity and/or levels). In some embodiments, such proteins or polypeptides that inhibit activity and/or protein levels of an inhibitor of SIRPα and/or CD47 protein inhibits a negative regulator of SIRPα and/or CD47 protein activity and/or levels.

In some embodiments, disclosed herein are compositions and methods that relate to the use of proteins or polypeptides that act to promote activity and/or protein levels of SIRPα and/or CD47. In some embodiments, such a protein or polypeptide specifically promotes activity and/or protein levels or neuronal SIRPα and/or neuronal CD47.

As used herein, a “protein” or “polypeptide” refers to a molecule comprising at least five amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In some embodiments, technologies provided herein comprise use of an anti-SIRPα antibody. In some embodiments, an anti-SIRPα antibody can be but is not limited to one or more of ADU-1805 (see e.g., Voets, E., Paradé, M., Lutje Hulsik, D, et al. Functional characterization of the selective pan-allele anti-SIRPα antibody ADU-1805 that blocks the SIRPα-CD47 innate immune checkpoint. j, immunotherapy cancer 7, 340 (2019)), humanized AB21 (hAB21) (see e.g., Kuo. T. C., Chen, A., Harrabi, O, et al. Targeting the myeloid checkpoint receptor SIRPα potentiates innate and adaptive immune responses to promote anti-tumor activity. J Hematol Oncol 13, 160 (2020)), humanized 1H9 (see e.g., Liu et al., JCI Insight. 2020; 5 (12): e134728), and BI 765063 (OSE-172) (see e.g., “OSE immunotherapeutics” clinical trial NCT03990233).

In some embodiments, technologies provided herein comprise use of an anti-CD47, antibody. In some embodiments, an anti-CD47 antibody can be but is not limited to one or more of Magrolimab (see e.g., Sallman et al., 2020, J, of Clin Oncology, Vol 38, Issue 15), Hu5F9-G4, CC-90002. TTI-621, ALX148, SRF231, SHR-1603, and IBI188 (see e.g., Zhang et al., 2020. Advances in Anti-Tumor treatments targeting the CD47/SIRPα Axis. Front. Immunol.).

In some embodiments, a protein or polypeptide is a mimetic, antagonist, and/or agonist of CD47 and/or SIRPα. In some embodiments, a CD47 and/or SIRPα inhibitor comprises the polypeptide Velcro-CD47 (N3612) (see e.g., Ho et al., “Velcro” Engineering of High Affinity CD47 ectodomain as signal regulatory protein alpha antagonist that enhances antibody-dependent cellular phagocytosis. J Biol Chem 2015 May 15;290 (20): 12650-12663). Velcro-CD47 binds with high affinity to the two most prominent human SIRPα alleles with greatly increased affinity relative to wild-type CD47 and potently antagonizes CD47 binding to SIRPα on human macrophages. In some embodiments, Velcro-CD47 synergizes with monoclonal antibodies to enhance phagocytosis of cells.

In certain embodiments the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of any of SEQ ID Nos: 1-10.

In some embodiments, the protein or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) of any of SEQ ID Nos: 3-6 or 9-10.

In some embodiments, the protein, polypeptide, or nucleic acid may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) or more contiguous amino acids or nucleic acids from any of SEQ ID NOs: 1-10.

In some embodiments, the polypeptide, protein, or nucleic acid may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleic acids of any of SEQ ID NOs: 1-10 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of any of SEQ ID NOs: 1-10.

In some aspects there is a nucleic acid molecule or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 of any of SEQ ID NOs: 1-10 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleic acids of any of SEQ ID NOs: 1-10.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

It is contemplated that in compositions of the disclosure, the concentration of oligonucleotide in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

In some embodiments, the amino acid subunits of a protein are modified to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

### C. Vectors and Transgenes

In some embodiments, disclosed herein are compositions and methods that relate to the use of vectors that inhibit gene activity, protein levels, and/or protein activity of SIRPα and/or CD47. In some embodiments, such vectors that inhibit gene activity, protein levels, and/or protein activity inhibits activity and/or protein levels or neuronal SIRPα and/or neuronal CD47.

In some embodiments, disclosed herein are compositions and methods that relate to the use of vectors that inhibit activity and/or protein levels of an inhibitor of SIRPα and/or CD47 protein activity and/or levels (e.g., said protein or polypeptide acts in a promotive manner for SIRPα and/or CD47 protein activity and/or levels). In some embodiments, such vectors that inhibit activity and/or protein levels of an inhibitor of SIRPα and/or CD47 protein inhibits a negative regulator of SIRPα and/or CD47 protein activity and/or levels.

In some embodiments, disclosed herein are compositions and methods that relate to the use of vectors that act to promote activity and/or protein levels of SIRPα and/or CD47. In some embodiments, such a vector specifically promotes activity and/or protein levels or neuronal SIRPα and/or neuronal CD47.

In some embodiments, a vector comprises a transgene. As used herein, a “transgene” is any exogenous oligonucleotide that acts as a template for transcription and/or translation of a functional product.

Suitable methods for nucleic acid delivery to effect expression of compositions are anticipated to include virtually any method by which a nucleic acid (e.g., DNA, RNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. No. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550.318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. No. 5,591,616 and 5.563.055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

In some embodiments, a pharmaceutical composition for use as described herein comprises a vector. In some embodiments, a vector is an oligonucleotide vector (e.g., a plasmid, a recombinant viral genome (e.g., a retrovirus genome, e.g., an adeno associated virus (AAV) genome), an artificial chromosome (e.g., a bacterial artificial chromosome, a yeast artificial chromosome, a human artificial chromosome, etc.), etc.), that encodes a functional molecule of interest (e.g., an RNA molecule, a protein, a polypeptide, etc.).

In some embodiments, a vector is a nucleic acid molecule. In some embodiments, a vector may be used to express quantities of proteins and/or polypeptides. In some embodiments, if the nucleic acid molecules are derived from a non-human animal, a sequence may be humanized and/or otherwise rendered suitable for administration to a human. In some embodiments, a nucleic acid molecule may comprise a coding region that has been codon optimized for expression in a human.

As used herein, the term “cDNA” refers to complementary DNA and corresponds to a DNA molecule, usually synthesized from a single-stranded RNA (such as, e.g., a messenger RNA [mRNA] or a microRNA [miRNA] template in a reaction catalyzed by a reverse transcriptase. In particular, when a cDNA is obtained from reverse transcription of a mRNA, it does not comprise an entire gene coding from a protein, but only the coding sequence of said protein (e.g., exons without introns). In some embodiments, a fragment of cDNA can comprise a part of said cDNA encoding the N-terminal part or the C-terminal part of a protein. Such fragment could be useful, e.g., in the case of large cDNAs which cannot be carried by a single viral vector and would thus require the use of, e.g., dual, triple. quadruple, etc., viral vector systems.

In some embodiments, a vector includes at least a fragment of a cDNA sequence comprising a sequence encoding a functional and/or structural portion of an RNA molecule. In some embodiments, such an RNA molecule may be a ribosomal RNA, transfer RNA, small nuclear RNA, small nucleolar RNA, micro RNA, long non-coding RNA, short interfering RNA, guide RNA, and/or any functional RNA species.

In some embodiments, vectors comprise a nucleic acid molecule encoding a polypeptide of a desired sequence or a portion thereof (e.g., a fragment containing one or more active and/or characteristic regions of a polypeptide, e.g., ligand binding domains, inhibitory domains, CDRs, variable region domains, etc.).

In some embodiments, vectors comprising nucleic acid molecules may encode an antibody heavy chain, light chain, alpha chain, beta chain, antigen-binding portion thereof, or any suitable combination thereof. In some embodiments, vectors comprising nucleic acid molecules may encode fusion proteins, modified antibodies, antibody fragments, and probes thereof. In addition, in some embodiments, a vector comprising nucleic acid molecules can comprise control sequences that govern transcription, translation, sub-cellular localization, tissue expression, temporal expression, etc. In some embodiments, vectors and expression vectors may contain nucleic acid sequences that serve other functions as well.

In a non-limiting example, to express one or more proteins or polypeptides suitable for use in the present disclosure, nucleic acids (e.g., DNA or RNA) encoding said protein or polypeptides are inserted into expression vectors such that the coding region for said protein or polypeptide is operatively linked to one or more transcriptional and/or translational control sequences. For example, in some embodiments, a vector encodes a functionally complete human CH immunoglobulin. CL immunoglobulin, and/or TCR sequence.

In some embodiments, where appropriate, vectors used herein contain sequences for plasmid or virus genome maintenance and for cloning and expression of exogenous nucleotide sequences. In some embodiments, such sequences, collectively are referred to as “flanking sequences”, and typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

In some embodiments, a promoter is a ubiquitous promoter. In some embodiments, a ubiquitous promoter can be but is not limited to CMV, CBA (including its derivatives CAG, CBh, and the like), EF-la, PGK, UBC, GUSB (hGBp), and UCOE promoters.

In some embodiments, tissue- or cell-specific expression elements can be used to restrict expression to certain cell types, such as CNS promoters which can be used to restrict expression to the nervous system, neurons, subtypes of neurons, or glial cells such as astrocytes or oligodendrocytes.

In some embodiments, a promoter is a nervous system specific promoter. In some embodiments, a promoter is a neuron-specific promotor. In some embodiments, a promoter is a microglia-specific promotor. In some embodiments, nervous system specific promoters can be one or more of but is not limited to, neuron-specific enolase (NSE) promoter, platelet-derived growth factor (PDGF) promoter, platelet-derived growth factor B-chain (PDGFB) promoter, synapsin (Syn) promoter, methyl-CpG binding protein 2 (MeCP2) promoter, Ca2+/calmodulin-dependent protein kinase II (CaMKII) promoter, metabotropic glutamate receptor 2 (mGluR2) promoter, neurofilament light (NFL) promoter, neurofilament heavy (NFH) promoter, β-globin minigene ηβ2 promoter, preproenkephalin (PPE) promoter, enkephalin (Enk) promoter, and excitatory amino acid transporter 2 (EAAT2) promoter.

In some embodiments, a promoter is a SIRPα endogenous promoter.

In some embodiments, a promoter is a CD47 endogenous promoter.

In some embodiments, a promoter is a CAG promoter.

In some embodiments, a promoter is a CMV promoter.

In some embodiments, a vector is a recombinant viral vector, e.g., a recombinant adeno-associated virus (AAV). In one embodiment, an AAV vector according to the present disclosure is selected from natural serotypes AAV1, AAV2, AAV3, AAV4, AAV5, AAV6. AAV7, AAV8, AAV9, AAV10. AAV11, and AAV12; or pseudotypes, chimeras, and variants thereof.

As used herein, the term “pseudotype” when referring to an AAV vector, or a “pseudotyped AAV vector”, refers to an AAV vector which comprises the genome of one AAV serotype packaged in the capsid of another AAV serotype. These pseudotypes are denoted using a slash or a hyphen, so that “AAV2/5” or “AAV2-5” indicates an AAV vector comprising a serotype 2 genome, packaged into a serotype 5 capsid. Examples of pseudotyped AAV vectors include, but are not limited to, AAV2/1, AAV2/2, AAV2/3, AAV2/4, AAV2/5, AAV2/6. AAV2/7, AAV2/8 and AAV2/9.

As used herein, the term “chimera” when referring to an AAV vector, or a “chimeric AAV vector”, refers to an AAV vector which comprises a capsid containing VP1, VP2, and VP3 proteins from at least two different AAV serotypes; or alternatively, which comprises VP1, VP2, and VP3 proteins, at least one of which comprises at least a portion from another AAV serotype. Examples of chimeric AAV vectors include, but are not limited to, AAV-DJ, AAV2G9, AAV218. AAV2i8G9, AAV8G9, and AAV9i1.

In some embodiments, AAV variants include vectors which have been genetically modified, e.g., by substitution, deletion or addition of one or several amino acid residues in one of the capsid proteins. Examples of such variants include, but are not limited to, AAV2 with one or more of Y444F. Y500F. Y730F and/or S662V mutations; AAV3 with one or more of Y705F, Y731F and/or T492V mutations; AAV6 with one or more of S663V and/or T492V mutations, etc.

In some embodiments, a viral vector may be modified to comprise at least one surface-bound modification such as but not limited to a surface bound saccharide, lipid, carbohydrate, small molecule, etc.

In some embodiments, a viral vector suitable for use according to the present disclosure, is to be administrated at a dose ranging from about 10⁸ viral genomes (vg) to about 10¹⁵ vg, such as from about 10⁸ vg to about 10¹⁴ vg, from about 10⁸ vg to about 10¹³ vg, from about 10⁸ vg to about 10¹² vg, from about 10⁸ vg to about 10¹¹ vg, from about 10⁸ vg to about 10¹⁰ vg, from about 10⁸ vg to about 10⁹ vg, from about 10⁹ vg to about 10¹⁵ vg, from about 10⁹ vg to about 10¹⁴ vg, from about 10⁹ vg to about 10¹³ vg, from about 10⁹ vg to about 10¹² vg, from about 10⁹ vg to about 10¹¹ vg, from about 10⁹ vg to about 10¹⁰ vg, from about 10¹⁰ vg to about 10¹⁵ vg, from about 10¹⁰ vg to about 10¹⁴ vg, from about 10¹⁰ vg to about 10¹³ vg, from about 10¹⁰ vg to about 10¹² vg, from about 10¹⁰ vg to about 10¹¹ vg, from about 10¹¹ vg to about 10¹⁵ vg, from about 10¹¹ vg to about 10¹⁴ vg, from about 10¹¹ vg to about 10¹³ vg, from about 10¹¹ vg to about 10¹² vg, from about 10¹² vg to about 10¹⁵ vg, from about 10¹² vg to about 1014 vg, from about 10¹² vg to about 10¹³ vg, from about 10¹³ vg to about 10¹⁵ vg.

The term “vector genome”, abbreviated as “vg”, refers to one or more polynucleotides comprising a set of the polynucleotide sequences of a vector, e.g., a viral vector. A vector genome may be encapsidated in a viral particle. Depending on the particular viral vector, a vector genome may comprise single-stranded DNA, double-stranded DNA, or single-stranded RNA, or double-stranded RNA. A vector genome may include endogenous sequences associated with a particular viral vector and/or any heterologous sequences inserted into a particular viral vector through recombinant techniques (e.g., a transgene). In some embodiments, the nucleic acid titer of a viral vector may be measured in terms of vg/mL. Methods suitable for measuring this titer are known in the art, and include, e.g., quantitative PCR.

In some embodiments, a dose of viral vector (e.g., AAV vector) required to achieve a desired effect or a therapeutic effect will vary based on several factors including, but not limited to, the specific route of administration, the level of gene, RNA or protein expression required to achieve a therapeutic effect, the specific disease being treated, and the stability of the gene, RNA or protein product.

In some embodiments, the volume of a viral vector administered to a subject is of about 1 μL±0.5μ, about 2 μL±0.5μ, about 3 μL±0.5μ, about 4 μL±0.5μ, about 5 μL±0.5 μL, about 6 μL±0.5 L, about 7 μL±0.5 μL, about 8 μL±0.5 μL, about 9 μL±0.5 μL, about 10 μL±0.5 μL, about 15 μL±5 μL, about 20 μL±5 μL, about 25 μL±5 μL, about 30 μL±5 μL, about 35 μL±5 μL, about 40 L±5 μL, about 45 μL±5 μL, about 50 μL±5 μL, about 55 μL±5 μL. about 60 μL±5 μL, about 65 μL±5 μL, about 70 μL±5 μL, about 75 μL±5 μL, about 80 μL±5 μL, about 85 μL±5 μL, about 90 μL±5 μL, about 95 μL±5 μL, about 100 μL±5 μL, about 150 μL±50 μL, about 200 μL±50 μL, about 250 μL±50 μL, about 300 μL±50 μL, about 350 μL±50 μL, about 400 μL±50 μL, about 450 μL±50 μL, about 500 μL±50 μL, about 550 μL±50 μL, about 600 μL±50 μL, about 650 μL±50 μL, about 700 μL±50 μL, about 750 μL±50 μL, about 800 μL±50 μL, about 850 μL±50 μL, about 900 μL±50 μL, about 950 μL±50 μL, about 1000 μL±50 μL, about 1.5 mL±250 μL, about 2 mL±250 μL, about 2.5 mL±250 μL, about 3 mL±250 μL, about 3.5 mL±250 μL, about 4 mL±250 μL, about 4.5 mL±250 μL, about 5 mL±250 μL. about 5.5 mL±250 μL, about 6 mL±250 μL, about 6.5 mL±250 μL, about 7 mL±250 μL, about 7.5 mL±250 μL, about 8 mL±250 μL, about 8.5 mL±250 μL, about 9 mL±250 μL, about 9.5 mL±250 μL, about 10 mL±250 μL.

In some embodiments, the rate of administration of a viral vector administered to a subject will also depend, among other things, on the size of the subject, the dose of the viral vector, the volume of the viral vector, and the route of administration. In one non-limiting example, for intracerebral administration, a rate of administration ranging from about 0.1 μL/min to about 1 μL/min or from about 1 μL/min to about 5 μL/min or from about 1 μL/min to about 10 μL/min may be used.

In some embodiments, the rate of administration of a viral vector administered to a subject is about 0.1 L/min±0.05 μL/min, about 0.2 μL/min±0.05 μL/min, about 0.3 L/min±0.05 μL/min, about 0.4 μL/min±0.05 μL/min, about 0.5 μL/min±0.05 μL/min, about 0.6 μL/min±0.05 μL/min, about 0.7 μL/min±0.05 μL/min, about 0.8 μL/min±0.05 μL/min, about 0.9 μL/min±0.05 μL/min, about 1 μL/min±0.5 μL/min, about 2 μL/min±0. μL/min, about 3 μL/min±0.5 μL/min, about 4 μL/min±0.5 μL/min, about 5 μL/min±0.5 L/min, about 6 μL/min±0.5 L/min, about 7 μL/min±0.5 μL/min, about 8 μL/min±0.5 L/min, about 9 μL/min±0.5 L/min, or about 10 μL/min±0.5 L/min.

In some embodiments, a total dose or total volume of viral vectors may be administered continuously (e.g., wherein the total dose or total volume of viral vector is injected in a single shot or infusion); or discontinuously (e.g., wherein fractions of the total dose or total volume of viral vectors are injected with intermittent periods between each shot, preferably with short intermittent periods such as periods of time of 15 seconds, 30 seconds, 45 seconds, 1 minute, 2 minutes, 3 minutes, 4 minutes, or 5 minutes between each shot or infusion).

### D. Small Molecules

In some embodiments, disclosed herein are compositions and methods that relate to the use of small molecules that inhibit gene activity, protein levels, and/or protein activity of SIRPα and/or CD47. In some embodiments, such small molecules that inhibit gene activity, protein levels, and/or protein activity inhibits activity and/or protein levels or neuronal SIRPα and/or neuronal CD47.

In some embodiments, disclosed herein are compositions and methods that relate to the use of small molecules that inhibit activity and/or protein levels of an inhibitor of SIRPα and/or CD47 protein activity and/or levels (e.g., said small molecule acts in a promotive manner for SIRPα and/or CD47 protein activity and/or levels). In some embodiments, such small molecules that inhibit activity and/or protein levels of an inhibitor of SIRPα and/or CD47 protein inhibits a negative regulator of SIRPα and/or CD47 protein activity and/or levels.

In some embodiments, disclosed herein are compositions and methods that relate to the use of small molecules that act to promote activity and/or protein levels of SIRPα and/or CD47. In some embodiments, such a small molecule specifically promotes activity and/or protein levels of neuronal SIRPα and/or neuronal CD47, and/or microglial SIRPα.

In some embodiments, a small molecule inhibitor of CD47 and/or SIRPα is utilized in conjunction with one or more additional therapeutic agents.

In some embodiments, a small molecule inhibitor of CD47 and/or SIRPα protein comprises 4-methylumbelliferone (4Mu) (see e.g., Rodriquez et al., 4Mu decreases CD47 expression on hepatic cancer stem cells and primes a potent antitumor T cell response induced by interleukin-12. Mol Ther. (2018)). 4Mu induces apoptosis and reduces inflammation, steatosis, and the expression of cancer stem cells markers. In the presence of cancer stem cells, 4Mu has been shown to downregulate the expression of CD47 on cells and promotes phagocytosis of antigen presenting cells.

In some embodiments, a small molecule inhibitor of CD47 and/or SIRPα protein activity is RRx-001 (also called ABDNAZ; from EpicentRx), which has the chemical name 2-bromo-1-(3,3-dinitroazetidin-1-yl) ethan-1-one. RRx-001 a small cyclic nitro compound that has previously been found to induce a number of enzymatic and epigenetic alterations in tumor cells. RRx-001 has been used clinically in combination with chemotherapy and/or radiation as a chemo- and radiosensitizer and is described in, for example, international patent application publication WO 2007/022225 describing various compounds and their use in treating medical disorders, such as cancer. Exemplary scientific publications describing benefits observed in human clinical trials evaluating efficacy of RRx-001 in treating patients suffering from cancer include Carter et al, in Respir. Med. Case Rep. (2016) vol. 18, pages 62-65; Kim et al, in Transl. Oncol. (2016) vol. 9 (2), pages 108-113; and Reid et al, in Case Rep. Oncol. (2014) vol. 7 (1), pages 79-85.

### E. Cells

In some embodiments, technologies provided herein utilize cells. In some embodiments, one or more recombinant expression vectors are and/or have been introduced to a cell. In some embodiments, functional proteins or polypeptides (e.g., Antibodies and/or fragments thereof) can be expressed in cell types suitable for administration to a subject. In some embodiments, a cell suitable for administration to a subject is created using methods known in the art. For example, with stable transfection of mammalian cells, it is known, depending upon the expression vector and transfection technique used, that only a small fraction of cells may integrate the foreign DNA into their genome. Therefore, in some embodiments, in order to identify and select these integrants, a selectable marker (e.g., for resistance to antibiotics) can also be introduced into the cells along with a gene of interest. Cells stably transfected with the introduced nucleic acid can be identified by drug selection (e.g., cells that have incorporated the selectable marker gene will survive, while the other cells die), among other methods known in the arts.

In particular embodiments, cells of the present disclosure may be specifically formulated and/or they may be cultured in a particular medium. In some embodiments, cells may be formulated in such a manner as to be suitable for delivery to a recipient without deleterious effects.

In some embodiments, cell growth medium can be prepared using a medium used for culturing animal cells as their basal medium, such as any of AIM V, X-VIVO-15, NeuroBasal, EGM2. TeSR, BME, BGJb, CMRL 1066, Glasgow MEM, Improved MEM Zinc Option, IMDM, Medium 199, Eagle MEM, aMEM, DMEM, Ham, RPMI-1640, and Fischer's media, as well as any combinations thereof, but the medium may not be particularly limited thereto as far as it can be used for culturing animal cells. Particularly, in some embodiments, the medium may be xeno-free or chemically defined.

In some embodiments, cell medium can be a serum-containing or serum-free medium, or xeno-free medium. From the aspect of preventing contamination with heterogeneous animal-derived components, serum can be derived from the same animal as that of the target subject. In some embodiments, serum-free medium refers to medium with no unprocessed or unpurified serum and accordingly, can include medium with purified blood-derived components or animal tissue-derived components (such as growth factors).

In some embodiments, cell medium may contain or may not contain any alternatives to serum. In some embodiments, alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. In some embodiments, alternatives to serum can be prepared by the method disclosed in International Publication No. 98/30679, for example (incorporated herein in its entirety). Alternatively, in some embodiments, any commercially available suitable materials can be used for more convenience. In some embodiments, commercially available materials include knockout Serum Replacement (KSR). Chemically-defined Lipid concentrated (Gibco), and/or Glutamax (Gibco).

In certain embodiments, cell medium may comprise one, two, three, four, five, six, seven, eight, nine, ten, 11, 12, 13, 14, 15, 16, 17, 18, 19, 20 or more of the following: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HCl; Linoleic Acid; Linolenic Acid; Progesterone; Putrescine 2HCl; Sodium Selenite; and/or T3 (triodo-I-thyronine). In specific embodiments, one or more of the aforementioned chemicals may be explicitly excluded.

In some embodiments, cell medium further comprises vitamins. In some embodiments, the medium comprises 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or the medium includes combinations thereof or salts thereof. In some embodiments, the medium comprises or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B12. In some embodiments, the vitamins include or consist essentially of biotin, DL alpha tocopherol acetate, DL alpha-tocopherol, vitamin A, or combinations or salts thereof. In some embodiments, the medium further comprises proteins. In some embodiments, the proteins comprise albumin or bovine serum albumin, a fraction of BSA, catalase, insulin, transferrin, superoxide dismutase, or combinations thereof. In some embodiments, the medium further comprises one or more of the following: corticosterone, D-Galactose, ethanolamine, glutathione. L-carnitine, linoleic acid, linolenic acid, progesterone, putrescine, sodium selenite. or triodo-I-thyronine, or combinations thereof. In some embodiments, the medium comprises one or more of the following: a B-27® supplement, xeno-free B-27® supplement, GS21TM supplement, or combinations thereof. In some embodiments, the medium comprises or further comprises amino acids, monosaccharides, inorganic ions. In some embodiments, the amino acids comprise arginine, cystine, isoleucine, leucine, lysine, methionine, glutamine, phenylalanine, threonine, tryptophan, histidine, tyrosine, or valine, or combinations thereof. In some embodiments, the inorganic ions comprise sodium, potassium, calcium, magnesium, nitrogen, or phosphorus, or combinations or salts thereof. In some embodiments, the medium further comprises one or more of the following: molybdenum, vanadium, iron, zinc, selenium, copper, or manganese, or combinations thereof. In certain embodiments, the medium comprises or consists essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triodo-I-thyronine, a B-27® supplement, xeno-free B-27® supplement, GS21TM supplement, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese. In specific embodiments, one or more of the aforementioned chemicals may be explicitly excluded.

In some embodiments, cell medium can also contain one or more externally added fatty acids or lipids, amino acids (such as non-essential amino acids), vitamin(s), growth factors, cytokines, antioxidant substances, 2-mercaptoethanol, pyruvic acid, buffering agents, and/or inorganic salts. In specific embodiments, one or more of these aforementioned chemicals may be explicitly excluded.

In some embodiments, one or more cell medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein.

In specific embodiments, cells of the immediate disclosure are specifically formulated. In some embodiments, they may or may not be formulated as a cell suspension. In specific cases, cells are formulated in a single dose form. In some embodiments, cells can be formulated for systemic or local administration. In some cases, cells are formulated for storage prior to use. In some embodiments, cell formulations may comprise one or more cryopreservation agents, such as DMSO (e.g., in 1% DMSO, 2% DMSO, 3% DMSO, 4% DMSO, 5% DMSO, etc.). In some embodiments, cell formulations may comprise albumin, including human albumin (e.g., 1%, 1.5%, 2%, or 2.5% human albumin). In some embodiments, cells may be formulated specifically for intravenous administration; for example, they are formulated for intravenous administration over less than one hour. In some embodiments, cells may be formulated specifically for intracerebroventricular administration. In particular embodiments, the cells are in a formulated cell suspension that is stable at room temperature for 1, 2, 3, or 4 hours or more from time of thawing.

In particular embodiments, cells of the present disclosure comprise an exogenous T cell receptor (TCR), which may be of a defined antigen specificity. In some embodiments, the TCR can be selected based on absent or reduced alloreactivity to the intended recipient. In the example where the exogenous TCR is non-alloreactive, during T cell differentiation the exogenous TCR suppresses rearrangement and/or expression of endogenous TCR loci through a developmental process called allelic exclusion, resulting in T cells that express only the non-alloreactive exogenous TCR and are thus non-alloreactive. In some embodiments, the choice of exogenous TCR may not necessarily be defined based on lack of alloreactivity. In some embodiments, the endogenous TCR genes have been modified by genome editing so that they do not express a protein. Methods of gene editing such as methods using the CRISPR/Cas system are known in the art and described briefly herein.

In some embodiments, cells of the immediate disclosure further comprise one or more chimeric antigen receptors (CARs). In some embodiments, a CAR may be directed to SIRPα. In some embodiments, a CAR may be directed to CD47. In some embodiments, a CAR may be directed to SIRPα and CD47. A CAR may be a first, second, third, or more generation CAR. In some embodiments, a CAR may be bispecific for any two nonidentical antigens, or it may be specific for more than two nonidentical antigens.

In some embodiments, when a CAR is directed to SIRPα, one or more antigen recognition domains can be derived from an anti-SIRPα antibody. In some embodiments, an anti-SIRPα antibody is ADU-1805 (see e.g., Voets, E., Paradé, M., Lutje Hulsik, D, et al. Functional characterization of the selective pan-allele anti-SIRPα antibody ADU-1805 that blocks the SIRPα-CD47 innate immune checkpoint. j, immunotherapy cancer 7, 340 (2019)), humanized AB21 (hAB21) (see e.g., Kuo. T. C., Chen, A., Harrabi, O, et al. Targeting the myeloid checkpoint receptor SIRPα potentiates innate and adaptive immune responses to promote anti-tumor activity. J Hematol Oncol 13, 160 (2020)), humanized 1H9 (see e.g., Liu et al., JCI Insight. 2020; 5 (12): e134728), or BI 765063 (OSE-172) (scc e.g., “OSE immunotherapeutics” clinical trial NCT03990233).

In some embodiments, when a CAR is directed to CD47, one or more antigen recognition domains can be derived from an anti-CD47 antibody. In some embodiments, an anti-CD47 antibody is Magrolimab (see e.g., Sallman et al., 2020. J, of Clin Oncology. Vol 38. Issue 15), Hu5F9-G4, CC-90002. TTI-621, ALX148, SRF231, SHR-1603, or IBI188 (see e.g., Zhang et al., 2020. Advances in Anti-Tumor treatments targeting the CD47/SIRPα Axis. Front. Immunol.).

In some embodiments, additional CARs may be utilized, for example a CAR may be directed include at least 5T4, 8H9, avB6 integrin, BCMA, B7-H3, B7-H6, CAIX, CA9, CD19, CD20, CD22, CD30, CD33, CD38, CD44, CD44v6, CD44v7/8, CD70, CD123, CD138. CD171, CEA, CSPG4, EGFR, EGFR family including ErbB2 (HER2), EGFRVIII, EGP2, EGP40, ERBB3, ERBB4, ErbB3/4, EPCAM, EphA2, EpCAM, folate receptor-a. FAP. FBP, fetal AchR. FR. GD2. G250/CAIX, GD3, Glypican-3 (GPC3). Her2. IL-13R2. Lambda. Lewis-Y, Kappa, KDR, MAGE, MCSP, Mesothelin, Muc1, Muc16, NCAM, NKG2D Ligands, NY-ESO-1, PRAME, PSC1, PSCA, PSMA, RORI, SP17, Survivin. TAG72. TEMs, carcinoembryonic antigen. HMW-MAA, AFP. CA-125, ETA. Tyrosinase, MAGE, laminin receptor, HPV E6, E7, BING-4, Calcium-activated chloride channel 2, Cyclin-B1,9D7, EphA3. Telomerase, SAP-1, BAGE family, CAGE family, GAGE family, MAGE family, SAGE family. XAGE family, NY-ESO-1/LAGE-1, PAME. SSX-2, Melan-A/MART-1, GP100/pmel17. TRP-1/-2, P, polypeptide, MC1R, Prostate-specific antigen, β-catenin, BRCA1/2, CML66, Fibronectin, MART-2. TGF-βRII, or VEGF receptors (e.g., VEGFR2), for example.

### F. Genome Editing Systems

In some embodiments, it is contemplated that a genome editing systems, e.g., those comprising nucleases, can be utilized to modify SIRPα and/or CD47 gene activity in the nervous system.

In some embodiments, genome (or gene) editing systems of the present disclosure include at least two components adapted from naturally occurring CRISPR systems: a guide RNA (gRNA) and an RNA-guided nuclease. These two components form a complex that is capable of associating with a specific nucleic acid sequence and editing the DNA in or around that nucleic acid sequence, for instance by making one or more single-strand breaks (a SSB or nick), a double-strand break (a DSB), and/or a point mutation.

Classically, naturally occurring CRISPR systems are organized evolutionarily into two classes and five (or potentially six) types (Makarova et al. Nat Rev Microbiol. 2011 June; 9 (6): 467-477 (“Makarova”)), and while genome editing systems of the present disclosure may adapt components of any type or class of naturally occurring CRISPR system, the embodiments presented herein are generally adapted from Class 2, and type II or V CRISPR systems. Class 2 systems, which encompass types II and V, are characterized by relatively large, multidomain RNA-guided nuclease proteins (e.g., Cas9 or Cpf1) and one or more guide RNAs (e.g., a crRNA and, optionally, a tracrRNA) that form ribonucleoprotein (RNP) complexes that associate with (i.e., target) and cleave specific loci complementary to a targeting (or spacer) sequence of the crRNA. Genome editing systems according to the present disclosure similarly target and edit cellular DNA sequences, but differ significantly from CRISPR systems occurring in nature. For example, the unimolecular guide RNAs described herein do not occur in nature, and both guide RNAs and RNA-guided nucleases according to this disclosure may incorporate any number of non-naturally occurring modifications.

Genome editing systems can be implemented (e.g, administered or delivered to a cell or a subject) in a variety of ways, and different implementations may be suitable for distinct applications. For instance, a genome editing system is implemented, in certain embodiments, as a protein/RNA complex (a ribonucleoprotein, or RNP), which can be included in a pharmaceutical composition that optionally includes a pharmaceutically acceptable carrier and/or an encapsulating agent, such as a lipid or polymer micro- or nano-particle, micelle, liposome, etc. In certain embodiments, a genome editing system is implemented as one or more nucleic acids encoding the RNA-guided nuclease and guide RNA components described above (optionally with one or more additional components); in certain embodiments, the genome editing system is implemented as one or more vectors comprising such nucleic acids, for instance a viral vector such as an adeno-associated virus (AAV); and in certain embodiments, the genome editing system is implemented as a combination of any of the foregoing. Additional or modified implementations that operate according to the principles set forth herein will be apparent to the skilled artisan and are within the scope of this disclosure.

It should be noted that the genome editing systems of the present disclosure can be targeted to a single specific nucleotide sequence, or may be targeted to—and capable of editing in parallel-two or more specific nucleotide sequences through the use of two or more guide RNAs. The use of multiple gRNAs is referred to as “multiplexing”, and can be employed to target multiple, unrelated target sequences of interest, or to form multiple SSBs or DSBs within a single target domain and, in some cases, to generate specific edits within such target domain. For example, International Patent Publication No. WO 2015/138510 by Maeder et al. (“Maeder”) describes a genome editing system for correcting a point mutation (C.2991+1655A to G) in the human CEP290 gene that results in the creation of a cryptic splice site, which in turn reduces or eliminates the function of the gene. The genome editing system of Maeder utilizes two guide RNAs targeted to sequences on either side of (i.e., flanking) the point mutation, and forms DSBs that flank the mutation. This, in turn, promotes deletion of the intervening sequence, including the mutation, thereby eliminating the cryptic splice site and restoring normal gene function.

As another example, WO 2016/073990 by Cotta-Ramusino, et al. (“Cotta-Ramusino”) describes a genome editing system that utilizes two gRNAs in combination with a Cas9 nickase (a Cas9 that makes a single strand nick such as S, pyogenes D10A), an arrangement termed a “dual-nickase system.” The dual-nickase system of Cotta-Ramusino is configured to make two nicks on opposite strands of a sequence of interest that are offset by one or more nucleotides, which nicks combine to create a double strand break having an overhang (5′ in the case of Cotta-Ramusino, though 3′ overhangs are also possible). The overhang, in turn, can facilitate homology directed repair events in some circumstances. And, as another example WO 2015/070083 by Palestrant et al. (“Palestrant”) describes a gRNA targeted to a nucleotide sequence encoding Cas9 (referred to as a “governing RNA”), which can be included in a genome editing system comprising one or more additional gRNAs to permit transient expression of a Cas9 that might otherwise be constitutively expressed, for example in some virally transduced cells. These multiplexing applications are intended to be exemplary, rather than limiting, and the skilled artisan will appreciate that other applications of multiplexing are generally compatible with the genome editing systems described here.

Genome editing systems can, in some instances, form double strand breaks that are repaired by cellular DNA double-strand break mechanisms such as NHEJ or HDR. These mechanisms are described throughout the literature, for example by Davis & Maizels, PNAS, 111 (10): E924-932. Mar. 11, 2014 (“Davis”) (describing Alt-HDR): Frit et al. DNA Repair 17 (2014) 81-97 (“Frit”) (describing Alt-NHEJ); and Iyama and Wilson III, DNA Repair (Amst.) 2013-Aug; 12 (8): 620-636 (“Iyama”) (describing canonical HDR and NHEJ pathways generally).

Where genome editing systems operate by forming DSBs, such systems optionally include one or more components that promote or facilitate a particular mode of double-strand break repair or a particular repair outcome. For instance. Cotta-Ramusino also describes genome editing systems in which a single stranded oligonucleotide “donor template” is added; the donor template is incorporated into a target region of cellular DNA that is cleaved by the genome editing system, and can result in a change in the target sequence. In certain embodiments, a donor template is added that provides a sequence with gain or loss of function characteristics.

In certain embodiments, genome editing systems modify a target sequence, or modify expression of a target gene in or near the target sequence, without causing single- or double-strand breaks. For example, a genome editing system may include an RNA-guided nuclease fused to a functional domain that acts on DNA, thereby modifying the target sequence or its expression. As one example, an RNA-guided nuclease can be connected to (e.g., fused to) a cytidine deaminase functional domain, and may operate by generating targeted C-to-A substitutions. Exemplary nuclease/deaminase fusions are described in Komor et al. Nature 533, 420-424 (19 May 2016) (“Komor”). Alternatively, a genome editing system may utilize a cleavage-inactivated (i.e., a “dead”) nuclease, such as a dead Cas9 (dCas9), and may operate by forming stable complexes on one or more targeted regions of cellular DNA, thereby interfering with functions involving the targeted region(s) including, without limitation, mRNA transcription, chromatin remodeling, etc.

Guide RNAs (gRNAs) of the present disclosure may be unimolecular (comprising a single RNA molecule, and referred to alternatively as chimeric), or modular (comprising more than one, and typically two, separate RNA molecules, such as a crRNA and a tracrRNA, which are usually associated with one another, for instance by duplexing), gRNAs and their component parts are described throughout the literature, for instance in Briner et al. (Molecular Cell 56 (2), 333-339, Oct. 23, 2014 (“Briner”)), and in Cotta-Ramusino.

In bacteria and archaea, type II CRISPR systems generally comprise an RNA-guided nuclease protein such as Cas9, a CRISPR RNA (crRNA) that includes a 5′ region that is complementary to a foreign sequence, and a trans-activating crRNA (tracrRNA) that includes a 5′ region that is complementary to, and forms a duplex with, a 3′ region of the crRNA. While not intending to be bound by any theory, it is thought that this duplex facilitates the formation of—and is necessary for the activity of—the Cas9/gRNA complex. As type II CRISPR systems were adapted for use in gene editing, it was discovered that the crRNA and tracrRNA could be joined into a single unimolecular or chimeric guide RNA, in one non-limiting example, by means of a four nucleotide (e.g., GAAA) “tetraloop” or “linker” sequence bridging complementary regions of the crRNA (at its 3′ end) and the tracrRNA (at its 5′ end). (Mali et al. Science. 2013 Feb. 15; 339 (6121): 823-826 (“Mali”); Jiang et al. Nat Biotechnol.2013 March; 31 (3): 233-239 (“Jiang”); and Jinek et al., 2012 Science Aug.17; 337 (6096): 816-821 (“Jinek 2012”)).

Guide RNAs, whether unimolecular or modular, include a “targeting domain” that is fully or partially complementary to a target domain within a target sequence, such as a DNA sequence in the genome of a cell where editing is desired. Targeting domains are referred to by various names in the literature, including without limitation “guide sequences” (Hsu et al., Nat Biotechnol.2013 September; 31 (9): 827-832, (“Hsu”)). “complementarity regions” (Cotta-Ramusino), “spacers” (Briner) and generically as “crRNAs” (Jiang). Irrespective of the names they are given, targeting domains are typically 10-30 nucleotides in length, and in certain embodiments are 16-24 nucleotides in length (for instance, 16, 17, 18, 19, 20, 21, 22, 23 or 24 nucleotides in length), and are at or near the 5′ terminus of in the case of a Cas9 gRNA, and at or near the 3′ terminus in the case of a Cpf1 gRNA.

In addition to the targeting domains, gRNAs typically (but not necessarily, as discussed below) include a plurality of domains that may influence the formation or activity of gRNA/Cas9 complexes. For instance, as mentioned above, the duplexed structure formed by first and secondary complementarity domains of a gRNA (also referred to as a repeat: anti-repeat duplex) interacts with the recognition (REC) lobe of Cas9 and can mediate the formation of Cas9/gRNA complexes. (Nishimasu et al., Cell 156, 935-949. Feb. 27, 2014 (“Nishimasu 2014”) and Nishimasu et al., Cell 162, 1113-1126 Aug. 27, 2015 (“Nishimasu 2015”)). It should be noted that the first and/or second complementarity domains may contain one or more poly-A tracts, which can be recognized by RNA polymerases as a termination signal. The sequence of the first and second complementarity domains are, therefore, optionally modified to eliminate these tracts and promote the complete in vitro transcription of gRNAs, for instance through the use of A-G swaps as described in Briner, or A-U swaps. These and other similar modifications to the first and second complementarity domains are within the scope of the present disclosure.

Along with the first and second complementarity domains, Cas9 gRNAs typically include two or more additional duplexed regions that are involved in nuclease activity in vivo but not necessarily in vitro. (Nishimasu 2015). A first stem-loop one near the 3′ portion of the second complementarity domain is referred to variously as the “proximal domain,” (Cotta-Ramusino) “stem loop 1” (Nishimasu 2014 and 2015) and the “nexus” (Briner). One or more additional stem loop structures are generally present near the 3′ end of the gRNA, with the number varying by species: s. pyogenes gRNAs typically include two 3′ stem loops (for a total of four stem loop structures including the repeat: anti-repeat duplex), while S, aureus and other species have only one (for a total of three stem loop structures). A description of conserved stem loop structures (and gRNA structures more generally) organized by species is provided in Briner.

While the foregoing description has focused on gRNAs for use with Cas9, it should be appreciated that other RNA-guided nucleases have been (or may in the future be) discovered or invented which utilize gRNAs that differ in some ways from those described to this point. For instance, Cpf1 (“CRISPR from Prevotella and Franciscella 1”) is a recently discovered RNA-guided nuclease that does not require a tracrRNA to function. (Zetsche et al., 2015, Cell 163, 759-771 Oct. 22, 2015 (“Zetsche I”)). A gRNA for use in a Cpf1 genome editing system generally includes a targeting domain and a complementarity domain (alternately referred to as a “handle”). It should also be noted that, in gRNAs for use with Cpf1, the targeting domain is usually present at or near the 3′ end, rather than the 5′ end as described above in connection with Cas9 gRNAs (the handle is at or near the 5′ end of a Cpf1 gRNA).

Those of skill in the art will appreciate, however, that although structural differences may exist between gRNAs from different prokaryotic species, or between Cpf1 and Cas9 gRNAs, the principles by which gRNAs operate are generally consistent. Because of this consistency of operation, gRNAs can be defined, in broad terms, by their targeting domain sequences, and skilled artisans will appreciate that a given targeting domain sequence can be incorporated in any suitable gRNA, including a unimolecular or chimeric gRNA, or a gRNA that includes one or more chemical modifications and/or sequential modifications (substitutions, additional nucleotides, truncations, etc.). Thus, for economy of presentation in this disclosure, gRNAs may be described solely in terms of their targeting domain sequences.

More generally, skilled artisans will appreciate that some aspects of the present disclosure relate to systems, methods and compositions that can be implemented using multiple RNA-guided nucleases. For this reason, unless otherwise specified, the term gRNA should be understood to encompass any suitable gRNA that can be used with any RNA-guided nuclease, and not only those gRNAs that are compatible with a particular species of Cas9 or Cpf1. By way of illustration, the term gRNA can, in certain embodiments, include a gRNA for use with any RNA-guided nuclease occurring in a Class 2 CRISPR system, such as a type II or type V or CRISPR system, or an RNA-guided nuclease derived or adapted therefrom.

Methods for selection and validation of target sequences as well as off-target analyses have been described previously, e.g., in Mali; Hsu; Fu et al., 2014 Nat Biotechnol 32 (3): 279-84, Heigwer et al., 2014 Nat methods 11 (2): 122-3; Bae et al. (2014) Bioinformatics 30 (10): 1473-5; and Xiao A et al. (2014) Bioinformatics 30 (8): 1180-1182. As a non-limiting example, gRNA design may involve the use of a software tool to optimize the choice of potential target sequences corresponding to a user's target sequence, e.g., to minimize total off-target activity across the genome. While off-target activity is not limited to cleavage, the cleavage efficiency at each off-target sequence can be predicted, e.g., using an experimentally-derived weighting scheme. These and other guide selection methods are described in detail in Maeder and Cotta-Ramusino.

For example, methods for selection and validation of target sequences as well as off-target analyses can be performed using cas-offinder (Bae S, Park J, Kim J-S. Cas-OFFinder: a fast and versatile algorithm that searches for potential off-target sites of Cas9 RNA-guided endonucleases. Bioinformatics.2014; 30:1473-5). Cas-offinder is a tool that can quickly identify all sequences in a genome that have up to a specified number of mismatches to a guide sequence.

As another example, methods for scoring how likely a given sequence is to be an off-target (e.g., once candidate target sequences are identified) can be performed. An exemplary score includes a Cutting Frequency Determination (CFD) score, as described by Doench JG, Fusi N, Sullender M, Hegde M, Vaimberg E W. Donovan K F, et al. Optimized sgRNA design to maximize activity and minimize off-target effects of CRISPR-Cas9. Nat Biotechnol.2016:34:184-91.

In certain embodiments, gRNAs as used herein may be modified or unmodified gRNAs. In certain embodiments, a gRNA may include one or more modifications. In certain embodiments, the one or more modifications may include a phosphorothioate linkage modification, a phosphorodithioate (PS2) linkage modification, a 2′-O-methyl modification, or combinations thereof. In certain embodiments, the one or more modifications may be at the 5′ end of the gRNA, at the 3′ end of the gRNA, or combinations thereof.

In certain embodiments, a gRNA modification may comprise one or more phosphorodithioate (PS2) linkage modifications.

In some embodiments, a gRNA used herein includes one or more or a stretch of deoxyribonucleic acid (DNA) bases, also referred to herein as a “DNA extension.” In some embodiments, a gRNA used herein includes a DNA extension at the 5′ end of the gRNA, the 3′ end of the gRNA, or a combination thereof. In certain embodiments, the DNA extension may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 DNA bases long. For example, in certain embodiments, the DNA extension may be 1, 2, 3, 4, 5, 10, 15, 20, or 25 DNA bases long. In certain embodiments, the DNA extension may include one or more DNA bases selected from adenine (A), guanine (G), cytosine (C), or thymine (T). In certain embodiments, the DNA extension includes the same DNA bases. For example, the DNA extension may include a stretch of adenine (A) bases. In certain embodiments, the DNA extension may include a stretch of thymine (T) bases. In certain embodiments, the DNA extension includes a combination of different DNA bases.

In certain embodiments, a gRNA used herein includes a DNA extension as well as a chemical modification, e.g., one or more phosphorothioate linkage modifications, one or more phosphorodithioate (PS2) linkage modifications, one or more 2′-O-methyl modifications, or one or more additional suitable chemical gRNA modification disclosed herein, or combinations thereof. In certain embodiments, the one or more modifications may be at the 5′ end of the gRNA, at the 3′ end of the gRNA, or combinations thereof.

Without wishing to be bound by theory, it is contemplated that any DNA extension may be used with any gRNA disclosed herein, so long as it does not hybridize to the target nucleic acid being targeted by the gRNA and it also exhibits an increase in editing at the target nucleic acid site relative to a gRNA which does not include such a DNA extension.

In some embodiments, a gRNA used herein includes one or more or a stretch of ribonucleic acid (RNA) bases, also referred to herein as an “RNA extension.” In some embodiments, a gRNA used herein includes an RNA extension at the 5′ end of the gRNA, the 3′ end of the gRNA, or a combination thereof. In certain embodiments, the RNA extension may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 RNA bases long. For example, in certain embodiments, the RNA extension may be 1, 2, 3, 4, 5, 10, 15, 20, or 25 RNA bases long. In certain embodiments, the RNA extension may include one or more RNA bases selected from adenine (rA), guanine (rG), cytosine (rC), or uracil (rU), in which the “r” represents RNA. 2′-hydroxy. In certain embodiments, the RNA extension includes the same RNA bases. For example, the RNA extension may include a stretch of adenine (rA) bases. In certain embodiments, the RNA extension includes a combination of different RNA bases. In certain embodiments, a gRNA used herein includes an RNA extension as well as one or more phosphorothioate linkage modifications, one or more phosphorodithioate (PS2) linkage modifications, one or more 2′-O-methyl modifications, one or more additional suitable gRNA modification, e.g., chemical modification, disclosed herein. or combinations thereof. In certain embodiments, the one or more modifications may be at the 5′ end of the gRNA, at the 3′ end of the gRNA, or combinations thereof.

It is contemplated that gRNAs used herein may also include an RNA extension and a DNA extension. In certain embodiments, the RNA extension and DNA extension may both be at the 5′ end of the gRNA, the 3′ end of the gRNA, or a combination thereof. In certain embodiments, the RNA extension is at the 5′ end of the gRNA and the DNA extension is at the 3′ end of the gRNA. In certain embodiments, the RNA extension is at the 3′ end of the gRNA and the DNA extension is at the 5′ end of the gRNA.

In some embodiments, a gRNA which includes a modification, e.g., a DNA extension at the 5′ end and/or a chemical modification as disclosed herein, is complexed with a RNA-guided nuclease, e.g., an AsCpf1 nuclease, to form an RNP, which is then employed to edit a target cell, e.g., a pluripotent stem cell, daughter cell thereof, neuronal progenitor. neuronal cell, or immune cell.

Additional suitable gRNA modifications will be apparent to those of ordinary skill in the art based on the present disclosure. Suitable gRNA modifications include, for example, those described in PCT application PCT/US2018/054027, filed on Oct. 2, 2018, and entitled “MODIFIED CPF1 GUIDE RNA;” in PCT application PCT/US2015/000143, filed on Dec. 3, 2015, and entitled “GUIDE RNA WITH CHEMICAL MODIFICATIONS;” in PCT application PCT/US2016/026028, filed Apr. 5, 2016, and entitled “CHEMICALLY MODIFIED GUIDE RNAS FOR CRISPR/CAS-MEDIATED GENE REGULATION;” and in PCT application PCT/US2016/053344, filed on Sep. 23, 2016, and entitled “NUCLEASE-MEDIATED GENOME EDITING OF PRIMARY CELLS AND ENRICHMENT THEREOF;” the entire contents of each of which are incorporated herein by reference.

Certain exemplary modifications discussed in this section can be included at any position within a gRNA sequence including, without limitation at or near the 5′end (e.g., within 1-10, 1-5, or 1-2 nucleotides of the 5′ end) and/or at or near the 3′ end (e.g., within 1-10, 1-5, or 1-2 nucleotides of the 3′ end). In some cases, modifications are positioned within functional motifs, such as the repeat-anti-repeat duplex of a Cas9 gRNA, a stem loop structure of a Cas9 or Cpf1 gRNA, and/or a targeting domain of a gRNA.

As one example, the 5′ end of a gRNA can include a eukaryotic mRNA cap structure or cap analog (e.g., a G (5′) ppp (5′) G cap analog, a m7G (5′) ppp (5′) G cap analog, or a 3′-O-Me-m7G (5′) ppp (5′) G anti reverse cap analog (ARCA)).

Along similar lines, in some embodiments, the 5′ end of the gRNA can lack a 5′ triphosphate group. For instance, in vitro transcribed gRNAs can be phosphatase-treated (e.g., using calf intestinal alkaline phosphatase) to remove a 5′ triphosphate group.

Another common modification involves the addition, at the 3′ end of a gRNA, of a plurality (e.g., 1-10, 10-20, or 25-200) of adenine (A) residues referred to as a polyA tract. The polyA tract can be added to a gRNA during chemical or enzymatic synthesis, using a polyadenosine polymerase (e.g., E, coli Poly(A) Polymerase).

Guide RNAs can be modified at a 3′ terminal U ribose. For example, the two terminal hydroxyl groups of the U ribose can be oxidized to aldehyde groups and a concomitant opening of the ribose ring to afford a modified nucleoside.

Guide RNAs can contain 3′ nucleotides that can be stabilized against degradation, e.g., by incorporating one or more of the modified nucleotides described herein. In certain embodiments, uridines can be replaced with modified uridines, e.g., 5-(2-amino) propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein.

In certain embodiments, sugar-modified ribonucleotides can be incorporated into a gRNA, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH2, alkylamino, dialkylamino, heterocyclyl, arylamino, diarylamino, heteroarylamino, diheteroarylamino, or amino acid); or cyano (—CN). In certain embodiments, the phosphate backbone can be modified as described herein, e.g., with a phosphothioate (PhTx) group. In certain embodiments, one or more of the nucleotides of the gRNA can each independently be a modified or unmodified nucleotide including, but not limited to 2′-sugar modified, such as 2′-O-methyl, 2′-O-methoxyethyl, or 2′-Fluoro modified including, e.g., 2′-F or 2′-O-methyl, adenosine (A), 2′-F or 2′-O-methyl, cytidine (C). 2′-F or 2′-O-methyl, uridine (U), 2′-F or 2′-O-methyl, thymidine (T), 2′-F or 2′-O-methyl, guanosine (G), 2′-O-methoxyethyl-5-methyluridine (Teo), 2′-O-methoxyethyladenosine (Aco), 2′-O-methoxyethyl-5-methylcytidine (m5Cco), and any combinations thereof. Guide RNAs can also include “locked” nucleic acids (LNA) in which the 2′ OH-group can be connected, e.g., by a C1-6 alkylene or C1-6 heteroalkylene bridge, to the 4′ carbon of the same ribose sugar. Any suitable moiety can be used to provide such bridges, including without limitation methylene, propylene, ether, or amino bridges; O-amino (wherein amino can be, e.g., NH2, alkylamino, dialkylamino. heterocyclyl, arylamino, diarylamino, heteroarylamino, or diheteroarylamino, ethylenediamine, or polyamino) and aminoalkoxy or O(CH2) n-amino (wherein amino can be, e.g., NH2, alkylamino, dialkylamino, heterocyclyl, arylamino, diarylamino, heteroarylamino, or diheteroarylamino, ethylenediamine, or polyamino).

In certain embodiments, a gRNA can include a modified nucleotide which is multicyclic (e.g., tricyclo; and “unlocked” forms, such as glycol nucleic acid (GNA) (e.g., R-GNA or S-GNA, where ribose is replaced by glycol units attached to phosphodiester bonds), or threose nucleic acid (TNA, where ribose is replaced with α-L-threofuranosyl-(3′->2′)).

Generally, gRNAs include the sugar group ribose, which is a 5-membered ring having an oxygen. Exemplary modified gRNAs can include, without limitation, replacement of the oxygen in ribose (e.g., with sulfur(S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). Although the majority of sugar analog alterations are localized to the 2′ position, other sites are amenable to modification, including the 4′ position. In certain embodiments, a gRNA comprises a 4′-S, 4′-Se or a 4′-C-aminomethyl-2′-O-Me modification.

In certain embodiments, deaza nucleotides, e.g., 7-deaza-adenosine, can be incorporated into a gRNA. In certain embodiments, O- and N-alkylated nucleotides, e.g., N6-methyl adenosine, can be incorporated into a gRNA. In certain embodiments, one or more or all of the nucleotides in a gRNA are deoxynucleotides.

In certain embodiments, guide RNAs can also include one or more cross-links between complementary regions of the crRNA (at its 3′ end) and the tracrRNA (at its 5′ end) (e.g., within a “tetraloop” structure and/or positioned in any stem loop structure occurring within a gRNA). A variety of linkers are suitable for use. For example, guide RNAs can include common linking moieties including, without limitation, polyvinylether, polyethylene, polypropylene, polyethylene glycol (PEG), polyvinyl alcohol (PVA), polyglycolide (PGA), polylactide (PLA), polycaprolactone (PCL), and copolymers thereof.

In some embodiments, a bifunctional cross-linker is used to link a 5′ end of a first gRNA fragment and a 3′ end of a second gRNA fragment, and the 3′ or 5′ ends of the gRNA fragments to be linked are modified with functional groups that react with the reactive groups of the cross-linker. In general, these modifications comprise one or more of amine, sulfhydryl. carboxyl, hydroxyl, alkene (e.g., a terminal alkene), azide and/or another suitable functional group. Multifunctional (e.g, bifunctional) cross-linkers are also generally known in the art, and may be either heterofunctional or homofunctional, and may include any suitable functional group, including without limitation isothiocyanate, isocyanate, acyl azide, an NHS ester, sulfonyl chloride, tosyl ester, tresyl ester, aldehyde, amine, epoxide, carbonate (e.g., Bis(p-nitrophenyl) carbonate), aryl halide, alkyl halide, imido ester, carboxylate, alkyl phosphate, anhydride, fluorophenyl ester, HOBt ester, hydroxymethyl phosphine, O-methylisourea, DSC, NHS carbamate, glutaraldehyde, activated double bond, cyclic hemiacetal, NHS carbonate, imidazole carbamate, acyl imidazole, methylpyridinium ether, azlactone, cyanate ester, cyclic imidocarbonate, chlorotriazine, dehydroazepine, 6-sulfo-cytosine derivatives, maleimide, aziridine. TNB thiol, Ellman's reagent, peroxide, vinylsulfone, phenylthioester, diazoalkanes, diazoacetyl, epoxide, diazonium, benzophenone, anthraquinone, diazo derivatives, diazirine derivatives, psoralen derivatives, alkene, phenyl boronic acid, etc. In some embodiments, a first gRNA fragment comprises a first reactive group and the second gRNA fragment comprises a second reactive group. For example, the first and second reactive groups can each comprise an amine moiety, which are crosslinked with a carbonate-containing bifunctional crosslinking reagent to form a urea linkage. In other instances, (a) the first reactive group comprises a bromoacetyl moiety and the second reactive group comprises a sulfhydryl moiety, or (b) the first reactive group comprises a sulfhydryl moiety and the second reactive group comprises a bromoacetyl moiety, which are crosslinked by reacting the bromoacetyl moiety with the sulfhydryl moiety to form a bromoacetyl-thiol linkage. These and other cross-linking chemistries are known in the art, and are summarized in the literature, including by Greg T. Hermanson, Bioconjugate Techniques, 3rd Ed.2013, published by Academic Press.

Additional suitable gRNA modifications will be apparent to those of ordinary skill in the art based on the present disclosure. Suitable gRNA modifications include, for example, those described in PCT application PCT/US2018/054027, filed on Oct. 2, 2018, and entitled “MODIFIED CPF1 GUIDE RNA;” in PCT application PCT/US2015/000143, filed on Dec. 3, 2015, and entitled “GUIDE RNA WITH CHEMICAL MODIFICATIONS;” in PCT application PCT/US2016/026028, filed Apr. 5, 2016, and entitled “CHEMICALLY MODIFIED GUIDE RNAS FOR CRISPR/CAS-MEDIATED GENE REGULATION;” and in PCT application PCT/US2016/053344, filed on Sep. 23, 2016, and entitled “NUCLEASE-MEDIATED GENOME EDITING OF PRIMARY CELLS AND ENRICHMENT THEREOF;” the entire contents of each of which are incorporated herein by reference.

In some embodiments, an exemplary gRNA targets SIRPα. In some embodiments, an exemplary gRNA targets a SIRPα negative regulator. In some embodiments, an exemplary gRNA targets a SIRPα positive regulator.

In some embodiments, an exemplary gRNA targets CD47. In some embodiments, an exemplary gRNA targets a CD47 negative regulator. In some embodiments, an exemplary gRNA targets a CD47 positive regulator.

### G. Other Agents

It is contemplated that other agents may be used in combination with certain aspects of the present embodiments to improve therapeutic efficacy of a treatment regimen. These additional agents include agents that alter drug efficacy, vector expression, blood-brain-barrier permeability, cell specificity, antigen-binding capacity, etc.

### III. Administration of Therapeutic Compositions

In some embodiments, a therapy provided herein may comprise administration of a single therapy. In some embodiments, a therapy provided herein may comprise administration of a combination of therapeutic agents, such as a first therapy and a second therapy. In some embodiments, one or more therapies may be administered in any suitable manner known in the art. For example, in some embodiments, a first and a second treatment may be administered sequentially (at different times) or concurrently (at the same time). In some embodiments, a first and a second therapy are administered in a separate composition. In some embodiments, a first and a second therapy are in the same composition.

In some embodiments, a therapy is administered more than once. In some embodiments, a first therapy and a second therapy are administered substantially simultaneously. In some embodiments, a first therapy and a second therapy are administered sequentially. In some embodiments, a first therapy, a second therapy, and a third therapy are administered sequentially. In some embodiments, a first therapy is administered before administering a second therapy. In some embodiments, a first therapy is administered after administering a second therapy.

In some embodiments, the present disclosure relates to compositions and methods comprising therapeutic compositions. In some embodiments, different therapies may be administered in one composition or in more than one composition, such as 2 compositions, 3 compositions, 4 compositions, 5 compositions, and so on. In some embodiments, various combinations of therapeutic agents (e.g., inhibitory oligonucleotides, proteins and polypeptides, vectors, transgenes, small molecules, cells, genome editing systems, etc.) described herein may be employed.

In some embodiments, therapeutic agents of the present disclosure may be administered by the same route of administration or by different routes of administration. In some embodiments, a therapy is administered intravenously, intramuscularly, subcutaneously, topically, orally, transdermally, intraperitoneally, intraorbitally, topically to the eye by implantation, by inhalation, intrathecally, intraventricularly, or intranasally. In some embodiments, more than one therapeutic agents are administered intravenously, intramuscularly, subcutaneously, topically, orally, transdermally, intraperitoneally. intraorbitally, topically to the eye, by implantation, by inhalation, intrathecally, intraventricularly, or intranasally. The appropriate dosage may be determined based on the type of disease to be treated, severity and course of the disease, the clinical condition of the individual, the individual's clinical history and response to treatment(s), and the discretion of the attending physician.

In some embodiments, a treatment may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

In some embodiments, a first therapy comprises a first protein, a nucleic acid encoding for a first protein, a vector comprising a nucleic acid encoding for a first protein, or a cell comprising a first protein. In some embodiments, a second therapy comprises a second protein, a nucleic acid encoding for a second protein, a vector comprising a nucleic acid encoding for a second protein, or a cell comprising a second protein. In some embodiments, a single dose a protein therapy is administered. In some embodiments, multiple doses of a protein therapy are administered. In some embodiments, a protein is administered at a dose of between 1 mg/kg and 5000 mg/kg. In some embodiments, a protein is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 mg/kg.

In some embodiments, a single dose of a therapy is administered. In some embodiments, multiple doses of the one or more therapies are administered. In some embodiments, an at least second therapy is administered at a dose of between 1 mg/kg and 100 mg/kg. In some embodiments, an at least second therapy is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the preventative, ameliorative, curative, and/or risk reducing capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, months, or years.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 M; or about 1 μM to 40 μM; or about 1 μM to 30 μM; or about 1 μM to 20 μM; or about 1 μM to 10 μM; or about 10 μM to 150 M; or about 10 μM to 100 μM; or about 10 μM to 50 μM; or about 25 μM to 150 μM; or about 25 μM to 100 μM; or about 25 μM to 50 u M; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent a therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of a composition, e.g., 2, 3, 4, 5, 6 or more administrations. In some embodiments, administrations can be at 1, 2, 3, 4, 5, 6, 7, 8, to 5, 6, 7, 8, 9, 10, 11, or 12 week intervals, including all ranges there between. In some embodiments, administrations can be every six months, year, two years, five years, or ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into compositions.

In some embodiments, active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

In some embodiments, pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be reasonably stable under the conditions of manufacture and storage and must be reasonably preserved against the contaminating action of microorganisms and/or other contaminants, such as bacteria, fungi, etc.

In some embodiments, proteinaceous compositions may be formulated into a neutral or salt form. In some embodiments, pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. In some embodiments, salts formed with the free carboxyl groups can also be derived from inorganic bases such as, for example, sodium, potassium, ammonium, calcium, or ferric hydroxides, and such organic bases as isopropylamine, trimethylamine, histidine, procaine, and the like.

In some embodiments, a pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In some embodiments, sterile injectable solutions are prepared by incorporating one or more active compounds in a required amount in an appropriate solvent with various other ingredients enumerated herein or otherwise known to be suitable by a practitioner in the art, as required, followed by filtered sterilization or an equivalent procedure. Generally, dispersions are prepared by incorporating the various sterilized one or more active ingredients into a sterile vehicle which contains the basic dispersion medium and the required other ingredients from those enumerated herein or otherwise known to be suitable by a practitioner in the art. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

In some embodiments, administration of the compositions will typically be via any common route. In some embodiments, administration routes include but are not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients. In some embodiments, administration routes are those suited for crossing the blood brain barrier. In some embodiments, administration routes are via central nervous system injection.

In some embodiments, upon formulation, solutions will be administered in a manner compatible with the dosage formulation and in such amount as is therapeutically or prophylactically effective. In some embodiments, formulations are administered in a variety of dosage forms, such as the type of formulations described herein.

### Pharmaceutical Compositions

In certain aspects, compositions or agents for use in line with the present disclosure, (e.g., such as small molecules, antibodies, inhibitory oligonucleotides, vectors, cells, etc.,) are suitably contained in a pharmaceutically acceptable carrier. A carrier is non-toxic. biocompatible and is selected so as not to detrimentally affect one or more biological activities of an agent. In some embodiments, agents as described in the present disclosure may be formulated into preparations for local delivery (i.e, to a specific location of the body, such as nervous system (e.g., the central nervous system (CNS), e.g., the brain, the spinal cord, the retina, etc., or the peripheral nervous system (PNS)) or systemic delivery, in solid, semi-solid, gel, liquid and/or gaseous forms such as tablets, capsules, powders, granules, ointments, solutions, depositories, inhalants and/or injections allowing for oral, parenteral and/or surgical administration. Certain aspects of the disclosure also contemplate local administration of compositions and/or execution of methods by coating medical devices and the like.

In some embodiments, suitable carriers for parenteral delivery via injectable. infusion or irrigation and topical delivery include but are not limited to distilled water, physiological phosphate-buffered saline, normal or lactated Ringer's solutions, dextrose solution, Hank's solution, or propanediol. In addition, in some embodiments, sterile, fixed oils may be employed as a solvent or suspending medium, for such a purpose any biocompatible oil may be employed including synthetic mono- or diglycerides. In addition, in some embodiments, fatty acids such as oleic acid find use in the preparation of injectables. In some embodiments, a carrier and agent may be compounded as a liquid, suspension, polymerizable or non-polymerizable gel, paste, and/or salve.

In some embodiments, a carrier may also comprise a delivery vehicle to sustain (e.g., extend, delay, regulate, etc.) the delivery of one or more agents, and/or to enhance delivery, uptake, stability or pharmacokinetics of one or more therapeutic agents. Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

In certain aspects, an actual dosage amount of a composition administered to a patient or subject can be determined by physical and physiological factors such as body weight, severity of condition, type of disease being treated, previous or concurrent therapeutic interventions, idiopathy of the patient, and/or intended route of administration. In some embodiments, a practitioner responsible for administration will, in any event, determine the concentration of active ingredient(s) in a composition and appropriate dose(s) for the individual subject.

In some embodiments, solutions of pharmaceutical compositions can be prepared in water suitably mixed with a surfactant (e.g., hydroxypropylcellulose, etc.). In some embodiments, dispersions can be prepared in glycerol, liquid polyethylene glycols, mixtures thereof, and/or in oils. In some embodiments, under ordinary conditions of storage and use, these preparations can contain a preservative to prevent growth of microorganisms and/or other contaminants.

In certain aspects, pharmaceutical compositions are advantageously administered in the form of injectable compositions either as liquid solutions or suspensions; solid forms suitable or solution in, or suspension in, liquid prior to injection may also be prepared. In some embodiments, preparations also may be emulsified. A typical composition for such purpose comprises a pharmaceutically acceptable carrier. For example, a composition may contain 10 mg or less. 25 mg. 50 mg or up to about 100 mg of human serum albumin per milliliter of phosphate buffered saline. Other pharmaceutically acceptable carriers include but are not limited to aqueous solutions, non-toxic excipients, including salts, preservatives, buffers, etc.

In some embodiments, non-aqueous solvents include but are not limited to propylene glycol, polyethylene glycol, vegetable oil, injectable organic esters such as ethyloleate, and/or combinations thereof. In some embodiments, aqueous carriers include but are not limited to water, alcoholic/aqueous solutions, saline solutions, parenteral vehicles such as sodium chloride, Ringer's dextrose, etc. In some embodiments, intravenous vehicles include but are not limited to fluid and/or nutrient replenishers. In some embodiments, preservatives include but are not limited to antimicrobial agents, antifungal agents, anti-oxidants, chelating agents, inert gases, and/or mixtures thereof. In some embodiments, pH and exact concentrations of various components comprised in a pharmaceutical composition are adjusted according to well-known parameters.

In some embodiments, additional formulations are suitable for oral administration. Oral formulations include but are not limited to typical excipients as, for example, pharmaceutical grades of mannitol, lactose, starch, magnesium stearate, sodium saccharine, cellulose, magnesium carbonate and the like. In some embodiments, compositions take the form of solutions, suspensions, tablets, pills, capsules, sustained release formulations or powders.

In further aspects, pharmaceutical compositions may include classic pharmaceutical preparations. In some embodiments, administration of pharmaceutical compositions according to certain aspects may be via any common route so long as the target tissue is available via that route. For example, this may include oral, nasal, buccal, rectal, vaginal, topical, or ophthalmological.

In some embodiments, pharmaceutical compositions may be administered by orthotopic, intradermal, subcutaneous, intramuscular, intraperitoneal or intravenous injection. In some embodiments, pharmaceutical compositions are administered to the nervous system, e.g., directly or indirectly. In some embodiments, pharmaceutical compositions are administered by intracerebroventricular injection. In some embodiments, pharmaceutical compositions are administered directly to certain CNS tissues, including but not limited to the striatum, the thalamus, the substantia nigra, the parietal cortices, the retina, the hippocampus, and/or the globus pallidus.

In some embodiments, a pharmaceutical composition according to the present invention is to be administrated intraocularly (i.e., directly into the eye), intrastriatally (i.e., in the striatum, such as, e.g., in the putamen, caudate nucleus, nucleus accumbens, olfactory tubercle, external globus pallidus and/or internal globus pallidus), intrathalamically (i.e., in the thalamus), and/or intracisternally (i.e., in the subarachnoid cisterns, such as, e.g., in the cisterna magna, pontine cistern, interpeduncular cistern, chiasmatic cistern, cistern of lateral cerebral fossa, superior cistern and/or cistern of lamina terminalis.

In some embodiments, compositions can normally be administered as pharmaceutically acceptable compositions that include physiologically acceptable carriers, buffers or other excipients.

In some embodiments, pulmonary delivery may be appropriate, and an aerosol delivery mechanism can be used, wherein a volume of aerosol may be between about 0.01 ml and 0.5 ml, for example.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Additional details regarding subsets of the experiments described herein are comprised in the publication “Neuronal signal regulatory protein alpha drives microglial phagocytosis in the developing retina by limiting microglial interaction with CD47” by Melanie Samuel et al., (2022, IMMUNITY), which is incorporated herein by reference in its entirety.

When applicable, we have included information about the experimental design, sample size, rules for stopping data collection, selection of endpoints, experimental replicates, and randomization for each individual experiment in the Materials and Methods below.

### Materials and Methods

Unless otherwise noted, methods were conducted as described below.

Mice: SIRPα^{F/F} mice were kindly provided by Dr. Beth Steven, Boston Children's Hospital. To broadly delete SIRPα from retinal neurons, SIRPα^{F/F} mice were crossed to Six3^{Cre} mice (Furuta et al., 2000), referred here as SIRPα^{NEURON} mice. To delete SIRPα in microglia, SIRPα^{F/F} mice were crossed to TNFRSF11A^{Cre} mice (Maeda et al., 2012) to generate animals referred here as SIRPα^{MICROGLIA} mice. TNFRSF11A^{Cre} is expressed in and targets yolk sac-derived erythro-myeloid progenitors (Jordao et al., 2019), which in the brain are comprised of microglia. For these lines, SIRPα^{F/F} littermates were used as controls. To deplete microglia, Cx3crl^{CreER} mice (Yona et al., 2013) were crossed to ROSA26^{iDTR}mice (Buch et al., 2005) to generate animals referred here as Cx3crl^{CreER}; Rosa26^{iDTR}mice. C57BL/6 mice, Cx3Crl^{GFP/+} mice, CD47F/F, and CD47′-mice were obtained from Jackson Labs. SIRPα^{NEURON}. CD47NEURON double knockouts were generated by crossing SIRPα^{F/F} and CD47F/F mice to Six3^{Cre} mice. For this line, SIRPα^{FF}; CD47FF littermates were used as controls. All mice were used at the ages specified in the experimental procedures outlined below, and a mixture of male and female mice were used. Experiments were carried out in accordance with the recommendations in the Guide for the Care and Use of Laboratory Animals of the NIH under protocols approved by the BCM Institutional Animal Care and Use Committee.

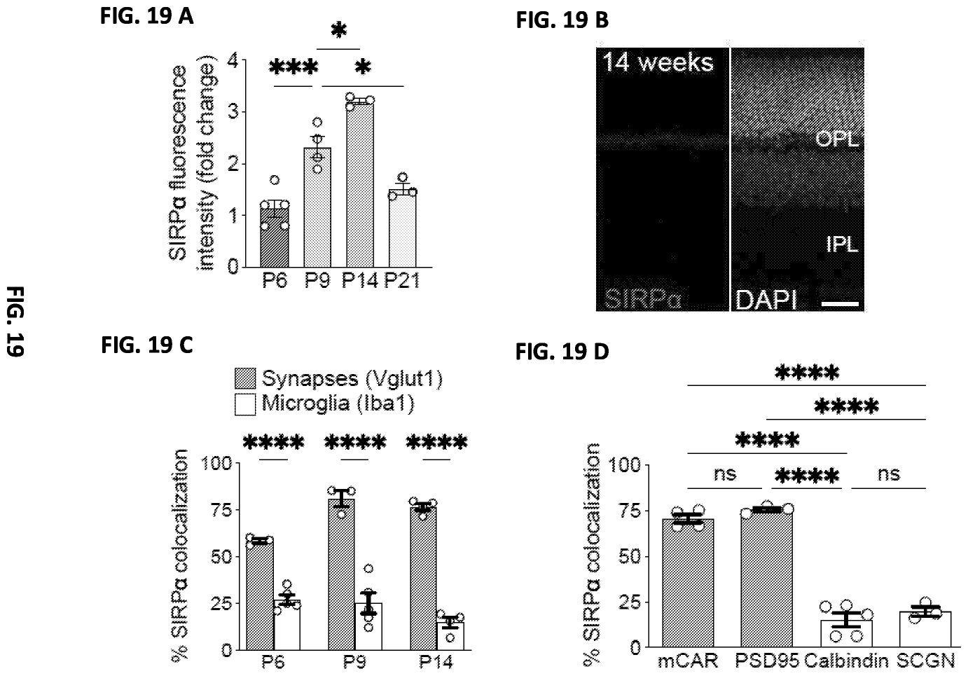

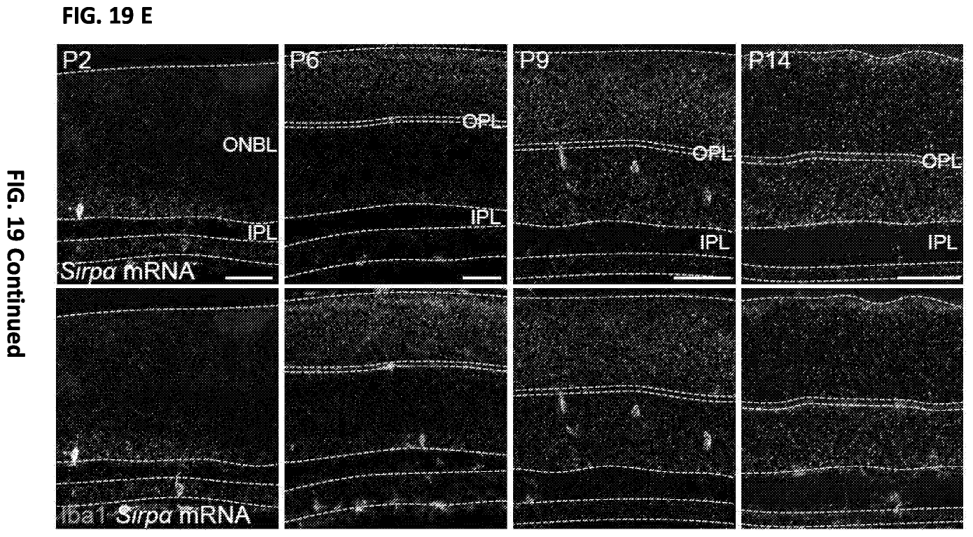

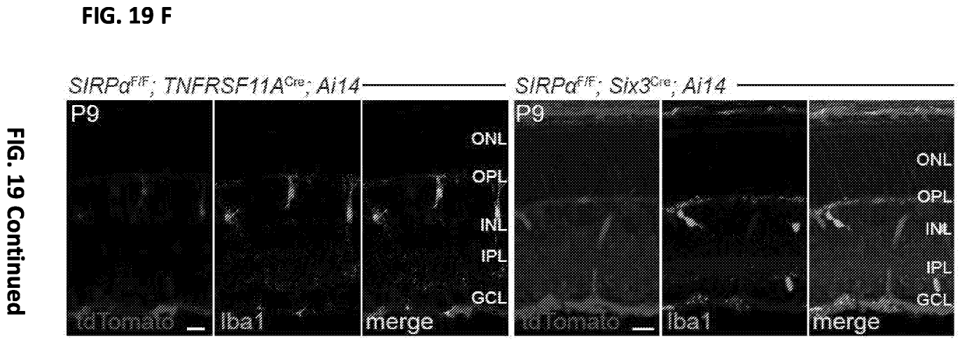

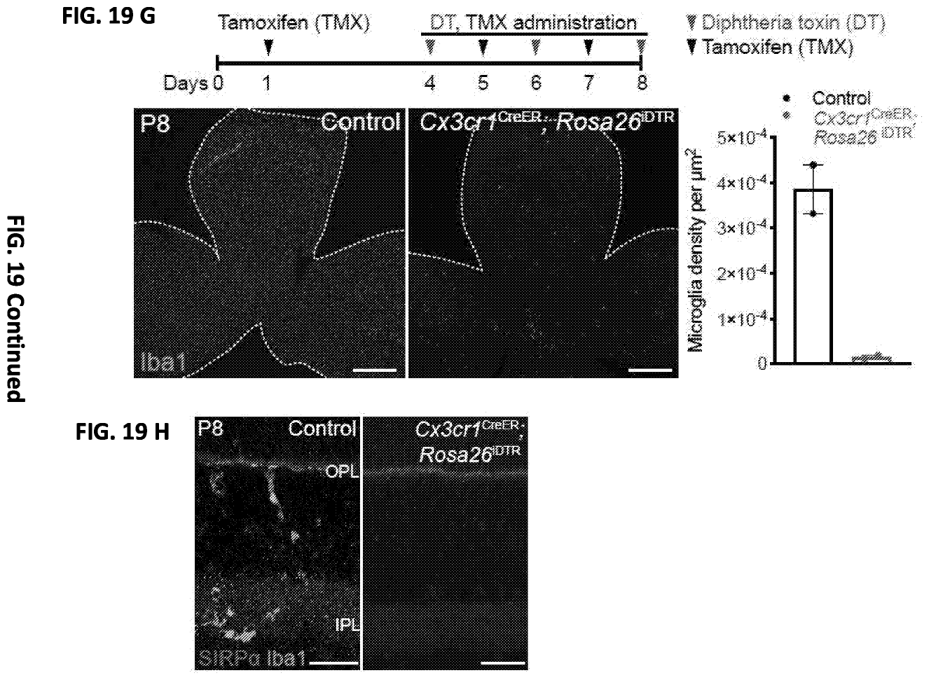

Microglia depletion: For microglia ablation experiments, the ROSA26^{iDTR}line (Buch et al., 2005) was crossed to the Cx3crl^{CreER} line (Yona et al., 2013) to generate animals referred here as Cx3erI^{CreER}; Rosa26i^{DTR}. To ablate microglia for longer periods of time and prevent repopulation of microglia, we administered tamoxifen and diphtheria toxin as previously described (Punal et al., 2019). In brief, 100 μg of tamoxifen was administered via intraperitoneal (IP) injection to neonatal pups at P1, P5, and P7, and single doses of 80 ng of Diphtheria toxin were administered at P4, P6, and P8. Depletion (˜96% compared to control) was confirmed by staining with the microglial marker Iba1 at P8 (FIG. 19 G).

Immunohistochemistry: Immunohistochemistry was performed as previously described (Jiang et al., 2020). Briefly, eyes were harvested from mice at P2, P6, P9, P14, P21. and 14 weeks and fixed in 4% PFA for 45 min at room temperature. For cross-section analysis, eye cups were dissected, and the cornea and lens were removed. Following cryoprotection in 30% sucrose, eyes were embedded in OCT compound (VWR) and sectioned at 20 μm thickness. Cryosections were incubated with blocking buffer (3% normal donkey serum and 0.3% Triton X-100 in PBS) for 1 h, and then with primary antibodies diluted in blocking buffer overnight at 4° C. After washing, secondary antibodies were applied and incubated for 1 h at room temperature. Slides were then washed again and mounted with Vectashield (Vector Labs). For whole-mount preparations, the retinas were removed from the eye cups and blocked with a 10% normal donkey serum and 0.5% Triton X-100 solution in PBS for 1 h before proceeding with incubation with primary antibodies diluted in blocking buffer for 5 days followed by washes and staining with secondary antibodies for 3 days at 4° C. All images were acquired using an Olympus Fluoview FV1200 confocal microscope and processed using FIJI.

RNAscope: RNAscope single-molecule fluorescence RNA in situ hybridization (smFISH) was performed on 20 μm sections of retina collected as described for immunohistochemistry using Probe-Mm-SIRPα (837091) and Probe-Mm-CD47-C2 (515461-C2, ACD-bio). RNAscope fluorescent multiplex assays were performed according to the manufacturer's instructions (ACD-bio) with the following modifications. Tissue samples were dehydrated using an ethanol gradient of 10%, 30%, 50%, 70%, and 100% (3 min each), and the boiling time in target retrieval solution was modified to 5 min. After RNAscope, slides were co-stained with Iba1, Calbindin, RBPMS, and AP2 to visualize microglia, horizontal cells, ganglion cells and amacrine cells, respectively.

Quantitative Real-Time PCR: Total RNA was isolated from whole retinas of P9 control and SIRPα^{NEURON} animals using the RNeasy Mini kit (Qiagen) according to the manufacturer's instructions. 100 ng of RNA was used to generate cDNA by reverse transcription using the iScript Reverse Transcription Supermix (BioRad). qRT-PCR was subsequently performed using the iTaq Universal SYBR Green Supermix (BioRad) on a CFX384 Touch Real-Time PCR Detection System with primer sequences listed below.

Table-Primer Sequences

| qRT-PCR | | SEQ |
|---|---|---|
| primers | Sequence | ID NO: |
| β actin FWD | TGAGAGGGAAATCGTGCGTG | 11 |
| β actin REV | TCGTTGCCAATAGTGATGACCTG | 12 |
| Cx3cr1 FWD | AAAAACACTGGATTTCAGGGGC | 13 |
| Cx3cr1 REV | CAACCAACACAGGAACAGGGAG | 14 |
| Mertk FWD | CGCTCTGGAGTGGAGGCAC | 15 |
| Mertk REV | AAACGCAACAGGAGGTAGGAG | 16 |
| CD68 FWD | GGACACTTCGGGCCATGTTT | 17 |
| CD68 REV | CTTACACAGTGGACTGGGGC | 18 |
| C1qb FWD | ATGGATGCGTAATCACGGGG | 19 |
| C1qb REV | GTCTGGGTTTCAGGCAGTCAAG | 20 |
| C3 FWD | TCTGACCTCTGGGGAGAAAAGC | 21 |
| C3 REV | TGGGACAACCATAAACCACCATAG | 22 |
| Cd11b FWD | TGTGGACTCTCATGCCTCCT | 23 |
| Cd11b REV | TGGTCATCTCTGAAGCCGTG | 24 |
| VNR FWD | CGTCCTCCAGGATGTTTCTCC | 25 |
| VNR REV | GCTTTGACCTCACAGAGGC | 26 |
| Ccr2 FWD | GCTGTGTTTGCCTCTCTACCAG | 27 |
| Ccr2 REV | CAAGTAGAGGCAGGATCAGGCT | 28 |
| Mfge8 FWD | GAGCAACAGTGCCAAGGAATGG | 29 |
| Mfge8 REV | ACTGTGGGCTACCTTGTAGGAC | 30 |
| Tyrobp FWD | GGACCCGGAAACAACACATTG | 31 |
| Tyrobp REV | GATCCCAGAGAGGGCTTGTT | 32 |
| LRP FWD | GTGTCCAACTGCACAGCAAG | 33 |
| LRP REV | GCAGACGTGAATGTCGCAAT | 34 |

Plasmid construction: pCAG and pCAG-GFP vectors were kindly provided by Dr. Elizabeth Zuniga-Sanchez at Baylor College of Medicine. In brief, the pCAG vector was generated by cloning the promoter region of the original pCAG-IRES-GFP (Matsuda and Cepko, 2004; 2007) plasmid into the pcDNA3.1 vector (Invitrogen). The pCAG-GFP construct was generated by adding GFP to the pCAG (in pcDNA3.1) vector. Coding sequences for either SIRPα and CD47 (MG208194 and MG204706. Origene) were removed and cloned downstream of the CAG promoter in the pCAG vector. These vectors were then expressed in combination with the pCAG-GFP to allow for fluorescent visualization.

Electroporation: For SIRPα and CD47 over-expression, retinas of neonatal pups (12-24 h) were electroporated with the expression plasmids detailed above using a modified version of the protocol developed by Cepko and colleagues (Matsuda and Cepko, 2004). Briefly, sharp end glass micropipettes (Sutter Instrument) were loaded with 5-8 μl of DNA (diluted to a final concentration of 4 μg/μl) mixed with Fast Green Dye (0.2×) and were used to deliver 2-3 μl DNA into the subretinal space. Following injection, five current pulses (80V, 50 ms duration. 950 ms interval) were applied across the pup head using Tweezer electrodes (Harvard Apparatus).

Electroretinography: P21 SIRPα^{NEURON} (n=5), SIRPαMICROGLIA (n=3), and respective littermate control (n=4, n=7) animals were dark-adapted overnight and then anesthetized with isoflurane (3% induction, 1.5% maintenance) carried in oxygen at a flow rate of 1 L/min using a vaporizer. Animals were placed on a heated platform, and eyes were dilated with phenylephrine hydrochloride and tropicamide. A contact lens-style electrode in contact with Gonak solution was placed on each cornea. A reference electrode was placed at the forehead, and a ground electrode was placed at the hip. Scotopic responses were elicited in the dark with flashes ranging from 0.003 cd*s/m2 to 20 cd*s/m2 using the Diagnosys Celeris ERG system. Electroretinograms were recorded from both eyes simultaneously.

Ex vivo phagocytosis assay: Ex vivo phagocytosis assays were performed as previously described (Wang et al., 2021b). In brief, freshly dissected retinas from P9 control and SIRPα^{NEURON}; Cx3cr1^{GPF+} animals were incubated in 1 mg/mL pHrodo Red-conjugated zymosan bioparticles (Thermo Fisher Scientific) resuspended in culture media of 1:1 mixture of DMEM and F-12 supplemented with B27 (50×). BDNF (50×), and penicillin-streptomycin (100×) at 37° C., with gentle agitation. Retinas were subsequently washed three times with PBS and dissociated using cysteine-activated papain for 8 min at 37° C. Digestion was inactivated by the addition of medium containing ovomucoid (1.5 mg/mL), BSA (1.5 mg/mL) and DNase I (67 U/mL), followed with gentle mechanical dissociation by pipetting up and down with a P1000 tip. The sample was spun at 30 g for 20 sec, and supernatant containing cells was passed through a 40 μm strainer. This process was repeated until all cells were dissociated. Cells were then spun at 350 g at 4° C., for 10 min and resuspended in 500 μl, of MEM-B (no glutamine with 4% Bovine Serum Albumin media) with 0.5 μg/mL DAPI. Flow cytometry data were collected in a BD LSR II Cytometer and analyzed using FlowJo 9 to compute the percentage of GFP814 positive microglia that were also positive for pHrodo Red.

Immunoblotting analysis: WT (P4, P6, P9, P14), SIRPα^{NEURON} (P9), and SIRPα^{MICROGLIA} (P9) retinas were dissected and snap frozen on dry ice. Frozen tissues were then transferred into a RIPA buffer containing complete protease inhibitor (Roche, 1:50). phosphatase inhibitor I (Calbiochem, 1:100), and phosphatase inhibitor II (Calbiochem, 1:100). Samples were manually homogenized with a Kimble Kontes Pellet Pestle homogenizer (DWK Life Sciences). For each sample. 10 μg of protein was loaded and separated by SDS-PAGE on 10% tris-glycine gels before transferred onto nitrocellulose membranes. Blots were blocked in blocking buffer (5% BSA, 0.05% Tween 20 in TBS) for 1 h and then probed with primary antibodies overnight at 4° C., in 5% BSA. Blots were subsequently washed and stained with secondary antibodies for 1 h at room temperature. FIJI was used to perform densitometry analysis of bands.

STORM imaging: Samples were prepared and imaged as described in Albrecht et al., 2021. In brief, eyes were harvested from P9 animals and fixed in 4% PFA for 45 min at room temperature. Eye cups were subsequently dissected, and the cornea and lens were removed. Following cryoprotection in 30% sucrose, eyes were embedded in OCT compound (VWR) and sectioned at 10 μm thickness. Cryosections were incubated with a 3% normal donkey serum and 0.3% Triton X-100 solution in PBS for 1 h, and then with primary antibodies overnight at 4° C. After washing, secondary antibodies were applied and incubated for 1 h at room temperature. Images were acquired on a Bruker Vutara 352 (Bruker, Billerica, MA) using a 60×water objective (UPLSAPO60XW) at an axial step size of 200 nm. 3D-structured images of OPL synapses were generated using the Ordering Points to Identify the Clustering Structure (OPTICS) algorithm. To analyze images, a general particle distance of 0.16 μm and a particle count per cluster of 25 was used for all channels on all images.

Histological quantification, Microglia density quantification: To quantify wildtype microglia cell density at P6, P9, and P14, three independent fields of view (635.90 μm×635.90 μm) from one retina were imaged per animal, and three animals were imaged (n=3). The number of microglia was subsequently counted in each field, and density of microglia was calculated by dividing the total number of microglia in each field by the image area.

Histological quantification, Microglia morphology quantification: To assess microglia morphology at P9, whole-mount retinas were stained for Iba1. For each genotype, n≥3 animals were imaged. Three 635.90 μm×635.90 μm image fields were sampled in each animal. The number of microglia process endpoints and the total branch length were quantified as previously described (Young and Morrison, 2018). In brief, each image was skeletonized after optimization and transformed into a binary image. Individual microglia endpoints and branch length were summed and divided by the total number of microglia using the Analyze Skeleton Plugin in FIJI. Microglia soma size was measured using the Free-hand selection and Measure tools in FIJI. A minimum of ten randomly selected microglia were measured for soma size in each image. Phagocytic cups were identified as cup-shaped invaginations at the tip of Iba1-positive microglial processes and were quantified using the Cell Counter tool in FIJI. The average number of phagocytic cups per cell was calculated by dividing the total number of phagocytic cups by the total number of microglia with cups in each image. The percentage of microglia with cups was calculated by dividing the number of microglia with cups by the total number of microglia in a given image.

Histological quantification, Engulfment analysis: P9 retinas were harvested using the same methods described for immunohistochemistry and were stained for Iba1 and CD68 in whole-mount preparations. For each genotype, n≥3 animals were imaged. For each animal, at least 15 microglia residing in the OPL were imaged on an Olympus Fluoview FV1200 confocal microscope at 20×using a step size (Z) of 0.5 μm. The images were then processed and analyzed using FIJI and IMARIS (Bitplane) as previously described (Schafer et al., 2014). Briefly. Iba1-positive microglia and CD68-positive lysosomes were 3D-reconstructed using the volume surface rendering function in IMARIS 9.2, and their respective volumes were determined. Any CD68 signal outside the Iba1-positive microglia was masked in the image using the mask function. The percent volume of CD68-positive lysosomes was determined by dividing the volume of the internal CD68 staining (μm³) by the volume of the Iba1-positive microglia (μm³). The CD68 mean fluorescence intensity was determined by dividing the total CD68 signal by the image field area after background signal was subtracted. In the over-expression experiment, engulfment of GFP-positive neural materials inside Iba1-positive microglia was 3D-reconstructed using the same method. The percent volume of GFP-positive neural material inside microglia was determined by dividing the volume of the internal GFP staining by the volume of the microglia. All analyses were performed blind to the experimental conditions.

Histological quantification, Synapse quantification: Immunohistochemistry with the ribbon synapse marker RIBEYE was performed on P21 retina cryosections as described above. For each genotype, n>3 animals were imaged and three independent fields of view in the OPL were captured per animal (60× objective. 2× zoom) using a 20 μm Z-stack comprised of a 0.5 μm step size. Images were subsequently quantified for the number of RIBEYE-positive ribbon synapses in every fifth Z-plane using the Cell Counter tool in FIJI. Synapse numbers were then averaged per animal. RIBEYE mean fluorescence intensity was determined by dividing the total RIBEYE signal by the OPL area after background signal was subtracted using the Frechand and Measure tools in FIJI. All analyses were performed blind to the genotype.

Histological quantification, Colocalization quantification: To quantify the degree to which SIRPα or CD47 co-localized with either presynaptic markers (mCAR, PSD95, Vglut1) or postsynaptic markers (Calbindin. SCGN), we calculated the Manders' Colocalization Coefficients (MCC) for each combination of markers using the FIJI plugin JACOP (Just Another Co-localization Plug-in) (Dunn et al., 2011), n≥3 animals were imaged, and at least two independent fields of view in the OPL were captured per animal using an Olympus Fluoview FV1200 confocal microscope.

Statistical analysis: Statistical analyses of the mean fluorescence intensity, the number of RIBEYE synapses, the number of process endpoints per microglia, the summed process length of microglia, microglia soma size, the percent CD68 and engulfment volume, the percentage of microglia with phagocytic cups, the number of phagocytic cups per microglia, the percent colocalization, and scotopic responses were performed using either unpaired Student's t-test, one-way ANOVA followed by Bonferroni correction, or two-way ANOVA followed by Bonferroni correction in Prism GraphPad 8.0, P values <0.05 were considered statistically significant.

### Example 1—the Impact of Neuronal SIRPα on Temporally Restricted Microglia Activity and Homeostatic State

The following example discusses and elucidates the impacts of neuronal SIRPα on microglia activation and associated neural synapse refinement and synapse maintenance.

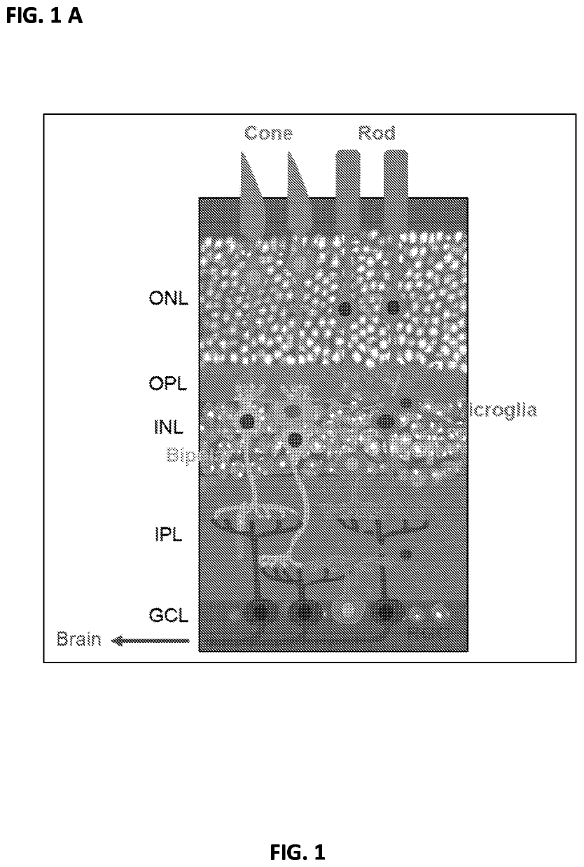

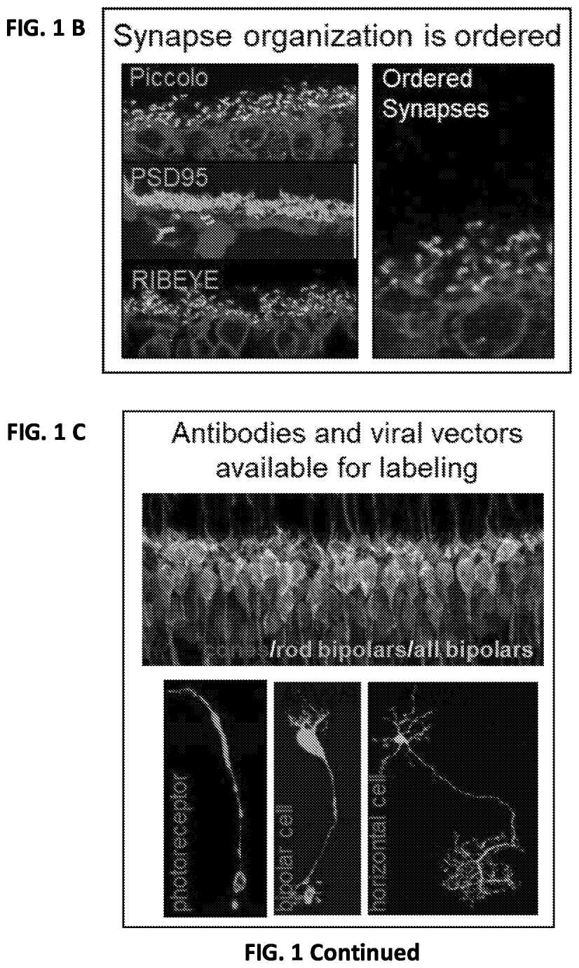

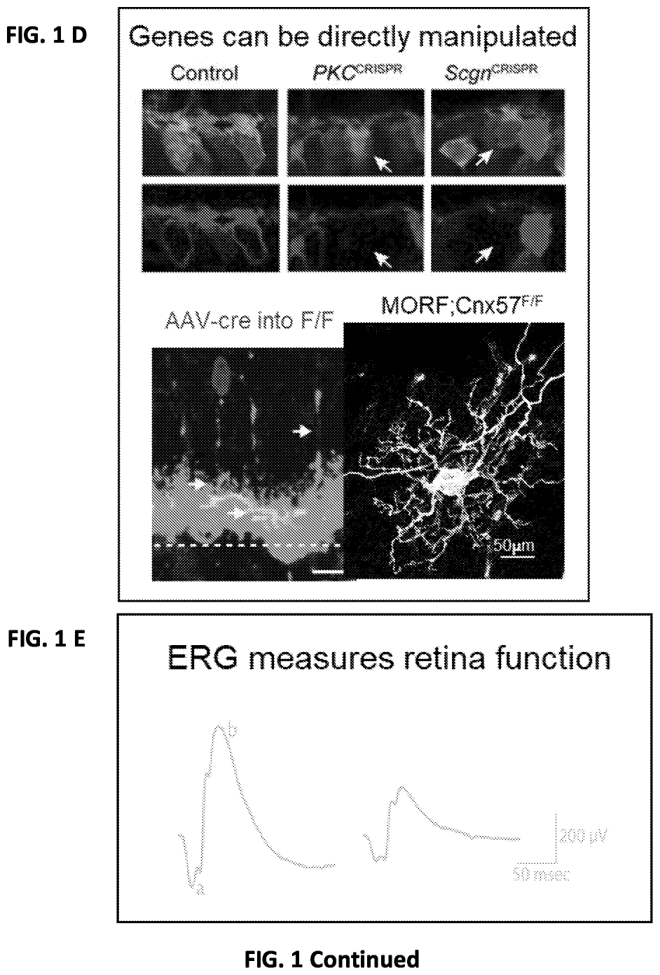

The murine retina provides a robust and accessible system for tracking microglia engulfment, tools for cell type-specific neural and microglia manipulation, and a defined topographic circuit arrangement with high spatial and temporal resolution (FIG. 1A-E).

Studies shown herein illustrate how neurons have coopted an innate immune signaling pathway controlled by signal regulatory protein alpha (SIRPα) to temporally enable and modulate developmental and adult microglial phagocytosis of synapses.

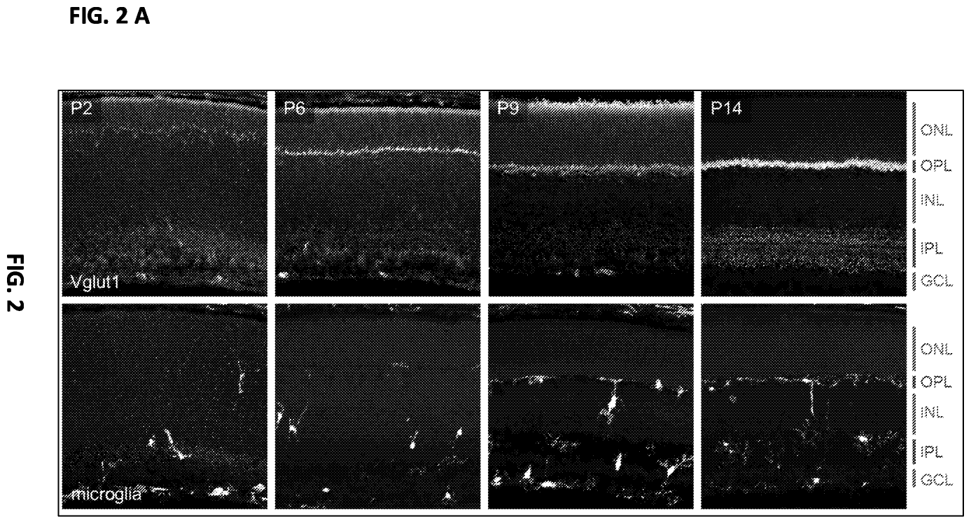

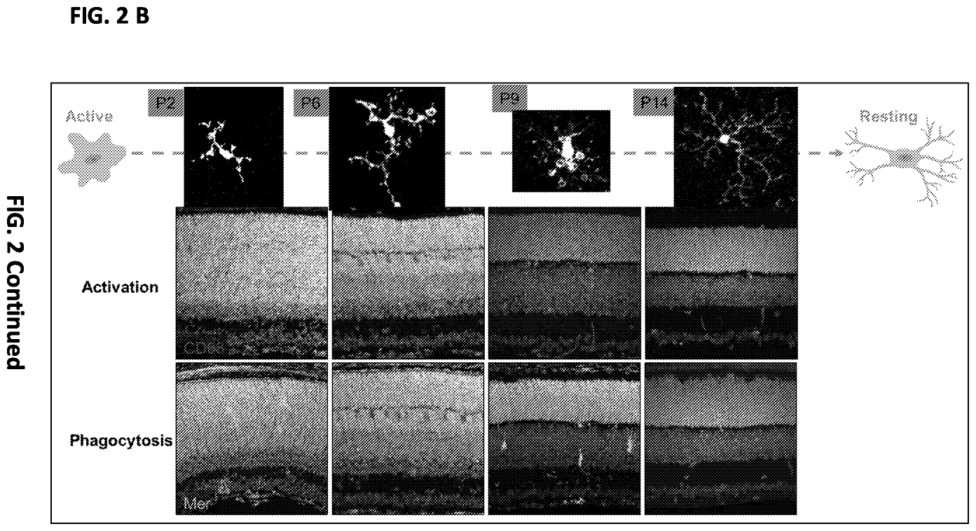

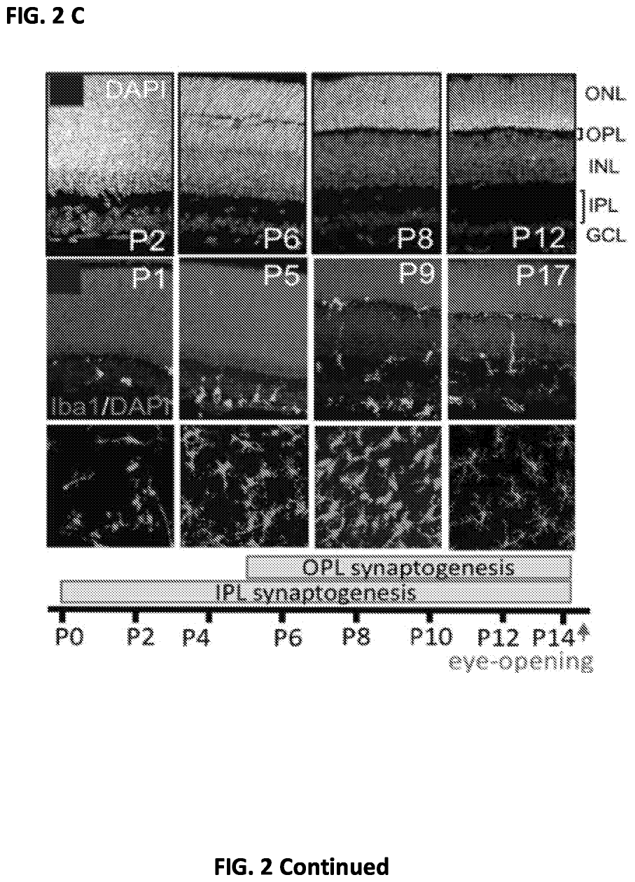

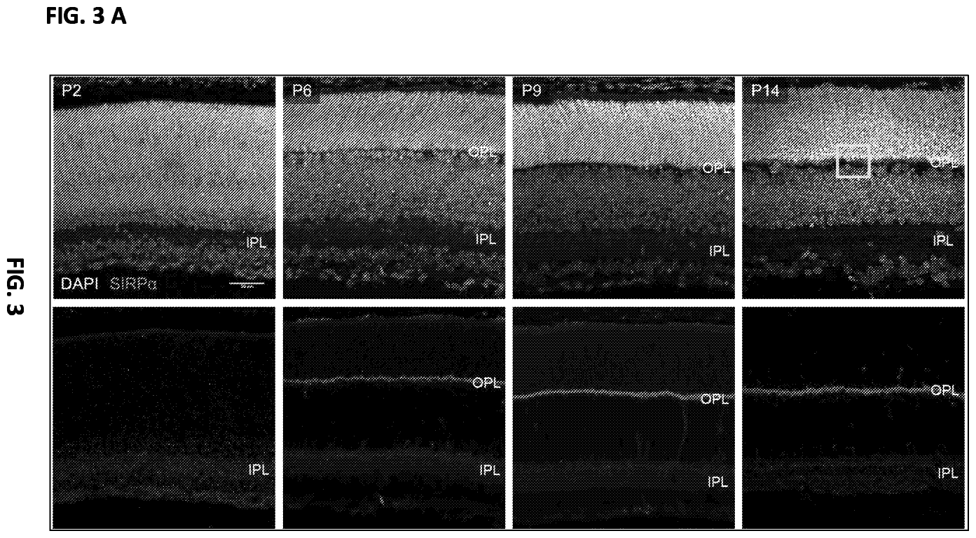

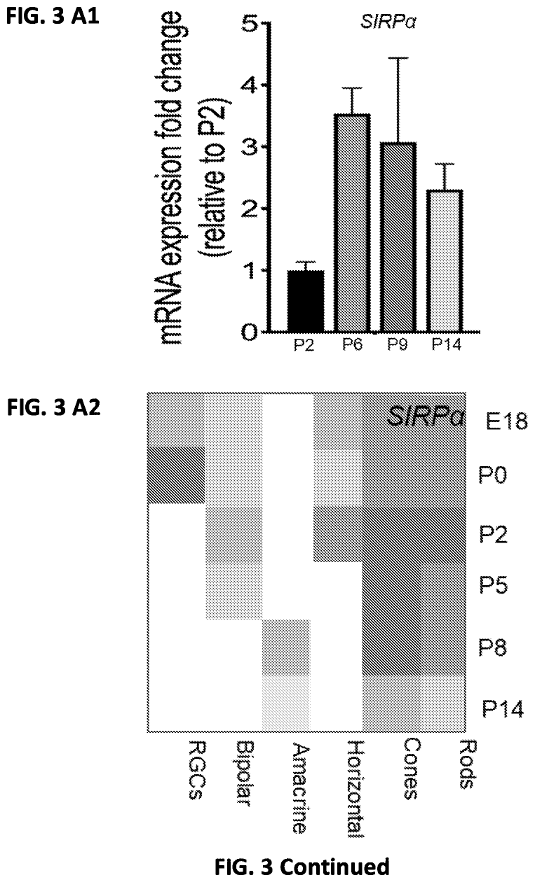

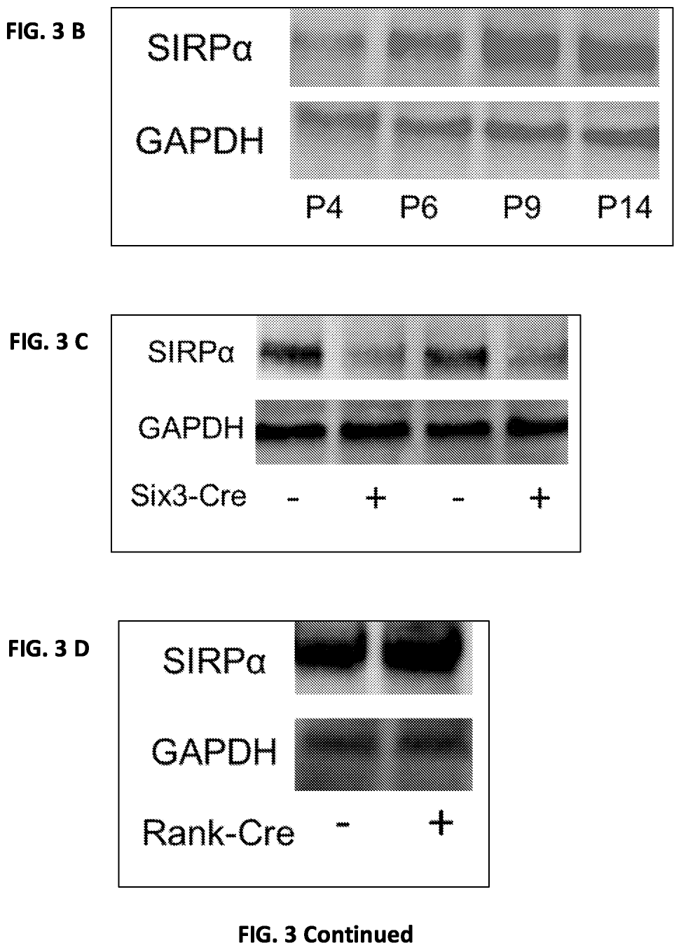

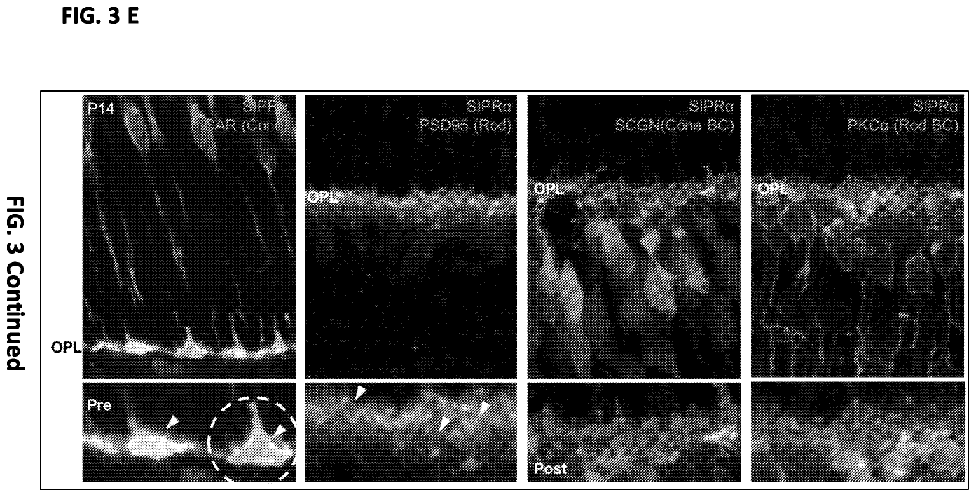

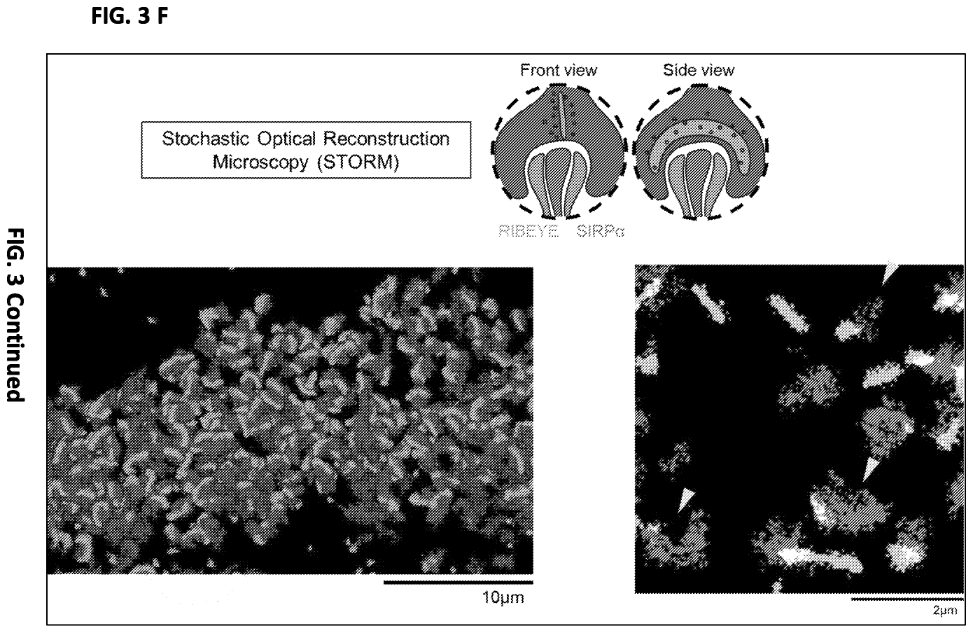

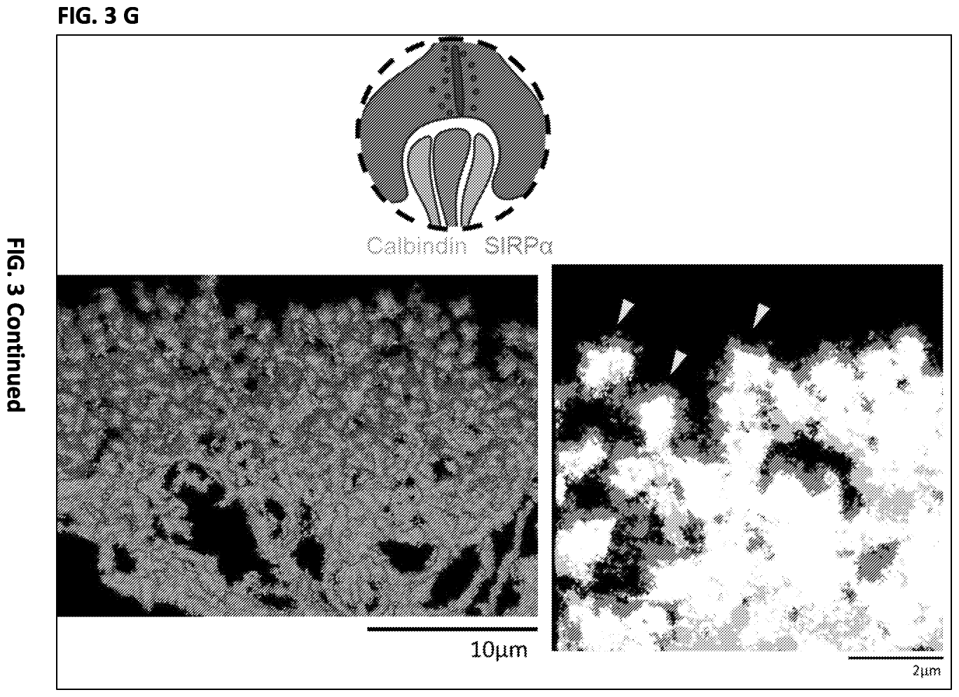

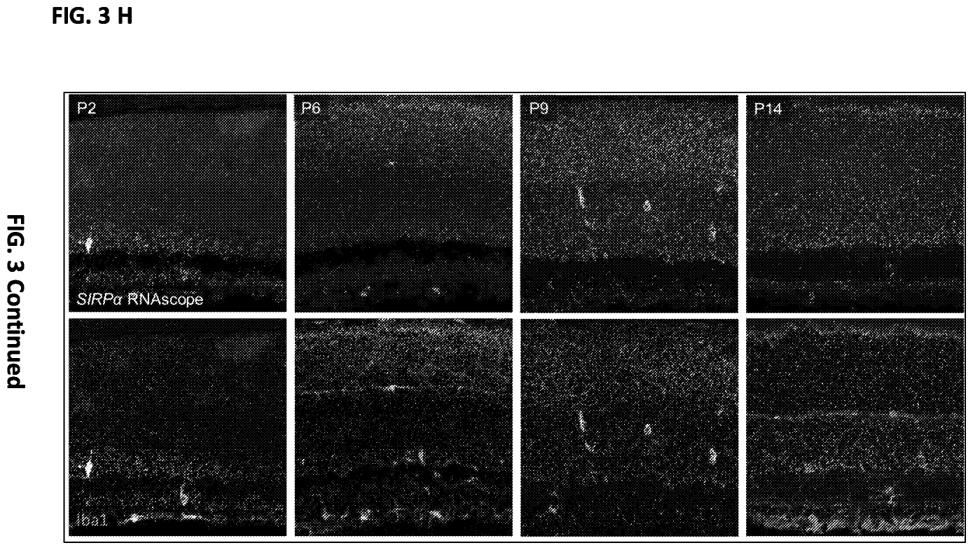

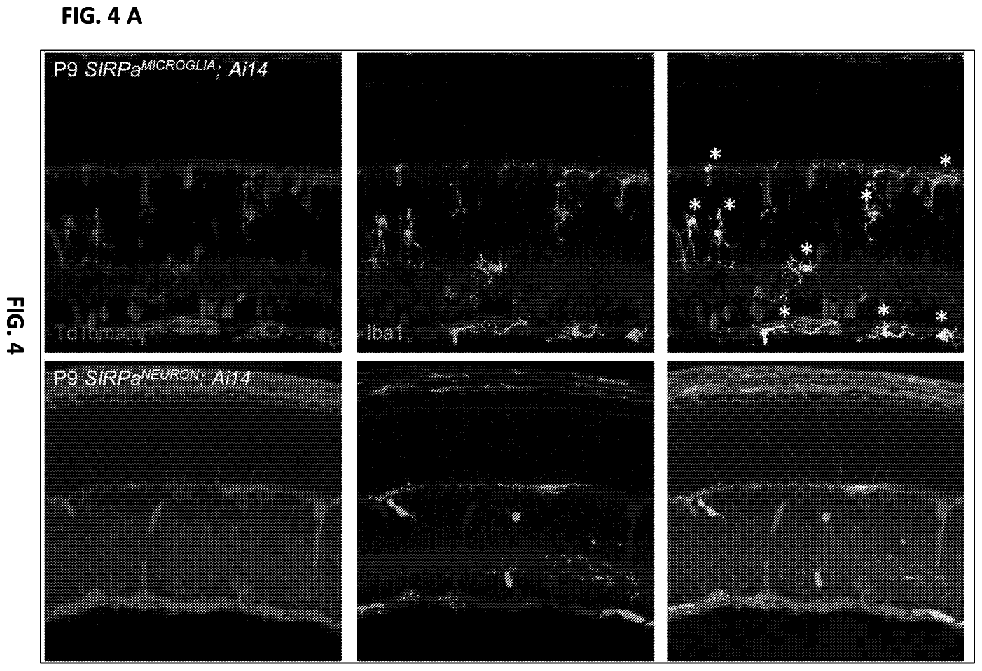

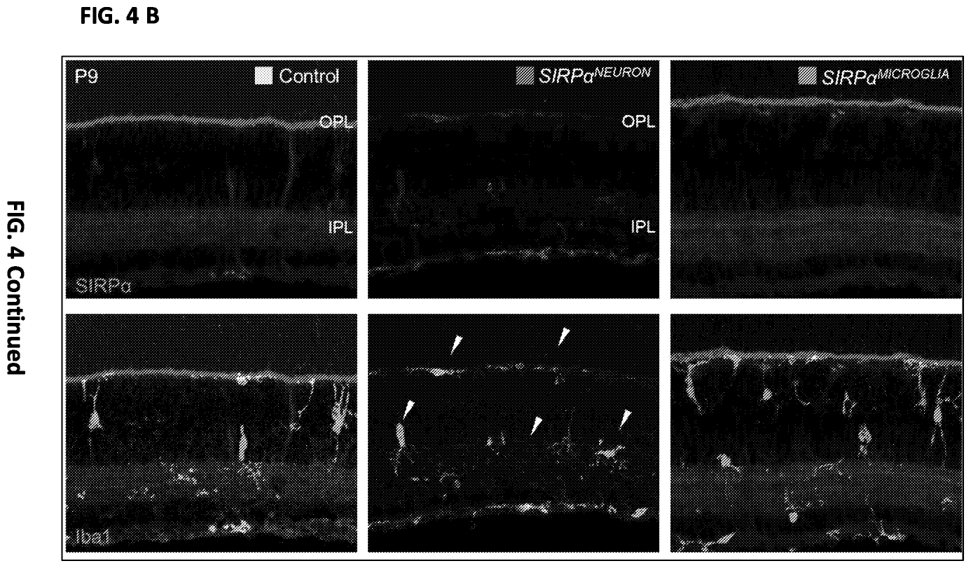

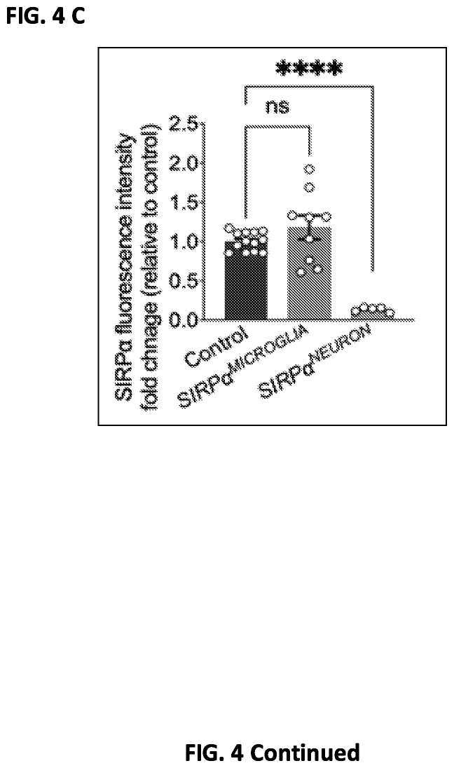

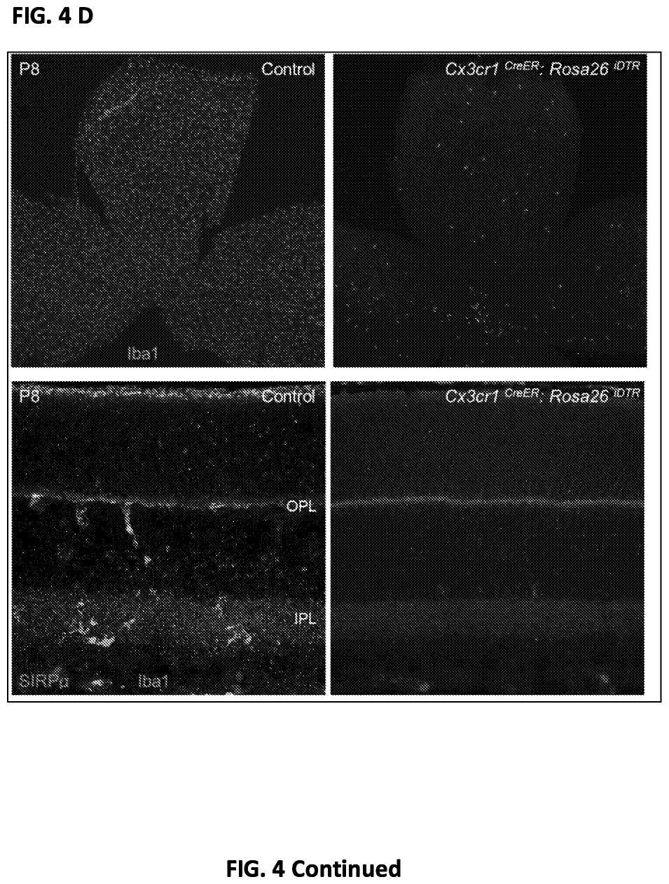

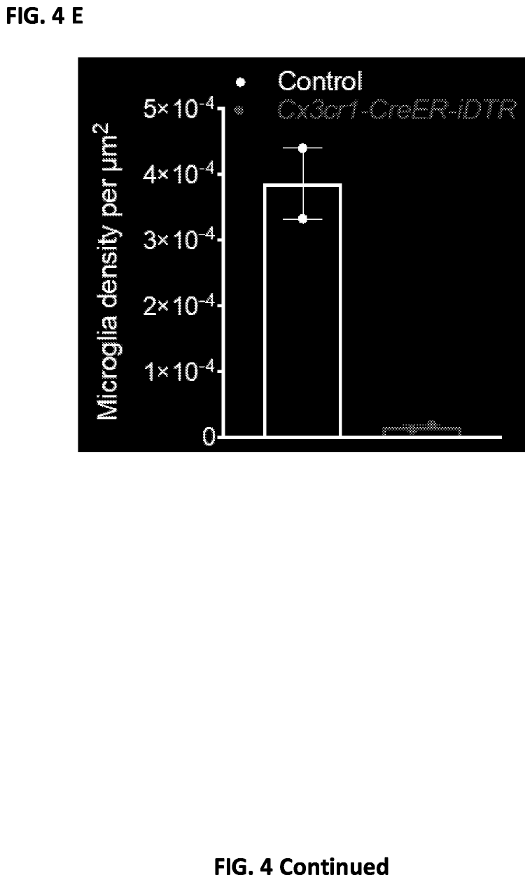

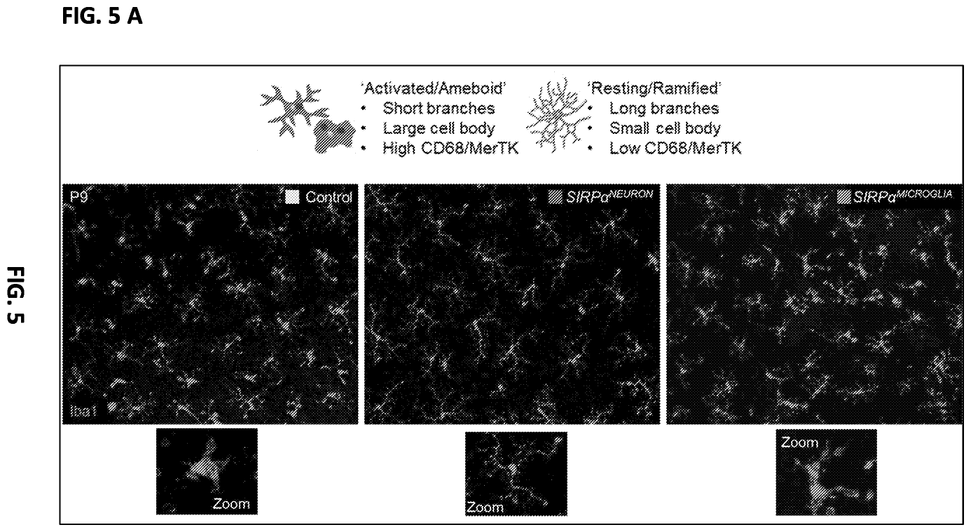

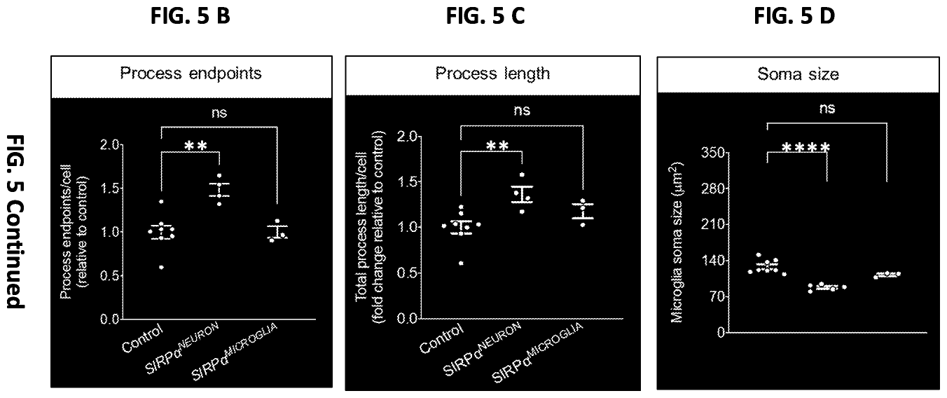

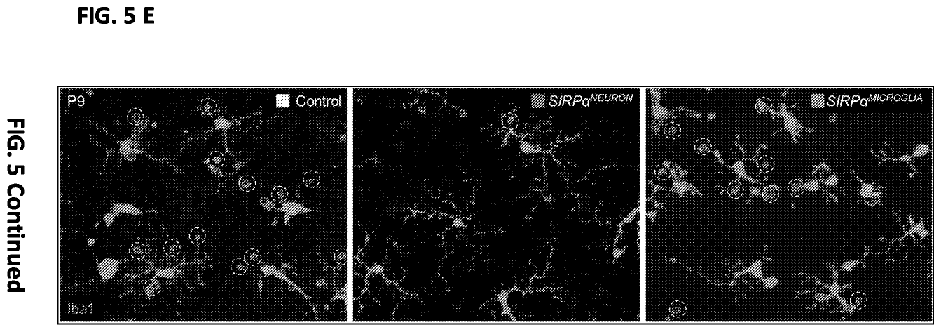

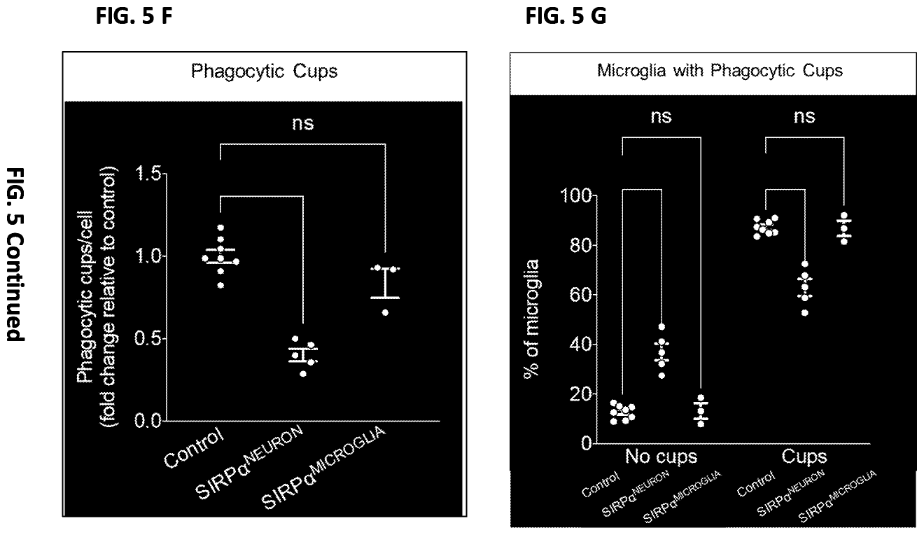

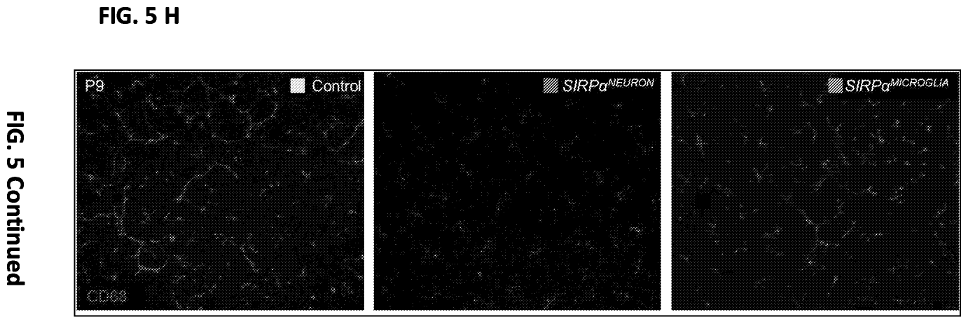

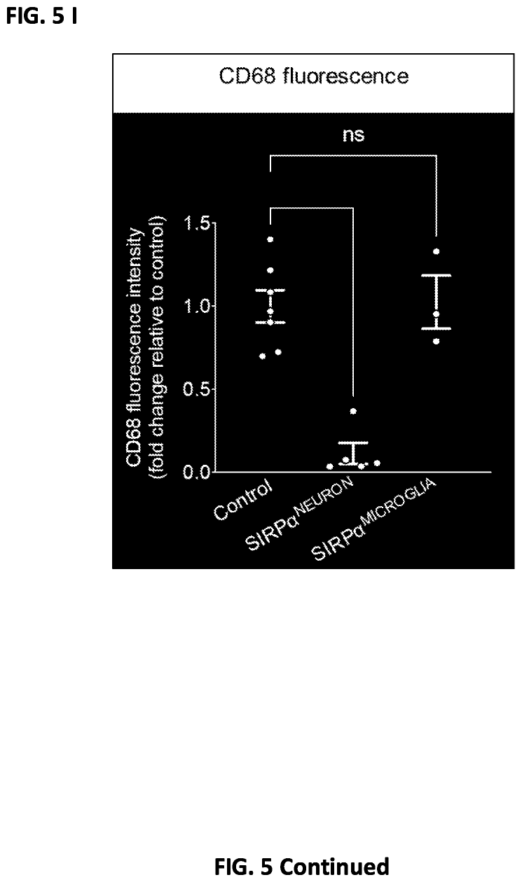

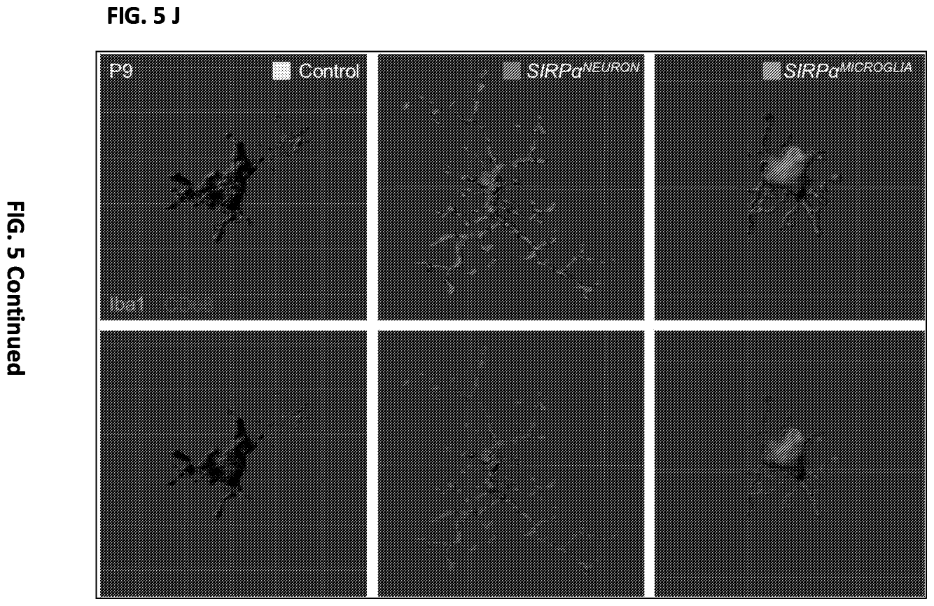

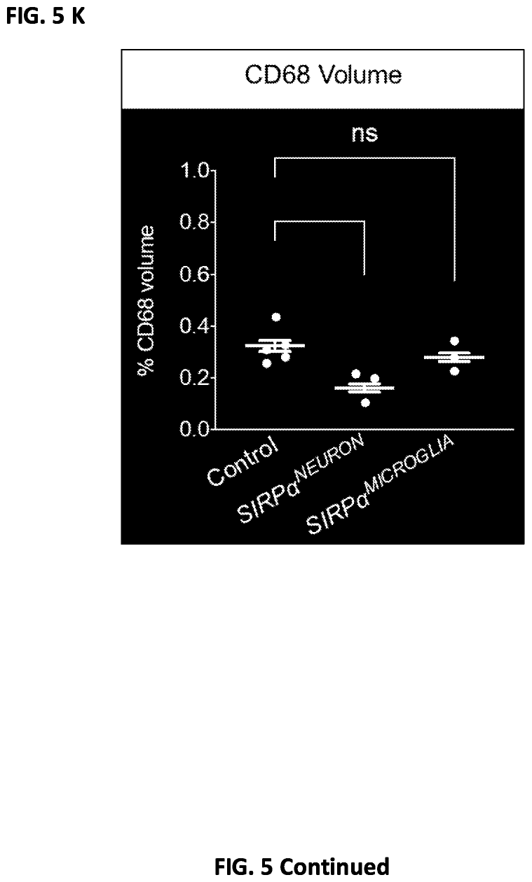

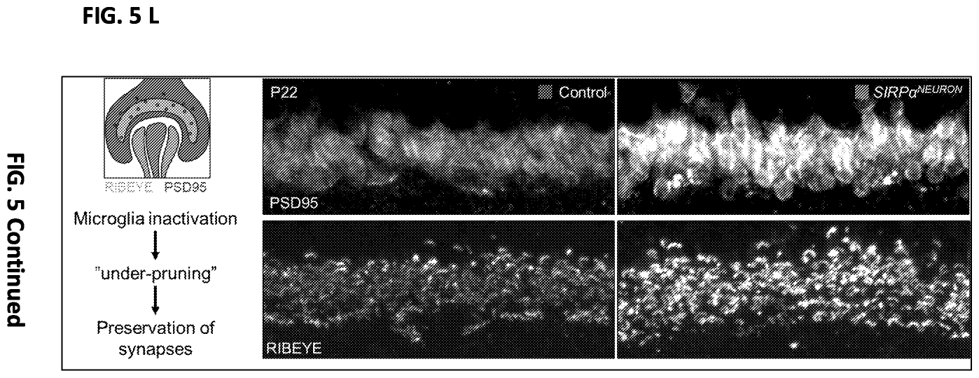

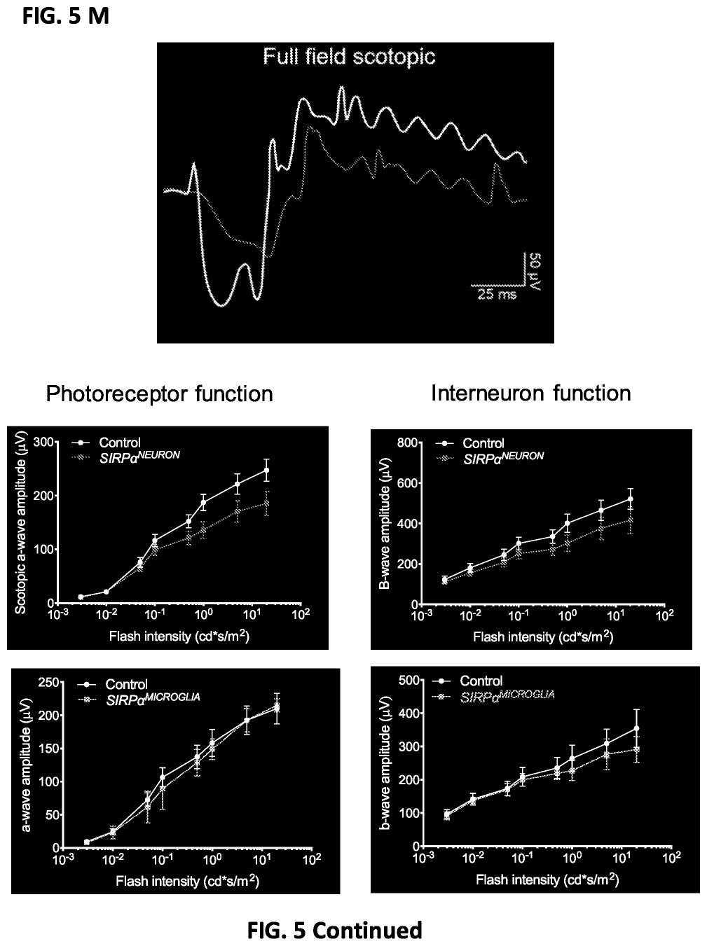

As shown herein, neuronal SIRPα expression correlates with peak pruning and microglia engulfment (FIG. 2A-C and FIG. 3A-B). Neurons are the primary cellular source of SIRPα throughout development (FIG. 3 B-D and FIG. 4 B-C). Neuron-derived SIRPα is required for microglial phagocytosis (FIG. 4 B-C), and deletion of SIRPα from neurons drastically dampens microglial phagocytosis, limits neurite engulfment, and increases synapse number during periods in which microglia are otherwise highly active (FIG. 5A-M). Together, these results suggested neuronal SIRPα was required for microglia engulfment and/or phagocytosis, and that neuronal SIRPα acts as a temporal cue to regulate developmental microglial phagocytosis.

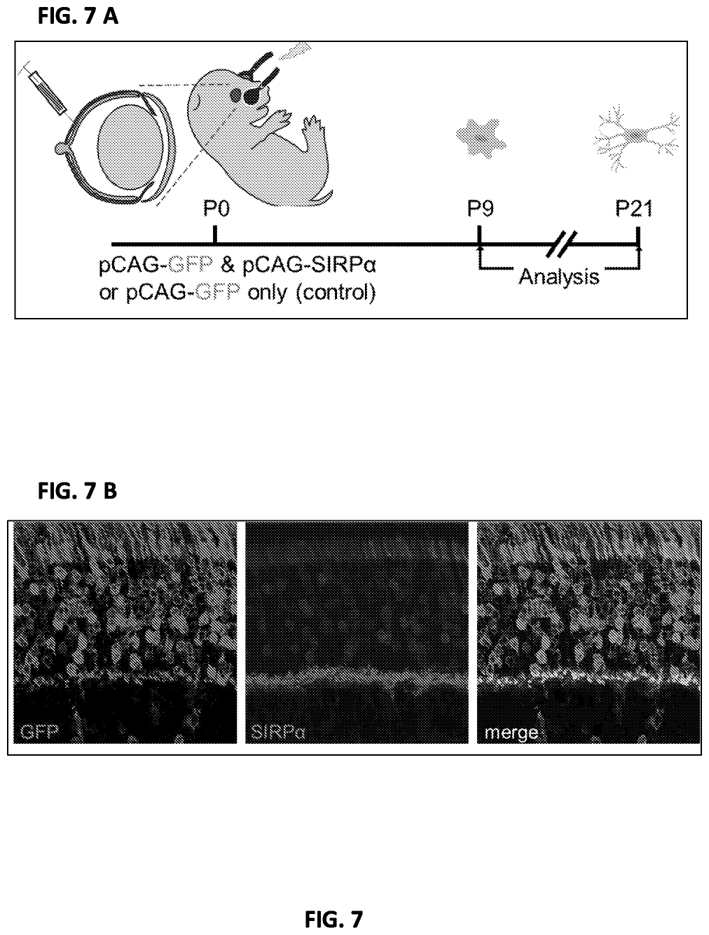

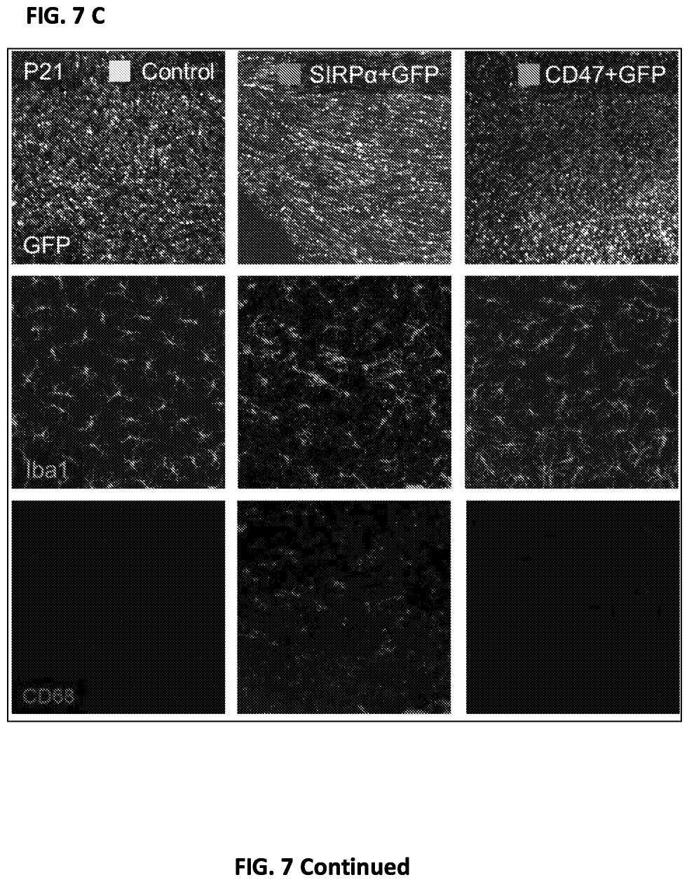

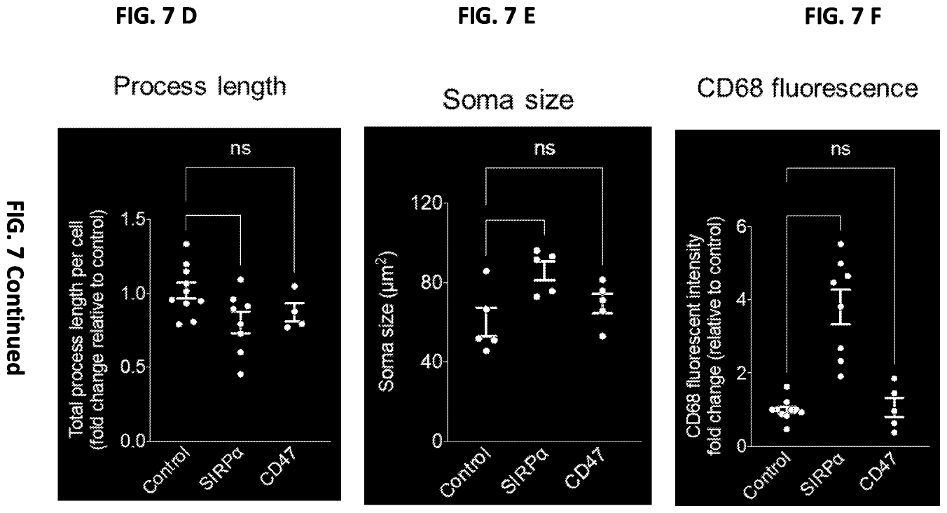

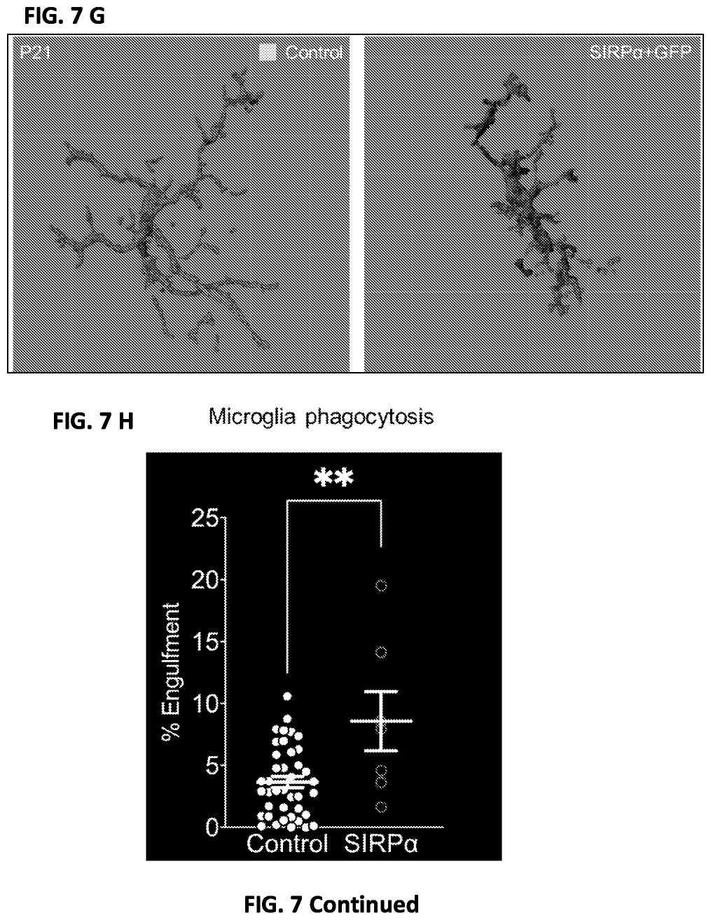

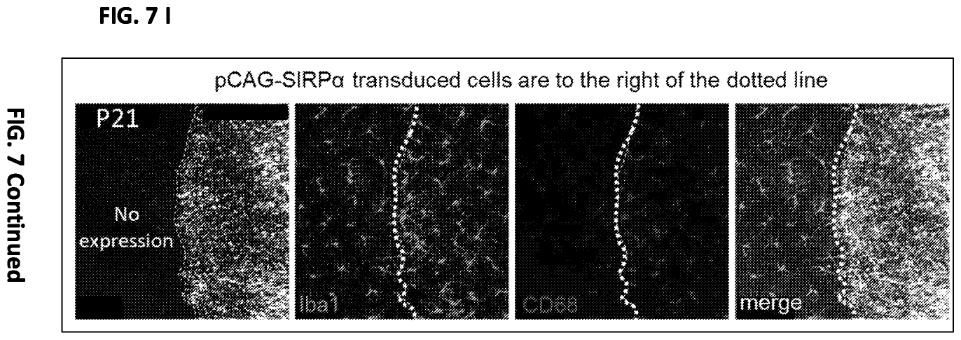

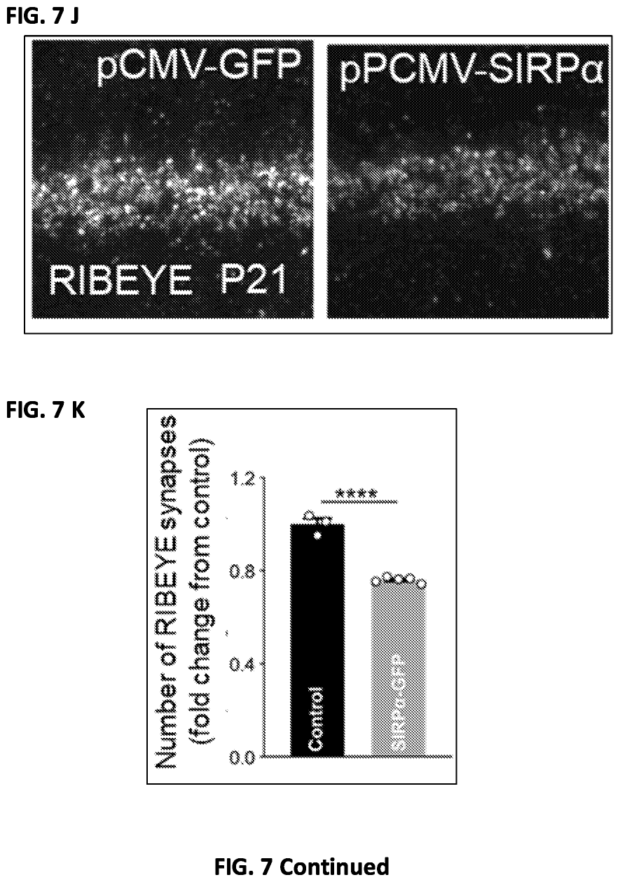

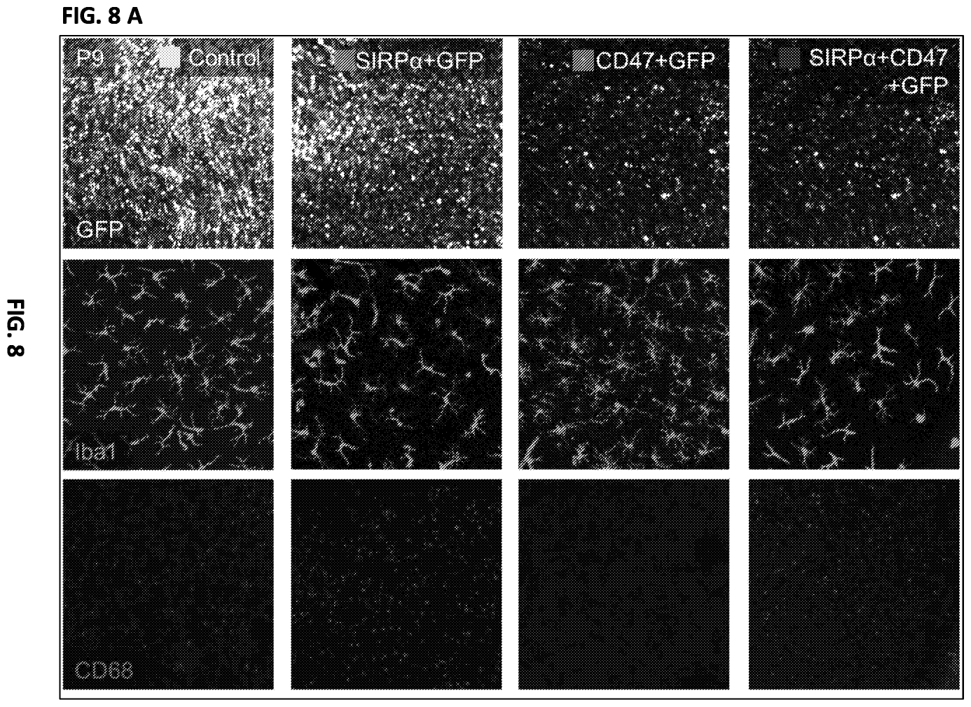

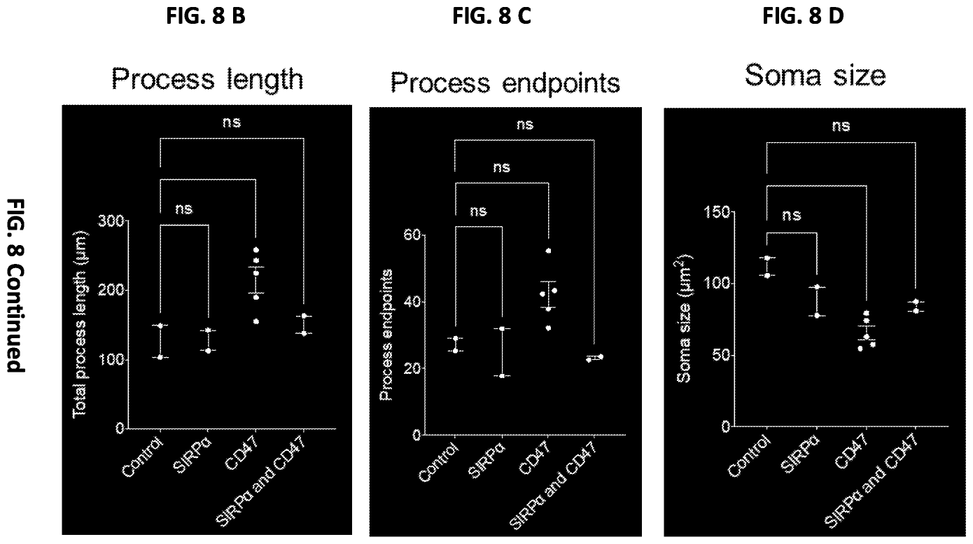

As shown herein, neuronal SIRPα expression is sufficient to locally extend the window of microglia activation after development is complete (FIG. 7A-K and FIG. 8A-D). Together, these results suggested neuronal SIRPα acts locally to impact microglia engulfment and refinement outcomes both in development and in adulthood.

SIRPα was genetically ablated from three cellular sources (e.g., retina neurons, microglia, and all CNS neurons). To remove SIRPα from retina neurons, two different cre lines were employed (see Table 1), both lines express during embryogenesis in retinal progenitor cells and therefore target all neurons but do not target microglia. To remove SIRPα from microglia, the microglia cre line TnfrsfllaCre was utilized, complete microglia removal was also tested, and to remove SIRPα from all CNS neurons (including those in the brain), the pan neuron Cre line Nestin^{Cre} was utilized (see Table 1). A number of other cell specific gene ablations, cell specific gene expressions, or cell specific ablation models were utilized (see Table 1). Cell specific proteins and/or cell localization specific proteins were utilized for immunofluorescent staining, target cells and antibodies utilized are shown in Table 2.

TABLE 1
certain mouse lines for cell selective gene ablation, cell
specific gene expression, or cell specific ablation.

| Mouse lines | | Targeted cell types |
|---|---|---|
| SIRPα^{F/F}; Six3^{Cre} (abbr. as SIRPα^{NEURON}, | | Retina Neurons |
| aka SIRPα^{R-NEURON}) | | |
| SIRPα^{F/F}; Chx10^{Cre} (abbr. as SIRPα^{Chx10}) | | |
| SIRPα^{F/F}; Rank^{Cre} (abbr. as SIRPα^{MICROGLIA}) | | Microglia |
| Cx3cr1^{CreER}; Rosa26^{iDTR} | | |
| SIRPα^{F/F}; Nestin^{Cre} (abbr. as SIRPα^{PAN-NEURON}) | | All CNS Neurons |
| Mouse Line | Specificity | Reference ID |
| SIPRα^{F/F} | Conditional SIPRα removal | SIPRαTM1IMPC |
| CD47^{-/-} | Deletion of CD47 | JAX: 003173 |
| Six3^{Cre} | All retina neuron deletion (E9) | JAX: 019755 |
| Chx10^{Cre} | Most retina neurons (E11.5) | JAX: 005105 |
| Rdpsi^{Cre} | Rod specific deletion (P7) | PMID: 15682388 |
| HRGP^{Cre} | Cone specific deletion (P8) | PMID: 15635292 |
| Pcp2^{Cre} | Rod bipolar cell deletion (P15) | JAX: 010536 |
| Cxn57i^{Cre} | Horizontal cell deletion (P0) | PMID: 27022629 |
| Ptf1a^{Cre} | Amacrine cell Cre (E10.5) | JAX: 023329 |
| Brb3b^{Cre} | RGC cell deletion (P0) | JAX: 030357 |
| MORF | Sparse cre-dependent GFP | PMID: 32795398 |
| CXC3R1 | Microglia specific GFP | JAX: 005582 |
| FoxP2^{Cre} | F-RGC subtype Cre | JAX: 030541 |
| RANK^{Cre} | Microglia specific Cre | PMID: 22344299 |

TABLE 2
Antibodies utilized to manipulate and/or identify
specific target cells or cellular localizations.

| Target cell | Antibody target proteins |
|---|---|
| Rods | Rhodopsin |
| Cones | Cone Arrestin, OPN1SW |
| Amacrines subsets | Syntaxin, Chat, VGLUT3, TH, GAD65/67, GlyT1 |
| Ganglion cell | Brn3a, Brn3b, Brn3c, Melanopsin, KV4.2, Cart, |
| subsets | SM132, RBPMs |
| Horizontal cells | Calbindin |
| Rod bipolars | PKCα, Ch10 |
| Cone bipolars | Csen, HCN4, Syt2, Scgn, Chx10 |
| Muler glia | Sox9 |
| Synapses | Bassoon, PDS95, Gephryn, Synaptotagmin2, |
| | Piccolo, Ribeye, Synaptophysin, Vglut1-3 |
| Microglia | Iba1, CD68, MerTk |

As described herein assays comprising spatially and temporally restricted neuronal SIRPα loss- and gain-of-function approaches were coupled with histological assessments of measures of microglia subtypes, and were used to determine the relationship between local SIRPα production, microglial phagocytosis, homeostatic state, and development.

In the periphery, it is well established that professional phagocytes can express SIRPα, and binding of its receptor CD47 on host cells serves as a ‘don't cat me signal’ associated with reduced phagocytosis. In line with this idea, various cancers upregulate CD47 to limit phagocyte recognition and evade the immune system. However, the communities view of these interactions is now beginning to expand with the demonstration that SIRPα is also expressed by cancer cells, and downregulation of SIRPα likewise enhances cancer immune evasion. Thus, the relative levels of SIRPα in phagocytic and non-phagocytic cells appear important for modulating immune outcomes.

As described herein, dual roles for different cellular sources of SIRPα is present in the nervous system. As described herein, neuronal SIRPα is required for microglial phagocytosis during development, while microglia SIRPα is dispensable (FIG. 5A-M).

Understanding the molecular mechanisms underlying these effects can fundamentally change the fields understanding of neuron-microglia interactions and define a new role for cell type-specific SIRPα signaling and modulation within the nervous system (e.g., the central nervous system (CNS) (e.g., the retina, spinal cord, brain, etc.) and/or the peripheral nervous system (PNS). Published data and data presented herein showed at microglia function in retina mirrors that in the brain. Determining how neurons signal to microglia to temporally control their phagocytic capacity, requires an accessible and robust system for mapping microglial phagocytosis, well established genetic tools, and a defined circuit with excellent spatial and temporal resolution. The retina and its brain targeting axons in the dLGN elegantly meet these criteria.

A large body of evidence suggests that retina microglia are structurally, functionally, and developmentally analogous to those in the brain. Refinement of the retina's diverse neuron types (˜146 different types of neurons) occurs during a key postnatal window when neurites become restricted to two synapse layers (FIG. 1A).

The inner plexiform layer (IPL) appears first, followed by the outer plexiform layer (OPL). Refinement peaks at approximately day P9 of age and proceeds until day P14 of age when neurons have adopted their adult morphologies. Retina microglia appear to play key roles in this process (FIG. 2 C). First, the location, numbers, and phagocytic state of microglia all coincide precisely with retina synapse refinement-microglia activity peaks at P9 and is restricted at P14 (FIG. 2A-C). Second, most microglia are present in retina synaptic layers (>80%), and their numbers are highest during refinement (FIG. 2A-C). Third, genetic or pharmacological modulation of microglia can alter retina neuron fate, function, and connectivity. Finally, our previous work has shown that microglia-mediated synapse modifying pathways in the brain are conserved in the retina. Together, these data make the critical point that microglia function and phagocytic state are conserved in the retina and temporally align with neuronal maturation.

SIRPα is a cell surface receptor with a cytosolic tyrosine-based inhibitory motif (ITIM). To date, the only known ligand for SIRPα is CD47. Interaction between CD47 and SIRPα causes ITEM phosphorylation, recruitment and activation of SHP1 and SHP2, and inhibition of cytoskeleton rearrangement and phagocyte engulfment. Traditionally, CD47 on host cells is thought to bind SIRPα on phagocytes leading to decreased engulfment. However, SIRPα can also be expressed by nonphagocytic host cells where it has the potential to modulate immune outcomes through trans or cis interactions with CD47. In particular, SIRPα loss-of-function increases pathogenesis and immune evasion while gain-of-function reduces pathogenesis for a range of cancers including prostate cancer, astrocytomas, and liver cancers. These data are consistent with the notion that the SIRPα in host cells can directly influence immune-dependent outcomes.

Evidence is also emerging for neuron-neuron and neuron-microglia SIRPα signaling. In the CNS SIRPα is found at high levels on both neurons and microglia, while CD47 is present primarily in neurons. Multiple lines of evidence suggest that CD47 interacts with both neuronal SIRPα and microglia SIRPα. In the former case, neuronal SIRPα interacts with CD47 to regulate presynaptic maturation. In the latter case, CD47 and SIRPα signaling limit exuberant neural refinement by microglia based on whole-body knockouts of each protein. Yet, how and whether neuronal SIRPα influences the ability of microglia to sense and respond to CD47 was unknown. Given that ‘don't eat me’ CD47 levels are high in development when microglia are most phagocytic, such interactions are considered likely to occur. Indeed, as described herein, neuronal SIRPα instructs microglial morphology, activation state, and phagocytic capacity. Together, these findings make the critical point that neuronal SIRPα is poised to influence microglia-dependent phagocytosis.

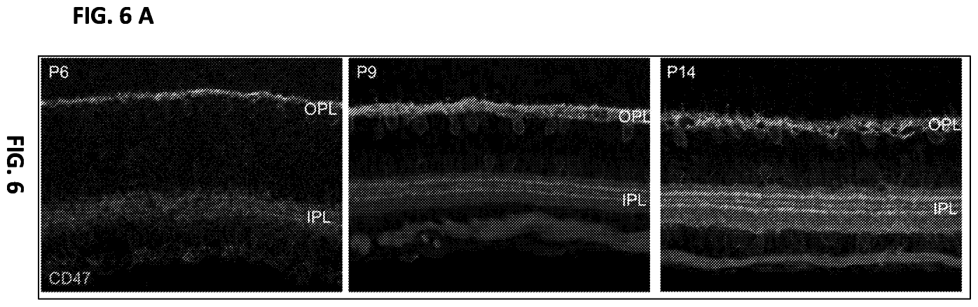

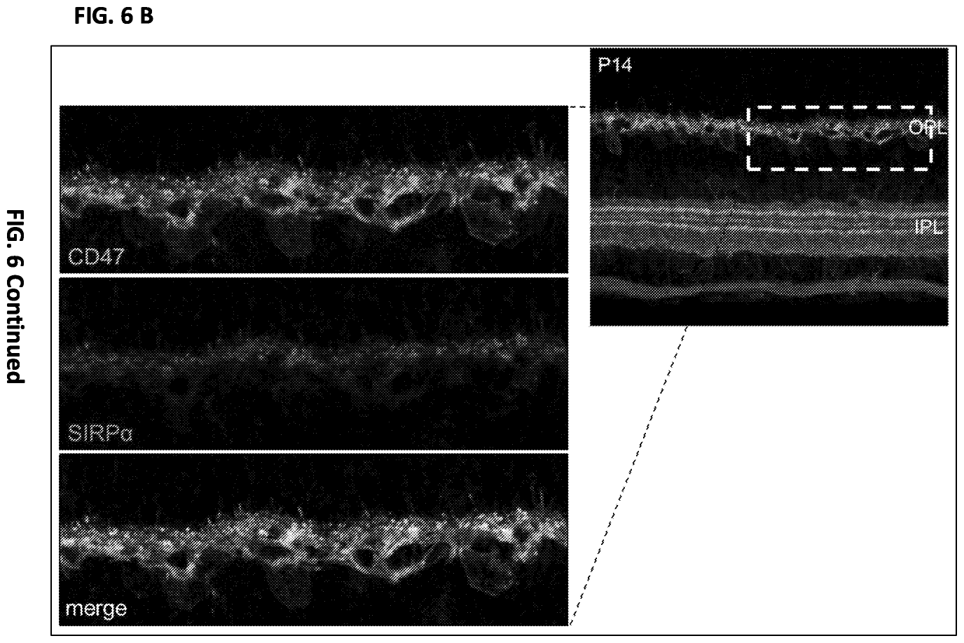

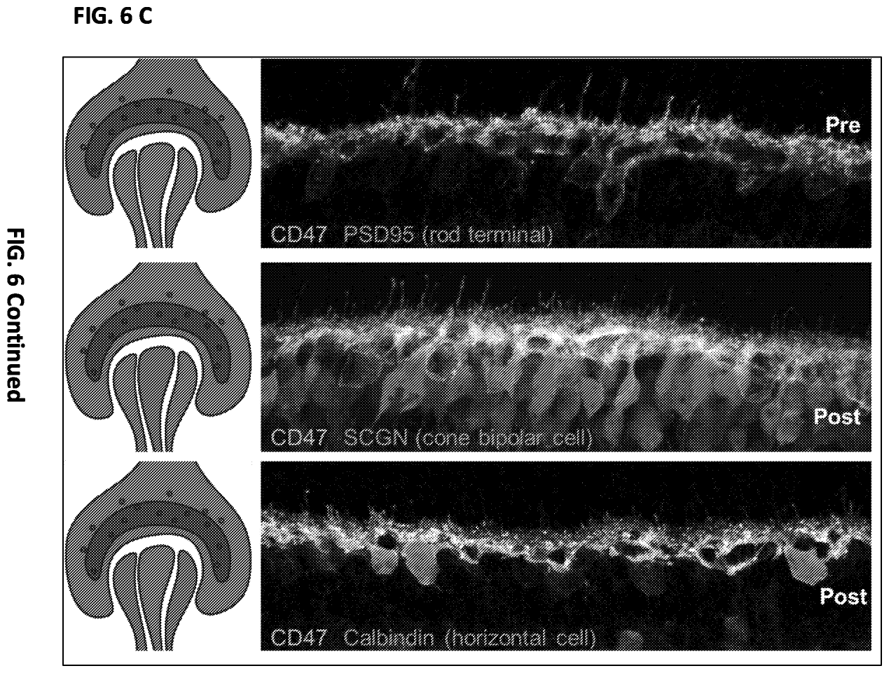

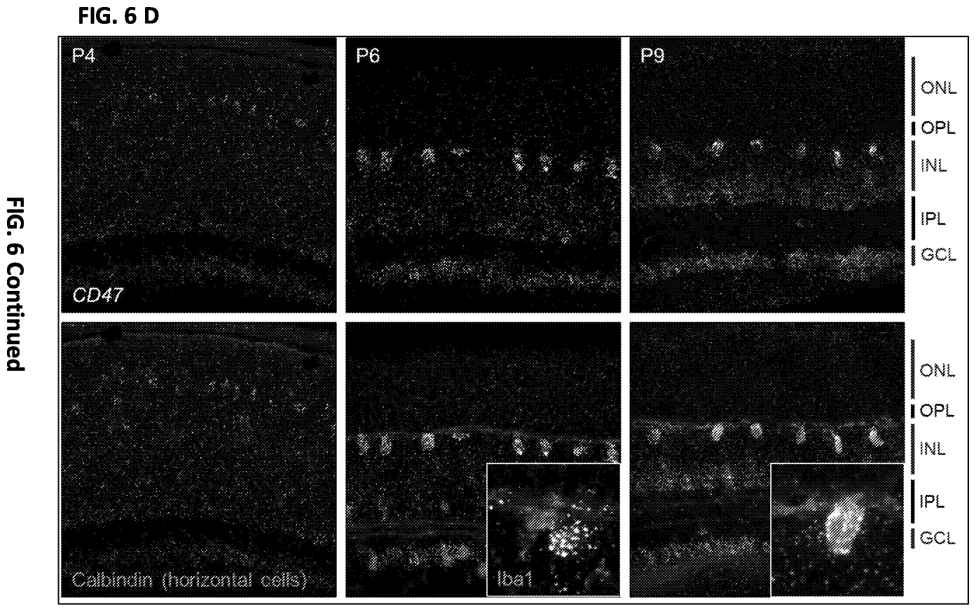

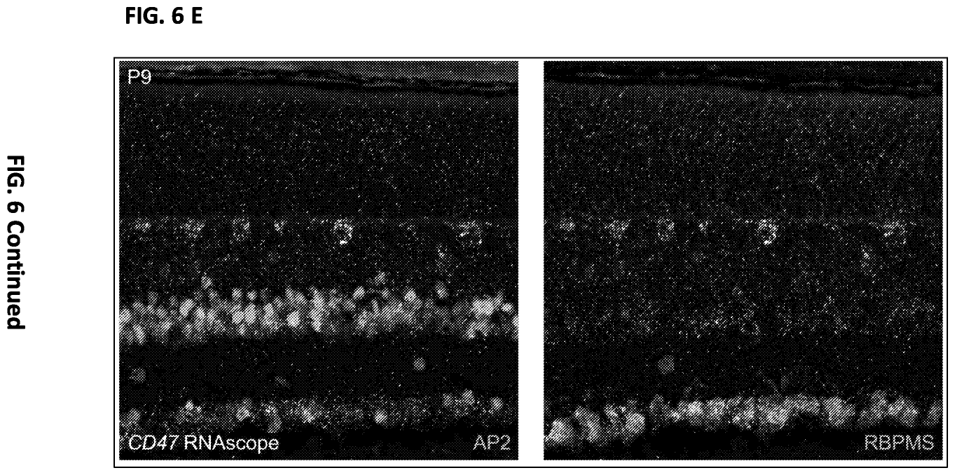

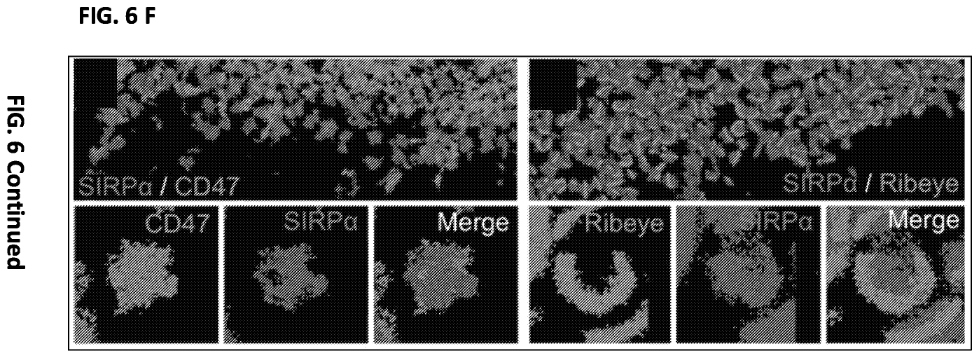

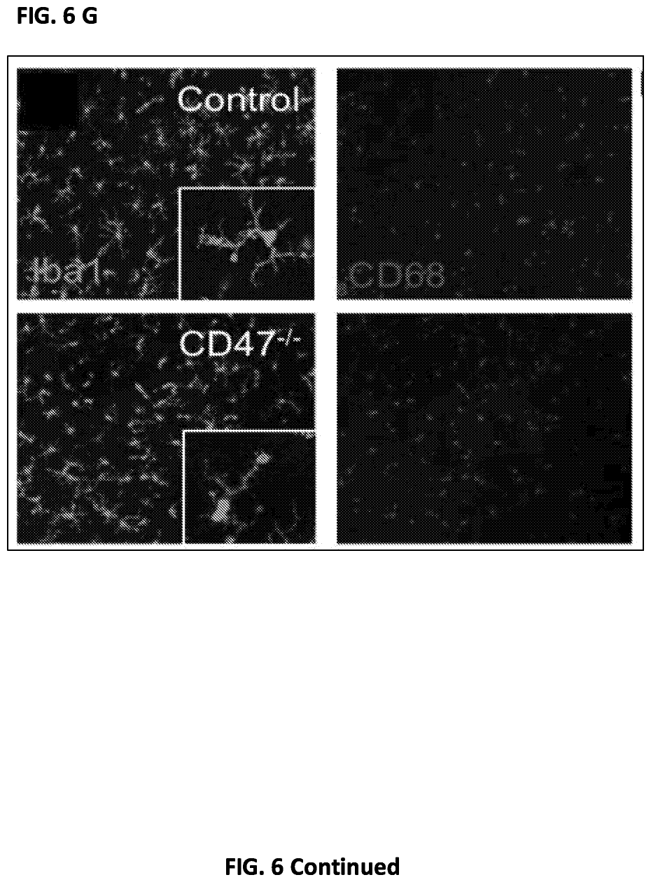

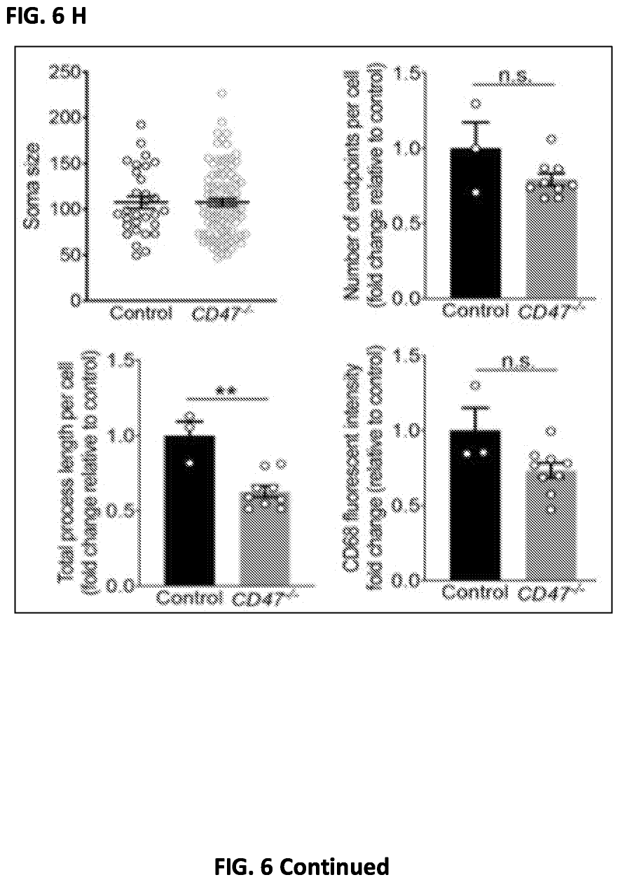

Provided herein are data illustrating that neuronal SIRPα is required for microglial phagocytosis and acts as an important cue to regulate engulfment. First, the precise timing and spatial distribution of SIRPα in neuron populations was shown (FIG. 3A-B). Second, the critical role of neurons as a significant cellular source of SIRPα was revealed (FIG. 3A-D, and FIG. 4 B-C). Third, neuronal SIRPα was demonstrated to be required for microglial phagocytosis, while microglia SIRPα was shown to be dispensable (FIG. 4 B-E, and FIG. 5A-M). Fourth, how neuronal SIRPα-dependent changes in microglia impacted engulfment and synapse refinement was illustrated (FIG. 5A-M). Fifth, CD47 was shown to be highly colocalized with neuronal SIRPα (FIG. 6A-E). Together with the inventors published work, these data showed that neuronal SIRPα production spatially and temporally aligned with microglial phagocytic state.

Previously the inventors documented significant SIRPα expression in both inner and outer retina synapse layers using a reporter line, validating a prior report. To determine whether SIRPα was at the right place and time to modulate microglia outcomes, the inventors mapped the histological distribution of SIRPα over development (FIG. 3A-H). SIRPα was present at low but detectible levels in microglia, showing dim co-staining with the microglia marker Iba1 (FIG. 3 H). However, the bulk of the SIRPα signal was localized to retinal synapses (FIG. 3A, and FIG. 3 E-G). SIRPα first appeared in these regions as the inner (IPL) and outer plexiform layers (OPL) emerged, and its expression peaked as they refined, (P2 -P14), coinciding with high levels of microglial phagocytosis (FIG. 3A-I). After refinement ended, total SIRPα levels declined, though some SIRPα remained in the OPL in adults. The inventors confirmed SIRPα localization at synapses by staining with pre- and post-synaptic markers, analysis was focused on the OPL, where synapses occur at one distal location, and pre- and post-synaptic inputs can be readily distinguished (FIG. 3 E-G). The inventors found that SIRPα colocalized with pre-synaptic cone and rod terminal markers (mCAR and PSD95) but not with postsynaptic rod and cone bipolar cell terminals (SCGN and PKCa). Together. these results demonstrated that, as in the brain, SIRPα was found in both neurons and microglia during neuron refinement but that the majority of SIRPα was associated with synapses and levels peak when microglia are most active. Thus, neuronal SIRPα was in the right place at the right time to impact microglial phagocytosis.

Provided herein are data showing that neurons produce high levels of SIRPα. SIRPα can be cleaved and secreted such that its histological localization may not necessarily reflect its primary cellular source. Accordingly, the cellular source of SIRPα over development was determined using in situ hybridization analysis for SIRPα mRNA localization (FIG. 3 H). Even early in development (P2). SIRPα mRNA was present in both neurons and microglia, and this pattern persisted throughout refinement. From P14. SIRPα mRNA signal appears largely restricted to neurons. To confirm and extend these findings, the inventors genetically assessed which cells produce SIRPα by selectively eliminating SIRPα in microglia or neurons. Conditional SIRPαFff mice were crossed to either a retina neuron-specific Cre line (Six3^{Cre}) or the tumor necrosis factor receptor superfamily member 11A. (TNFRSF11A) Cre line. Similar to Cx3crICR. TNERSFILACre is expressed in, and targets. yolk sac-derived erythro-myeloid progenitors which in the brain are comprised of microglia. For simplicity, herein these lines are described as SIRPα^{MICROGLIA} (selective removal of SIRPα from microglia) and SIRPα^{NEURON} (selective removal of SIRPα from retinal neurons) respectively. The inventors found that in the absence of microglia-derived SIRPα, total SIRPα levels are relatively unaffected (FIG. 4 B-C). In contrast, in the absence of retina neuron-derived SIRPα, SIRPα levels were significantly decreased (FIG. 4 B-C), resulting in a largescale reduction (˜90%) and no SIRPα signal at synapses. Further, the low levels of microglia-localized SIRPα remained visible in SIRPα^{NEURON} mice, while neuron-associated SIRPα was unaltered in SIRPα^{MICROGLIA} mice, further validating the specificity of these models (FIG. 4 B). To independently confirm these results, the inventors utilized the microglia depletion model Cx3Cr1^{CreER}; Rosa26^{iDIR} which showed high SIRPα levels comparable to that in controls (FIG. 4 D-E). Together these data indicated that neurons were responsible for all synapse-associated SIRPα and the majority of total SIRPα production.

The inventors have found that neuronal SIRPα robustly regulates microglial phagocytosis. Given the high levels of SIRPα derived from neurons, the relative roles of neuronal and microglia-derived SIRPα in modulating microglia was determined. To examine this, microglia were assessed over development in SIRPα^{NEURON} and SIRPα^{MICROGLIA} mouse models using seven independent measures of microglial phagocytosis and activity (FIG. 5A-K). These measurements included cell shape and size (soma size, process length, and endpoint number), phagocytic markers (total CD68 levels and 3D reconstruction of CD68 volume per cell), and phagocytosis measures (presence and level of phagocytic cups). In control SIRPα^{F/F} mice, microglial phagocytic activity peaked at P9, and cells correspondingly displayed short neurites, large somas, and high levels of CD68 (FIG. 5A-K). Strikingly, microglial phagocytic activity was largely absent in SIRPα^{NEURON} mice during this same period. Microglia were highly ramified at P9 with long, extensive processes and displayed significant alterations to every measure, including 1) increased process endpoints, 2) increased process length, 3) reduced soma size, and 4) markedly reduced staining with the phagocytic marker CD68 when measured either globally or in single reconstructed cells (FIG. 5A-K). In addition, significantly fewer microglia in SIRPα^{NEURON} mice displayed phagocytic cups, and those with cups contained half the number per cell relative to controls (FIG. 5 E-G). By marked contrast, microglial phagocytic state was unaffected in SIRPα^{MICROGLIA} retina as measured by all seven measures (FIG. 5A-K). Microglia in these animals displayed ameboid morphology, comparable total process endpoints number and length, soma size, CD68 staining and internalized volume, and phagocytic cups indistinguishable from that in controls. These results strongly supported the finding that neuronal, but not microglial, SIRPα was required for microglia activity and engulfment during neuronal refinement.

As described above, the requirement of neuronal SIRPα for microglia engulfment and appropriate synapse refinement was determined. As such, neuronal SIRPα-mediated alterations to microglial phagocytic state should, in turn, impact the degree to which microglia refine synapses. To examine this, synapses in SIRPα ^{NEURON} and SIRPα^{MICROGLIA} mice were examined with the aid of the helpful organizational features of the OPL. In this layer, most synapses are derived from rods, and individual synapses can be quantified due to their size and arrangement. In SIRPα^{NEURON} mice, decreased microglial phagocytosis was associated with an increase in both the relative levels of RIBEYE (an OPL synapse marker, alternative markers include e.g., PSD95 and Bassoon) fluorescence and the total number of RIBEYE positive synapses (FIG. 4 F-G), while synapses were unaffected in SIRPα^{MICROGLIA} mice. To determine if these changes altered visual responses, electroretinogram recordings were performed, SIRPα^{NEURON} mice showed altered scotopic neuron function (FIG. 5 L-M). Together, these data indicated that neuronal SIRPα-dependent microglial phagocytosis directly influenced microglia engulfment and synapse refinement.

It is known that CD47 is a ligand for SIRPα, and that CD47 is highly colocalized with neuronal SIRPα. To elucidate the cellular mechanisms through which neuronal SIRPα may interface with CD47, the inventors determined where and when CD47 was present in the retina.

Immunostaining for CD47 revealed that it was apparent specifically in synapse layers as refinement is initiated, increased at P9 during the peak of microglia-mediated refinement, and was maintained at synapses into adulthood (FIG. 6A-E). Further CD47 was highly co-localized with SIRPα, and deletion of neuronal SIRPα markedly reduced this co-localization, suggesting the neuronal SIRPα was poised to modify CD47 accessibility (FIG. 6 B), CD47 synapse localization was confirmed in the OPL by staining with pre- and post-synaptic neuronal markers (FIG. 6 C), CD47 overlapped with horizontal cells and was located post-synaptically where it interdigitated with pre-synaptic SIRPα (FIG. 6 B-C). Fluorescent in situ hybridization (FISH) was performed for CD47 RNA to determine the cells responsible for CD47 production (FIG. 6 E). Co-staining for cell type-specific markers confirmed dominant CD47 expression in postsynaptic horizontal cells in the OPL, with signal also present in the inner nuclear layer and ganglion cell layer. Together, these data suggested that CD47 was highly colocalized with neuronal SIRPα and that high levels of this inhibitory cue were present during peak microglial phagocytosis.

The impact of neuronal SIRPα on temporally restricted microglia signaling and reactive state was determined. As described above, the data indicated that neuronal SIRPα was necessary for microglial phagocytosis, but whether it is sufficient to spatially and temporally instruct homeostatic or reactive subtypes remained unknown. To assess sufficiency, neuronal SIRPα was overexpressed, and microglia engulfment was analyzed to determine if it persisted into periods when microglia are normally quiescent (FIG. 7A-I).

To determine if prolonging neuronal SIRPα expression extends microglial phagocytic periods, neuronal SIRPα was tested to determine if it alone is sufficient to define when microglia are active, to this end, gain-of-function assays were performed wherein transgenic SIRPα was introduced by electroporation of plasmid DNA at P0 (FIG. 7A-I). This method transfected dividing cells, which consist mainly of photoreceptors. Because microglia are born embryonically, they were not transfected, ensuring this method only targets neurons. As shown in FIG. 7 C, transduced regions comprised photoreceptors, but no microglia, and transduced cells expressed high SIRPα and/or GFP levels (FIG. 7 B-C).

Wildtype SIRPα^{NEURON}, and SIRPα^{MICROGLIA} animals were transfected, and microglial phagocytosis at P21, when microglia are normally quiescent, was tested using the previously described measures of activity (e.g., process length and number, soma size, CD68 levels globally and in single cells, phagocytic cup presence and number). High levels of neuronal SIRPα can define the window in which microglia are phagocytic, and an increase in observed microglia activation at P21 was apparent in all three models relative to pCAG-GFP+control animals.

Notably, these data show that neuronal SIRPα alone is sufficient to determine when microglia are phagocytic in both development and adulthood (e.g., overexpression of SIRPα in otherwise wildtype animals is sufficient to alter microglia activation state). Microglia in SIRPα transfected wildtype mice appeared ameboid, displayed less ramification, and showed significantly increased levels of CD68 globally and in individual cells relative to controls at P21 (FIG. 7 C-I). These data were consistent with the idea that neuronal SIRPα was sufficient to instruct the timing of microglial phagocytosis.

To determine the impact of neuronal SIRPα on the nature of microglia activity, additional experiments are conducted. Previously discussed data is indicative of decreased phagocytosis as quantified by reduced phagocytic cups and lower CD68. This phenomenon is further examined by broadly assessing homeostatic and reactive microglia markers. First, relative levels of P2RY12, a marker of homeostatic microglia, as well as Clec7a, a neurodegeneration-associated marker are histologically quantified. Second, transcriptomic analysis using established technologies (e.g., STATE-seq method) are used to broadly examine neuronal-SIRPα dependent reactive and homeostatic microglia states. This approach enables high fidelity RNA recovery after fixation and staining for intracellular markers followed by antibody-based FACS sorting. Iba1 staining is used to isolate purified microglia populations from SIRPα^{NEURON} animals, SIRPα^{MICROGLIA} animals, and control animals at P9 and P14. Bulk and single cell RNA-seq is carried out for each age and genotype. In addition to unbiased clustering, known homeostatic and reactive microglia markers are examined (e.g. P2RY12 and TMEM119 versus LEC7A, SPPI, APOE, GPNMB, CD163, and CD74, respectively).

Prolonging neuronal SIRPα expression will extend the period in which microglia are phagocytic, and deletion will correspondingly influence markers of microglia homeostasis and reactivity.

The question of if neuronal SIRPα acted as a local or non-local cue to affect microglial phagocytic state was raised. It was known that neuronal SIRPα can be cleaved and secreted and thus has the potential to act locally or non-locally to alter microglial phagocytosis. To test this, the fact that electroporation targets the retina regionally when DNA levels are titrated was exploited. As shown in FIG. 7 I, patches of high transgene expression adjacent to control non-transfected regions that contain no transgene expression levels were observed. Microglia cell phagocytosis capacity at P21 within transformed patches was compared to microglial phagocytosis for cells outside the transformed patches. As shown in FIG. 7 I, increased microglial phagocytosis (as represented by CD68 expression) appeared restricted precisely to regions in which transgene expression is present, and adjacent un-transfected regions showed normal, quiescent microglia that did not differ from GFP transfected controls (FIG. 7 I). These data were consistent with the idea that neuronal SIRPα may be sufficient to instruct the local phagocytic state of microglia.

The necessity of local SIRPα production to permit microglial phagocytosis is tested. Wild type mice at P0 of age are electroporated with a SIRPα targeting CRISPR construct (pSpCas9 (BB)-2A-GFP. PX458). This CRISPR method has been previously shown to effectively delete genes from the retina and results in labeled neurons in which SIRPα is removed. Deletion is confirmed by SIRPα antibody staining. In each case, microglial phagocytosis at P9 in SIRPα deletion patches is compared to both control un-transfected and control pCMV-GFP patches. Local SIRPα production is necessary to permit microglial phagocytosis, and microglia display a decrease in markers of phagocytic activity in regions in which SIRPα is removed.

The question of if locally increasing microglial phagocytosis impacts refinement outcomes was raised. These data showed that locally increasing microglial phagocytic activity increased engulfment and decreased synapse number in targeted regions. Assays comprising evaluation of microglial engulfment and synapse refinement outcomes in pCAG-SIRPα-GFP electroporated patches relative to pCAG-GFP control regions were analyzed. In each case, three-dimensional reconstruction was used to identify GFP⁺ neuronal material engulfed by individual microglia. Additionally staining for the ribbon marker RIBEYE facilitated quantification of the number of OPL synapses. Prolonging microglial phagocytic activity via neuronal SIRPα impacted refinement outcomes, engulfment increased and synapse number decreased in SIRPα-electroporated regions.

Microglia in SIRPα-electroporated patches showed significantly increased levels of engulfment relative to those in GFP+control regions (FIG. 7 H). Further, increased engulfment was associated with decreased synapse number, as both the relative levels of RIBEYE fluorescence and the number of RIBEYE positive synapses were lower in SIRPα-GFP regions relative to controls (FIG. 7 J). These data were consistent with the idea that neuronal SIRPα acts as a locally restricted cue to impact microglia engulfment and refinement outcomes. Locally increasing SIRPα promoted microglial phagocytosis within transformed regions relative to adjacent control areas and was accompanied by local increased engulfment and decreased synapse number.

SIRPα was identified as a key neuron-derived cue for determining when, where, and how microglia are active during development and adulthood. This analysis established neuronal SIRPα necessity and sufficiency for microglial phagocytosis.

It is possible that modulating levels of neuronal SIRPα could result in indirect effects on microglia SIRPα production. These effects could conceivably contribute to the previously described results. While the staining analyses in SIRPα ^{NEURON} animals and SIRPα^{MICROGLIA} animals do not broadly indicate this (FIG. 5A-K), the cell-specific levels of SIRPα is assessed following the proposed manipulations via antibody staining and flow cytometry to control for this possibility.

Alternative OPL synapse markers (e.g., PSD95 and Bassoon) can be used for quantification of retina synapses.

### Example 2—Cell-Specific Pathways Involved in SIRPα-Mediated Microglial Phagocytosis

The following example discusses and elucidates the cell-specific and localized interactions between SIRPα and CD47 in the nervous system.

The only known ligand for SIRPα is the widely distributed anti-phagocytic protein, leukocyte surface antigen CD47 (CD47), CD47 is expressed at high levels on neurons, but it has also been reported on microglia. Thus, neuronal SIRPα may influence microglial phagocytosis indirectly by controlling the accessibility of neuronal CD47 or directly by binding microglia CD47. To assess these possibilities, global and conditionally deleted CD47 animals are created with neuron and microglia-specific cre lines.

Microglia are thought to regulate developmental neural refinement through precise temporal control of their activity. Microglial phagocytosis peaks during postnatal neural remodeling and is reduced as refinement ends. This creates a ‘critical period’ of microglia action where they display hallmarks of phagocytic activity: enlarged somas, decreased branching, high levels of the lysosomal protein CD68, and large numbers of phagocytic cups. While recent studies have uncovered new roles for immune signaling molecules in dictating the removal of individual synapses, significantly less clear was whether and how this cross-talk converges on microglia to impact their phagocytic and inflammatory state.

Also previously unknown was how these signals were temporally regulated to ensure microglia are active only at the right time and place. As described above, the data indicate that SIRPα is upregulated during periods of high microglial phagocytosis and then decreases as microglia become quiescent. Manipulation of the duration and levels of neuronal SIRPα and/or neuronal CD47 can be used to control the timing of microglial phagocytosis (FIG. 8A-B). These findings facilitate better understanding of the processes that open and close microglia engulfment windows and will provide rational for better therapeutic regimens for associated pathologies.

Subcellular interactions between SIRPα with CD47 are identified using CD47 deletion within neurons and/or microglia, with and without paired SIRPα co-deletion. These experiments determine whether cell-specific pathways involved in SIRPα-mediated microglial phagocytosis are direct or indirect and test the requirement for CD47. Animals with co-deletion of SIRPα and CD47 are analyzed.

As discussed above, the data suggest that SIRPα and CD47 are both present at retinal synapses (FIG. 6 B), however, resolution of confocal microscopy is too poor to quantitatively determine the overlap between neuronal versus microglia SIRPα with CD47. The validated RAIN-STORM imaging protocol is used to confirm the confocal microscopy results. This method enables three-dimensional nanoscopic imaging of synapses in tissue, ensuring that native arrangements are maintained. For neurons, individual rod terminals are visualized using PDS95, and within these regions, the percent CD47 occupancy of SIRPα at P2, P9, and P14 is quantified. Individual microglia processes are imaged using Iba1 and the percent CD47 occupancy of microglia SIRPα is quantified. Parallel analyses are completed in SIRPαMICROGLIA animals and SIRPα^{NEURON} animals.

CD47 and SIRPα binding are directly assessed using co-immunoprecipitation from SIRPα^{MICROGLIA} animals, SIRP«NEURON animals, and in age matched littermate controls. Retinas from P2, P9, and P14 animals are solubilized and subjected to immunoprecipitation with anti-CD47 and anti-SIRPα antibodies. If neurons provide the dominant source of SIRPα that interacts with CD47, then binding levels between the two proteins are comparable in control and SIRPα MICROGLIA mice but significantly reduced in SIRPαNEURON mice.

If neuronal SIRPα is the primary ligand of CD47, overlap is greatest between rod-terminal associated SIRPα and CD47 while microglia SIRPα shows less CD47 occupancy.

The data presented in FIG. 7 J suggested both precise SIRPα co-labeling with the ribbon protein RIBEYE, and overlap with CD47, validating the approach and SIRPα association with CD47 at synaptic terminals (FIG. 7 J).

CD47 and SIRPα binding is directly assessed using co-immunoprecipitation in SIRPα^{MICROGLIA} animals, SIRPα^{NEURON} animals, and age matched littermate controls. Retinas from P2, P9, and P14 animals are solubilized and subjected to immunoprecipitation with anti-CD47 and anti-SIRPα antibodies. If neurons provide the dominant source of SIRPα that interacts with CD47, binding levels between the two proteins are comparable in control and SIRPα MICROGLIA mice but significantly reduced in SIRPα^{NEURON} mice. If neuronal SIRPα instructs microglial phagocytosis by limiting neuronal CD47 accessibility, neuronal SIRPα binds and occupies the majority of CD47. As an alternative to co-immunoprecipitation, dimerization-dependent fluorescent protein (ddFP) domains are utilized to test interactions.

Neuronal SIRPα may indirectly enable microglial phagocytosis by limiting microglia SIRPα binding to inhibitory neuronal CD47. In such a situation, decreasing neuronal CD47 levels has little impact on microglial phagocytic activity because the signal is normally blocked by neuronal SIRPα. In contrast, removing both neuronal CD47 and neuronal SIRPα can restore microglial phagocytosis, while co-overexpression of neuronal CD47 can prohibit SIRPα-dependent increases in phagocytosis.

To determine if decreasing neuronal CD47 alters SIRPα-dependent microglial phagocytic activity, two methods can be utilized. First, the levels of CD47 in neurons that lack SIRPα are decreased by sparsely electroporating a CD47 CRISPR targeting plasmid (e.g., pCMV-CD47-CRISPR-GFP) in SIRPα^{NEURON} mice. Second. SIRPα^{NEURON} mice and CD47H/F mice are crossed to generate SIRPα; CD47NEURON double KO mice. These animals are compared to CD47 null mice (CD47^{-/-}) and controls. Markers of microglia phagocytosis in retinas are assessed at P2, P9, and P14. If neuronal SIRPα enables microglia activation by limiting microglia SIRPα binding to inhibitory CD47, then eliminating CD47 alone has relatively little impact on microglia activation.

The initial data support this idea, as microglia maintained high levels of phagocytosis in CD471-mice during synapse remodeling, with only minor changes relative to those in controls (FIG. 6 G-H).

If it is observed that removing neuronal CD47 from SIRPαNEURON mice restores microglia activation, this would support an indirect model in which SIRPα limits neuronal CD47 accessibility.

To determine whether co-overexpression of neuronal CD47 prohibits the increase in microglial phagocytosis observed with neuronal SIRPα expression alone, both SIRPα (pCMV-SIRPα-GFP) and CD47 (pCMV-CD47-RFP) are introduced into the retina by electroporation at P0. As plasmids that are administered together tend to transfect the same cells, it is suitable to assume both plasmids are expressed in the same neurons. The established markers of microglia activation are assessed in retinas harvested at P9 and P21. If neuronal SIRPα temporally enables microglia activation by limiting microglia SIRPα binding to CD47, increasing CD47 alone can promote quiescence at P9, while SIRPα and CD47 co-expression can restore microglia activation.

Notably, the inventors observed that neuronal SIRPα temporally enabled microglia activation by limiting microglia SIRPα binding to CD47. Increasing CD47 alone promoted quiescence at P9, while SIRPα and CD47 co-expression restored microglia activation, indicating co-expression rescued activation (FIG. 8A-D).

It is noted that in electroporation experiments, expression levels can vary from experiment to experiment. To assess and control for this, only regions with similar expression as quantified by GFP, CD47, and SIRPα staining are compared.

Another mechanism by which neuronal SIRPα could enable microglial phagocytosis is by binding to CD47 on microglia. While CD47 lacks a substantial cytoplasmic signaling domain, it is possible that SIRPα-dependent lateral CD47 interactions with other receptors can play important roles. In certain embodiments, such a model indicates that decreasing CD47 in microglia can induce microglia quiescence similar to that in SIRPα^{NEURON} mice while co-deletion of neuronal SIRPα and microglia CD47 can restore normal microglial phagocytosis.

As microglia are spared in electroporation and AAV transduction, a CD47F/F line is suitable for removal of CD47, CD47 in microglia is removed by crossing these animals to TNFRSF11A^{Cre} to generate CD47MICROGLIA mice. These mice are assessed using the previously outlined markers of microglia activation in retinas harvested at P2, P9, and P14. If neuronal SIRPα temporally enables microglia activation by binding directly to microglia CD47, microglia in this model are quiescent when they normally are active, resembling those in SIRPα^{NEURON} mice. To determine if microglia CD47 interacts with neuronal SIRPα to control microglial phagocytosis, CD47MICROGLIA; SIRPα^{NEURON} animals are generated. If microglia CD47 controls microglia activation via neuronal SIRPα, normal microglia activity is restored. If microglia CD47 directly binds neuronal SIRPα, microglia specific removal of CD47 impacts microglial phagocytosis, and these effects are mitigated by joint removal of microglia CD47 and neuronal SIRPα.

Although CD47 is the only known receptor for SIRPα, an unknown ligand may exist. If neither neuronal nor microglia CD47 manipulation rescue microglia defects in SIRPα^{NEURON} animals, additional SIRPα interacting partners are identified in samples harvested at day P9 using proximity labeling (BioID). Plasmid pcDNA3.1 MCS-BirA (R118G)-HA is used to insert a coding sequence for SIRPα in-frame with BirA to create a SIRPα-BirA fusion protein.

These experiments and results are valuable and elucidate how a ‘don't eat me’ pathway can be regulated and/or modified to permit developmental microglia activation.

### Example 3—Neuronal SIRPα-Mediated Microglial Phagocytosis Extends to the Central Brain

The following example discusses and elucidates the applicability of neuronal SIRPα mediated microglial phagocytosis and neural synapse engulfment in the central brain.

The retina is an extension of the brain, and principles that govern its development are often conserved in other brain regions. In line with this idea, Lehrman et al. (2018) found using global knockouts that CD47 and SIRPα can limit excessive neural refinement by microglia in the dorsal lateral geniculate nucleus (dLGN), a key retinorecipient area. The relative contribution of neuronal or microglia SIRPα to these outcomes was previously unknown. Microglia engulfment and ganglion cell axon refinement can be assessed in the dLGN using neuron and microglia-specific Cre lines to determine whether neuronal SIRPα-mediated microglia activity extends to the brain.

Microglia are increasingly implicated in most CNS diseases and injuries, notably including Alzheimer's disease, frontal temporal dementia viral infections, autism, and psychiatric diseases such as schizophrenia. In each case, excessive microglia engulfment can occur and contribute to aberrant synapse removal and declines in neural function. Thus, there is a need for improved methods to tune microglial phagocytosis and thereby limit disease burden. The present data indicate that local increases or decreases in SIRPα levels impact microglial phagocytosis only within the regions in which it is manipulated. These findings identify neuronal SIRPα as a novel target for precisely altering local microglial phagocytic capacity.

Lehrman et al, showed enhanced microglia engulfment and pruning when either SIRPα or CD47 whole body knockouts were analyzed, but whether these effects are due to neuronal or microglia SIRPα was unclear and unknown. Also unclear was whether global removal of these proteins may mask cell type-specific functions due to reciprocal interactions.

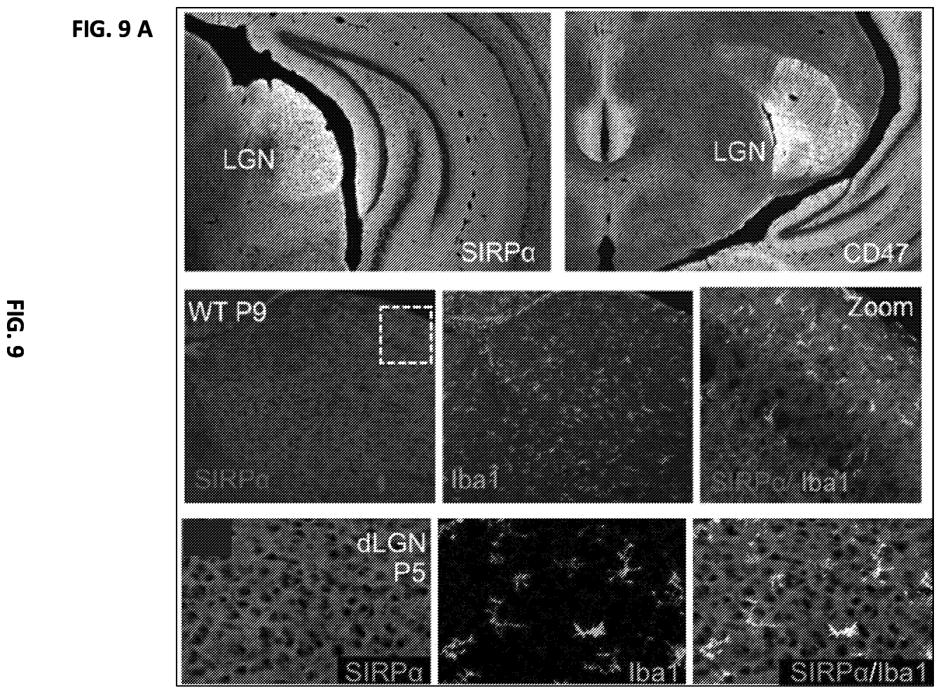

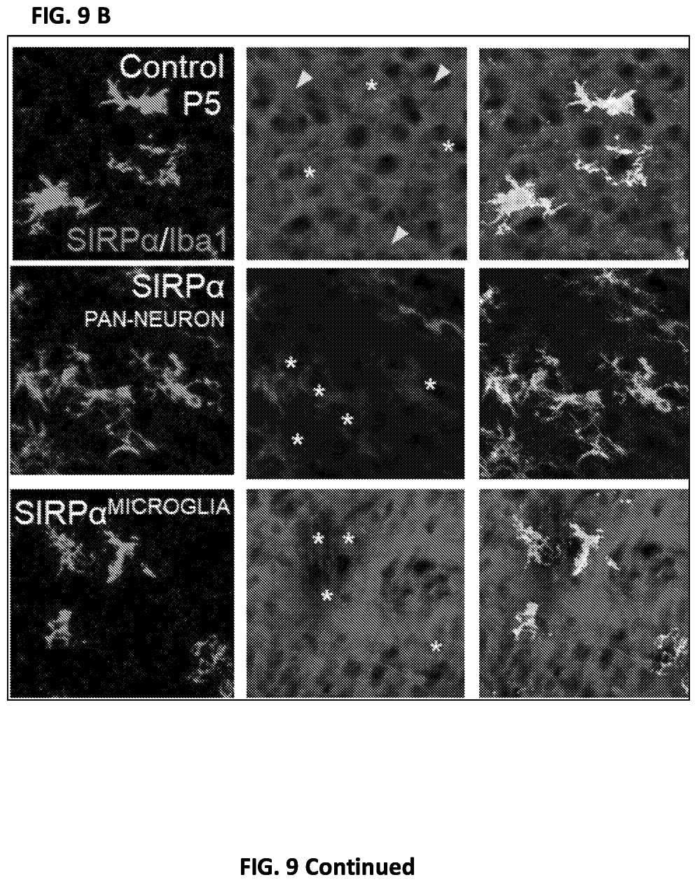

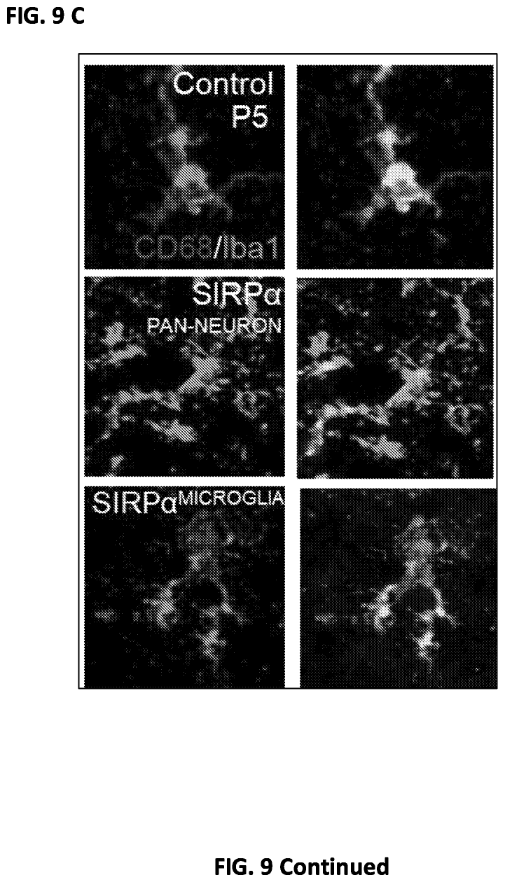

The SIRPα^{MICROGLIA} line works robustly and specifically in brain microglia. To generate a brain neuron SIRPα deletion line, the pan-neuron Cre line Nestin^{cre} was crossed to SIRPα^{F/F} mice. The specificity of the resultant line SIRPαPAN-NEURON, was confirmed (FIG. 9 A-C).

dLGN microglia activation and refinement following cell-specific SIRPα deletion is analyzed. Experimental and age-matched littermate control mice at P5, P9, and P14, postnatal ages that encompass dLGN refinement are tested using methods described herein. These animals are further compared SIRPα^{NEURON} animals, which specifically lack SIRPα derived from RGC axons.

To determine the relative contribution of neurons and microglia to total SIRPα production in the dLGN, levels and localization of SIRPα in the dLGN of SIRPα^{MICROGLIA} animals, SIRPαPAN-NEUR animals, and SIRPαNEUR animals are compared to levels in control animals. SIRPα levels and localization are analyzed by co-staining for SIRPα, Iba1 (microglia), and Vglut2 (presynaptic marker specific for RGC terminals in the dLGN). To quantify the overlap of SIRPα with RGC axons directly, RGC axons in the dLGN will undergo anterograde labeling using CTB-568 and CTB-647 injected into the left and right eye, respectively.

Data showed that, as in the retina, SIRPα is present at both neurons and microglia (FIG. 9A-C), validating a previous report.

Data also indicated that neurons were the dominant SIRPα source in the brain, as SIRPα levels were markedly reduced in SIRPαPAN-NEURON mice, whereas SIRPα^{MICROGLIA} mice showed little SIRPα reduction (FIG. 9A-C).

To assess the role of neuronal SIRPα in dLGN microglia activation, the previously described seven markers of microglia activation (e.g., ramification, nuclear size, CD68 levels, and phagocytic cups) are measured and compared between the dI.GN of SIRPαPAN-NEURON animals, SIRPα^{MICROGLIA} animals, SIRPα^{NEURON} animals, and age-matched littermate control animals. The inventors observe that neuronal SIRPα mediated microglia activation appears to extend to the dLGN, phagocytosis is reduced in the SIRPαPAN-NEURON line but relatively unaltered in SIRPα^{MICROGLIA} animals. Comparisons to SIRPα^{NEURON} mice reveal the contributions of RGC derived SIRPα to these outcomes.

Notably, the data suggest that, as in the retina, microglial phagocytosis in the dLGN of SIRPα^{MICROGLIA} animals did not significantly differ from controls during refinement (FIG. 9 A-C). Further, microglia phagocytosis appeared to be reduced in SIRPαPAN-NEURON animals. consistent with the idea of a conserved role for neuronal SIRPα in modulating microglia phagocytosis in the brain.

To determine how neuronal SIRPα impacts microglia engulfment and refinement outcomes, defects in microglia activation in the absence of neuronal SIRPα are further assessed to determine if there are alterations to engulfment and refinement. Ipsilateral and contralateral RGC inputs are labeled by intraocular injection of CTB-647 and CTB594, respectively, in SIRPα^{NEURON} animals. SIRPα^{MICROGLIA} animals, SIRPαPAN-NEURON animals, and control animals. Animals are sacrificed 24 hours after intraocular injection, and overlap between contralateral and ipsilateral RGC projection territories in dLGN is quantified. In this paradigm, an increase in the percent overlap between the projection territories is indicative of a synaptic pruning deficit. To measure engulfment, microglia are stained and single microglia are reconstructed with Imaris to quantify the relative levels of CTB-647 and CTB594 within individual cells. If neuronal SIRPα modulates refinement outcomes, the inventors observe reduced microglial activity in SIRPα^{PAN-NEURON} and/or SIRPα^{NEURON} mice results in delayed and/or defective eye-specific segregation, and/or reduced engulfment.

To determine if increased neuronal SIRPα expression extends the period in which dLGN microglia are phagocytic, two gain-of-function approaches can be utilized. First, global increases in neuronal SIRPα are generated sing intracerebroventricular injection of neonatal mouse brain with AAV2/9-SIRPα-GFP or an AAV2/9-GFP control. These injections result in persistent and widespread neuronal transduction throughout the brain as AAVs move freely from the cerebral ventricles at this age. Second, RGC-derived SIRPα in the dLGN is elevated by infecting retinas with AAV2/9-SIRPα-GFP at P0. While most retinas are transduced, only RGC project to the brain, enabling RGC-specific analysis. Wildtype mice, which express normal SIRPα levels, together with SIRPαPAN-NEURON animals, SIRPα^{NEURON} animals, and SIRPα^{MICROGLIA} animals are transduced. Microglia activation at P21 is assessed, during this time period, microglia are normally quiescent when assessed using the previously discussed measures of microglial activation. If high levels of dLGN neuronal SIRPα are capable of extending the window in which microglia are phagocytic, increased activation at P21 in all three experimental models relative to GFP+controls is observed. If neuronal SIRPα function extends to the dLGN, neuronal SIRPα is both necessary and sufficient to instruct the degree and duration of microglial phagocytosis and engulfment.

Together, these experiments and results are important for addressing and/or confirming whether there are region-specific pathways by which neurons alter microglial phagocytosis and activity.

The results and insights provided by these works elucidated mechanisms that prevent or permitted microglia activity and lay the groundwork for new therapeutic options in the nervous system (e.g., the CNS).

### Example 4—Retinal Neuron Refinement Coincided with Heightened Microglia Phagocytosis

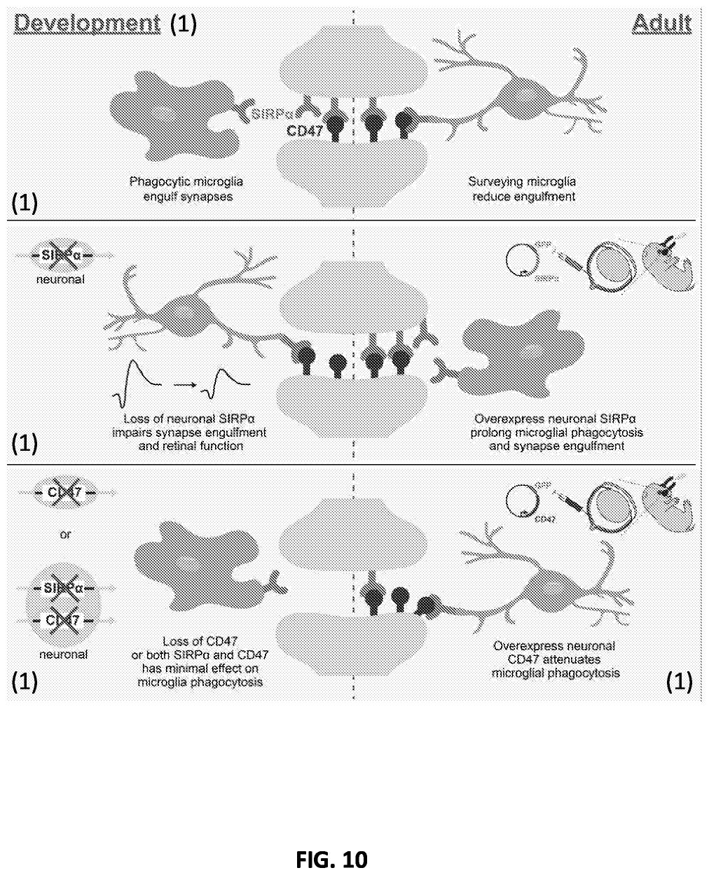

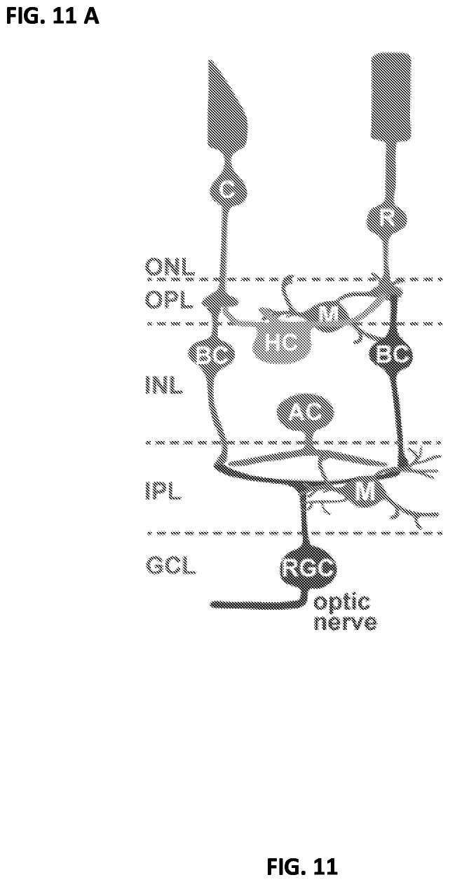

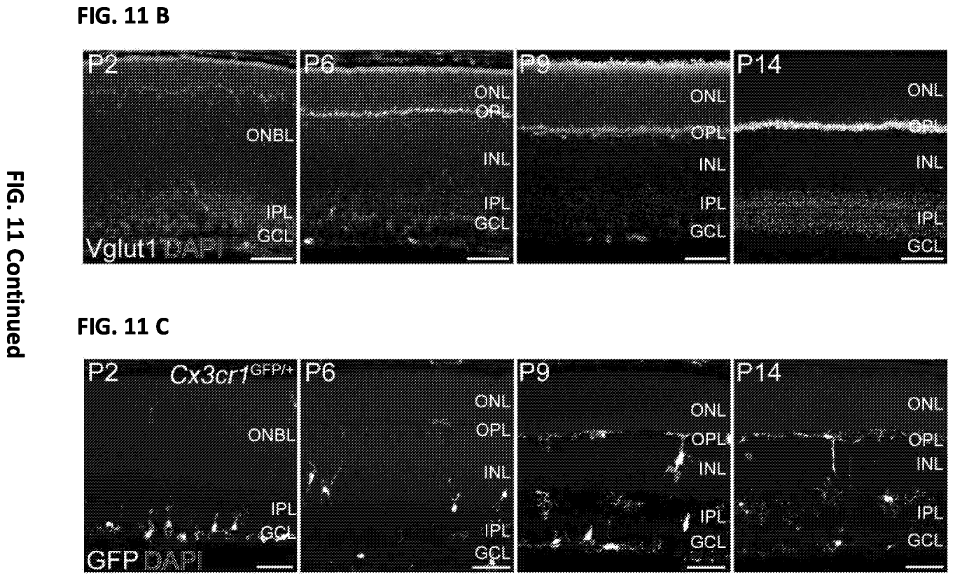

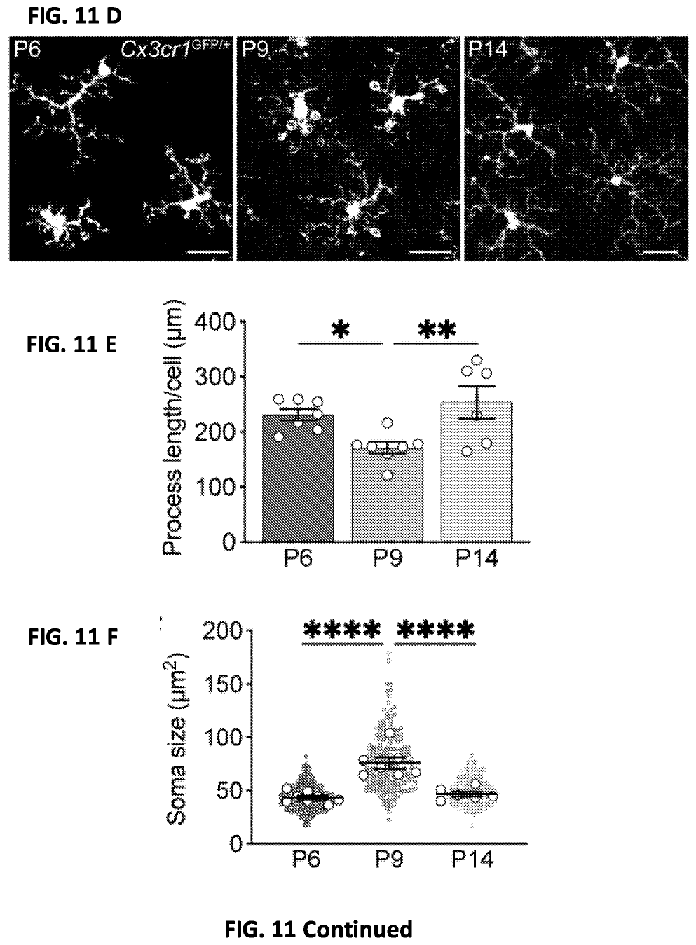

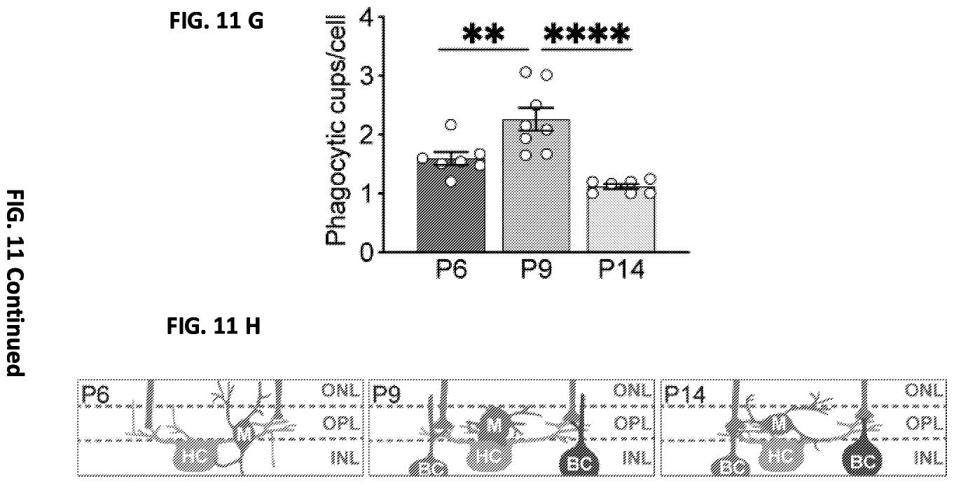

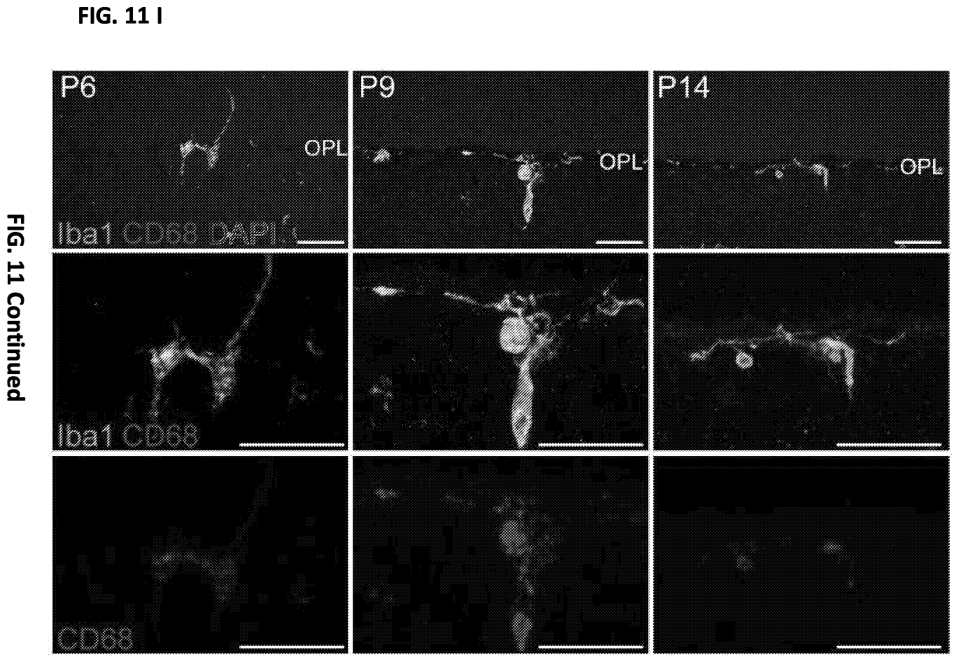

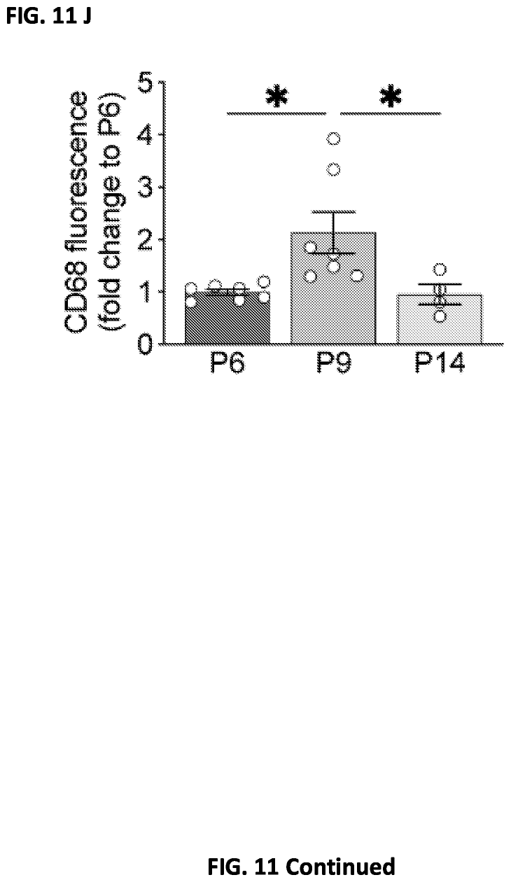

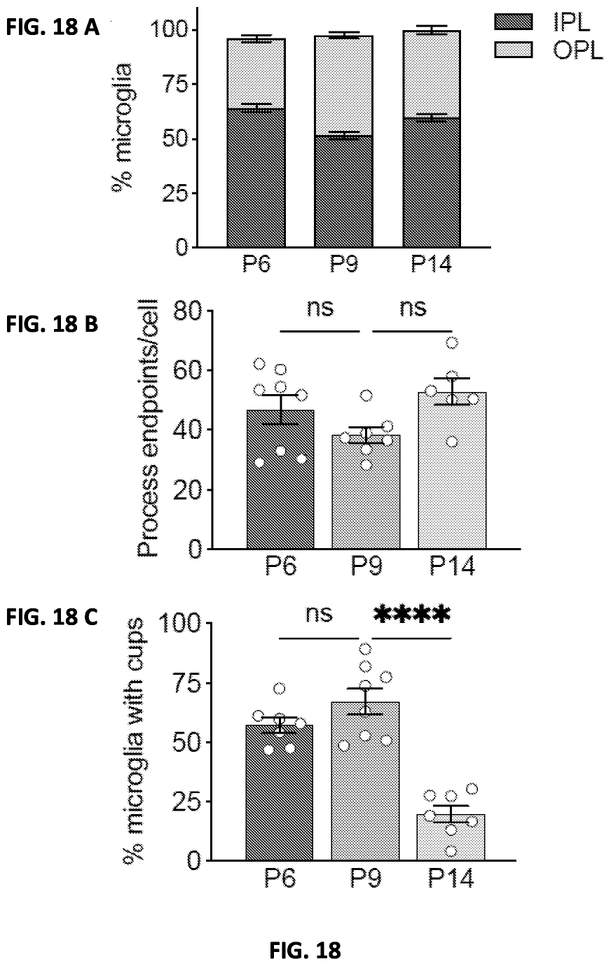

As in the brain, developmental refinement of the retina's diverse neuron types occurs during the first two postnatal weeks when neurites become restricted to two synapse layers (Kim et al., 2010; Wong and Ghosh, 2002) (FIG. 10A-B). The inner plexiform layer (IPL) appears first (˜P2), followed by the outer plexiform layer (OPL) at P5. By P14, neurons have largely adopted their adult morphologies. To examine microglia during this period, Cx3cr1GFP/+reporter mice in which microglia are selectively labeled with GFP (Jung et al., 2000), were utilized. It was found that the location and number of retinal microglia coincided precisely with synapse refinement (FIG. 10 C. FIG. 18A). At P9, 97% of microglia were present in retinal synapse layers (FIG. 18A). Because OPL synapses between presynaptic photoreceptors and postsynaptic horizontal and bipolar cells are particularly large and highly ordered, this region was focused on for analyses. The inventors found that high levels of microglia phagocytosis accompanied OPL synapse refinement. At P9, microglia adopted a morphology characteristic of active engulfment, with shorter process length, larger somas, and more phagocytic cups (round-shaped invaginations associated with phagocytosis (Swanson, 2008)) compared to time points prior to and after P9 (FIG. 11 D-H, and FIG. 18 B). Consistent with heightened phagocytosis, microglia in P9 retina displayed increased expression of the lysosomal membrane marker CD68 (FIG. 11 I). As refinement ended at P14, microglia adopted a morphology characteristic of more mature microglia with reduced engulfment. This included increased ramification, smaller somas, decreased CD68 protein expression, and fewer numbers of phagocytic cups (FIG. 11 F-G, FIG. 11 J, and FIG. 18 C). Together, these data showed that elevations in microglia phagocytic activity temporally and spatially aligned with retinal neuron refinement. As neurons matured and refinement concluded, microglial became ramified and lysosomal content declined, consistent with a decrease in phagocytic function.

### Example 5—Neuronal SIRPα was Enriched During Periods of Peak Microglia Phagocytosis

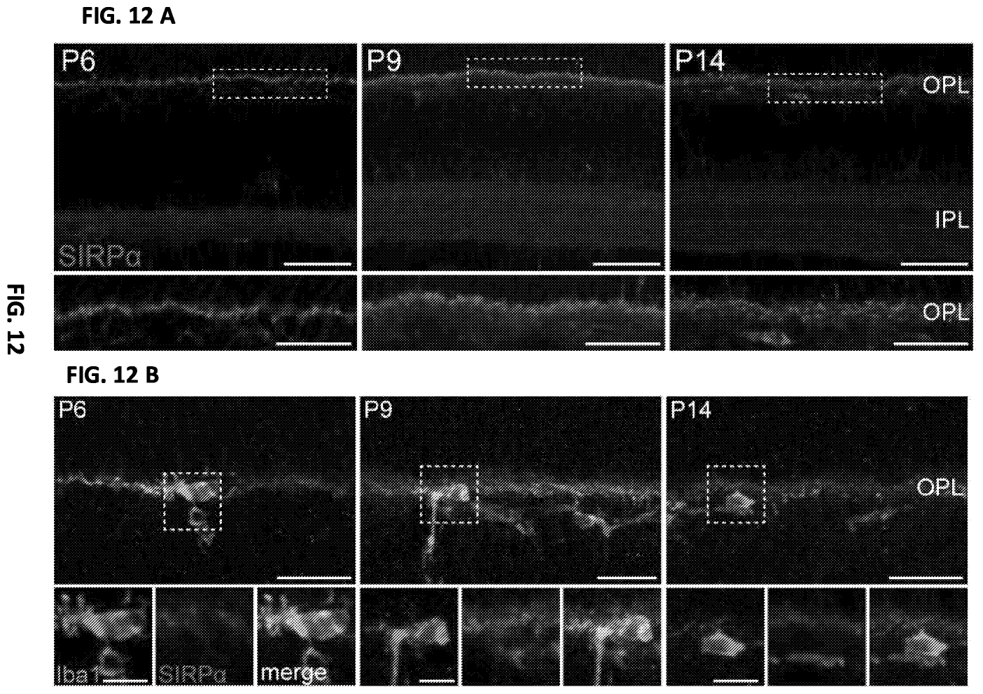

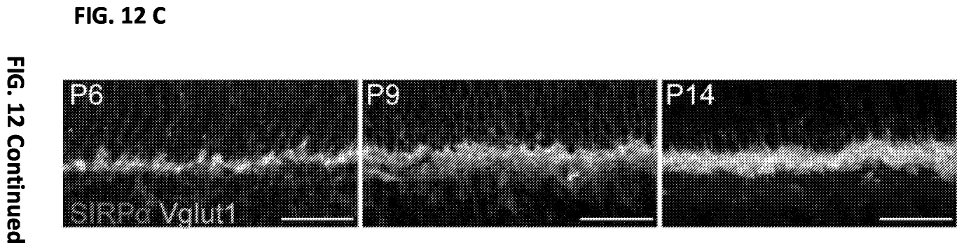

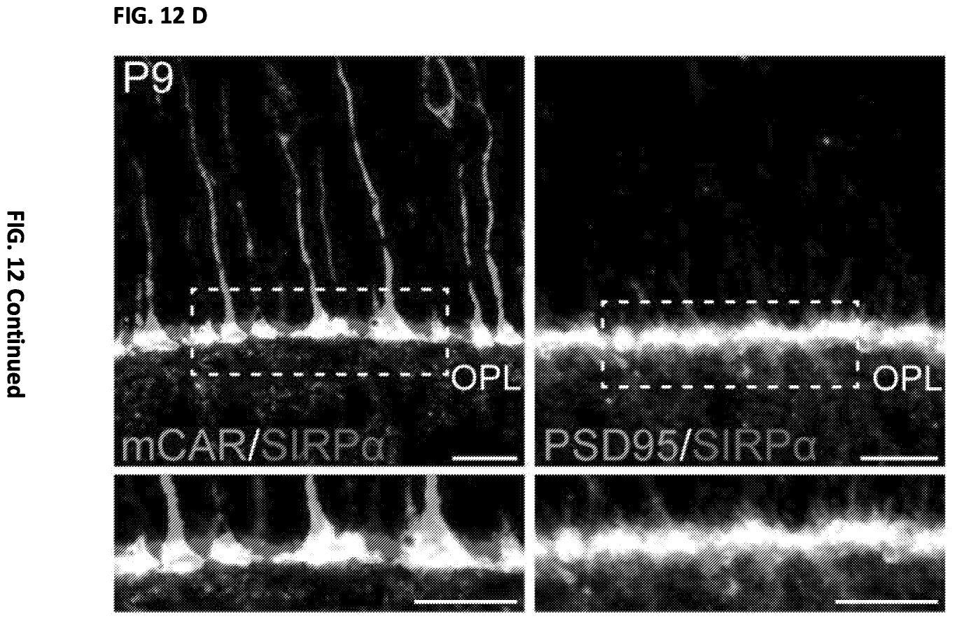

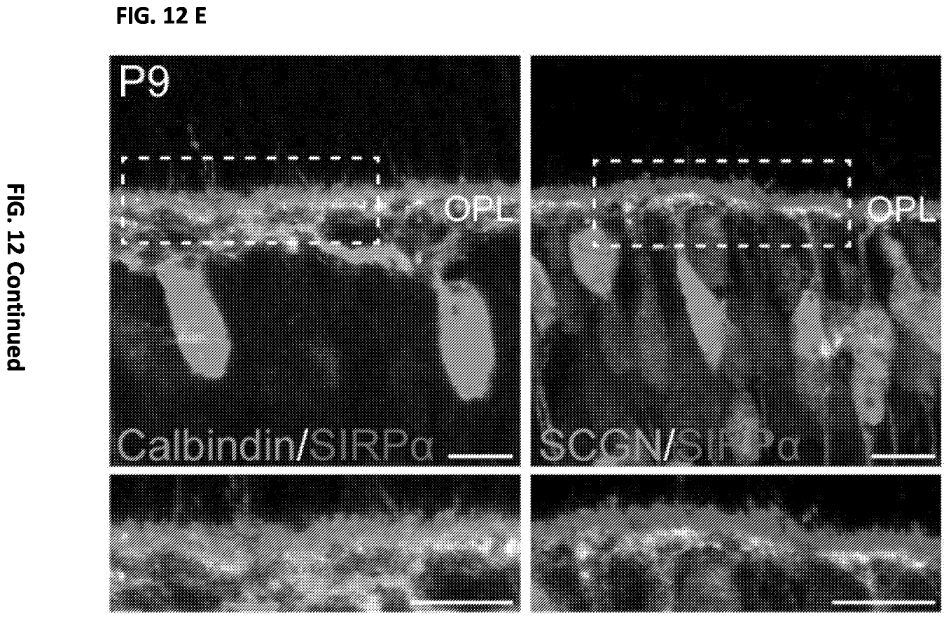

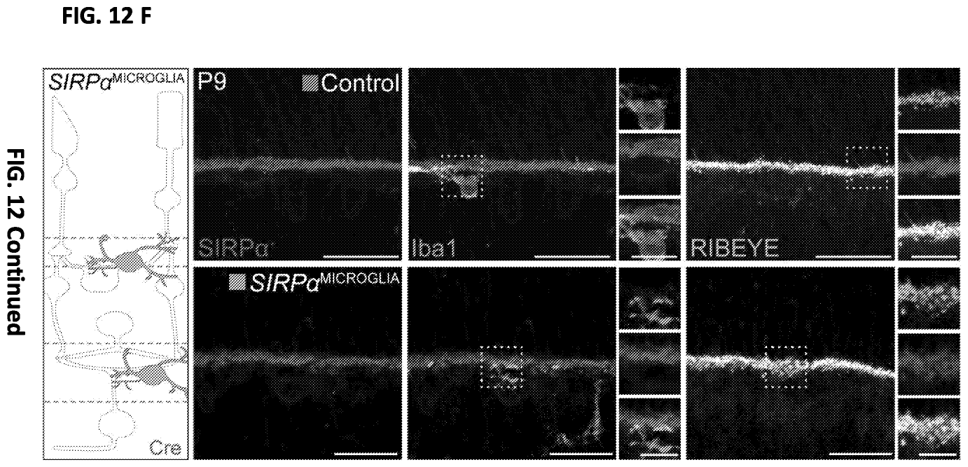

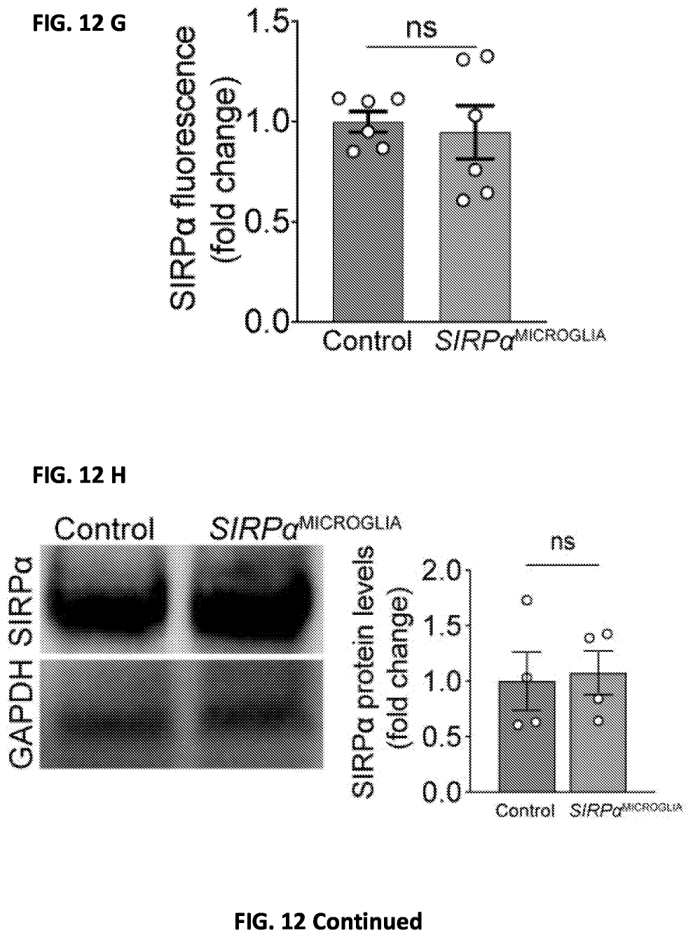

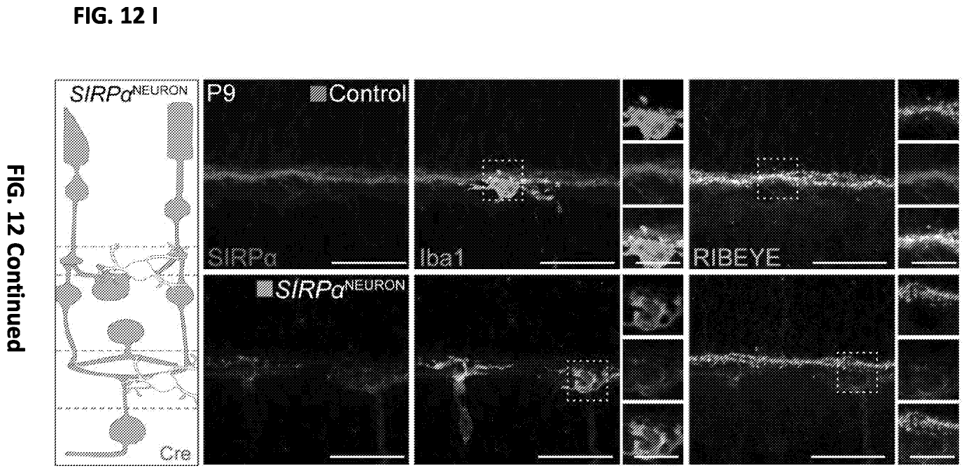

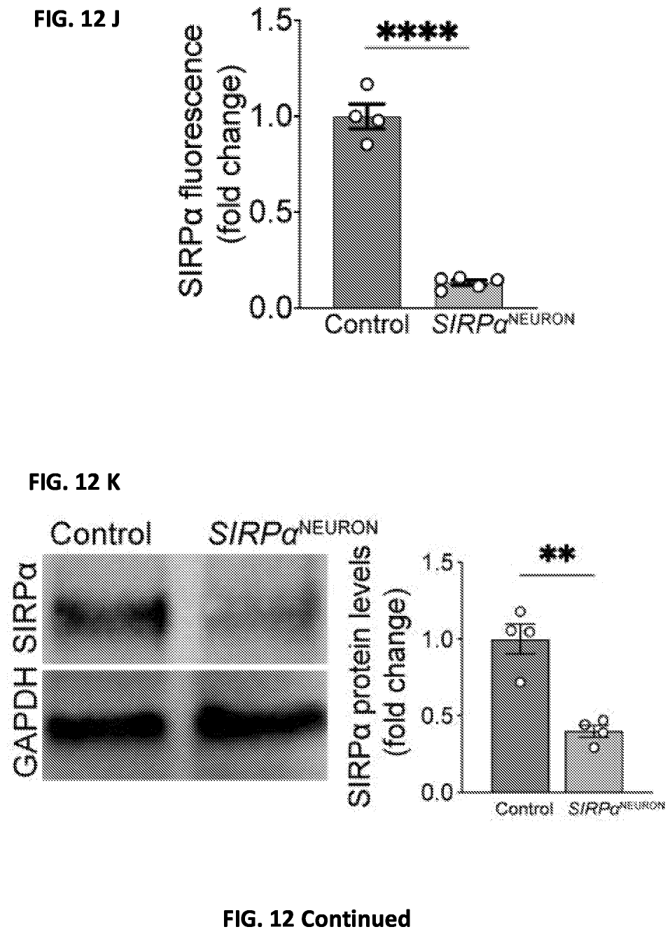

Through a screen for laminar-restricted molecules in the retina, the inventors previously uncovered SIRPα as a candidate regulator (Jiang et al., 2020). Significant SIRPα expression in both inner and outer retina synapse layers was identified using a beta-galactosidase reporter line (Jiang et al., 2020), validating a prior report (Mi et al., 2000). To determine whether SIRPα was at the right place and time to modulate microglia activity in the retina, the histological distribution of SIRPα over development was mapped. SIRPα first appeared as each retinal synapse layer emerged, and its expression increased as synapses refined (P9-14), coinciding with a high degree of microglial phagocytosis (FIG. 12A, and FIG. 19 A). At the conclusion of refinement at P14, SIRPα protein levels declined, though some SIRPα remained in the OPL (FIG. 12A, and FIG. 19 B). In microglia, SIRPα was present at low but detectable amounts, showing dim co-staining with the microglia marker Iba1 (FIG. 12 B). However, the bulk of SIRPα signal was localized to retinal synapse layers (FIG. 12 C). The inventors further confirmed SIRPα localization at synapses by staining with pre- and postsynaptic neuronal markers. It was found that SIRPα colocalized with presynaptic cone and rod terminal markers (mCAR and PSD95) but not with postsynaptic horizontal cell and cone bipolar cell terminals (Calbindin and SCGN, see FIG. 12 D-E and FIG. 19 C-D). Together, these results demonstrated that, as in the brain (Lehrman et al., 2018; Toth et al., 2013), SIRPα was found in both neurons and microglia in the retina during neuron refinement, but that the majority of SIRPα was associated with synapses. Further, the amount of neuronal SIRPα was highest when microglia were most phagocytic. Thus, neuronal SIRPα was in the right place at the right time to impact microglial phagocytosis.

SIRPα can be cleaved and secreted such that its histological localization may not necessarily reflect its primary cellular source (Nagappan-Chettiar et al., 2018; Toth et al., 2013). Accordingly, the inventors sought to determine the cellular source of SIRPα over development and performed single-molecule fluorescent in situ hybridization (smFISH) for Sirpα mRNA (FIG. 19 E). The inventors found that early in development (P2), Sirpα was present in both neurons and microglia, and this pattern persisted throughout refinement. From P14, Sirpα signal appears largely restricted to neurons. To confirm and extend these findings, the inventors genetically assessed which cells produce SIRPα by selectively eliminating SIRPα in microglia or neurons. To achieve this, the inventors crossed conditional SIRPα^{FF} mice (Skarnes et al., 2011) to either a yolk sac-derived erythro-myeloid progenitor Cre line TNFRSF11A^{Cre} (Macda et al., 2012), which in the brain are largely comprised of microglia (Jordao et al., 2019) or a retina neuron-specific Cre line Six3^{Cre} (Furuta et al., 2000). These mouse lines were termed SIRPαMICROGLIA and SIRPα^{NEURON}, respectively, and their specificity was confirmed (FIG. 19 F). It was found that in the absence of microglia-derived SIRPα, total SIRPα protein levels were unaffected (FIG. 12 F-H). In contrast, in the absence of neuron-derived SIRPα, protein levels were significantly decreased. A marked decrease of SIRPα immunofluorescent signal at synapses and in total protein levels was observed (FIG. 12 I-K). Low levels of microglia-localized SIRPα protein remained visible in SIRPα ^{NEURON} mice, while neuron-associated SIRPα was unaltered in SIRPα^{MICROGLIA} mice. To independently confirm these results, the microglia depletion model Cx3crl^{CreER}; Rosa26i^{DTR} (Zhao et al., 2019) was utilized. This model resulted in 96% microglia depletion at P8 (FIG. 19 G). Consistent with the results observed in SIRPα^{MICROGLIA} mice, synaptic SIRPα was intact in Cx3crl^{CreER}; Rosa26i^{DTR} animals, showing comparable staining and localization to that in controls (FIG. 19 H). Together, these data indicated that neurons were responsible for producing nearly all synapse-associated SIRPα and the majority of total SIRPα during neuron refinement.

### Example 6—Microglia Phagocytosis was Impaired in Neuronal SIRPα-Deficient Mice

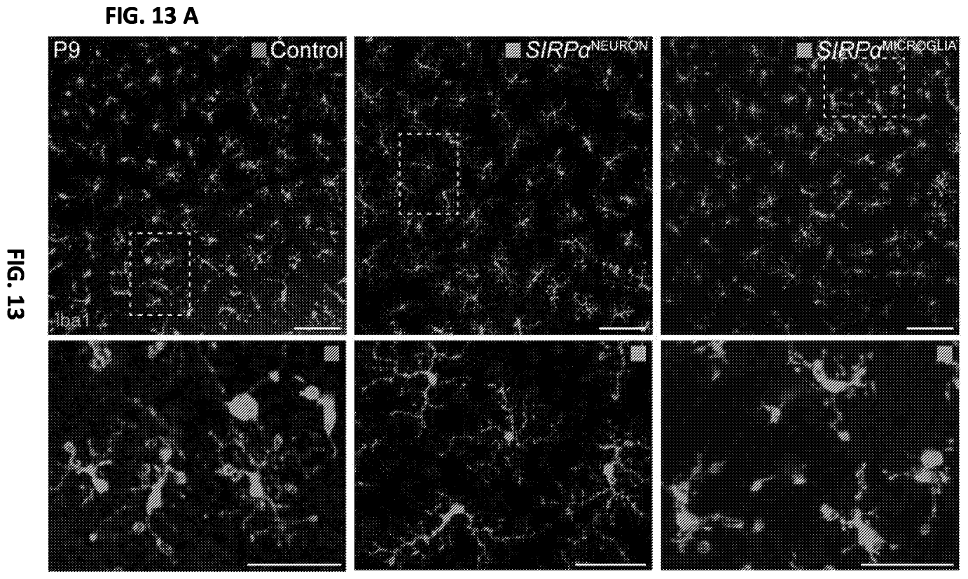

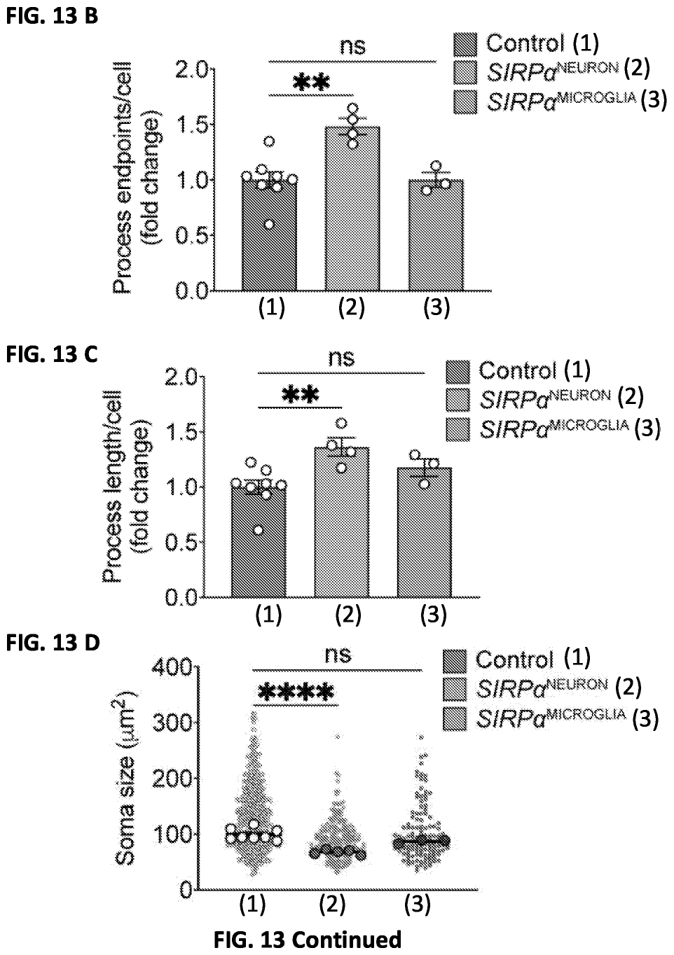

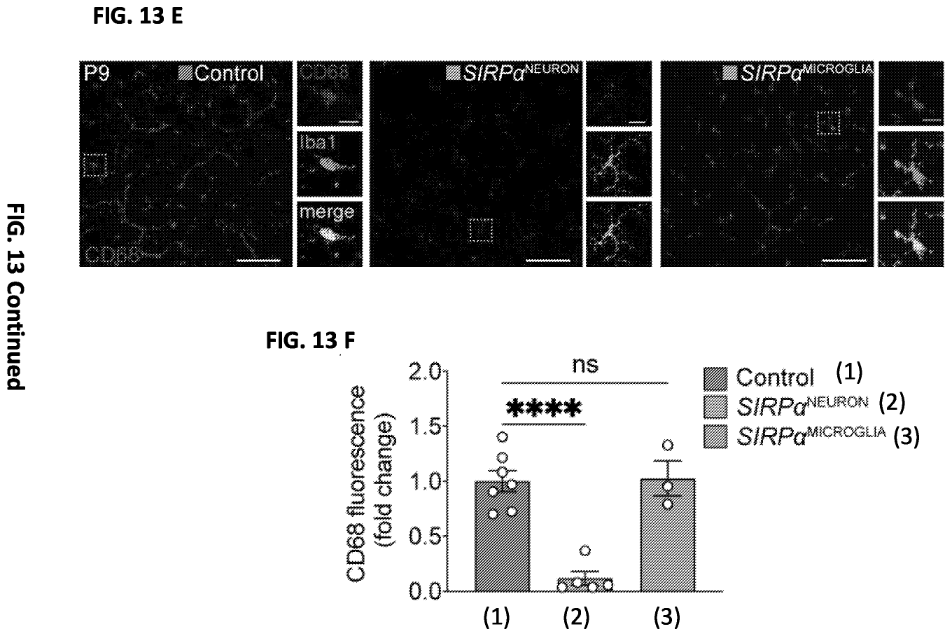

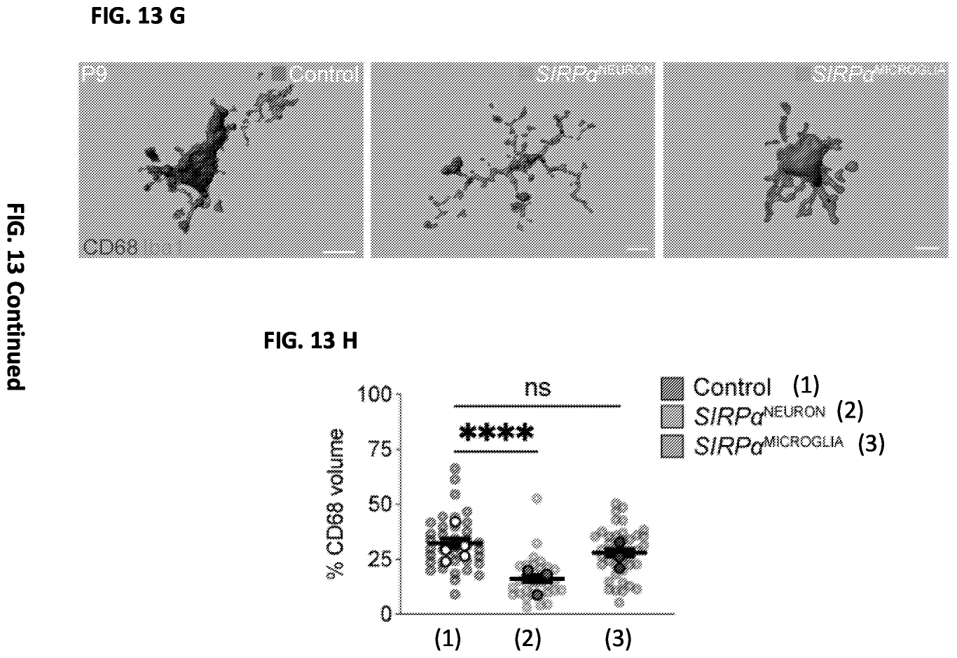

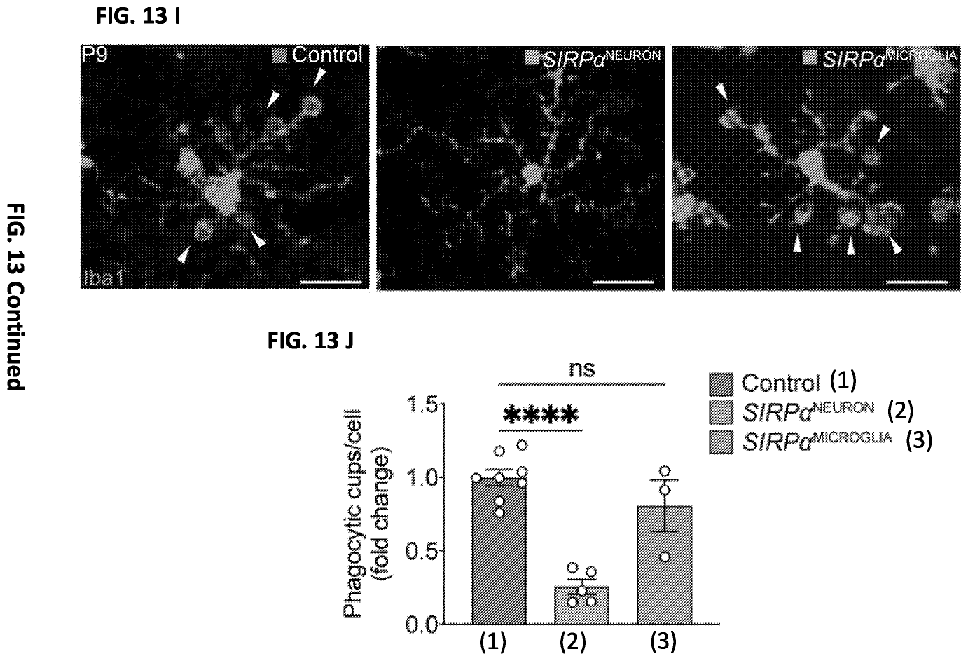

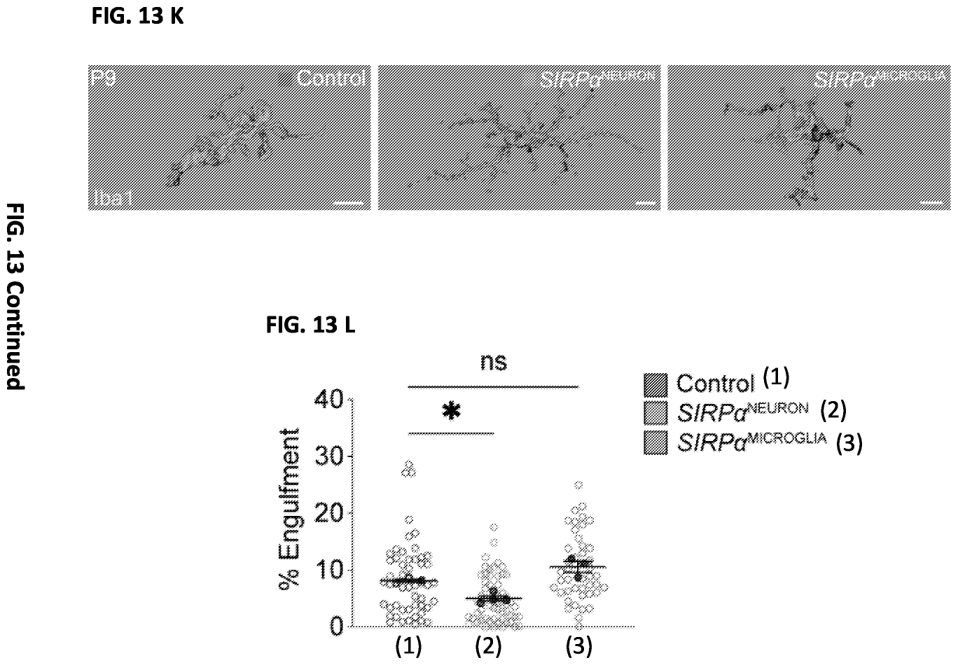

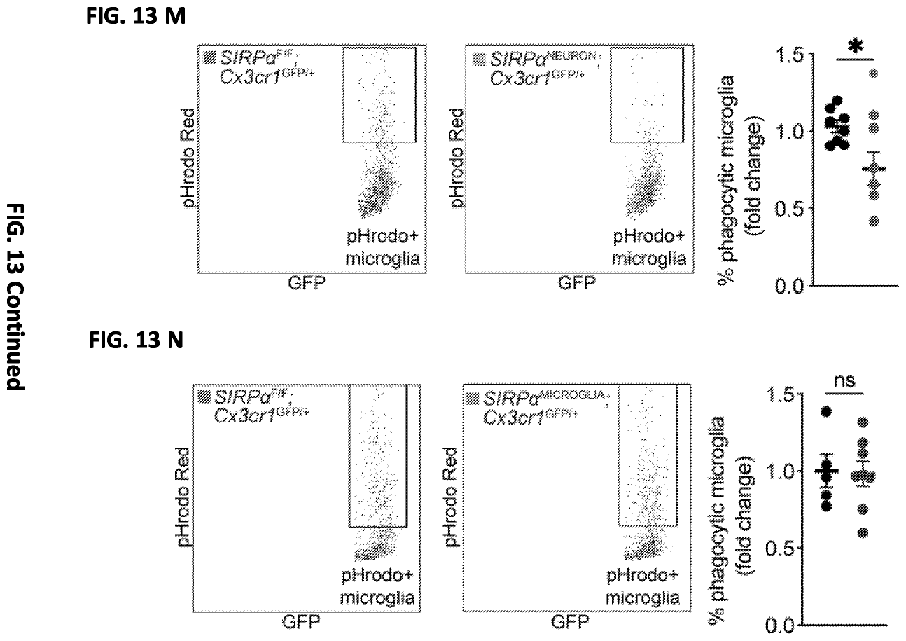

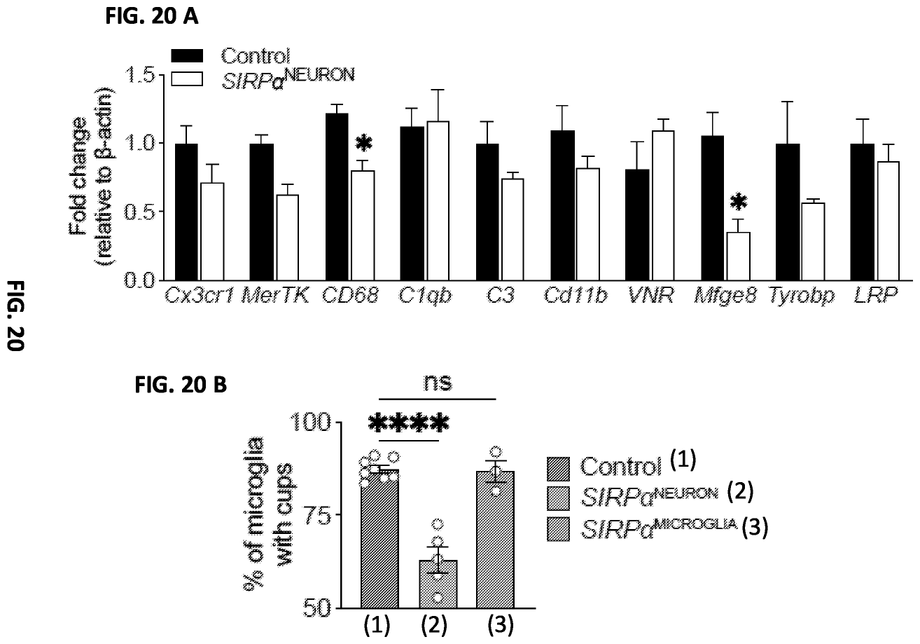

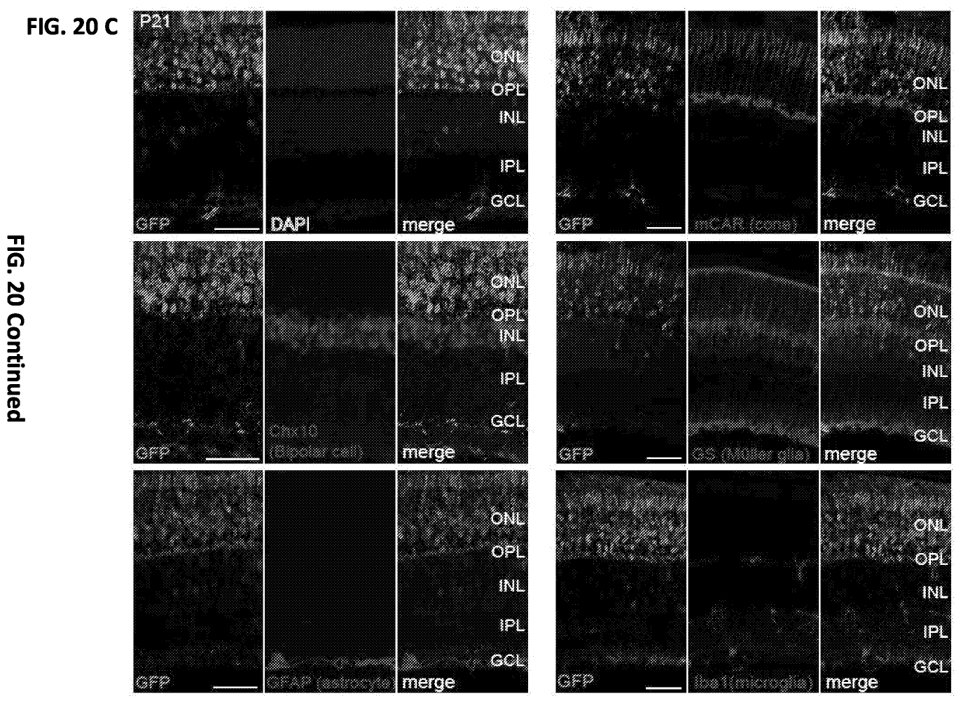

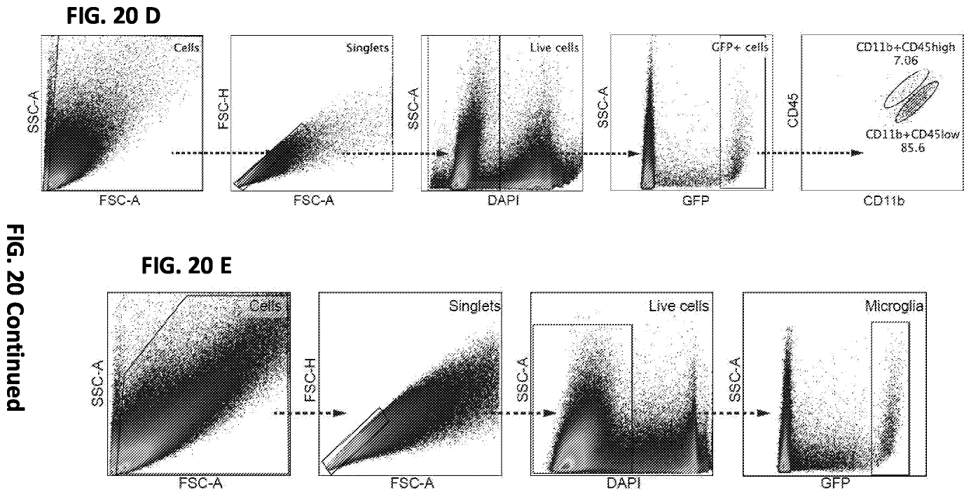

Given that neurons produced a high amount of SIRPα, the inventors sought to determine the relative roles of neuron- and microglia-derived SIRPα in modulating microglia activity. To assess this, microglia were examined at P9 in SIRPNEURON and SIRPα^{MICROGLIA} mice using seven independent measures. These included more general morphological features (soma size, process length, and process endpoint number) and phagocytic machinery markers (CD68 and prevalence of phagocytic cups). As expected at P9, microglia in control animals displayed shorter and less branched neurites, large somas, high amounts of the lysosomal marker CD68, and a high number of phagocytic cups. Hallmarks of microglia phagocytosis were largely absent in SIRPα^{NEURON} mice during this period. SIRPα^{NEURON} microglia were highly ramified at P9 with long, extensive processes resulting in a significant increase in total process endpoints and length relative to controls (FIG. 13A-C). Microglia in SIRPα^{NEURON} mice also had smaller somas (FIG. 13 D), and CD68 was drastically reduced (FIG. 13 E-F). In addition, individual microglia at P9 were imaged and reconstructed, and the average percent volume of CD68 within each cell was assessed (FIG. 13 G). SIRPα^{NEURON} mice displayed a significantly decreased volume of CD68 within microglia compared to controls (FIG. 13 H). Assessment of selected phagocytic pathway genes by qPCR using retinal RNA was largely consistent with the immunostaining data, showing reductions in SIRPα^{NEURON} mice (FIG. 20 A). Next, phagocytic cups were quantified. Significantly fewer microglia in SIRPα^{NEURON} mice displayed phagocytic cups, and those with cups contained half the number per cell compared to controls (FIG. 13 I-J, and FIG. 20 B). To further determine whether microglial phagocytic capacity was altered in the absence of neuronal SIRPα, GFP plasmids were electroporated into the retina at P0 and internalized GFP within microglia at P9 was assessed. This method only transfected dividing cells, which consisted primarily of photoreceptors at this age (FIG. 20 C) (Matsuda and Cepko, 2004). Because microglia are born embryonically outside the retina, they were not affected (Gomez Perdiguero et al., 2015; Mass et al., 2016). Consistent with previous results, microglial internalization of GFP+photoreceptor was significantly lower in SIRPα^{NEURON} mice when compared to controls (FIG. 13 K-L). Finally, the inventors generated SIRPα^{NEURON}; Cx3crl^{GFP/+}, SIRPα^{MICROGLIA}; Cx3crl^{GFP/+}, and SIRPαF/F; Cx3crl^{GFP/+} mice by crossing the cell type-specific knockouts of SIRPα with Cx3crIGFP animals, in which microglia express GFP. The inventors confirmed that most GFP+ cells in this mouse line were CD11b+ and CD45low, consistent with their microglial identity (FIG. 20 D) (Ford et al., 1995). Functional phagocytosis assays were then performed by measuring the engulfment of pHrodo-red-conjugated yeast particles in retina explants followed by dissociation and flow cytometry. This pH-sensitive dye conjugate only fluoresces upon lysosomal acidification allowing measures of phagocytosis in individual microglia (Miksa et al., 2009; Wang et al., 2021b). It was found that significantly fewer microglia from SIRPα^{NEURON}; Cx3crl^{GFP/+} retinas engulfed labeled particles relative to controls (FIG. 13 M, FIG. 20 E). Microglia phagocytosis was thus impaired in neuronal SIRPα-deficient mice.

By marked contrast, microglia phagocytic activity was largely unaffected in SIRPα^{MICROGLIA} retina. Microglial morphology in these animals was indistinguishable from that of P9 controls, and cells displayed comparable numbers of total process endpoints, length. and soma size (FIG. 13A-D). In addition, CD68 staining and the internalized volume of CD68 within 3D-reconstructed microglia were similar to P9 controls (FIG. 13 E-H), as were the percentage of microglia with phagocytic cups and the number of cups per cell (FIG. 13 I-J, and FIG. 20 B). Finally, microglia from SIRPα^{MICROGLIA}; Cx3crl^{GFP/+} retinas internalized GFP-labeled neuronal material (FIG. 13 K-L) and pHrodo-red-conjugated yeast (FIG. 13 N) at amounts similar to those of control microglia. Together these results suggested that neuronal, but not microglial SIRPα, was required to modulate microglia phagocytosis during development.

### Example 7—Neuronal SIRPα was Required for Synapse Refinement and Circuit Function in the Retina

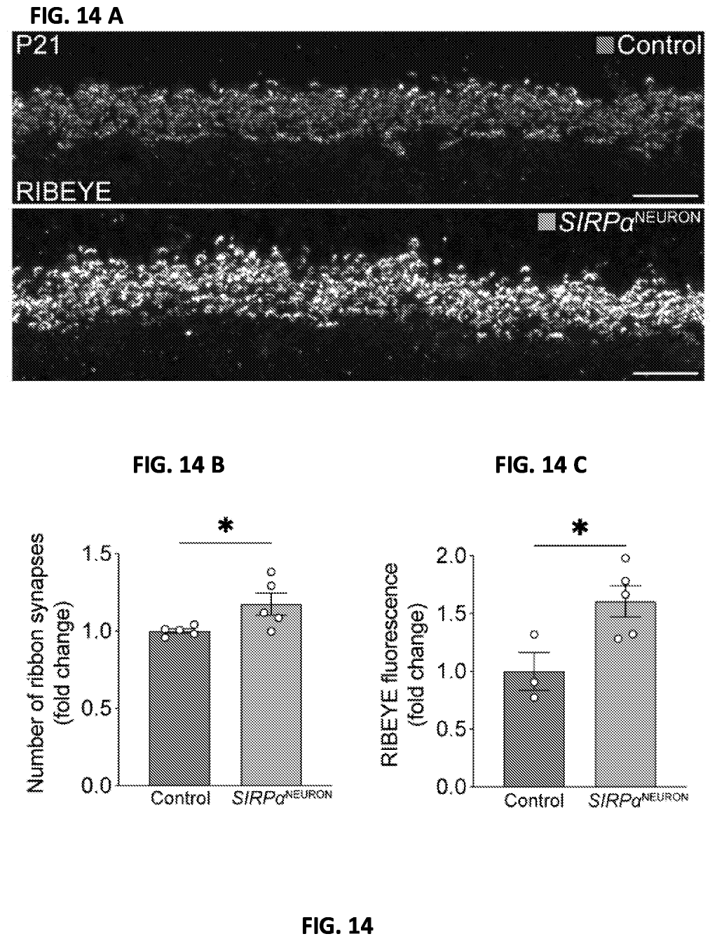

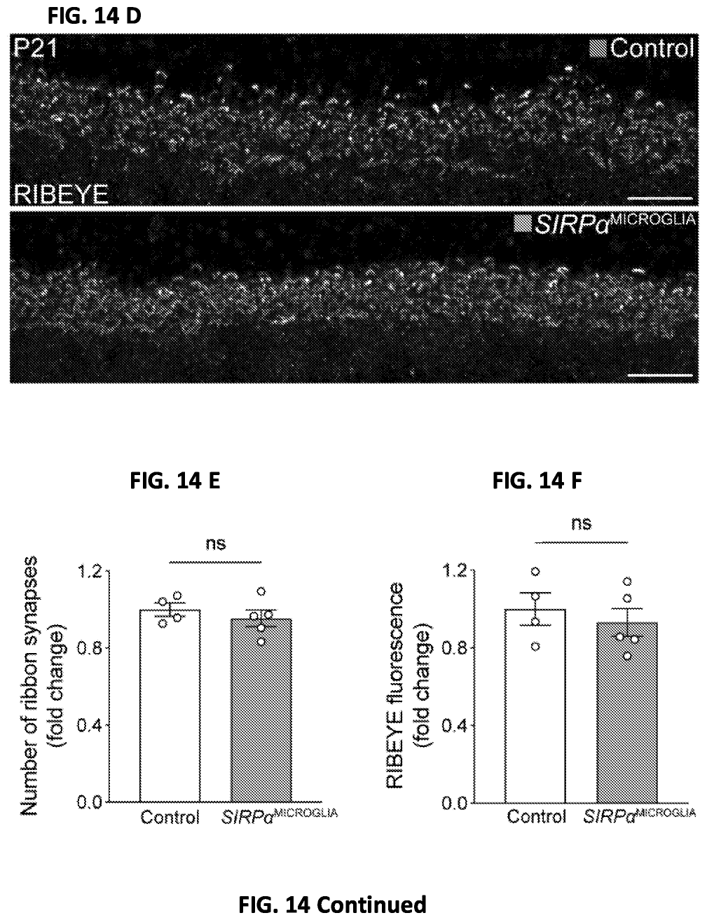

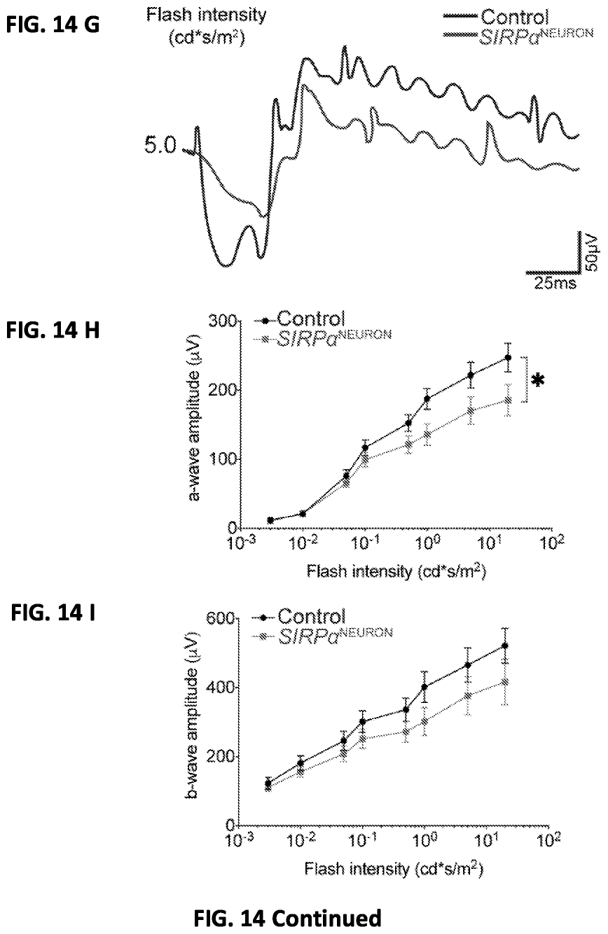

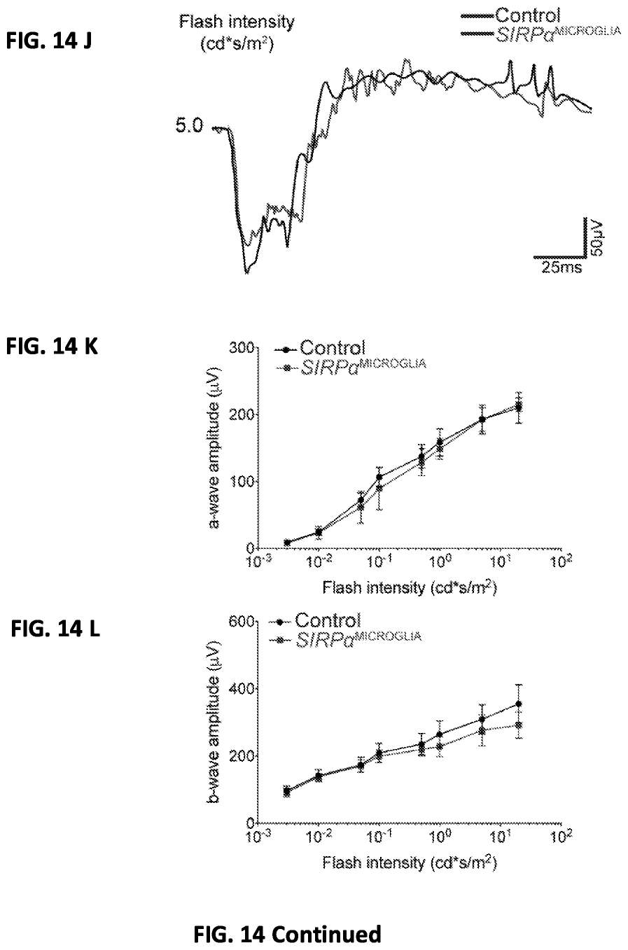

To investigate whether decreased phagocytosis could alter synapse refinement outcomes, the inventors next assayed synapses in neuronal and microglial SIRPα knockouts. The helpful organizational features of the OPL were utilized. Individual synapses in this layer can be quantified using the ribbon marker RIBEYE due to their large size and laminar arrangement (Samuel et al., 2011; Sarin et al., 2018). In SIRPα^{NEURON} retina, decreased microglia phagocytosis was associated with an increase in both RIBEYE fluorescence intensity and the total number of RIBEYE⁺ synapses. In contrast, synapses were largely unaffected in SIRPα^{MICROGLIA} mice (FIG. 14A-F). To assess whether these alterations in synapse number affected visual function, electroretinograms (ERGs) were recorded. The inventors found that SIRPα^{NEURON}, but not SIRPα^{MICROGLIA} mice, showed decreased scotopic a-wave amplitudes, which report directly on photoreceptor function (FIG. 4 G-L). These data indicated that neuronal SIRPα-dependent microglia phagocytosis directly influenced synapse refinement and circuit function.

### Example 8—Prolonging Neuronal SIRPα Expression Extended Microglial Phagocytosis

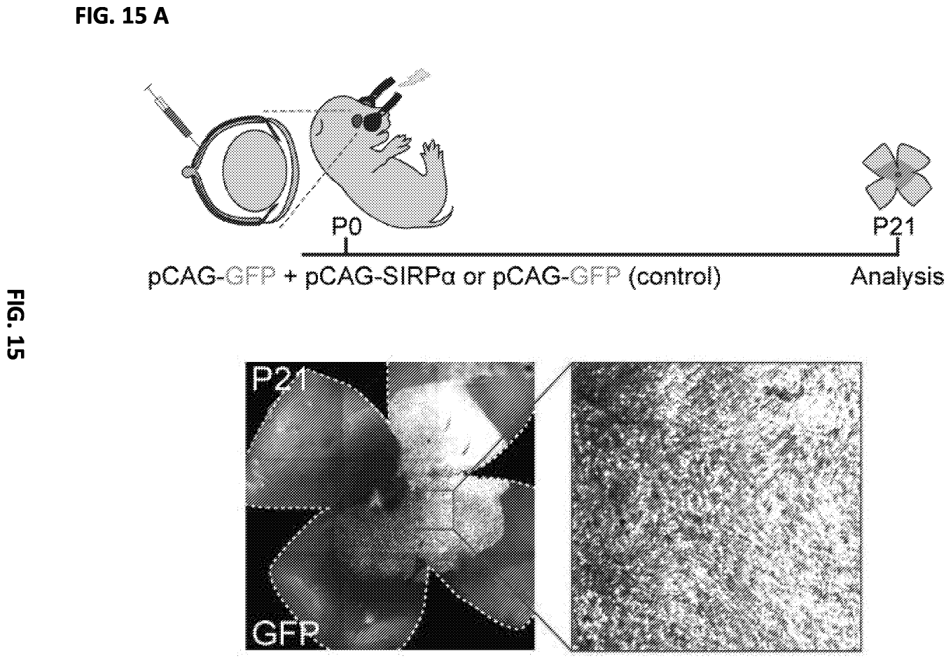

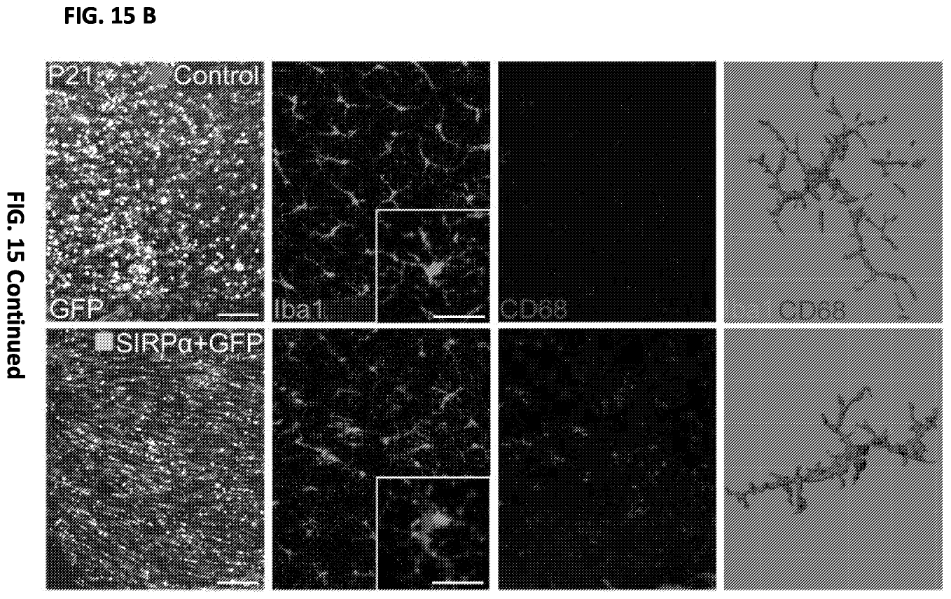

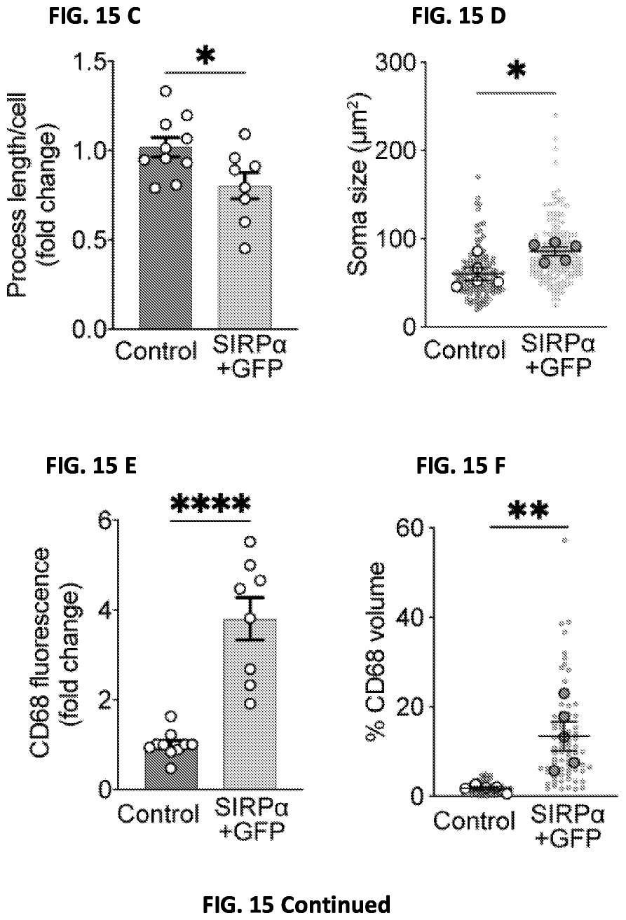

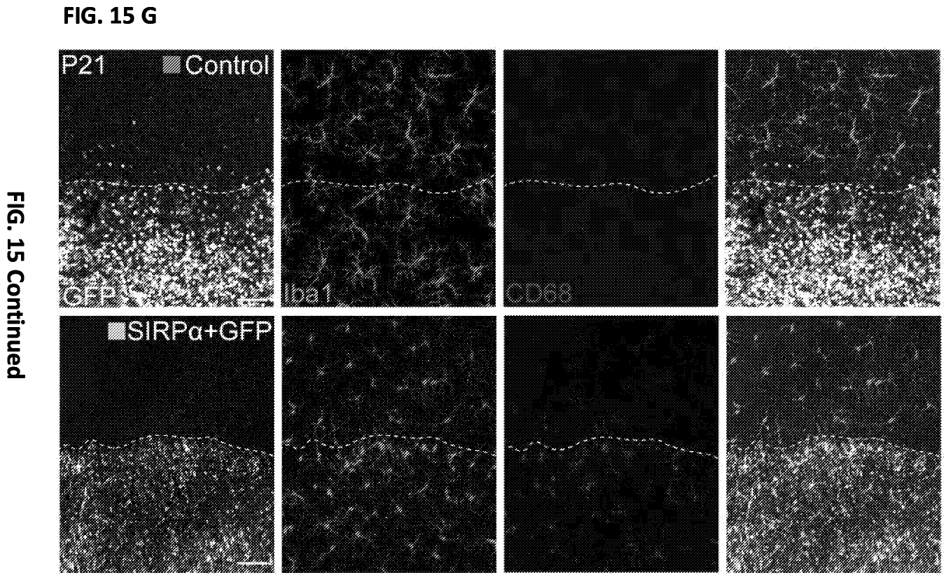

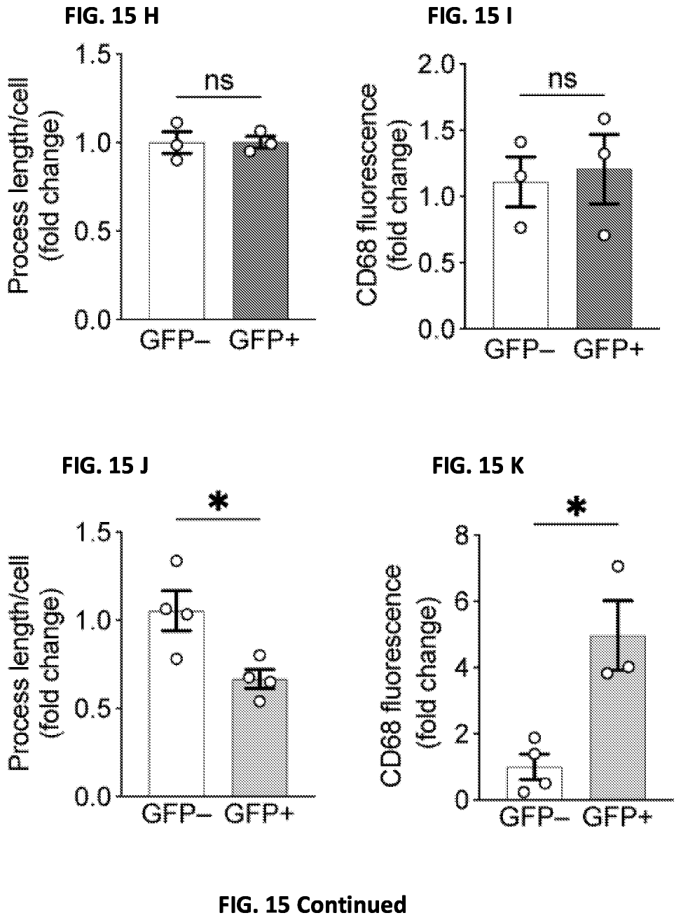

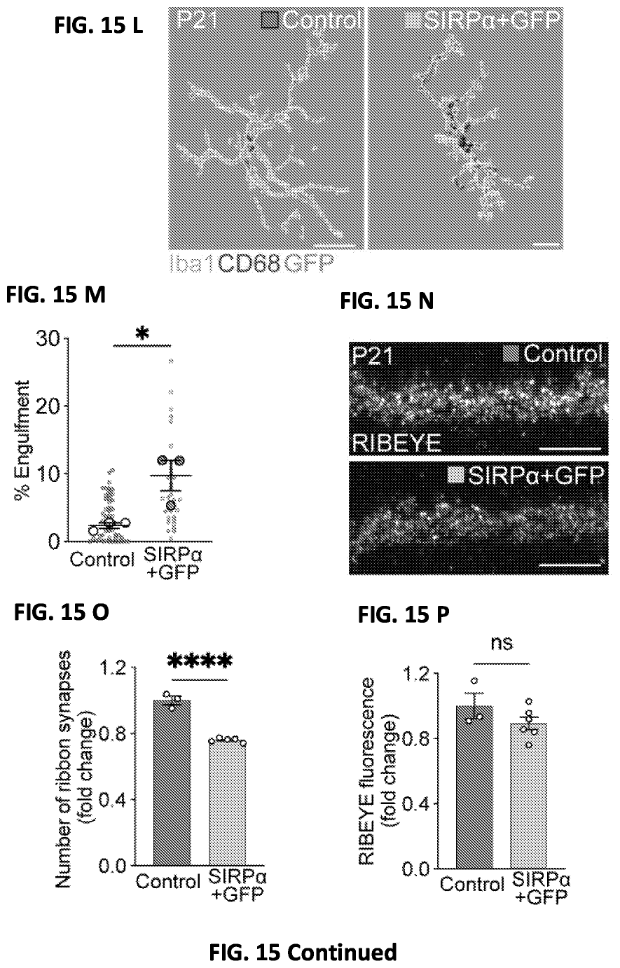

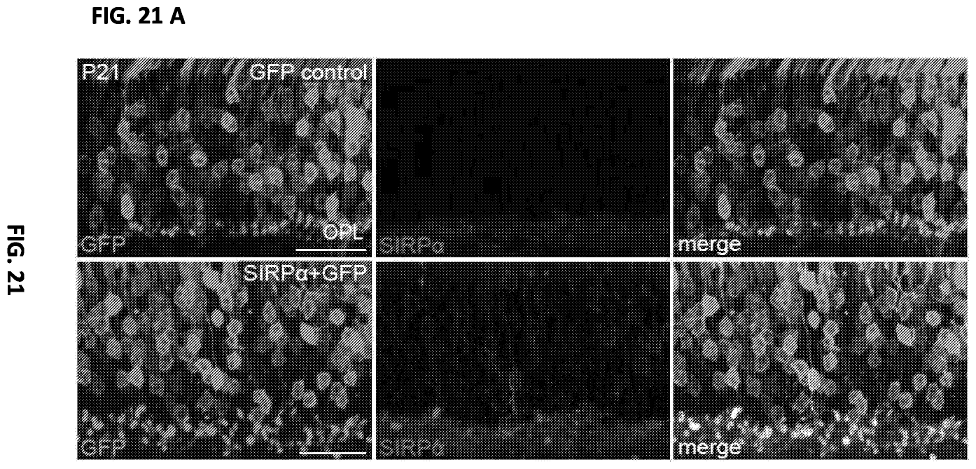

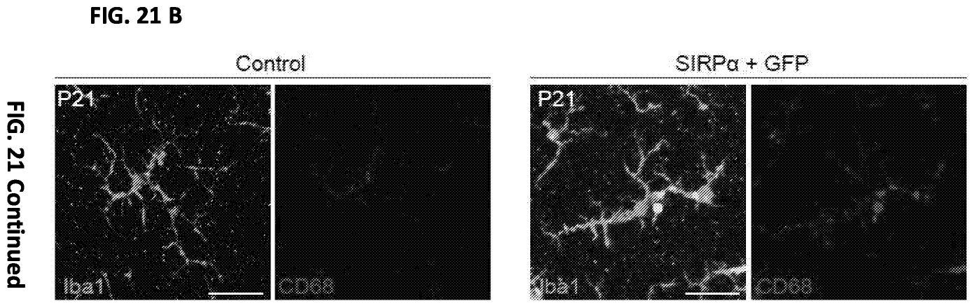

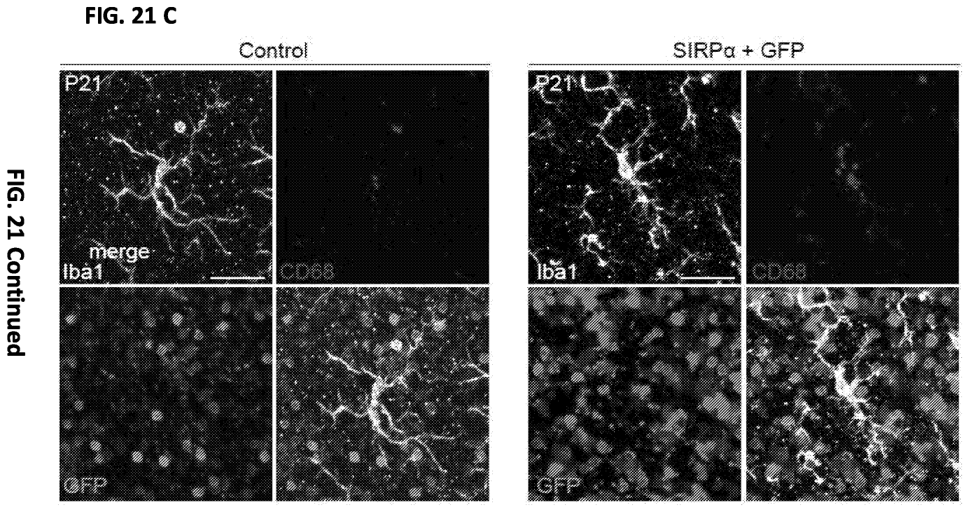

To test whether neuronal SIRPα alone was sufficient to define when and where microglia were phagocytic, the inventors utilized a gain-of-function approach in which SIRPα was introduced by electroporating plasmid DNA in the retina at P0. The results again confirmed that neurons, but not microglia, expressed plasmid DNA following electroporation and that this method successfully increased the amount of neuronal SIRPα (FIG. 15A, and FIG. 21A). To test whether neuronal SIRPα can define the window in which microglia are phagocytic, the inventors assessed microglial morphology and CD68 levels at P21 when microglia phagocytosis was low, cells were correspondingly ramified, and CD68 was reduced. Expression of SIRPα and GFP (but not GFP alone) resulted in a significant increase in markers of microglial phagocytosis (FIG. 15 B, and FIG. 21 B). These cells displayed shorter processes and larger somas and showed significantly increased levels of CD68 globally and in individual microglia (FIG. 15 C-F). Next, we asked whether neuronal SIRPα acted as a local cue to affect microglia phagocytosis. The inventors took advantage of the fact that electroporation targets the retina regionally (Matsuda and Cepko, 2004), generating patches of high neuronal SIRPα expression adjacent to control un-transfected regions that contain wildtype SIRPα expression. Notably, changes in microglial morphology and CD68 expression were restricted precisely to regions in which SIRPα was overexpressed, and adjacent un-transfected regions showed normal, ramified microglia that did not differ significantly from GFP-only transfected controls (FIG. 15 G-K). Thus, neuronal SIRPα appeared sufficient to instruct both the timing and location of microglial phagocytic activity.

To determine whether prolonging microglia phagocytosis beyond the normal developmental window impacted neuronal refinement, the inventors assayed synaptic engulfment and synapse density in SIRPα electroporated retinas and respective controls. The volume of engulfed GFP⁺ neuronal material was quantified through 3D reconstruction of individual microglia in SIRPα+GFP and control (GFP only) transfected retinas. Microglia in SIRPα+GFP patches showed significantly increased engulfed neural material relative to those in controls (FIG. 15 L-M, and FIG. 21 C). Increased engulfment was also associated with decreased synapse numbers, as the total number of RIBEYE+synapses was significantly lower in SIRPα+GFP regions relative to controls (FIG. 15 N-P). Together, these data suggested that neuronal SIRPα acted as a locally restricted cue that determined microglial phagocytosis and was sufficient to extend the developmental window in which neuronal material is engulfed by microglia.

### Example 9—Neuronal SIRPα is Juxtaposed with CD47 at Synapses During Development

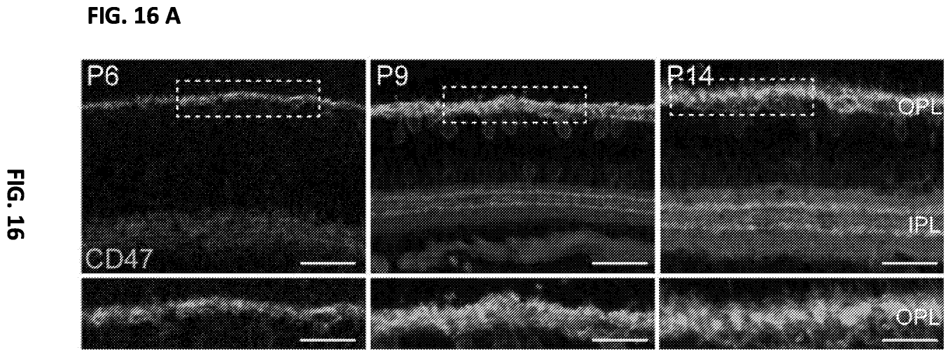

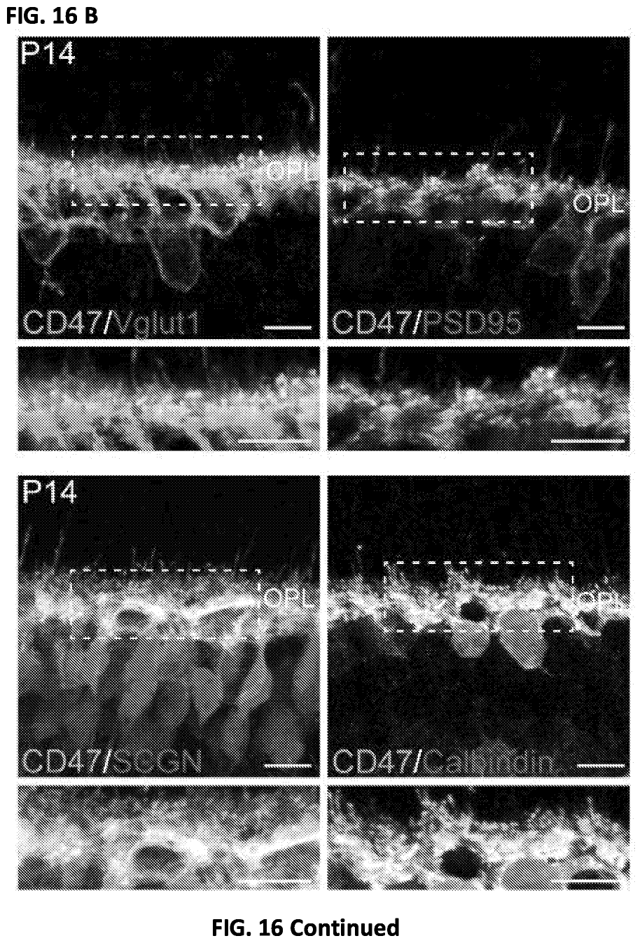

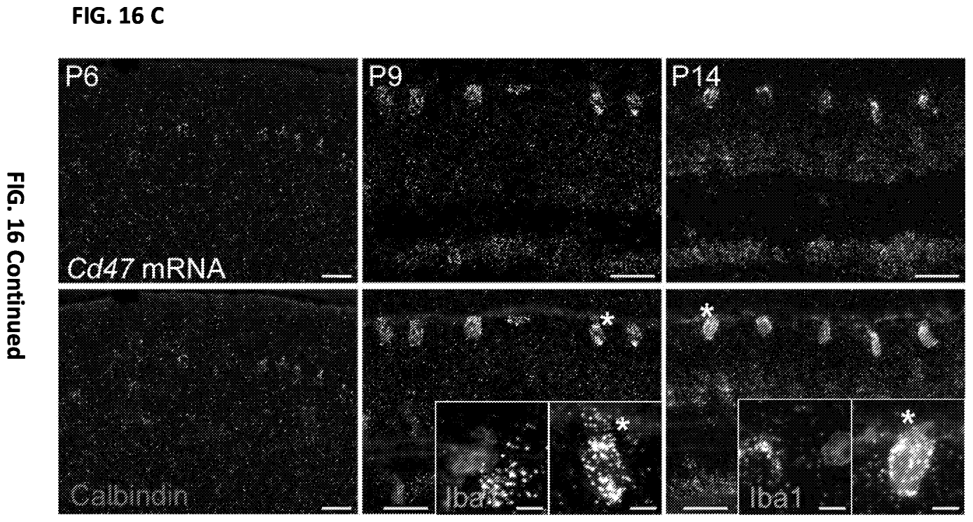

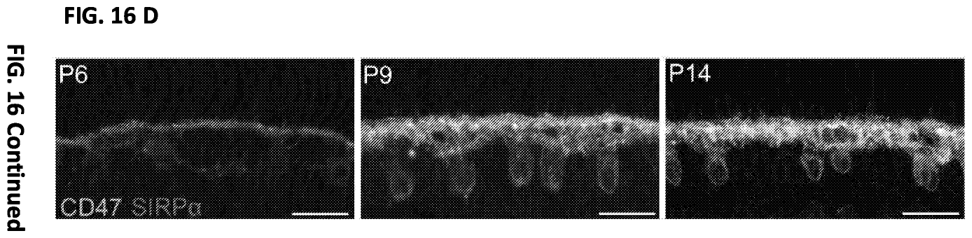

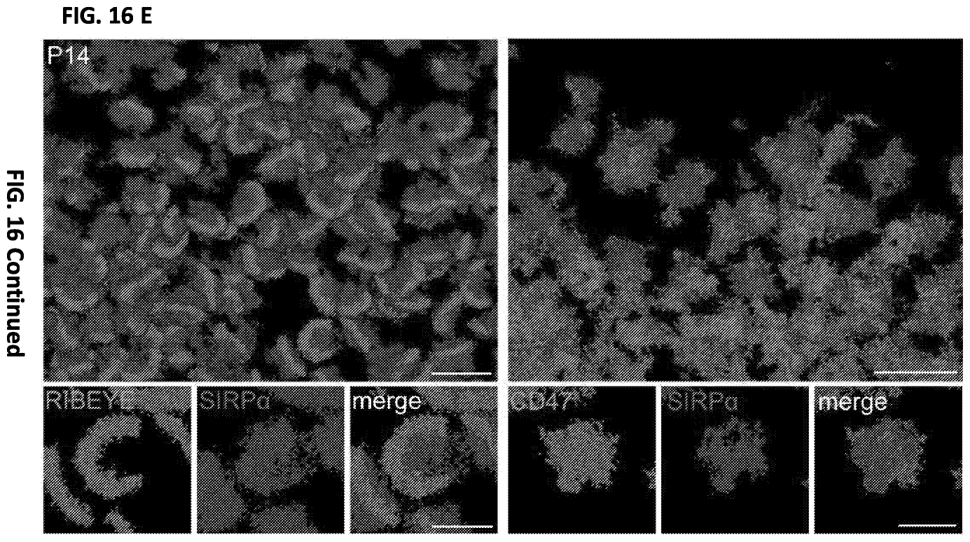

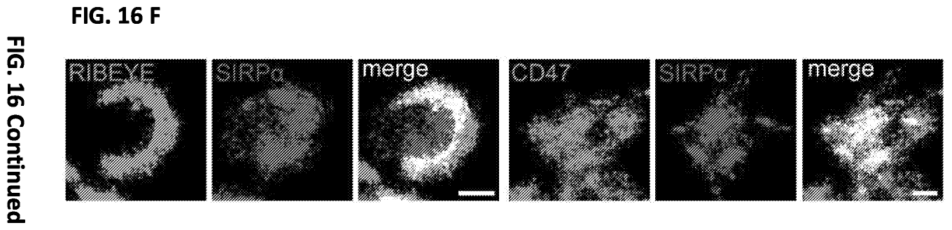

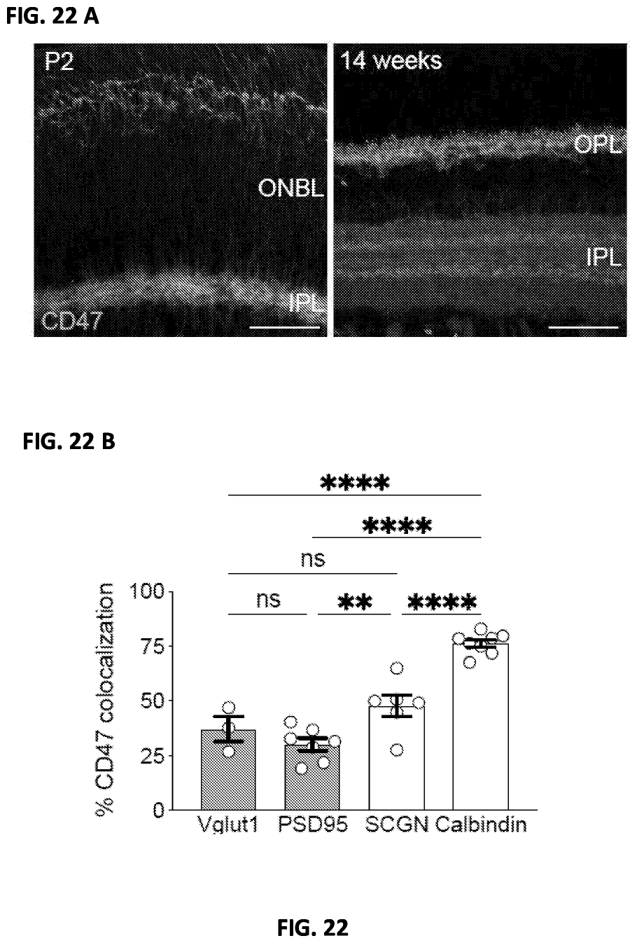

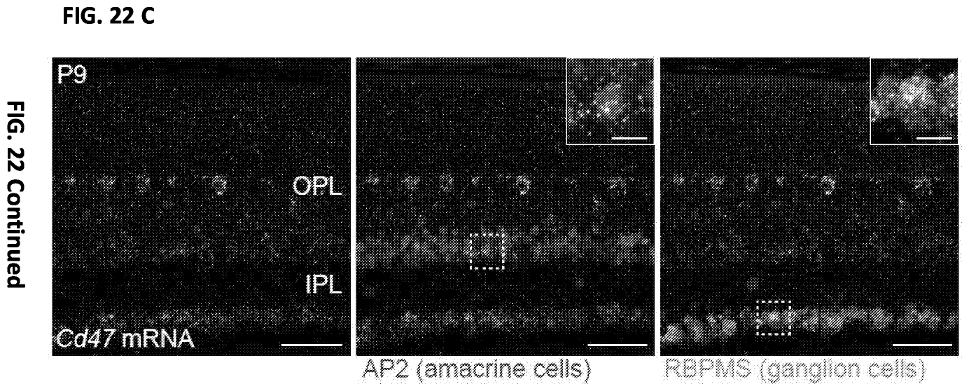

In the periphery, SIRPα is found on phagocytes and serves to limit engulfment through recognition of its only known ligand CD47, which has been characterized as a “don't cat me” signal (Ishikawa-Sekigami et al., 2006; Kojima et al., 2016; Willingham et al., 2012). To elucidate the cellular mechanisms through which neuronal SIRPα may impact microglia function, the inventors first determined where and when CD47 was present in the retina. Immunostaining for CD47 revealed that it was localized to synapse layers as refinement initiated at P2 and increased as refinement progressed (FIG. 16A, and FIG. 22A). Notably, high CD47 protein levels were present in both synapse layers at P9 during the peak of microglia-mediated neuron remodeling, and CD47 was further increased in these regions in adults, CD47 localization at synapses was confirmed by staining with pre- and postsynaptic protein markers in the OPL. Little CD47 colocalized with pre-synaptic markers (Vglut1 and PSD95). Instead, the bulk of CD47 signal overlapped with postsynaptic markers (Calbindin and SCGN), with a particular enrichment at horizontal cell terminals (FIG. 16 B, and FIG. 22 B). The inventors then performed smFISH to determine the cells responsible for CD47 mRNA production. Co-staining with cell type-specific markers confirmed high expression in postsynaptic horizontal cells (FIG. 16 C). Signal was also present in the INL and GCL but was largely absent from microglia (FIG. 16 C, and FIG. 22 C). Together, these data suggested that CD47 was localized postsynaptically in the outer retina and that high levels of this inhibitory cue were present during peak periods of microglia phagocytosis.

The inventors next sought to examine the structural localization of neuronal SIRPα relative to CD47. The inventors first co-labeled both proteins in the OPL over development and examined their structural arrangement via confocal microscopy. The data showed that at P6, P9, and P14, SIRPα and CD47 were concentrated in the OPL and were closely associated (FIG. 16 D). To examine this arrangement in more detail, stochastic optical reconstruction microscopy optimized for tissue imaging (Albrecht et al., 2022) was performed. Dual-color RAIN-STORM imaging confirmed that SIRPα expression was predominantly associated with RIBEYE labeled ribbon synapses and CD47 colocalized with SIRPα at synapses (FIG. 16 E-F). These data indicated that neuronal SIRPα overlapped with its binding partner CD47 at synapses during development

### Example 10—Neuronal SIRPα Promotes Microglia Phagocytosis by Interacting with CD47

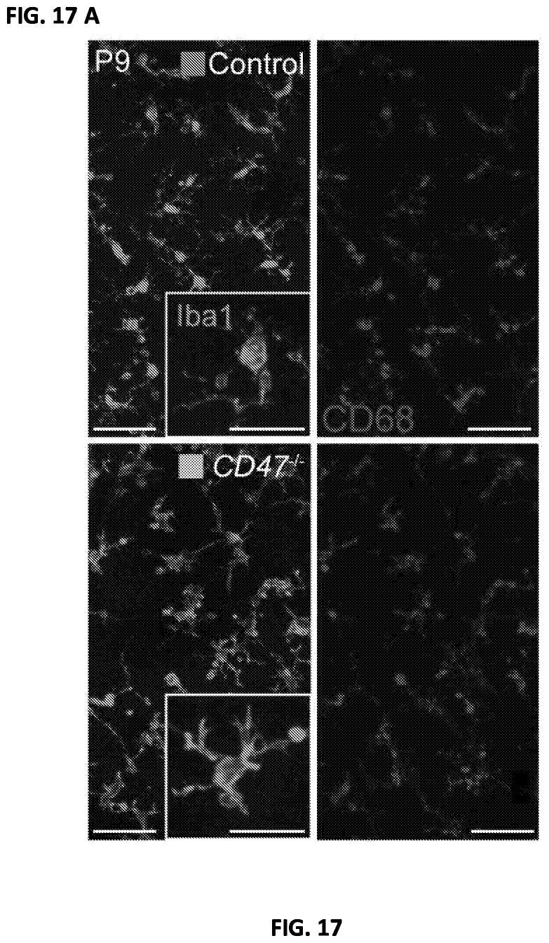

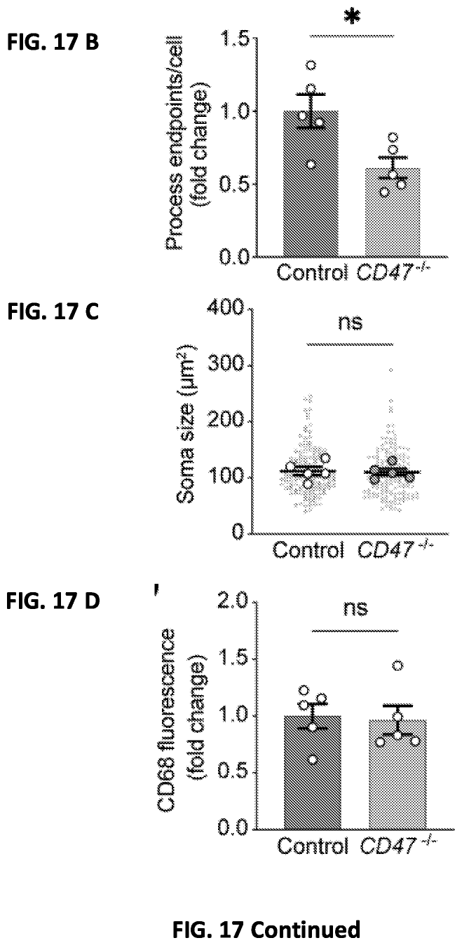

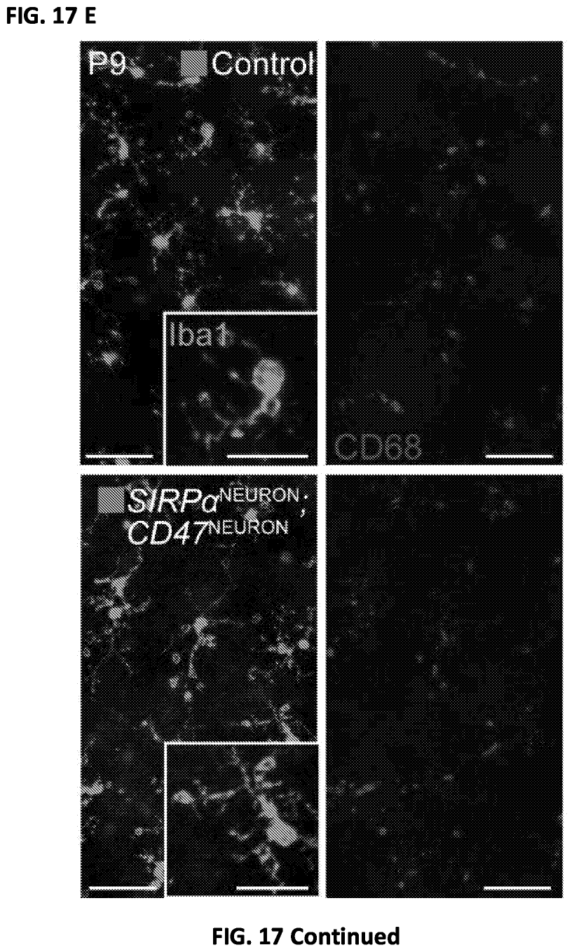

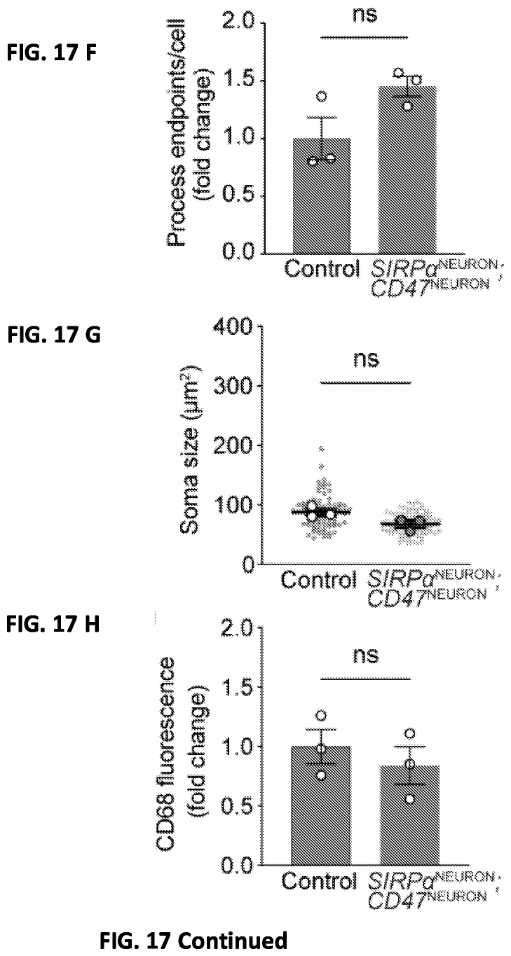

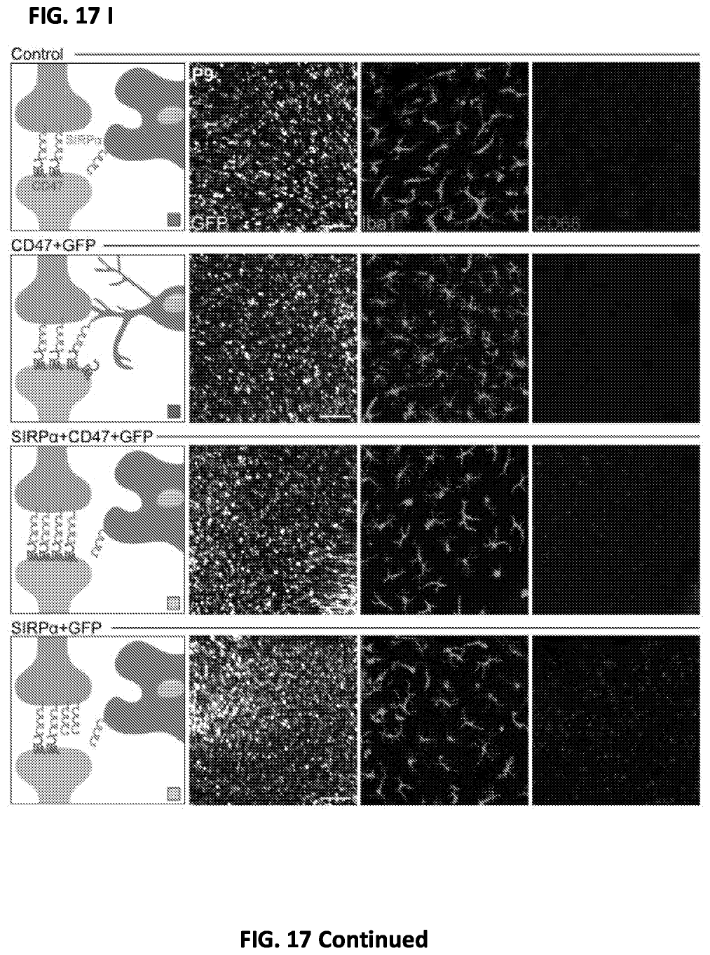

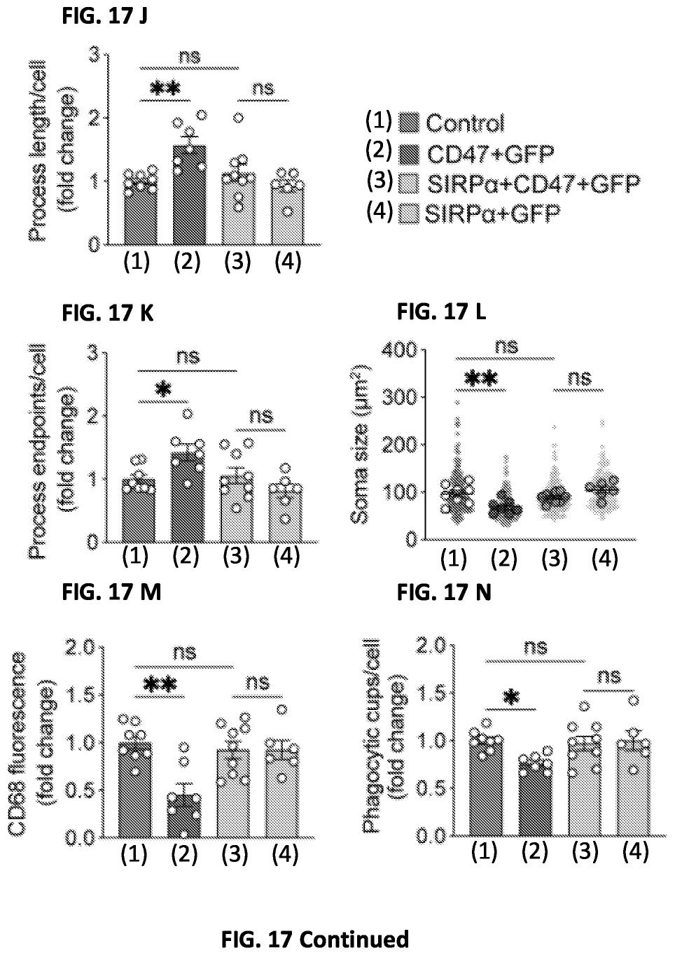

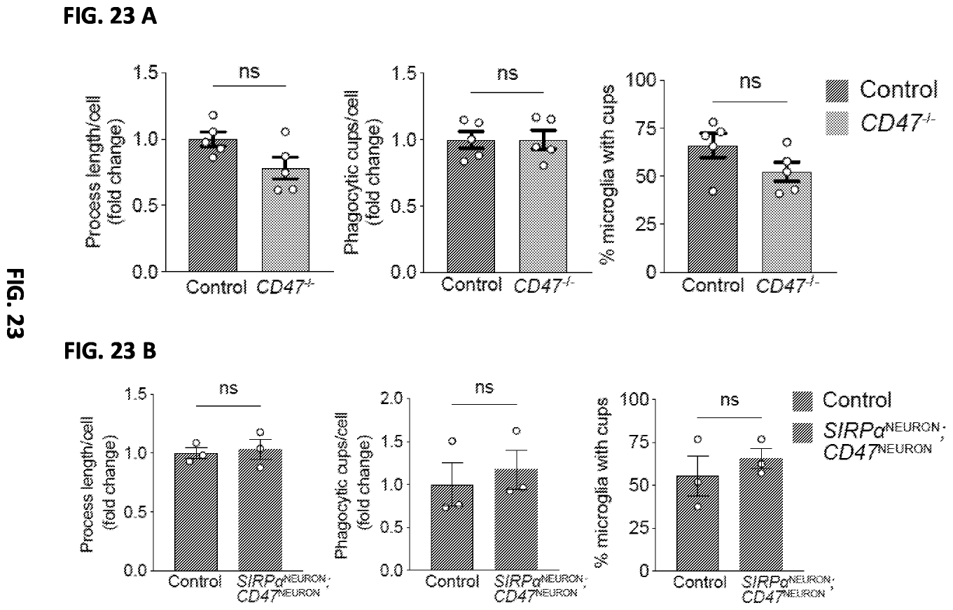

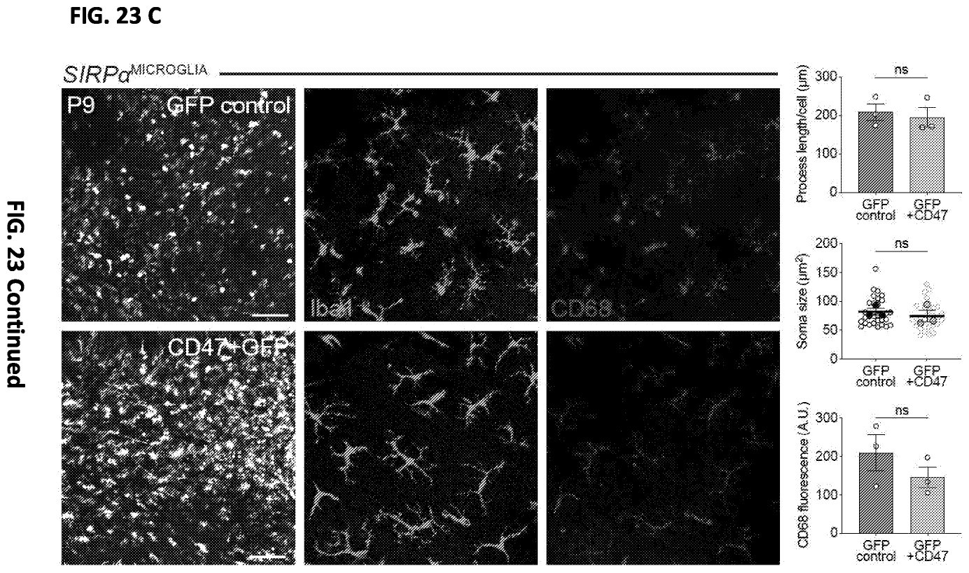

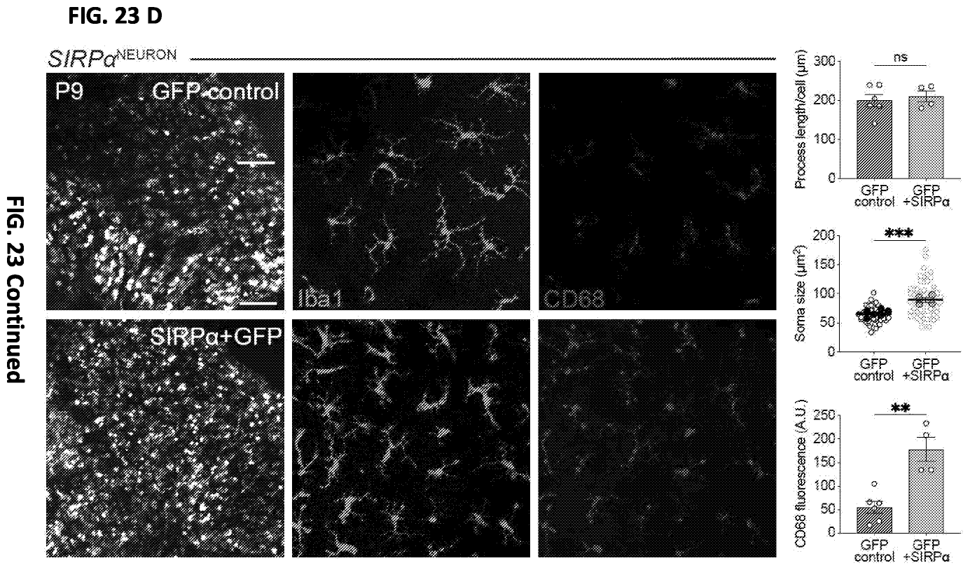

The inventors next investigated whether neuronal SIRPα instructed microglial phagocytosis through its interaction with CD47. To begin, the inventors asked if CD47 itself regulated developmental microglia phagocytosis. Microglia in CD47 null mice showed a small but significant change in the number of process endpoints (FIG. 17A-B). However, microglia process length, soma size, CD68 protein levels, and phagocytic cups did not differ significantly from that of controls (FIG. 17 C-D, and FIG. 23A). Based on these modest effects, it was considered whether neuronal SIRPα limits inhibitory CD47 signaling to microglia during development. This model predicted that removing both neuronal CD47 and SIRPα together would restore phagocytosis that is limited by removal of neuronal SIRPα alone. To test this, the inventors generated SIRPα^{FF}; CD47; Six3^{Cre} mice, termed SIRPα^{NEURON}, CD47NEURON in which both SIRPα and CD47 were removed only in neurons. The data showed that microglia in SIRPα^{NEURON}, CD47NEURON mice showed similar morphologies (FIG. 17 E-G), comparable CD68 expression (FIG. 17 H), and indistinguishable numbers and prevalence of phagocytic cups relative to controls (FIG. 23 B).

The model also predicted that increasing CD47 during development may limit microglia phagocytosis, while increasing CD47 and neuronal SIRPα together would restore microglial phagocytosis (FIG. 17 I). To examine this, the inventors overexpressed CD47 via electroporation at P0 and assayed microglial morphology and CD68 at P9 when microglia were highly phagocytic. Microglia in CD47+GFP patches appeared significantly more ramified relative to controls, with increased process length, process endpoints, and reduced soma size (FIG. 17 J-L). This was accompanied by decreased CD68 and a reduced number of phagocytic cups per cell (FIG. 17 M-N). The inhibitory effect of increasing neuronal CD47 on microglial phagocytic features was mitigated by co-elevating neuronal SIRPα (FIG. 17 I-N). Microglia in co-transfected regions displayed less ramified morphology, and the number of process endpoints, process length, and soma size were all indistinguishable from that in GFP control regions or regions in which SIRPα+GFP was transfected (FIG. 17 I-L). In addition, CD68 was unaltered, as was the number of phagocytic cups per cell (FIG. 17 M-N).

Finally, the model predicted important roles for microglia SIRPα in sensing neuronal CD47-mediated inhibition. In line with this, it was found that microglia SIRPα was required for neuronal CD47-mediated phagocytosis inhibition, CD47 overexpression limited microglia engulfment in controls but had no effect in SIRPα^{MICROGLIA} mice, and microglia displayed similar morphology and comparable CD68 expression (FIG. 23 C). The inventors validated the critical role of neuronal SIRPα in these interactions and confirmed that genetic models did not cause baseline alterations in microglia function by restoring neuronal SIRPα in SIRPα^{NEURON} animals via electroporation. Re-introduction of neuronal SIRPα at P0 significantly restored soma size and CD68 expression in SIRPα^{NEURON} animals (FIG. 23 D). Together, these results suggested that neuronal SIRPα promoted microglia phagocytosis in development by limiting the accessibility of neuronal CD47 to microglia SIRPα.

### Example 11—Discussion

As shown herein, microglia display defined windows of phagocytosis, with high engulfment during neural refinement that is restricted over time. Signals that limit phagocytosis as neurons mature remain largely unknown. Using the murine retina, the inventors have shown that neurons use the membrane glycoprotein SIRPα to tune the levels and timing of microglia phagocytosis. SIRPα localized to both neurons and microglia, and its expression correlated with peak developmental pruning. Using cell type-specific deletion models, it has been shown that while microglia-derived SIRPα is dispensable, neuron-derived SIRPα is required for elevated microglial phagocytosis during development. Deletion of neuronal SIRPα dampened microglia phagocytosis and increased retinal synapse number, while prolonging neuronal SIRPα extended the window of heightened microglial phagocytosis and reduced synapse number. Interactions between neuronal SIRPα and its binding partner CD47 drove these outcomes. The phagocytic inducing effects of prolonging neuronal SIRPα in development were restored by co-expression of neuronal CD47. Conversely, the phagocytic reducing effects of increasing neuronal CD47 were counteracted by increasing neuronal SIRPα. Finally, co-deletion of neuronal SIRPα and CD47 restored microglia phagocytosis. These results indicated that neuronal SIRPα permits microglia phagocytosis by limiting the accessibility of neuronal CD47. These results define unappreciated roles for cell type-specific SIRPα in modulating synapse engulfment.

The nervous system limits microglia engulfment to developmental periods in which neuron remodeling occurs to ensure proper circuit outcomes. The data presented herein indicated that neuronal SIRPα is sufficient to instruct the timing of microglia phagocytosis. In support of this idea, removing neuronal SIRPα caused microglia to adopt a homeostatic morphology in development. Conversely. SIRPα overexpression was sufficient to sustain microglia phagocytosis in normally homeostatic periods. These data raise important questions regarding cause and effect. Does neuronal SIRPα influence synapse-specific decisions that alter global microglial phagocytic capacity, or does microglia phagocytic capacity fundamentally rely on the amount of CD47-SIPRa signaling? The immediate data cannot completely rule out the former possibility but most strongly support the latter. Neuronal CD47 was sufficient to rescue the effects of increasing SIRPα on microglia phagocytosis. This suggests an indirect “decoy receptor” mechanism whereby interactions between presynaptic neuronal SIRPα and postsynaptic CD47 influence phagocytosis by modulating the ability of microglia SIRPα to detect neuronal CD47. In further support of this model, neuron-independent measures of microglia engulfment using labeled yeast particles confirmed a reduction in microglia phagocytic capacity in neuronal SIRPα mutants. Direct signaling mechanisms may also contribute. For example, neuronal SIRPα-dependent synapse loss may affect microglia, or neuronal SIRPα could bind directly to putative microglia CD47. While measurable CD47 in microglia was not detected, CD47 has been documented at low levels on peripheral phagocytes (Doucey et al., 2004; Hayes et al., 2020). Further, while CD47 lacks a substantial cytoplasmic signaling domain (Brooke et al., 2004; Matozaki et al., 2009), it is possible that SIRPα-dependent lateral CD47 interactions with other binding partners could play important roles.

In addition to temporal alignment with neuron remodeling, microglia activity is also spatially restricted. This is particularly easy to appreciate in the laminated retina, where most microglia processes are found within synaptic regions (Li et al., 2019; Rashid et al., 2019). How local neuron-derived cues spatially restrict microglia activity was hitherto unknown. The inventors assessed the spatial relationship between neuronal SIRPα and local microglia phagocytosis using electroporation to create restricted regions of neuronal SIRPα manipulation. Neuronal SIRPα was sufficient to locally instruct microglia activity only in the regions in which it was present. These results have a number of implications. First, they help explain how microglia phagocytosis can proceed during development despite high anti-phagocytic CD47 levels by locally controlling the degree to which microglia can detect this “don't eat me” cue. Second, they suggest that even though SIRPα can be cleaved and secreted (Toth et al., 2013), it does not appear to diffuse broadly beyond the neurons from which it is derived. Third, they suggest that despite the ability of microglia to migrate and dynamically survey diverse neural regions, movements might be limited such that the bulk of signaling occurs locally. It will be informative to determine how neuronal SIRPα influences the rate of microglia environmental sampling in real time.

The results presented herein raises important questions about the impact of local activity-dependent synapse refinement and microglia engulfment. Several cues that target synapses for removal appear to be regulated by activity. These include the complement proteins Clq and C3 (Burger et al., 2020; Schafer et al., 2012). TREM2 (triggering receptor expressed on myeloid cells 2) and its adaptor DAP12 (Filipello et al., 2018; Roumier et al., 2004), major histocompatibility complex (MHC) class I molecules (Datwani et al., 2009; Huh et al., 2000), and fractalkine and its receptor (Gunner et al., 2019; Paolicelli et al., 2011; Rogers et al., 2011). In line with these ideas, SIRPα can directly contribute to synapse maturation in an activity-dependent manner, while CD47 can serve as an activity-dependent “don't eat me” cue that modifies microglia-mediated synapse pruning (Lehrman et al., 2018; Toth et al., 2013). These models imply that the amounts of these synapse-associated proteins vary from synapse to synapse in a way that is predictive of whether a particular synapse will be removed or maintained. The results presented herein may help shed may light on these open questions. Using STORM-microscopy, the inventors found that nearly all synapses in the OPL contained both CD47 and SIRPα, and amounts did not appreciably differ from synapse to synapse. While it cannot be ruled out that minor differences in SIRPα may influence whether a synapse is lost or maintained, these results are more consistent with the idea that CD47-SIRPα signaling at a local population level rather than at single synapses impacts microglia phagocytic activity.

How might the present results be viewed in the context of SIRPα and CD47 whole-body knockout experiments in the brain? Lehrman et al, show limited microglia morphological changes but enhanced microglia engulfment when either SIRPα or CD47 is removed from all cells, resulting in a ˜20-30% decrease in synapse number in the dorsal lateral geniculate nucleus (Lehrman et al., 2018). Similarly, the inventors observed minor morphological changes in CD47 global knockouts with limited but measurable impacts on phagocytosis. Three factors might contribute to the observed brain outcomes in whole-body SIRPα and CD47 knockouts. First, global deletion may obscure the cell subtype-specific contribution of SIRPα on neurons and microglia. In certain embodiments, the model indicates that neuronal and microglial SIRPα have opposing roles. For example, the former is required to promote microglia phagocytosis by temporally limiting microglia SIRPα access to CD47, while the latter is required to limit microglia phagocytosis when neuronal SIRPα decreases, exposing CD47. Consistent with this idea, loss of microglial SIRPα worsens outcomes in a mouse model of Alzheimer's disease (Ding et al., 2021). Second, it is possible that the cellular mechanisms through which SIRPα and CD47 signal may differ between the retina and retinorecipient areas in the brain. However, the inventors view this as unlikely given that: 1) a large body of evidence suggests that retina microglia are structurally, functionally, and developmentally analogous to those in the brain (Anderson et al., 2019; Burger et al., 2020; Hooks and Chen, 2007; Hume et al., 1983; O'Koren et al., 2019; Punal et al., 2019; Schafer et al., 2012; Silverman and Wong, 2018; Stevens et al., 2007; Umpierre and Wu. 2021; Wang et al., 2016; Werneburg et al., 2017), and 2) the expression of these proteins in neurons and microglia are temporally and structurally conserved in the retina and the brain (Adams et al., 1998; Comu et al., 1997; Jiang et al., 2020; Mi et al., 2000). Studies aimed at addressing CD47 and SIRPα cell-specific signaling in the dLGN and other brain regions may aid in resolving these questions.

Finally, the immediate results have potential implications for neurodegenerative diseases. Microglia reactivation is increasingly implicated in the pathogenesis of a large number of both retina and brain diseases and injuries, including diabetic retinopathy, Alzheimer's disease, frontal temporal dementia, demyelinating diseases, and psychiatric diseases (Altmann and Schmidt. 2018; Estes and McAllister. 2015; Hong et al., 2016; Kinuthia et al., 2020; Lall et al., 2021; Lui et al., 2016; Perry et al., 2010; Salter and Stevens, 2017; Sellgren et al., 2019; Vasek et al., 2016; Werneburg et al., 2020). Might neuronal SIRPα and CD47 be involved in these outcomes? In certain embodiments, the model indicates that the answer may be a function of the timing of intervention and the regional amounts of neuronal SIRPα, microglial SIRPα, and CD47. For example, in certain embodiments, in a disease-affected region in which high amounts of neuronal SIRPα and CD47 are present, decreasing neuronal SIRPα may be sufficient to reduce microglia activity and improve neural outcomes. In contrast, in certain embodiments, for diseases of excess connectivity (e.g, autism), elevating neuronal SIRPα in otherwise low SIRPα regions may be sufficient to locally induce microglia phagocytosis. Similarly, in certain embodiments, reduced CD47 expression has been documented in patients with multiple sclerosis (Han et al., 2012; Koning et al., 2007), and experimental models suggest that CD47-SIRPα signaling plays dual roles in this disease (Azcutia et al., 2017; Han et al., 2012; Wang et al., 2021a). Given these results, understanding the regional, neuron-subtype, and synapse-specific consequences of CD47-SIRPα signaling may provide new therapeutic opportunities for precisely intervening in neurological disease progression.

### REFERENCES

The following references, to the extent that they provide exemplary procedural or other details supplementary to those set forth herein, are specifically incorporated herein by reference.
Adams, S., van der Laan, L. J 917., Vernon-Wilson, E. Renardel de Lavalette, C., Dopp. E. A., Dijkstra, C. D., Simmons, D. L., and van den Berg. T. K. (1998). Signal-regulatory protein is selectively expressed by myeloid and neuronal cells. J Immunol 161, 1853-1859.
Albrecht, N. E., Jiang, D., Akhanov, V., Hobson, R., Speer, C. M., Robichaux, M. A., and Samuel, M. A. (2022). Rapid 3D-STORM imaging of diverse molecular targets in tissue. Cell Rep Methods 2, 100253, 10.1016/j.crmeth.2022.100253.
Altmann, C., and Schmidt, M. H. H. (2018). The Role of Microglia in Diabetic Retinopathy: Inflammation, Microvasculature Defects and Neurodegeneration. Int J Mol Sci 19, 10.3390/ijms 19010110.
Anderson, S. R., Roberts, J. M., Zhang, J., Steele, M. R., Romero, C. O., Bosco, A., and Vetter, M. L. (2019). Developmental Apoptosis Promotes a Disease-Related Gene Signature and Independence from CSFIR Signaling in Retinal Microglia. Cell Rep 27, 2002-2013 e2005, 10.1016/j.celrep.2019.04.062.
Azcutia, V., Bassil, R., Herter, J. M., Engelbertsen, D., Newton, G., Autio, A., Mayadas. T., Lichtman, A. H., Khoury, S. J., Parkos, C. A., et al. (2017). Defects in CD4+ T cell LFA-1 integrin-dependent adhesion and proliferation protect Cd47−/-mice from EAE. J Leukoc Biol 101, 493-505, 10.1189/jlb.3A1215-546RR.
Barclay, A. N., and Brown, M. H. (2006). The SIRP family of receptors and immune regulation. Nat Rev Immunol 6, 457-464, 10.1038/nri1859.
Bessis, A., Bechade, C., Bernard. D., and Roumier, A. (2007). Microglial control of neuronal death and synaptic properties. Glia 55, 233-238, 10.1002/glia.20459.
Brooke, G., Holbrook, J. D., Brown, M. H., and Barclay, A. N. (2004). Human lymphocytes interact directly with CD47 through a novel member of the signal regulatory protein (SIRP) family. J Immunol 173, 2562-2570, 10.4049/jimmunol.173.4.2562.
Buch. T., Heppner, F. L.. Tertilt, C., Heinen. T. J., Kremer, M., Wunderlich, F. T., Jung, S., and Waisman, A. (2005). A Cre-inducible diphtheria toxin receptor mediates cell lineage ablation after toxin administration. Nat Methods 2, 419-426, 10.1038/nmeth762.
Burger, C. A., Jiang. D., Li, F., and Samuel, M. A. (2020). Clq Regulates Horizontal Cell Neurite Confinement in the Outer Retina. Front Neural Circuits 14, 583391, 10.3389/fncir.2020.583391.
Chao, M. P., Majeti, R., and Weissman, I. L. (2011). Programmed cell removal: a new obstacle in the road to developing cancer. Nat Rev Cancer 12, 58-67, 10.1038/nrc3171.
Chao, M. P., Weissman, I. L., and Majeti, R. (2012). The CD47-SIRPαlpha pathway in cancer immune evasion and potential therapeutic implications. Curr Opin Immunol 24, 225-232, 10.1016/j.coi.2012.01.010.
Chen. T. T., Brown, E. J., Huang, E. J., and Seaman, W. E. (2004). Expression and activation of signal regulatory protein alpha on astrocytomas. Cancer Res 64, 117-127, 10.1158/0008-5472.can-3455-2.
Chuang, W., and Lagenaur, C. F. (1990). Central nervous system antigen P84 can serve as a substrate for neurite outgrowth. Dev Biol 137, 219-232, 10.1016/0012-1606 (90) 90249-i.
Comu, S., Weng. W., Olinsky, S., Ishwad, P., Mi, Z., Hempel, J., Watkins, S.,
Lagenaur, C. F., and Narayanan, V. (1997). The murine P84 neural adhesion molecule is SHPS-1, a member of the phosphatase-binding protein family. J Neurosci 17, 8702-8710.
Datwani, A., McConnell, M. J., Kanold, P. O., Micheva, K. D., Busse, B., Shamloo, M., Smith, S. J., and Shatz, C. J. (2009). Classical MHCI molecules regulate retinogeniculate refinement and limit ocular Neuron dominance plasticity. 64, 463-470, 10.1016/j.neuron.2009.10.015.
Ding. X., Wang, J., Huang, M., Chen, Z., Liu, J., Zhang, Q., Zhang, C., Xiang, Y., Zen, K., and Li, L. (2021). Loss of microglial SIRPαlpha promotes synaptic pruning in preclinical models of neurodegeneration. Nat Commun 12, 2030, 10.1038/s41467-021-22301-1.
Doucey, M. A., Scarpellino, L., Zimmer, J., Guillaume, P., Luescher, I. F., Bron, C., and Held, W. (2004). Cis association of Ly49A with MHC class I restricts natural killer cell inhibition. Nat Immunol 5, 328-336, 10.1038/ni1043.
Dunn, K. W., Kamocka, M. M., and McDonald, J. H. (2011). A practical guide to evaluating colocalization in biological microscopy. Am J Physiol Cell Physiol 300, C723-742.10.1152/ajpcell.00462.2010.
Estes, M. L., and McAllister, A. K. (2015). Immune mediators in the brain and peripheral tissues in autism spectrum disorder. Nat Rev Neurosci 16, 469-486.10.1038/nrn3978.
Filipello, F., Morini, R., Corradini, I., Zerbi, V., Canzi, A., Michalski, B., Erreni, M., Markicevic, M., Starvaggi-Cucuzza, C., Otero, K., et al. (2018). The Microglial Innate Immune Receptor TREM2 Is Required for Synapse Elimination and Normal Brain Connectivity. Immunity 48, 979-991 e978, 10.1016/j.immuni.2018.04.016.
Ford, A. L., Goodsall, A. L., Hickey, W. F., and Sedgwick, J. D. (1995). Normal adult ramified microglia separated from other central nervous system macrophages by flow cytometric sorting. Phenotypic differences defined and direct ex vivo antigen presentation to myelin basic protein-reactive CD4+ T cells compared. J Immunol 154, 4309-4321.
Fu, R., Shen, Q., Xu. P., Luo, J. J., and Tang. Y. (2014). Phagocytosis of microglia in the central nervous system diseases. Mol Neurobiol 49, 1422-1434, 10.1007/s12035-013-8620-6. Furuta, Y., Lagutin, O., Hogan. B. L., and Oliver, G. C. (2000). Retina- and ventral forebrain-specific Cre recombinase activity in transgenic mice. Genesis 26, 130-132.
Gardai, S. J., McPhillips, K. A., Frasch, S. C., Janssen, W. J., Starefeldt, A., Murphy-Ullrich, J. E., Bratton, D. L., Oldenborg, P. A., Michalak, M., and Henson, P. M. (2005). Cell-surface calreticulin initiates clearance of viable or apoptotic cells through trans-activation of LRP on the phagocyte. Cell 123, 321-334, 10.1016/j.cell.2005.08.032.
Gomez Perdiguero, E., Klapproth. K., Schulz, C., Busch, K., Azzoni, E., Crozet, L., Garner, H.. Trouillet. C., de Bruijn, M. F., Geissmann, F., and Rodewald, H. R. (2015). Tissue-resident macrophages originate from yolk-sac-derived erythro-myeloid progenitors. Nature 518, 547-551, 10.1038/nature13989.
Gunner, G., Cheadle, L., Johnson, K. M., Ayata, P., Badimon. A., Mondo. E., Nagy, M. A., Liu, L., Bemiller, S. M., Kim, K. W., et al. (2019). Sensory lesioning induces microglial synapse elimination via ADAM10 and fractalkine signaling. Nat Neurosci 22, 1075-1088.10.1038/s41593-019-0419-y.
Han, M. H., Lundgren. D. H., Jaiswal, S. Chao, M., Graham. K. L., Garris. C. S., Axtell, R. C., Ho, P. P., Lock, C. B., Woodard, J. I., et al. (2012). Janus-like opposing roles of CD47 in autoimmune brain inflammation in humans and mice. J Exp Med 209, 1325-1334.10.1084/jem.20101974.
Hayes, B. H.. Tsai, R. K., Dooling, L. J., Kadu, S., Lee, J. Y., Pantano, D., Rodriguez, P. L., Subramanian. S., Shin, J. W., and Discher, D. E. (2020). Macrophages show higher levels of engulfment after disruption of cis interactions between CD47 and the checkpoint receptor SIRPαlpha. J Cell Sci 133, 10.1242/jcs.237800.
Hong. S., Beja-Glasser, V. F., Nfonoyim, B. M., Frouin, A. Li, S. Ramakrishnan, S., Merry, K. M., Shi, Q., Rosenthal, A., Barres, B. A., et al. (2016). Complement and microglia mediate early synapse loss in Alzheimer mouse models. Science 352, 712-716.10.1126/science.aad8373.
Hooks, B. M., and Chen, C. (2007). Critical periods in the visual system: changing views for a model of experience-dependent plasticity. Neuron 56, 312-326.10.1016/j.neuron.2007.10.003.
Huh. G. S., Boulanger, L. M., Du, H., Riquelme. P. A., Brotz. T. M., and Shatz, C. J. (2000). Functional requirement for class I MHC in CNS development and plasticity. Science 290, 2155-2159, 10.1126/science.290.5499.2155.
Hume, D. A., Perry, V. H., and Gordon, S. (1983). Immunohistochemical localization of a macrophage specific antigen in developing mouse retina: phagocytosis of dying neurons and differentiation of microglial cells to form a regular array in the plexiform layers. J Cell Biol 97, 253-257, 10.1083/jcb.97.1.253.
Ishikawa-Sekigami. T., Kaneko, Y., Okazawa, H.. Tomizawa. T., Okajo, J., Saito, Y., Okuzawa, C., Sugawara-Yokoo, M., Nishiyama, U., Ohnishi, H., et al. (2006). SHPS-1 promotes the survival of circulating erythrocytes through inhibition of phagocytosis by splenic macrophages. Blood 107, 341-348, 10.1182/blood-20May 5, 1896
Jiang, D., Burger. C. A., Casasent, A. K., Albrecht, N. E., Li, F., and Samuel, M. A. (2020). Spatiotemporal gene expression patterns reveal molecular relatedness between retinal laminae. J Comp Neurol 528, 729-755, 10.1002/cne.24784.
Jiang. P., Lagenaur. C. F., and Narayanan. V. (1999). Integrin-associated protein is a ligand for the P84 neural adhesion molecule. J Biol Chem 274, 559-562.10.1074/jbc.274.2.559.
Jordao, M. J. C., Sankowski. R., Brendecke, S. M., Sagar, Locatelli, G.. Tai, Y. H., Tay. T. L., Schramm, E., Armbruster, S., Hagemeyer, N., et al. (2019). Single-cell profiling identifies myeloid cell subsets with distinct fates during neuroinflammation. Science 363.10.1126/science.aat7554. Jung, S., Aliberti, J., Graemmel, P., Sunshine, M. J., Kreutzberg. G. W., Sher, A., and Littman, D. R. (2000). Analysis of fractalkine receptor CX (3) CR1 function by targeted deletion and green fluorescent protein reporter gene insertion. Mol Cell Biol 20, 4106-4114.10.1128/MCB.20.11.4106-4114.2000.
Kharitonenkov, A., Chen, Z., Sures, I., Wang, H., Schilling, J., and Ullrich, A. (1997). A family of proteins that inhibit signalling through tyrosine kinase receptors. Nature 386, 181-186, 10.1038/386181a0.
Kim, I. J., Zhang, Y., Meister, M., and Sanes, J. R. (2010). Laminar restriction of retinal ganglion cell dendrites and axons: subtype-specific developmental patterns revealed with transgenic markers. J Neurosci 30, 1452-1462, 10.1523/JNEUROSCI.4779-09.2010.
Kinuthia, U. M., Wolf, A., and Langmann. T. (2020). Microglia and Inflammatory Responses in Diabetic Retinopathy. Front Immunol 11, 564077, 10.3389/fimmu.2020.564077.
Kojima, Y., Volkmer, J. P., McKenna. K., Civelek, M., Lusis, A. J., Miller, C. L., Direnzo, D., Nanda, V., Ye, J., Connolly, A. J., et al. (2016), CD47-blocking antibodies restore phagocytosis and prevent atherosclerosis. Nature 536, 86-90, 10.1038/nature18935.
Koning. N., Bo. L., Hoek, R. M., and Huitinga, I. (2007). Downregulation of macrophage inhibitory molecules in multiple sclerosis lesions. Ann Neurol 62, 504-514.10.1002/ana.21220.
Lall, D., Lorenzini, I., Mota. T. A., Bell, S., Mahan. T. E., Ulrich, J. D., Davtyan, H., Rexach, J. E., Muhammad, A., Shelest, O., et al. (2021). C9orf72 deficiency promotes microglial-mediated synaptic loss in aging and amyloid accumulation. Neuron 109, 2275-2291 e2278, 10.1016/j.neuron.2021.05.020.
Lehrman, E. K., Wilton, D. K., Litvina, E. Y., Welsh. C. A., Chang, S. T., Frouin, A., Walker, A. J., Heller, M. D., Umemori, H., Chen, C., and Stevens, B. (2018), CD47 Protects Synapses from Excess Microglia-Mediated Pruning during Development. Neuron 100, 120-134 e126, 10.1016/j.neuron.2018.09.017.
Li, F., Jiang, D., and Samuel, M. A. (2019). Microglia in the developing retina. Neural Dev 14, 12, 10.1186/s13064-019-0137-x.
Lui, H., Zhang, J., Makinson, S. R., Cahill, M. K., Kelley, K. W., Huang, H. Y., Shang, Y., Oldham, M. C., Martens, L. H., Gao, F., et al. (2016). Progranulin Deficiency Promotes Circuit-Specific Synaptic Pruning by Microglia via Complement Activation. Cell 165, 921-935, 10.1016/j.cell.2016.04.001.
Maeda, K., Kobayashi, Y., Udagawa. N., Uehara, S., Ishihara, A., Mizoguchi. T., Kikuchi, Y.. Takada, I., Kato, S., Kani, S., et al. (2012). Wnt5a-Ror2 signaling between osteoblast-lineage cells and osteoclast precursors enhances osteoclastogenesis. Nat Med 18, 405-412, 10.1038/nm.2653.
Majeti, R., Chao, M. P., Alizadeh, A. A., Pang, W. W., Jaiswal, S., Gibbs. K. D., Jr., van Rooijen, N., and Weissman, I. L. (2009), CD47 is an adverse prognostic factor and therapeutic antibody target on human acute myeloid leukemia stem cells. Cell 138, 286-299.10.1016/j.cell.2009.05.045.
Mass, E., Ballesteros, I., Farlik, M., Halbritter, F., Gunther, P., Crozet, L., Jacome-Galarza, C. E., Handler, K., Klughammer, J., Kobayashi, Y., et al. (2016). Specification of tissue-resident macrophages during organogenesis. Science 353, 10.1126/science.aaf4238.
Matozaki. T., Murata, Y., Okazawa, H., and Ohnishi, H. (2009). Functions and molecular mechanisms of the CD47-SIRPαlpha signalling pathway. Trends Cell Biol 19, 72-80, 10.1016/j.tcb.2008.12.001.
Matsuda. T., and Cepko, C. L. (2004). Electroporation and RNA interference in the rodent retina in vivo and in vitro. Proc Natl Acad Sci USA 101, 16-22.10.1073/pnas.2235688100.
Matsuda. T., and Cepko, C. L. (2007). Controlled expression of transgenes introduced by in vivo electroporation. Proc Natl Acad Sci USA 104, 1027-1032.10.1073/pnas.0610155104.
Mi, Z. P., Jiang, P., Weng. W. L., Lindberg. F. P., Narayanan, V., and Lagenaur. C. F. (2000). Expression of a synapse-associated membrane protein, P84/SHPS-1, and its ligand, IAP/CD47, in mouse retina. J Comp Neurol 416, 335-344.
Miksa. M., Komura, H., Wu, R., Shah, K. G., and Wang. P. (2009). A novel method to determine the engulfment of apoptotic cells by macrophages using pHrodo succinimidyl ester. J Immunol Methods 342, 71-77, 10.1016/j.jim.2008.11.019.
Nagappan-Chettiar, S., Johnson-Venkatesh, E. M., and Umemori, H. (2018). Tyrosine phosphorylation of the transmembrane protein SIRPαlpha: Sensing synaptic activity and regulating ectodomain cleavage for synapse maturation. J Biol Chem 293, 12026-12042.10.1074/jbc.RA117.001488.
O'Koren, E. G., Yu, C., Klingeborn, M., Wong, A. Y. W., Prigge, C. L., Mathew, R., Kalnitsky, J., Msallam, R. A., Silvin, A., Kay, J. N., et al. (2019). Microglial Function Is Distinct in Different Anatomical Locations during Retinal Homeostasis and Degeneration. Immunity 50, 723-737 e727, 10.1016/j.immuni.2019.02.007.
Oldenborg, P. A., Gresham, H. D., and Lindberg, F. P. (2001), CD47-signal regulatory protein alpha (SIRPαlpha) regulates Fcgamma and complement receptor-mediated phagocytosis. J Exp Med 193, 855-862, 10.1084/jem. 193.7.855.
Oldenborg, P. A., Zheleznyak, A., Fang. Y. F., Lagenaur, C. F., Gresham, H. D., and Lindberg, F. P. (2000). Role of CD47 as a marker of self on red blood cells. Science 288, 2051-2054, 10.1126/science.288.5473.2051.
Paolicelli, R. C., Bolasco, G., Pagani, F., Maggi. L., Scianni, M., Panzanelli, P., Giustetto, M., Ferreira. T. A., Guiducci, E., Dumas, L., et al. (2011). Synaptic pruning by microglia is necessary for normal brain development. Science 333, 1456-1458.10.1126/science.1202529.
Perry, V. H., Nicoll. J. A., and Holmes, C. (2010). Microglia in neurodegenerative disease. Nat Rev Neurol 6, 193-201, 10.1038/nrneurol.2010.17.
Punal, V. M., Paisley, C. E., Brecha, F. S., Lee, M. A., Perelli, R. M., Wang, J., O'Koren, E. G., Ackley, C. R., Saban, D. R., Reese, B. E., and Kay, J. N. (2019). Large-scale death of retinal astrocytes during normal development is non-apoptotic and implemented by microglia. PLOS Biol 17, e3000492, 10.1371/journal.pbio.3000492.
Qin, J. M., Yan, H. X., Liu, S. Q., Wan, X. W., Zeng. J. Z., Cao, H. F., Qiu, X. H., Wu, M. C., and Wang. H. Y. (2006). Negatively regulating mechanism of Sirpalphal in hepatocellular carcinoma: an experimental study. Hepatobiliary Pancreat Dis Int 5, 246-251.
Rashid, K., Akhtar-Schaefer, I., and Langmann. T. (2019). Microglia in Retinal Degeneration. Front Immunol 10, 1975, 10.3389/fimmu.2019.01975.
Rogers, J. T., Morganti, J. M., Bachstetter, A. D., Hudson, C. E., Peters, M. M., Grimmig, B. A., Weeber, E. J., Bickford, P. C., and Gemma, C. (2011). CX3CR1 deficiency leads to impairment of hippocampal cognitive function and synaptic plasticity. J Neurosci 31.16241-16250, 10.1523/JNEUROSCI.3667-11.2011.
Roumier, A., Bechade, C., Poncer, J. C., Smalla, K. H.. Tomasello, E., Vivier, E., Gundelfinger, E. D.. Triller, A., and Bessis, A. (2004). Impaired synaptic function in the microglial KARAP/DAP12-deficient mouse. J Neurosci 24, 11421-11428.10.1523/JNEUROSCI.2251-04.2004.
Salter, M. W., and Stevens, B. (2017). Microglia emerge as central players in brain disease. Nat Med 23, 1018-1027, 10.1038/nm.4397.
Samuel, M. A., Zhang, Y., Meister, M., and Sanes, J. R. (2011). Age-related alterations in neurons of the mouse retina. J Neurosci 31, 16033-16044.10.1523/JNEUROSCI.3580-11.2011.
Sarin, S., Zuniga-Sanchez, E., Kurmangaliyev, Y. Z., Cousins, H., Patel, M., Hernandez, J., Zhang, K. X., Samuel, M. A., Morey, M., Sanes, J. R., and Zipursky, S. L. (2018). Role for Wnt Signaling in Retinal Neuropil Development: Analysis via RNA-Seq and In Vivo Somatic CRISPR Mutagenesis. Neuron 98, 109-126 e108, 10.1016/j.neuron.2018.03.004.
Schafer, D. P., Lehrman, E. K., Heller, C. T., and Stevens, B. (2014). An engulfment assay: a protocol to assess interactions between CNS phagocytes and neurons. J Vis Exp. 10.3791/51482.
Schafer, D. P., Lehrman, E. K., Kautzman, A. G., Koyama. R., Mardinly, A. R., Yamasaki, R., Ransohoff, R. M., Greenberg, M. E., Barres, B. A., and Stevens, B. (2012). Microglia sculpt postnatal neural circuits in an activity and complement-dependent manner. Neuron 74, 691-705, 10.1016/j.neuron.2012.03.026.
Sellgren, C. M., Gracias, J., Watmuff, B., Biag, J. D.. Thanos, J. M., Whittredge, P. B., Fu. T., Worringer, K., Brown, H. E., Wang, J., et al. (2019). Increased synapse elimination by microglia in schizophrenia patient-derived models of synaptic pruning. Nat Neurosci 22, 374-385, 10.1038/s41593-018-0334-7.
Sierra, A., Abiega, O., Shahraz, A., and Neumann, H. (2013). Janus-faced microglia: beneficial and detrimental consequences of microglial phagocytosis. Front Cell Neurosci 7, 6, 10.3389/fncel.2013.00006.
Silverman, S. M., and Wong, W. T. (2018). Microglia in the Retina: Roles in Development, Maturity, and Disease. Annu Rev Vis Sci 4, 45-77, 10.1146/annurev-vision-091517-034425.
Skarnes, W. C., Rosen, B., West, A. P., Koutsourakis, M., Bushell, W., Iyer, V., Mujica, A. O.. Thomas, M., Harrow, J., Cox. T., et al. (2011). A conditional knockout resource for the genome-wide study of mouse gene function. Nature 474, 337-342.10.1038/nature10163.
Stevens, B., Allen, N. J., Vazquez, L. E., Howell, G. R., Christopherson, K. S., Nouri, N., Micheva, K. D., Mehalow, A. K., Huberman, A. D., Stafford, B., et al. (2007). The classical complement cascade mediates CNS synapse elimination. Cell 131, 1164-1178.10.1016/j.cell.2007.10.036.
Swanson, J. A. (2008). Shaping cups into phagosomes and macropinosomes. Nat Rev Mol Cell Biol 9, 639-649, 10.1038/nrm2447.
Takahashi, S. (2018). Molecular functions of SIRPαlpha and its role in cancer. Biomed Rep 9,3-7, 10.3892/br.2018.1102.
Toth, A. B.. Terauchi, A., Zhang, L. Y., Johnson-Venkatesh, E. M., Larsen, D. J., Sutton. M. A., and Umemori, H. (2013). Synapse maturation by activity-dependent ectodomain shedding of SIRPαlpha. Nat Neurosci 16, 1417-1425, 10.1038/nn.3516.
Tremblay, M. E., Lowery, R. L., and Majewska, A. K. (2010). Microglial interactions with synapses are modulated by visual experience. PLOS Biol 8, e1000527.10.1371/journal.pbio.1000527.
Umemori, H., and Sanes, J. R. (2008). Signal regulatory proteins (SIRPS) are secreted presynaptic organizing molecules. J Biol Chem 283, 34053-34061.10.1074/jbc.M805729200.
Umpierre, A. D., and Wu, L. J. (2021). How microglia sense and regulate neuronal activity. Glia 69, 1637-1653, 10.1002/glia.23961.
van Beek, E. M., Cochrane, F., Barclay, A. N., and van den Berg. T. K. (2005). Signal regulatory proteins in the immune system. J Immunol 175, 7781-7787.10.4049/jimmunol. 175.12.7781.
Vasek, M. J., Garber, C., Dorsey, D., Durrant, D. M., Bollman, B., Soung, A., Yu, J., Perez-Torres, C., Frouin, A., Wilton, D. K., et al. (2016). A complement-microglial axis drives synapse loss during virus induced memory impairment. Nature 534, 538-543.10.1038/nature18283.
Wang, H., Newton, G., Wu, L., Lin, L. L., Miracco, A. S., Natesan, S., and Luscinskas, F. W. (2021a), CD47 antibody blockade suppresses microglia-dependent phagocytosis and monocyte transition to macrophages, impairing recovery in EAE. JCI Insight 6, 10.1172/jci.insight.148719.
Wang, S. K., Xue, Y., and Cepko, C. L. (2021b). Augmentation of CD47/SIRPαlpha signaling protects cones in genetic models of retinal degeneration. JCI Insight 6.10.1172/jci.insight.150796.
Wang, X., Zhao, L., Zhang, J., Fariss, R. N., Ma, W., Kretschmer, F., Wang, M., Qian, H. H., Badea. T. C., Diamond, J. S., et al. (2016). Requirement for Microglia for the Maintenance of Synaptic Function and Integrity in the Mature Retina. J Neurosci 36, 2827-2842, 10.1523/JNEUROSCI.3575-15.2016.
Weiskopf, K., Jahchan, N. S., Schnorr, P. J., Cristea, S., Ring, A. M., Maute, R. L., Volkmer, A. K., Volkmer, J. P., Liu, J., Lim, J. S., et al. (2016), CD47-blocking immunotherapies stimulate macrophage mediated destruction of small-cell lung cancer. J Clin Invest 126, 2610-2620, 10.1172/JCI81603.
Werneburg, S., Feinberg. P. A., Johnson, K. M., and Schafer, D. P. (2017). A microglia-cytokine axis to modulate synaptic connectivity and function. Curr Opin Neurobiol 47.138-145, 10.1016/j.conb.2017.10.002.
Werneburg. S., Jung, J., Kunjamma, R. B., Ha, S. K., Luciano, N. J., Willis, C. M., Gao, G., Biscola. N. P., Havton, L. A., Crocker, S. J., et al. (2020). Targeted Complement Inhibition at Synapses Prevents Microglial Synaptic Engulfment and Synapse Loss in Demyelinating Disease. Immunity 52, 167-182 e167, 10.1016/j.immuni.2019.12.004.
Willingham, S. B., Volkmer, J. P., Gentles. A. J., Sahoo, D., Dalerba, P., Mitra, S. S., Wang. J., Contreras-Trujillo, H., Martin, R., Cohen, J. D., et al. (2012). The CD47-signal regulatory protein alpha (SIRPα) interaction is a therapeutic target for human solid tumors. Proc Natl Acad Sci USA 109, 6662-6667, 10.1073/pnas. 1121623109.
Wilton, D. K., Dissing-Olesen, L., and Stevens, B. (2019). Neuron-Glia Signaling in Synapse Elimination. Annu Rev Neurosci 42, 107-127, 10.1146/annurev-neuro-070918-050306.
Wong. R. O., and Ghosh, A. (2002). Activity-dependent regulation of dendritic growth and patterning. Nat Rev Neurosci 3, 803-812, 10.1038/nrn941.
Wu, C. J., Chen, Z., Ullrich, A., Greene, M. I., and O'Rourke, D. M. (2000). Inhibition of EGFR mediated phosphoinositide-3-OH kinase (PI3-K) signaling and glioblastoma phenotype by signal regulatory proteins (SIRPs). Oncogene 19, 3999-4010.10.1038/sj.one. 1203748.
Wu, Y., Dissing-Olesen, L., Mac Vicar, B. A., and Stevens, B. (2015). Microglia: Dynamic Mediators of Synapse Development and Plasticity. Trends Immunol 36, 605-613.10.1016/j.it.2015.08.008.
Yan, H. X., Wang. H. Y., Zhang, R., Chen, L., Li, B. A., Liu, S. Q., Cao, H. F., Qiu, X. H., Shan, Y. F., Yan, Z. H., et al. (2004). Negative regulation of hepatocellular carcinoma cell growth by signal regulatory protein alpha1. Hepatology 40, 618-628, 10.1002/hep.20360.
Yao, C., Li, G., Cai, M., Qian, Y., Wang. L., Xiao, L.. Thaiss, F., and Shi, B. (2017). Prostate cancer downregulated SIRP-alpha modulates apoptosis and proliferation through p38-MAPK/NF1192 kappaB/COX-2 signaling. Oncol Lett 13, 4995-5001, 10.3892/ol.2017.6070.
Yona, S., Kim, K. W., Wolf, Y., Mildner, A. Varol. D., Breker, M., Strauss-Ayali, D., Viukov, S., Guilliams, M., Misharin, A., et al. (2013). Fate mapping reveals origins and dynamics of monocytes and tissue macrophages under homeostasis. Immunity 38, 79-91.10.1016/j.immuni.2012.12.001.
Young, K., and Morrison, H. (2018). Quantifying Microglia Morphology from Photomicrographs of Immunohistochemistry Prepared Tissue Using ImageJ. J Vis Exp. 10.3791/57648.
Zhao, H., Wang, J., Kong, X., Li, E., Liu, Y., Du, X., Kang, Z.. Tang, Y., Kuang, Y., Yang, Z., et al. (2016), CD47 Promotes Tumor Invasion and Metastasis in Non-small Cell Lung Cancer. Sci Rep 6, 29719, 10.1038/srep29719.
Zhao, X. F., Alam, M. M., Liao, Y., Huang. T., Mathur, R., Zhu, X., and Huang, Y. (2019). Targeting Microglia Using Cx3crl-Cre Lines: Revisiting the Specificity, eNeuro 6.10.1523/ENEURO.0114-19.2019.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method of treating, protecting against, and/or reducing the risk of a neurological disorder, the method comprising administration of one or more compositions that modulate neuronal SIRPα.

2. The method of claim 1, wherein the composition increases one or more of neuronal SIRPα gene activity, neuronal SIRPα protein levels, and/or neuronal SIRPα protein activity.

3. The method of claim 1, wherein the composition decreases one or more of neuronal SIRPα gene activity, neuronal SIRPα protein levels, and/or neuronal SIRPα protein activity.

4. The method of claim 1, wherein the neurological disorder is a central nervous system disorder (CNS) and/or a peripheral nervous system disorder (PNS).

5. (canceled)

6. The method of claim 3, wherein the composition comprises at least one inhibitory oligonucleotide, wherein the inhibitory oligonucleotide is small interfering RNA (siRNA), microRNA (miRNA), or inhibitory antisense oligonucleotides (ASOs).

7. (canceled)

8. The method of claim 6, wherein the inhibitory oligonucleotide has at least 80% sequence identity to a portion of, or a sequence complementary to, any of SEQ ID NOs: 1, 2, 7, or 8.

9. (canceled)

10. The method of claim 1, wherein the composition comprises a transgene, wherein the transgene encodes a SIRPα gene product and/or a CD47 gene product.

11. The method of claim 10, wherein the transgene encodes a protein at least 80% identical to a portion of any of SEQ ID NOs: 9 or 10 and/or a protein at least 80% identical to a portion of any of SEQ ID NOs: 3-6.

12-14. (canceled)

15. The method of claim 3, wherein the composition comprises at least one antibody or Fc fusion protein, wherein the at least one antibody or Fc fusion protein comprises anti-CD47 antibody and/or anti-SIRPα antibody.

16. The method of claim 15, wherein the anti-CD47 antibody is Magrolimab, Hu5F9-G4, CC-90002, TTI-621, ALX148, SRF231, SHR-1603, or IBI188_and/or wherein the anti-SIRPα antibody is ADU-1805, humanized AB21 (hAB21), humanized 1H9, or BI 765063 (OSE-172).

17-18. (canceled)

19. The method of claim 1, wherein the at least one inhibitory oligonucleotide, transgene, antibody, or Fc fusion protein is administered in the form of a nucleic acid vector.

20. The method of claim 3, wherein the composition comprises at least one SIRPα inhibitor, wherein the SIRPα inhibitor is one or more of the Velcro-CD47 (N3612) antagonist, the small molecule RRx-001, and/or the small molecule 4Mu.

21-24. (canceled)

25. The method of claim 1, wherein the composition comprises at least one cell comprising a Chimeric Antigen Receptor (CAR) expressing cell, such as an CAR NK-cell or CAR T-cell.

26. (canceled)

27. The method of claim 1, wherein the composition comprises a clustered regularly interspaced short palindromic repeats (CRISPR) associated (Cas) protein nuclease selected from a type I, type II, type III, type IV, type V, or type VI nuclease.

28. (canceled)

29. (canceled)

30. The method of claim 1, wherein the composition comprises a retrovirus.

31. The method of claim 30, wherein the retrovirus is an Adeno Associated Virus (AAV) is AAV2/1, AAV2/2, AAV2/5, or AAV2/9.

32. (canceled)

33. The method of claim 4, wherein the neurological disorder is characterized by loss of synapses selected from major depressive disorder, schizophrenia, Alzheimer's disease, Huntington disease, Parkinson's disease, amyotrophic lateral sclerosis (ALS), demyelinating diseases (e.g., multiple sclerosis (MS)), and aging and/or aberrant synapse pruning selected from autism spectrum disorders (ASDs), down syndrome, hyperekplexia, epilepsy, or other developmental neurological disorders (e.g., applicable rare but impactful neurological diseases, e.g., applicable diseases described by the National Institute of Neurological Disorders and Stroke).

34-36. (canceled)


SEQUENCE LISTING (37 sequences)

SEQ ID NO: 1 - DNA, 5292 bp, Homo sapiens
  source 1..5292: mol_type = genomic DNA
gcagcctgggcagtgggtcctgcctgtgacgcgcggcggcggtcggtcctgcctgtaacggcggcggcggctgctgctccggacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtccaa

SEQ ID NO: 2 - DNA, 972 bp, Homo sapiens
  source 1..972: mol_type = genomic DNA
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataa

SEQ ID NO: 3 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 4 - protein, 305 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 5 - protein, 312 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNE

SEQ ID NO: 6 - protein, 120 aa, synthetic construct
WQPPLLFNKTKSVEFTFGNDTVVIPCFVTNMEAQNTTEVYVKWKFKGRDIYTFDGQANKSTVPTDFSSAKIEVSQLLKGDASLKMDKSDAVSHTGNYTCEVTELTREGETIIELKYRVVS

SEQ ID NO: 7 - DNA, 4201 bp, Homo sapiens
  source 1..4201: mol_type = genomic DNA
tccggcccgcacccacccccaagaggggccttcagctttggggctcagaggcacgacctcctggggagggttaaaaggcagacgcccccccgccccccgcgcccccgcgccccgactccttcgccgcctccagcctctcgccagtgggaagcggggagcagccgcgcggccggagtccggaggcgaggggaggtcggccgcaacttccccggtccaccttaagaggacgatgtagccagctcgcagcgctgaccttagaaaaacaagtttgcgcaaagtggagcggggacccggcctctgggcagccccggcggcgcttccagtgccttccagccctcgcgggcggcgcagccgcggcccatggagcccgccggcccggcccccggccgcctcgggccgctgctctgcctgctgctcgccgcgtcctgcgcctggtcaggagtggcgggtgaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaaccgttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctgagaacactggatctaatgaacggaacatctatattgtggtgggtgtggtgtgcaccttgctggtggccctactgatggcggccctctacctcgtccgaatcagacagaagaaagcccagggctccacttcttctacaaggttgcatgagcccgagaagaatgccagagaaataacacaggacacaaatgatatcacatatgcagacctgaacctgcccaaggggaagaagcctgctccccaggctgcggagcccaacaaccacacggagtatgccagcattcagaccagcccgcagcccgcgtcggaggacaccctcacctatgctgacctggacatggtccacctcaaccggacccccaagcagccggcccccaagcctgagccgtccttctcagagtacgccagcgtccaggtcccgaggaagtgaatgggaccgtggtttgctctagcacccatctctacgcgctttcttgtcccacagggagccgccgtgatgagcacagccaacccagttcccggagggctggggcggtgcaggctctgggacccaggggccagggtggctcttctctccccacccctccttggctctccagcacttcctgggcagccacggccccctccccccacattgccacatacctggaggctgacgttgccaaaccagccagggaaccaacctgggaagtggccagaactgcctggggtccaagaactcttgtgcctccgtccatcaccatgtgggttttgaagaccctcgactgcctccccgatgctccgaagcctgatcttccagggtggggaggagaaaatcccacctcccctgacctccaccacctccaccaccaccaccaccaccaccaccaccactaccaccaccacccaactggggctagagtggggaagatttcccctttagatcaaactgccccttccatggaaaagctggaaaaaaactctggaacccatatccaggcttggtgaggttgctgccaacagtcctggcctcccccatccctaggctaaagagccatgagtcctggaggaggagaggacccctcccaaaggactggagacaaaaccctctgcttccttgggtccctccaagactccctggggcccaactgtgttgctccacccggacccatctctcccttctagacctgagcttgcccctccagctagcactaagcaacatctcgctgtggacgcctgtaaattactgagaaatgtgaaacgtgcaatcttgaaactgaggtgttagaaaacttgatctgtggtgttttgttttgttttttttcttaaaacaacagcaacgtgatcttggctgtctgtcatgtgttgaagtccatggttgggtcttgtgaagtctgaggtttaacagtttgttgtcctggagggattttcttacagcgaagacttgagttcctccaagtcccagaaccccaagaatgggcaagaaggatcaggtcagccactccctggagacacagccttctggctgggactgacttggccatgttctcagctgagccacgcggctggtagtgcagccttctgtgaccccgctgtggtaagtccagcctgcccagggctgctgagggctgcctcttgacagtgcagtcttatcgagacccaatgcctcagtctgctcatccgtaaagtggggatagtgaagatgacacccctccccaccacctctcataagcactttaggaacacacagagggtagggatagtggccctggccgtctatcctacccctttagtgaccgcccccatcccggctttctgagctgatccttgaagaagaaatcttccatttctgctctcaaaccctactgggatcaaactggaataaattgaagacagccagggggatggtgcagctgtgaagctcgggctgattccccctctgtcccagaaggttggccagagggtgtgacccagttaccctttaacccccacccttccagtcgggtgtgagggcctgaccgggcccagggcaagcagatgtcgcaagccctatttattcagtcttcactataactcttagagttgagacgctaatgttcatgactcctggccttgggatgcccaagggatttctggctcaggctgtaaaagtagctgagccatcctgcccattcctggaggtcctacaggtgaaactgcaggagctcagcatagacccagctctctgggggatggtcacctggtgatttcaatgatggcatccaggaattagctgagccaacagaccatgtggacagctttggccagagctcccgtgtggcatctgggagccacagtgacccagccacctggctcaggctagttccaaattccaaaagattggcttgtaaaccttcgtctccctctcttttacccagagacagcacatacgtgtgcacacgcatgcacacacacattcagtattttaaaagaatgttttcttggtgccattttcattttattttattttttaattcttggagggggaaataagggaataaggccaaggaagatgtatagctttagctttagcctggcaacctggagaatccacataccttgtgtattgaaccccaggaaaaggaagaggtcgaaccaaccctgcggaaggagcatggtttcaggagtttattttaagactgctgggaaggaaacaggccccattttgtatatagttgcaacttaaactttttggcttgcaaaatatttttgtaataaagatttctgggtaataatga

SEQ ID NO: 8 - DNA, 1515 bp, Homo sapiens
  source 1..1515: mol_type = genomic DNA
atggagcccgccggcccggcccccggccgcctcgggccgctgctctgcctgctgctcgccgcgtcctgcgcctggtcaggagtggcgggtgaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaaccgttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctgagaacactggatctaatgaacggaacatctatattgtggtgggtgtggtgtgcaccttgctggtggccctactgatggcggccctctacctcgtccgaatcagacagaagaaagcccagggctccacttcttctacaaggttgcatgagcccgagaagaatgccagagaaataacacaggacacaaatgatatcacatatgcagacctgaacctgcccaaggggaagaagcctgctccccaggctgcggagcccaacaaccacacggagtatgccagcattcagaccagcccgcagcccgcgtcggaggacaccctcacctatgctgacctggacatggtccacctcaaccggacccccaagcagccggcccccaagcctgagccgtccttctcagagtacgccagcgtccaggtcccgaggaagtga

SEQ ID NO: 9 - protein, 504 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 10 - protein, 508 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQVQSLDTNDITYADLNLPKGKKPAPQAAEPNNHTEYASIQTSPQPASEDTLTYADLDMVHLNRTPKQPAPKPEPSFSEYASVQVPRK

SEQ ID NO: 11 - DNA, 20 bp, synthetic construct
  source 1..20: mol_type = other DNA
tgagagggaaatcgtgcgtg

SEQ ID NO: 12 - DNA, 23 bp, synthetic construct
  source 1..23: mol_type = other DNA
tcgttgccaatagtgatgacctg

SEQ ID NO: 13 - DNA, 22 bp, synthetic construct
  source 1..22: mol_type = other DNA
aaaaacactggatttcaggggc

SEQ ID NO: 14 - DNA, 22 bp, synthetic construct
  source 1..22: mol_type = other DNA
caaccaacacaggaacagggag

SEQ ID NO: 15 - DNA, 19 bp, synthetic construct
  source 1..19: mol_type = other DNA
cgctctggagtggaggcac

SEQ ID NO: 16 - DNA, 21 bp, synthetic construct
  source 1..21: mol_type = other DNA
aaacgcaacaggaggtaggag

SEQ ID NO: 17 - DNA, 20 bp, synthetic construct
  source 1..20: mol_type = other DNA
ggacacttcgggccatgttt

SEQ ID NO: 18 - DNA, 20 bp, synthetic construct
  source 1..20: mol_type = other DNA
cttacacagtggactggggc

SEQ ID NO: 19 - DNA, 20 bp, synthetic construct
  source 1..20: mol_type = other DNA
atggatgcgtaatcacgggg

SEQ ID NO: 20 - DNA, 22 bp, synthetic construct
  source 1..22: mol_type = other DNA
gtctgggtttcaggcagtcaag

SEQ ID NO: 21 - DNA, 22 bp, synthetic construct
  source 1..22: mol_type = other DNA
tctgacctctggggagaaaagc

SEQ ID NO: 22 - DNA, 24 bp, synthetic construct
  source 1..24: mol_type = other DNA
tgggacaaccataaaccaccatag

SEQ ID NO: 23 - DNA, 20 bp, synthetic construct
  source 1..20: mol_type = other DNA
tgtggactctcatgcctcct

SEQ ID NO: 24 - DNA, 20 bp, synthetic construct
  source 1..20: mol_type = other DNA
tggtcatctctgaagccgtg

SEQ ID NO: 25 - DNA, 21 bp, synthetic construct
  source 1..21: mol_type = other DNA
cgtcctccaggatgtttctcc

SEQ ID NO: 26 - DNA, 19 bp, synthetic construct
  source 1..19: mol_type = other DNA
gctttgacctcacagaggc

SEQ ID NO: 27 - DNA, 22 bp, synthetic construct
  source 1..22: mol_type = other DNA
gctgtgtttgcctctctaccag

SEQ ID NO: 28 - DNA, 22 bp, synthetic construct
  source 1..22: mol_type = other DNA
caagtagaggcaggatcaggct

SEQ ID NO: 29 - DNA, 22 bp, synthetic construct
  source 1..22: mol_type = other DNA
gagcaacagtgccaaggaatgg

SEQ ID NO: 30 - DNA, 22 bp, synthetic construct
  source 1..22: mol_type = other DNA
actgtgggctaccttgtaggac

SEQ ID NO: 31 - DNA, 21 bp, synthetic construct
  source 1..21: mol_type = other DNA
ggacccggaaacaacacattg

SEQ ID NO: 32 - DNA, 20 bp, synthetic construct
  source 1..20: mol_type = other DNA
gatcccagagagggcttgtt

SEQ ID NO: 33 - DNA, 20 bp, synthetic construct
  source 1..20: mol_type = other DNA
gtgtccaactgcacagcaag

SEQ ID NO: 34 - DNA, 20 bp, synthetic construct
  source 1..20: mol_type = other DNA
gcagacgtgaatgtcgcaat

SEQ ID NO: 35 - RNA, 10 nt, synthetic construct
  source 1..10: mol_type = other RNA
  misc_feature 1..10: note = This sequence may comprise 1-10 adenine nucleotides
aaaaaaaaaa

SEQ ID NO: 36 - RNA, 20 nt, synthetic construct
  source 1..20: mol_type = other RNA
  misc_feature 1..20: note = This sequence may comprise 10-20 adenine nucleotides
aaaaaaaaaaaaaaaaaaaa

SEQ ID NO: 37 - RNA, 200 nt, synthetic construct
  source 1..200: mol_type = other RNA
  misc_feature 1..200: note = This sequence may comprise 25-200 adenine nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa